(19) United States
(12) Patent Application Publication
(10) Pub. No.: US 2022/0153734 A1
(43) Pub. Date: May 19, 2022

(54) Pyrazolopyridine Compounds For IRE1 Inhibition

(71) Applicant: Optikira, LLC, Cleveland, OH (US)

(72) Inventors: Richard Keenan, Cleveland, OH (US); Jon Sutton, Cleveland, OH (US); George Hynd, Cleveland, OH (US)

(21) Appl. No.: 17/432,842

(22) PCT Filed: Feb. 27, 2020
(86) PCT No.: PCT/US20/20162

§ 371 Date: Aug. 20, 2021

Related U.S. Application Data
(60) Provisional application No. 62/811,237, filed on Feb. 27, 2019.
(60) Provisional application No. 62/813,975, filed on Mar. 5, 2019.

(51) Int. Cl. C07D 471/04

(52) U.S. Cl. CPC C07D 471/04

(57) ABSTRACT

The present invention provides novel pyrazolopyridine compounds, compositions and methods for treating or preventing an IRE1α-related disease or disorder. In certain embodiments, the disease or disorder is selected from the group consisting of a neurodegenerative disease, a demyelinating disease, cancer, an eye disease, a fibrotic disease, and diabetes.

### CROSS-REFERENCE TO RELATED APPLICATIONS

The present application claims priority under 35 U.S.C. § 119(e) to U.S. Provisional Application No. 62/811,237, filed Feb. 27, 2019 and U.S. Provisional Patent Application No. 62/813,975, filed Mar. 5, 2019, all of which applications are incorporated herein by reference in their entireties.

### BACKGROUND OF THE INVENTION

Cells often experience conditions during which the workload on the endoplasmic reticulum (“ER”) protein folding machinery exceeds its capability, causing ER stress. ER stress can result from secretory work overload, expression of folding-defective secretory proteins, deprivation of nutrients or oxygen, changes in luminal calcium concentration, and deviation from resting redox state. Under ER stress, secretory proteins accumulate in unfolded forms within the organelle to trigger a set of intracellular signaling pathways called the Unfolded Protein Response (UPR). UPR signaling increases transcription of genes encoding chaperones, oxidoreductases, lipid-biosynthetic enzymes, and ER-associated degradation (ERAD) components.

In some instances, the ER stressed state remains too great, and cannot be remedied through the UPR's homeostatic outputs. In these situations, the UPR switches strategies and actively triggers apoptosis. Apoptosis of irremediably stressed cells is a quality control strategy that protects multicellular organisms from exposure to immature and damaged secretory proteins. Many deadly human diseases occur if too many cells die through this process. Conversely, many human diseases such as diabetes mellitus and retinopathies proceed from unchecked cell degeneration under ER stress.

IRE1α and IRE1β are ER-transmembrane proteins that become activated when unfolded proteins accumulate within the organelle. IRE1α is the more widely expressed family member. The bifunctional kinase/endoribonuclease IRE1α controls entry into the terminal UPR. IRE1α senses unfolded proteins through an ER luminal domain that becomes oligomerized during stress.

Under irremediable ER stress, positive feedback signals emanate from the UPR and become integrated and amplified at key nodes to trigger apoptosis. IRE1α is a key initiator of these pro-apoptotic signals. IRE1α employs auto-phosphorylation as a timer. Remediable ER stress causes low-level, transient auto-phosphorylation that confines RNase activity to XBP1 mRNA splicing. However, sustained kinase autophosphorylation causes IRE1a's RNase to acquire relaxed specificity, causing it to endonucleolytically degrade thousands of ER-localized mRNAs in close proximity to IRE1a. These mRNAs encode secretory proteins being co-translationally translocated (e.g., insulin in (3 cells). As mRNA degradation continues, transcripts encoding ER-resident enzymes also become depleted, thus destabilizing the entire ER protein-folding machinery. Once IRE1α's RNase becomes hyperactive, adaptive signaling through XBP1 splicing becomes eclipsed by ER mRNA destruction, which pushes cells into apoptosis.

A terminal UPR signature tightly controlled by IRE1α's hyperactive RNase activity causes (1) widespread mRNA degradation at the ER membrane that leads to mitochondrial apoptosis, (2) induction of the pro-oxidant thioredoxin-interacting protein (TXNIP), which activates the NLRP3 inflammasome to produce maturation and secretion of interleukin-1β, and consequent sterile inflammation in pancreatic islets leading to diabetes, and (3) degradation of pre-miRNA 17, leading to translational upregulation and cleavage of pre-mitochondrial caspase 2 and stabilization of the mRNA encoding TXNIP.

There is a need in the art for novel small molecule compounds that are capable of treating ER stress without resorting to UPR based apoptosis, thereby treating a wide range of disorders and diseases tied to ER stress. Such diseases include, for example, neurodegenerative diseases, demyelinating diseases, cancers, eye diseases, fibrotic diseases, and/or diabetes. The present invention meets these needs.

### BRIEF SUMMARY OF THE INVENTION

The present invention provides in one aspect compounds of formula (Ia):

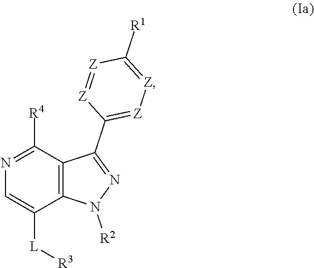

or a salt, solvate, enantiomer, diastereoisomer, isotopologue, or tautomer thereof, wherein the variables R¹-R⁴, Z, and L are defined elsewhere herein.

The present invention provides in one aspect compounds of formula (IIa):

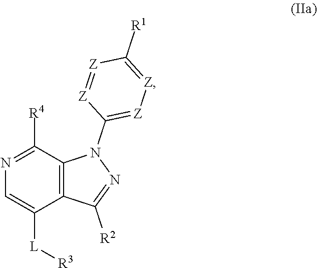

or a salt, solvate, enantiomer, diastereoisomer, isotopologue, or tautomer thereof, wherein the variables R¹-R⁴, Z, and L are defined elsewhere herein.

The present invention further provides methods of treating, ameliorating, and/or preventing diseases or disorders associated with ER stress, such as those selected from the group consisting of a neurodegenerative disease, demyelinating disease, cancer, eye disease, fibrotic disease, and diabetes. In certain embodiments, the disease or disorder is a neurodegenerative disease. In other embodiments, the disease or disorder is a demyelinating disease. In yet other embodiments, the disease or disorder is cancer. In yet other embodiments, the disease or disorder is eye disease. In yet other embodiments, the disease or disorder is a fibrotic disease. In yet other embodiments, the disease or disorder is diabetes.

### DETAILED DESCRIPTION OF THE INVENTION

The present invention relates in part to the unexpected discovery that novel inhibitors of IRE1α prevent oligomerization and/or allosterically inhibit its RNase activity.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “cancer” is defined as disease characterized by the rapid and uncontrolled growth of aberrant cells. Cancer cells can spread locally or through the bloodstream and lymphatic system to other parts of the body. Examples of cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “ED₅₀” or “ED50” refers to the effective dose of a formulation that produces about 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, a “patient” or “subject” may be a human or non-human mammal or a bird. Non-human mammals include, for example, livestock and pets, such as ovine, bovine, porcine, canine, feline and murine mammals. In certain other embodiments, the subject is human.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

The term “prevent,” “preventing,” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “solvate,” as used herein, refers to a compound formed by solvation, which is a process of attraction and association of molecules of a solvent with molecules or ions of a solute. As molecules or ions of a solute dissolve in a solvent, they spread out and become surrounded by solvent molecules.

The term “treat,” “treating,” or “treatment,” as used herein, means reducing the frequency or severity with which symptoms of a disease or condition are experienced by a subject by virtue of administering an agent or compound to the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C3-C6 means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “alkenylene”, employed alone or in combination with other terms, means, unless otherwise stated, a stable mono-unsaturated or di-unsaturated straight chain or branched chain hydrocarbon group having the stated number of carbon atoms wherein the group has two open valencies.

As used herein, the term “alkynylene”, employed alone or in combination with other terms, means, unless otherwise stated, a stable straight chain or branched chain hydrocarbon group with a triple carbon-carbon bond, having the stated number of carbon atoms wherein the group has two open valencies.

As used herein, the term “substituted alkyl”, “substituted cycloalkyl”, “substituted alkenyl”, “substituted alkynyl”, “substituted alkylene”, “substituted alkenylene”, “substituted alkynylene”, “substituted heteroalkyl”, “substituted heteroalkenyl”, “substituted heteroalkynyl”, “substituted aryl”, “substituted heteroaryl” or “substituted heterocycloalkyl” means alkyl, cycloalkyl, alkenyl, alkynyl, alkylene, alkenylene, alkynylene, heteroalkyl, heteroalkenyl, heteroalkynyl, aryl, heteroaryl, or heterocycloalkyl as defined above, substituted by one, two or three substituents selected from the group consisting of C₁-C₁₀ alkyl, halogen, perhaloakyl, ═O, —OH, alkoxy, —NH₂, —N(CH₃)₂, —NH(CH₃)₂, phenyl, benzyl, (1-methyl-imidazol-2-yl), pyridin-2-yl, pyridin-3-yl, pyridin-4-yl, —C(═O)OH, —OC(═O) (C₁-C₄)alkyl, —C(═O)(C₁-C₄)alkyl, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain other embodiments, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3 dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. Non-limiting examples of “substituted” groups include C₁-C₁₀ alkyl, halogen, perhaloakyl, ═O, —OH, alkoxy, —NH₂, —N(CH₃)₂, —NH(CH₃)₂, phenyl, benzyl, (1-methyl-imidazol-2-yl), pyridin-2-yl, pyridin-3-yl, pyridin-4-yl, —C(═O)OH, —OC(═O) (C₁-C₄)alkyl, —C(═O)(C₁-C₄)alkyl, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain other embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halogen, amino, acetamido and nitro. As used herein, where a substituent is an alkyl or alkoxy group, the carbon chain may be branched, straight or cyclic, with straight being preferred. The term “substituted heterocycle” and “substituted heteroaryl” as used herein refers to a heterocycle or heteroaryl group having one or more substituents including halogen, CN, OH, NO₂, amino, alkyl, cycloalkyl, carboxyalkyl (C(O)Oalkyl), trifluoroalkyl such as CF₃, aryloxy, alkoxy, aryl, or heteroaryl. A substituted heterocycle or heteroaryl group may have 1, 2, 3, or 4 substituents.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: Boc or BOC, tert-butyloxycarbonyl; Boc₂O, di-tert-butyl dicarbonate; (Bpin)₂, bis(pinacolato)diboron; CELITE®, diatomaceous earth; Cs₂CO₃, cesium carbonate; DCE, 1,2-dichloroethylene; DCM, dichloromethane; DEA, diethylamine; DIPEA, N,N-diisopropylethylamine; DMAP, 4-dimethylaminopyridine; DMF, dimethylformamide; DMSO, dimethyl sulfoxide; ER, endoplasmic reticulum; ERAD, endoplasmic reticulum-associated degradation; EtOAc, ethyl acetate; EtOH, ethanol; Et₂O, diethyl ether; h, hours; HATU, (1-[bis(dimethylamino)methylene]-1H-1,2,3-triazolo[4,5-b]pyridinium 3-oxide hexafluorophosphate; HPLC, high-performance liquid chromatography; IPA, 2-propanol; KOAc, potassium acetate; LC-MS, liquid chromatography-mass spectrometry; LiOH, lithium hydroxide; MDAP, mass-directed automated purification; MeCN, acetonitrile; MeOH, methanol; min, minutes; MgSO₄, magnesium sulfate; Na₂SO₄, sodium sulfate; NBS, N-bromosuccinimide; NCS, N-chlorosuccinimide; NIS, N-iodosuccinimide; Pd(dppf)Cl₂.DCM, [1,1′-Bis(diphenylphosphino)ferrocene]-dichloropalladium(II) DCM complex; NMR, nuclear magnetic resonance; Ph, phenyl; Ph₃P, triphenyllphosphine; RP, retinitis pigmentosa; RT, room temperature; Rₜ, retention time; SCX-2, Biotage Isolute—strong cationic ion-exchange resin; TEA, trimethylamine; TFA, trifluoroacetic acid; THF, tetrahydrofuran; TLC, thin layer chromatography; UPLC, ultra-high performance liquid chromatography; UPR, unfolded protein response.

### Compounds and Compositions

The invention includes a compound of formula (Ia) or (IIa), or a salt, solvate, enantiomer, diastereoisomer, isotopologue, or tautomer thereof:

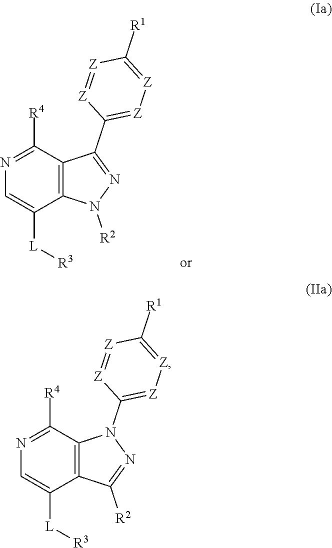

wherein:

R¹ is

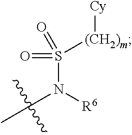

R² is selected from the group consisting of H, methyl, ethyl, propyl, isopropyl, butyl, isobutyl, sec-butyl, tert-butyl, CF₃, CHF₂, cyclopropyl, cyclobutyl, cyclopentyl, cyclohexyl, cycloheptyl, cyclooctyl, and 1-methylcyclopropyl;

L is selected from the group consisting of a bond, —CH₂—, —C(═O)—, —C(═O)NH, and —C(═O)N(C₁-C₆ alkyl);

R³ is selected from the group consisting of optionally substituted C₁-C₈ alkyl, optionally substituted C₃-C₈ cycloalkyl, optionally substituted C₂-C₈ alkenyl, optionally substituted C₂-C₈ cycloalkenyl, optionally substituted C₂-C₈ alkynyl, optionally substituted C₁-C₈ heteroalkyl (such as, but not limited to, N-linked C₁-C₈ aminoalkyl), optionally substituted C₃-C₈ heterocycloalkyl, optionally substituted C₂-C₈ heteroalkenyl, optionally substituted C₂-C₈ cycloheteroalkenyl, and optionally substituted heterocyclyl;

R⁴ is NH₂;

0-3 instances of Z are N and the remaining instances of Z are independently CR⁵;

each instance of R⁵ is independently selected from the group consisting of halogen, —OH, C₁-C₆ alkoxy, optionally substituted phenyl, optionally substituted C₁-C₆ alkyl, optionally substituted C₁-C₆ alkoxy, and optionally substituted heterocycloalkyl;

R⁶ is H;

Cy is selected from the group consisting of aryl (such as, but not limited to, phenyl or naphthyl), heteroaryl (such as, but not limited to, thiophenyl, pyridinyl, pyridazinyl, pyrimidinyl, or pyrazinyl), C₃-C₁₀ cycloalkyl, C₃-C₁₀ cycloalkenyl, C₃-C₁₀ heterocycloalkyl, C₃-C₁₀ heterocycloalkenyl, polycyclic aryl, polycyclic heteroaryl, polycyclic C₃-C₁₀ cycloalkyl, polycyclic C₃-C₁₀ cycloalkenyl, polycyclic C₃-C₁₀ heterocycloalkyl, and polycyclic C₃-C₁₀ heterocycloalkenyl;
wherein Cy is substituted with 0 to ‘n’ instances of X, each instance of X being independently selected from the group consisting of H, halogen, nitrile, optionally substituted C₁-C₄ alkyl, C₁-C₄ haloalkyl, optionally substituted C₁-C₄ alkoxy, optionally substituted aryl (such as, but not limited, phenyl or naphthyl), optionally substituted heteroaryl, and

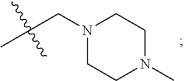 [SMILES: *CN1CCN(C)CC1]

m is an integer selected from the group consisting of 0, 1, and 2;

n is an integer selected from the group consisting of 0, 1, 2, 3, 4, and 5.

In certain embodiments, an optionally substituted group is unsubstituted. In other embodiments, an optionally substituted group is substituted with at least substituent contemplated herein.

In certain embodiments, each occurrence of optionally substituted alkyl, optionally substituted alkenyl, optionally substituted alkynyl, optionally substituted heteroalkyl, optionally substituted heterocycloalkyl, optionally substituted heteroalkenyl, optionally substituted benzyl, optionally substituted heterocyclyl, or optionally substituted cycloalkyl is independently optionally substituted with at least one substituent selected from the group consisting of C₁-C₆ alkyl, halogen, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

In certain embodiments, each occurrence of optionally substituted aryl or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —N(R^{c})(R^{c}), and C₁-C₆ alkoxycarbonyl, wherein each occurrence of R^{c} is independently H, C₁-C₆ alkyl, or C₃-C₈ cycloalkyl.

In certain embodiments, R¹ is

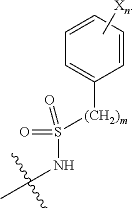

In certain embodiments, R¹ is

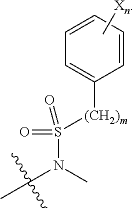

In certain embodiments, R¹ is

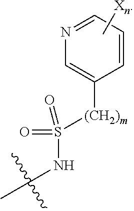

In certain embodiments, R¹ is

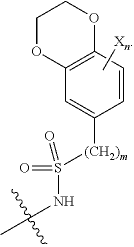

In certain embodiments, R¹ is

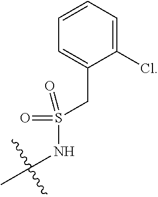 [SMILES: *NS(=O)(=O)Cc1ccccc1Cl]

In certain embodiments, R¹ is

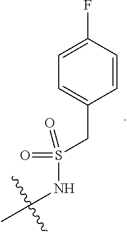 [SMILES: *NS(=O)(=O)Cc1ccc(F)cc1]

In certain embodiments, R¹ is

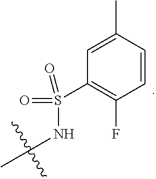 [SMILES: *NS(=O)(=O)c1cc(C)ccc1F]

In certain embodiments, R¹ is

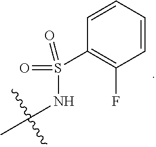 [SMILES: *NS(=O)(=O)c1ccccc1F]

In certain embodiments, R¹ is

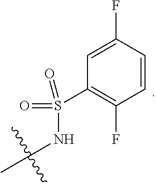 [SMILES: *NS(=O)(=O)c1cc(F)ccc1F]

In certain embodiments, R¹ is

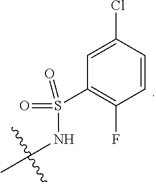 [SMILES: *NS(=O)(=O)c1cc(Cl)ccc1F]

In certain embodiments, R¹ is

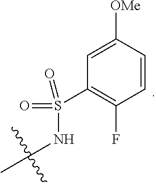 [SMILES: *NS(=O)(=O)c1cc(OC)ccc1F]

In certain embodiments, R¹ is

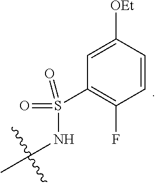 [SMILES: *NS(=O)(=O)c1cc(OCC)ccc1F]

In certain embodiments, R¹ is

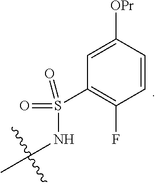

In certain embodiments, R¹ is

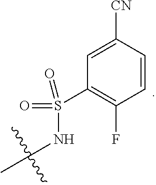 [SMILES: *NS(=O)(=O)c1cc(C#N)ccc1F]

In certain embodiments, R¹ is

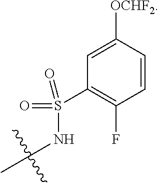 [SMILES: *NS(=O)(=O)c1cc(OC(F)F)ccc1F]

In certain embodiments, R¹ is

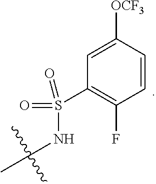 [SMILES: *NS(=O)(=O)c1cc(OC(F)(F)F)ccc1F]

In certain embodiments, R¹ is

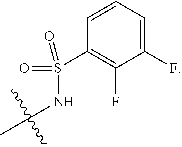 [SMILES: *NS(=O)(=O)c1cccc(F)c1F]

In certain embodiments, R¹ is

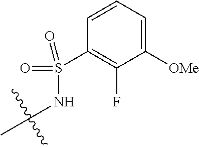 [SMILES: *NS(=O)(=O)c1cccc(OC)c1F]

In certain embodiments, R¹ is

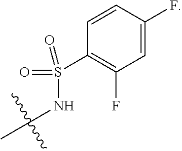 [SMILES: *NS(=O)(=O)c1ccc(F)cc1F]

In certain embodiments, R¹ is

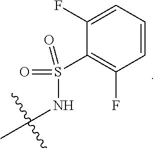 [SMILES: *NS(=O)(=O)c1c(F)cccc1F]

In certain embodiments, R¹ is

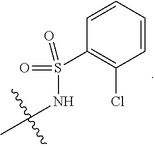 [SMILES: *NS(=O)(=O)c1ccccc1Cl]

In certain embodiments, R¹ is

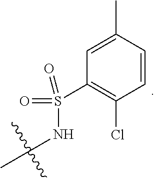 [SMILES: *NS(=O)(=O)c1cc(C)ccc1Cl]

In certain embodiments, R¹ is

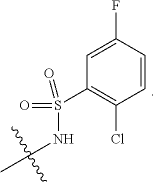 [SMILES: *NS(=O)(=O)c1cc(F)ccc1Cl]

In certain embodiments, R¹ is

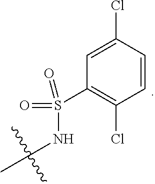 [SMILES: *NS(=O)(=O)c1cc(Cl)ccc1Cl]

In certain embodiments, R¹ is

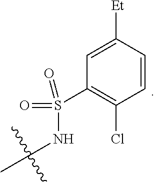 [SMILES: *NS(=O)(=O)c1cc(CC)ccc1Cl]

In certain embodiments, R¹ is

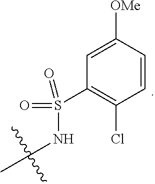 [SMILES: *NS(=O)(=O)c1cc(OC)ccc1Cl]

In certain embodiments, R¹ is

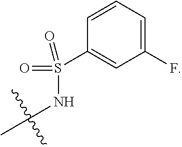 [SMILES: *NS(=O)(=O)c1cccc(F)c1]

In certain embodiments, R¹ is

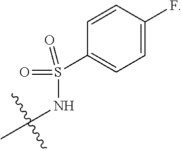 [SMILES: *NS(=O)(=O)c1ccc(F)cc1]

In certain embodiments, R¹ is

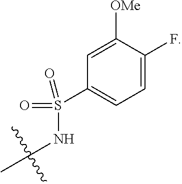 [SMILES: *NS(=O)(=O)c1ccc(F)c(OC)c1]

In certain embodiments, R¹ is

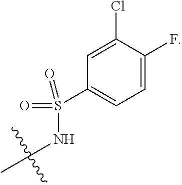 [SMILES: *NS(=O)(=O)c1ccc(F)c(Cl)c1]

In certain embodiments, R¹ is

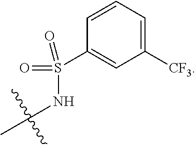 [SMILES: *NS(=O)(=O)c1cccc(C(F)(F)F)c1]

In certain embodiments, R¹ is

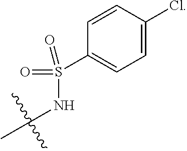 [SMILES: *NS(=O)(=O)c1ccc(Cl)cc1]

In certain embodiments, R¹ is

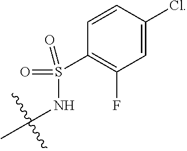 [SMILES: *NS(=O)(=O)c1ccc(Cl)cc1F]

In certain embodiments, R¹ is

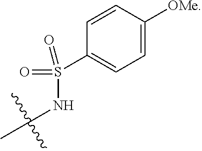 [SMILES: *NS(=O)(=O)c1ccc(OC)cc1]

In certain embodiments, R¹ is

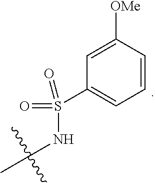 [SMILES: *NS(=O)(=O)c1cccc(OC)c1]

In certain embodiments, R¹ is

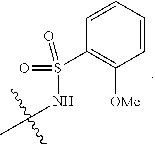 [SMILES: *NS(=O)(=O)c1ccccc1OC]

In certain embodiments, R¹ is

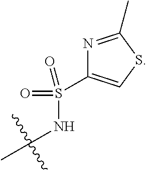 [SMILES: *NS(=O)(=O)c1csc(C)n1]

In certain embodiments, R² is H. In certain embodiments, R² is methyl. In certain embodiments, R² is ethyl. In certain embodiments, R² is propyl. In certain embodiments, R² is isopropyl. In other embodiments, R² is cyclopropyl. In certain embodiments, R² is CF₃. In certain embodiments, R² is CHF₂. In certain embodiments, R² is 1-methylcyclopropyl. In certain embodiments, R² is tert-butyl. In certain embodiments, R² is cyclobutyl. In certain embodiments, R² is cyclopentyl. In certain embodiments, R² is cyclohexyl. In certain embodiments, R² is cycloheptyl. In certain embodiments, R² is cyclooctyl.

In certain embodiments, R³ is

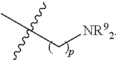

In certain embodiments, R³ is

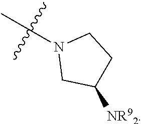

In certain embodiments, R³ is

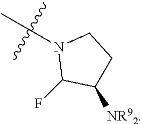

In certain embodiments, R³ is

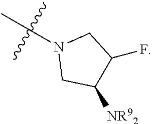

In certain embodiments, R³ is

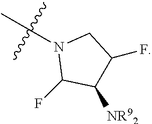

In certain embodiments, R³ is

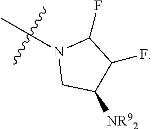

In certain embodiments, R³ is

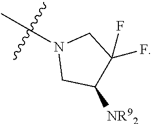

In certain embodiments, R³ is

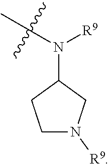

In certain embodiments, R³ is

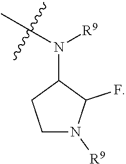

In certain embodiments, R³ is

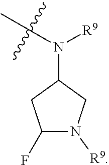

In certain embodiments, R³ is

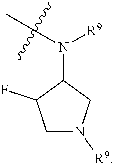

In certain embodiments, R³ is

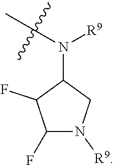

In certain embodiments, R³ is

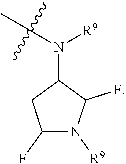

In certain embodiments, R³ is

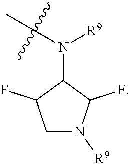

In certain embodiments, R³ is

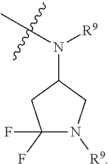

In certain embodiments, R³ is

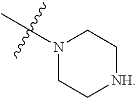 [SMILES: *N1CCNCC1]

In certain embodiments, R³ is

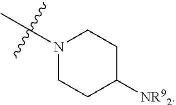

In certain embodiments, R³ is

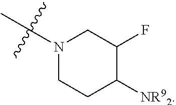

In certain embodiments, R³ is

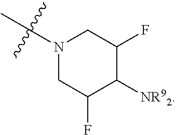

In certain embodiments, R³ is

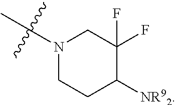

In certain embodiments, R³ is

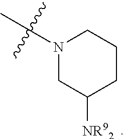

In certain embodiments, R³ is

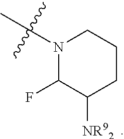

In certain embodiments, R³ is

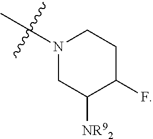

In certain embodiments, R³ is

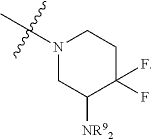

In certain embodiments, R³ is

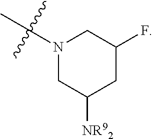

In certain embodiments, R³ is

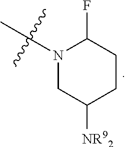

In certain embodiments, R³ is

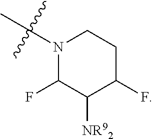

In certain embodiments, R³ is

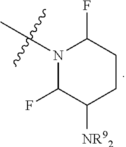

In certain embodiments, R³ is

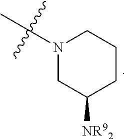

In certain embodiments, R³ is

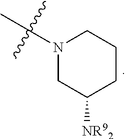

In certain embodiments, R³ is

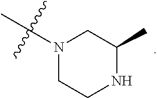 [SMILES: *N1CCN[C@H](C)C1]

In certain embodiments, R³ is

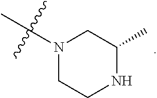 [SMILES: *N1CCN[C@@H](C)C1]

In certain embodiments, R³ is

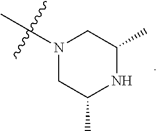 [SMILES: *N1C[C@@H](C)N[C@@H](C)C1]

In certain embodiments, R³ is

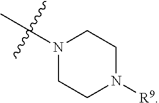

In certain embodiments, R³ is

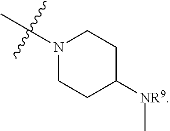

In certain embodiments, R³ is

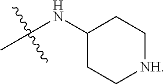 [SMILES: *NC1CCNCC1]

In certain embodiments, R³ is

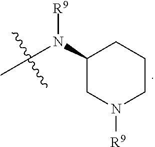

In certain embodiments, R³ is

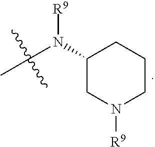

In certain embodiments, R³ is

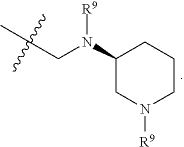

In certain embodiments, R³ is

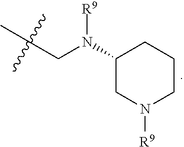

In certain embodiments, R³ is

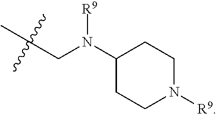

In certain embodiments, R³ is

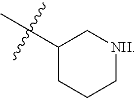 [SMILES: *C1CCCNC1]

In certain embodiments, R³ is

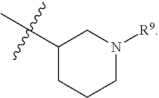

In certain embodiments, R³ is

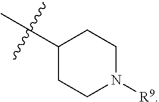

In certain embodiments, R³ is

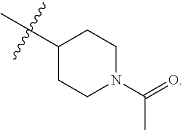 [SMILES: *C1CCN(C(C)=O)CC1]

In certain embodiments, R³ is

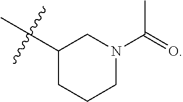 [SMILES: *C1CCCN(C(C)=O)C1]

In certain embodiments, R³ is

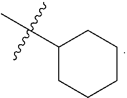 [SMILES: *C1CCCCC1]

In certain embodiments, R³ is

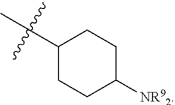

In certain embodiments, R³ is

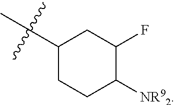

In certain embodiments, R³ is

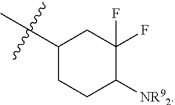

In certain embodiments, R³ is

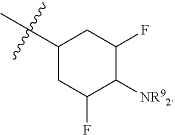

In certain embodiments, R³ is

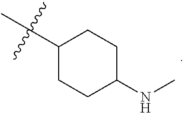 [SMILES: *C1CCC(NC)CC1]

In certain embodiments, R³ is

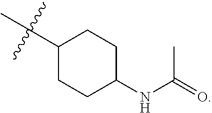 [SMILES: *C1CCC(NC(C)=O)CC1]

In certain embodiments, R³ is

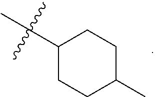 [SMILES: *C1CCC(C)CC1]

In certain embodiments, R³ is

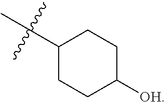 [SMILES: *C1CCC(O)CC1]

In certain embodiments, R³ is

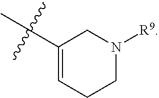

In certain embodiments, R³ is

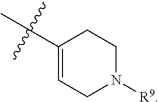

In certain embodiments, R³ is

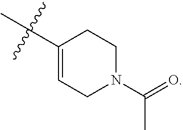 [SMILES: *C1=CCN(C(C)=O)CC1]

In certain embodiments, R³ is

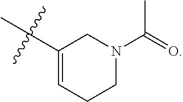 [SMILES: *C1=CCCN(C(C)=O)C1]

In certain embodiments, R³ is

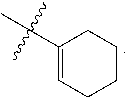 [SMILES: *C1=CCCCC1]

In certain embodiments, R³ is

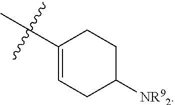

In certain embodiments, R³ is

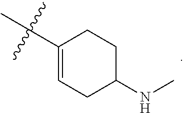 [SMILES: *C1=CCC(NC)CC1]

In certain embodiments, R³ is

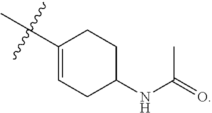 [SMILES: *C1=CCC(NC(C)=O)CC1]

In certain embodiments, R³ is

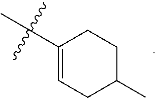 [SMILES: *C1=CCC(C)CC1]

In certain embodiments, R³ is

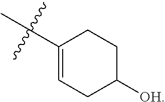 [SMILES: *C1=CCC(O)CC1]

In certain embodiments, R³ is

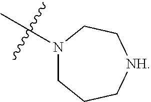 [SMILES: *N1CCCNCC1]

In certain embodiments, R³ is

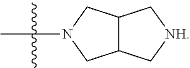 [SMILES: *N1CC2CNCC2C1]

In certain embodiments, R³ is

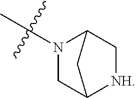 [SMILES: *N1CC2CC1CN2]

In certain embodiments, R³ is

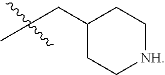 [SMILES: *CC1CCNCC1]

In certain embodiments, R³ is

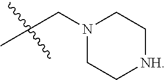 [SMILES: *CN1CCNCC1]

In certain embodiments, R³ is

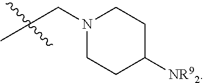

In certain embodiments, R³ is

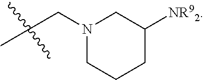

In certain embodiments, R³ is

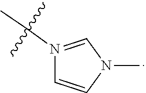

In certain embodiments, R³ is

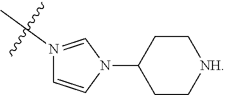

In certain embodiments, R³ is

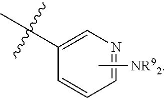

In certain embodiments, R³ is

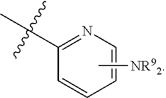

In certain embodiments, R³ is

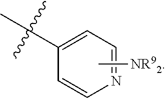

In certain embodiments, R³ is

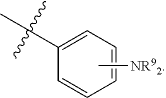

In certain embodiments, p is an integer from 0-5.

In certain embodiments, each occurrence of R⁹ is independently selected from the group consisting of H, oxetanyl, C₁-C₆ alkyl, C₃-C₈ cycloalkyl, C₁-C₆ hydroxyalkyl, C₁-C₆ (C₁-C₆ alkoxy)alkyl, C₁-C₆ haloalkyl, C₁-C₆ carboxamido alkyl, C₁-C₆ carboxy alkyl, C₁-C₆ [carboxy(C₁-C₆)alkyl] alkyl, C₁-C₆ cyano alkyl, and C₁-C₆ sulfonyl alkyl, wherein each R⁹ is independently optionally substituted with at least one of OH, halogen, C₁-C₆ alkoxy, cyano, carboxamide, carboxy, and sulfonyl.

In certain embodiments, the two R⁹ combine with the N to which they are bound to form an optionally substituted 3-8 heterocyclyl ring (such as, but not limited to, aziridine, azetidine, pyrrolidine, morpholine, piperazine, or piperidine), wherein each R⁹ is independently optionally substituted with at least one of OH, halogen, C₁-C₆ alkoxy, cyano, carboxamide, carboxy, and sulfonyl.

In certain embodiments, each occurrence of R⁹ is independently selected from the group consisting of H, methyl, ethyl, propyl, isopropyl, butyl, sec-butyl, isobutyl, tert-butyl, cyclobutyl, fluorocyclobutyl, difluorocyclobutyl, oxetanyl,

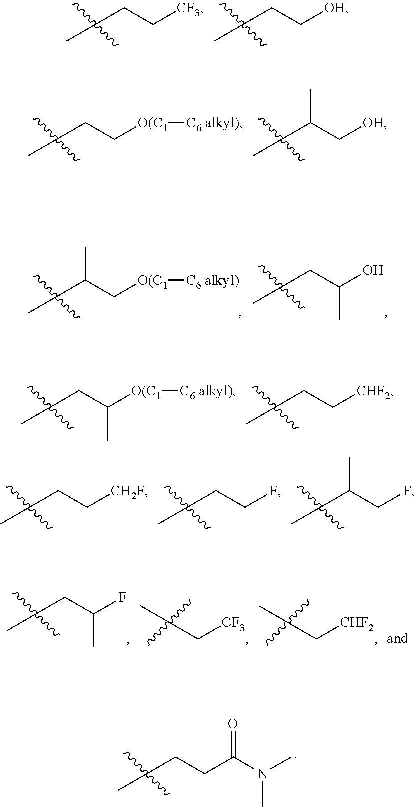

In certain embodiments, each occurrence of R⁹ is independently selected from the group consisting of: H, oxetanyl, C₁-C₈ alkyl,

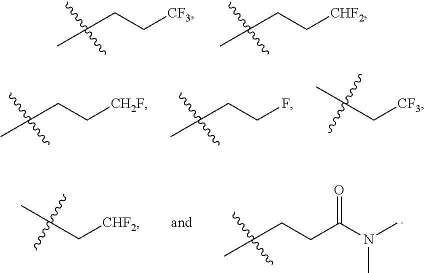

In certain embodiments, L=bond, and R³ is

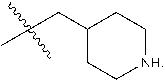 [SMILES: *CC1CCNCC1]

In certain embodiments, L=bond, and R³ is

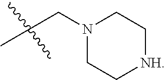 [SMILES: *CN1CCNCC1]

In certain embodiments, L=bond, and R³ is

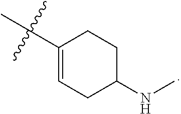 [SMILES: *C1=CCC(NC)CC1]

In certain embodiments, R³ is

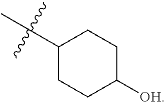 [SMILES: *C1CCC(O)CC1]

In certain embodiments, R³ is

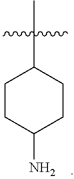 [SMILES: *C1CCC(N)CC1]

In certain embodiments, R³ is

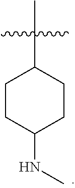 [SMILES: *C1CCC(NC)CC1]

In certain embodiments, R³ is

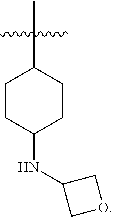 [SMILES: *C1CCC(NC2COC2)CC1]

In certain embodiments, R³ is

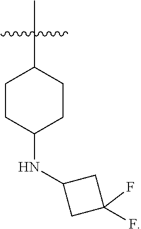 [SMILES: *C1CCC(NC2CC(F)(F)C2)CC1]

In certain embodiments, R³ is

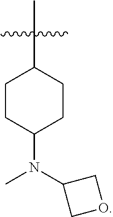 [SMILES: *C1CCC(N(C)C2COC2)CC1]

In certain embodiments, R³ is

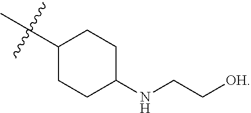 [SMILES: *C1CCC(NCCO)CC1]

In certain embodiments, R³ is

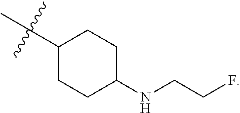 [SMILES: *C1CCC(NCCF)CC1]

In certain embodiments, R³ is

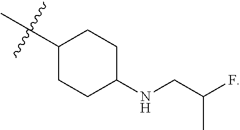 [SMILES: *C1CCC(NCC(C)F)CC1]

In certain embodiments, R³ is

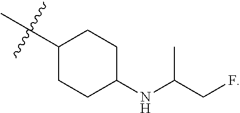 [SMILES: *C1CCC(NC(C)CF)CC1]

In certain embodiments, R³ is

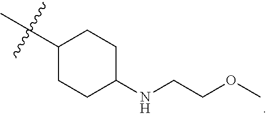 [SMILES: *C1CCC(NCCOC)CC1]

In certain embodiments, R³ is

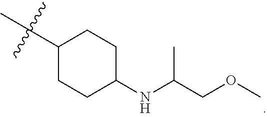 [SMILES: *C1CCC(NC(C)COC)CC1]

In certain embodiments, R³ is

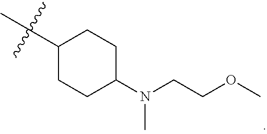 [SMILES: *C1CCC(N(C)CCOC)CC1]

In certain embodiments, R³ is

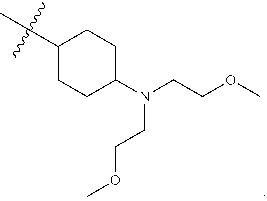 [SMILES: *C1CCC(N(CCOC)CCOC)CC1]

In certain embodiments, R³ is

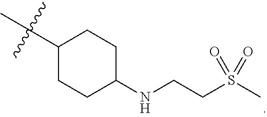 [SMILES: *C1CCC(NCCS(C)(=O)=O)CC1]

In certain embodiments, R³ is

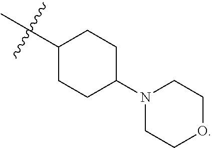 [SMILES: *C1CCC(N2CCOCC2)CC1]

In certain embodiments, R³ is

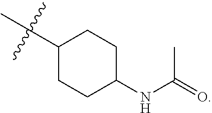 [SMILES: *C1CCC(NC(C)=O)CC1]

In certain embodiments, R³ is

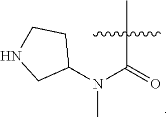 [SMILES: *C(=O)N(C)C1CCNC1]

In certain embodiments, R³ is

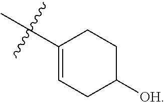 [SMILES: *C1=CCC(O)CC1]

In certain embodiments, R³ is

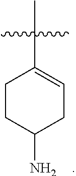 [SMILES: *C1=CCC(N)CC1]

In certain embodiments, R³ is

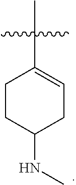 [SMILES: *C1=CCC(NC)CC1]

In certain embodiments, R³ is

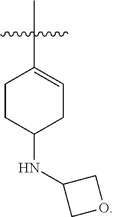 [SMILES: *C1=CCC(NC2COC2)CC1]

In certain embodiments, R³ is

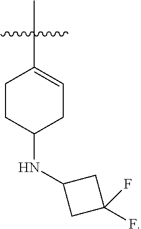 [SMILES: *C1=CCC(NC2CC(F)(F)C2)CC1]

In certain embodiments, R³ is

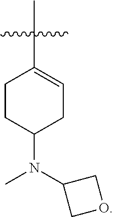 [SMILES: *C1=CCC(N(C)C2COC2)CC1]

In certain embodiments, R³ is

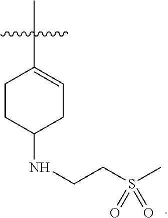 [SMILES: *C1=CCC(NCCS(C)(=O)=O)CC1]

In certain embodiments, R³ is

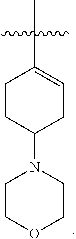 [SMILES: *C1=CCC(N2CCOCC2)CC1]

In certain embodiments, R³ is

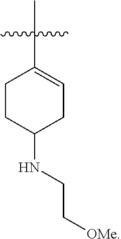 [SMILES: *C1=CCC(NCCOC)CC1]

In certain embodiments, R³ is

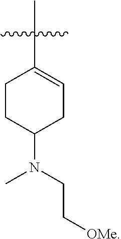 [SMILES: *C1=CCC(N(C)CCOC)CC1]

In certain embodiments, R³ is

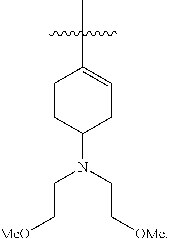 [SMILES: *C1=CCC(N(CCOC)CCOC)CC1]

In certain embodiments, R³ is

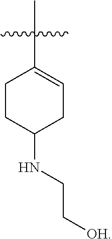 [SMILES: *C1=CCC(NCCO)CC1]

In certain embodiments, R³ is

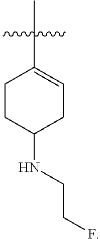 [SMILES: *C1=CCC(NCCF)CC1]

In certain embodiments, R³ is

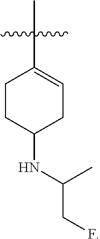 [SMILES: *C1=CCC(NC(C)CF)CC1]

In certain embodiments, R³ is

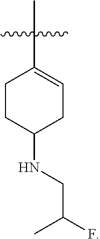 [SMILES: *C1=CCC(NCC(C)F)CC1]

In certain embodiments, R³ is

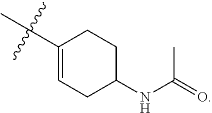 [SMILES: *C1=CCC(NC(C)=O)CC1]

In certain embodiments, R³ is at least one of the following:

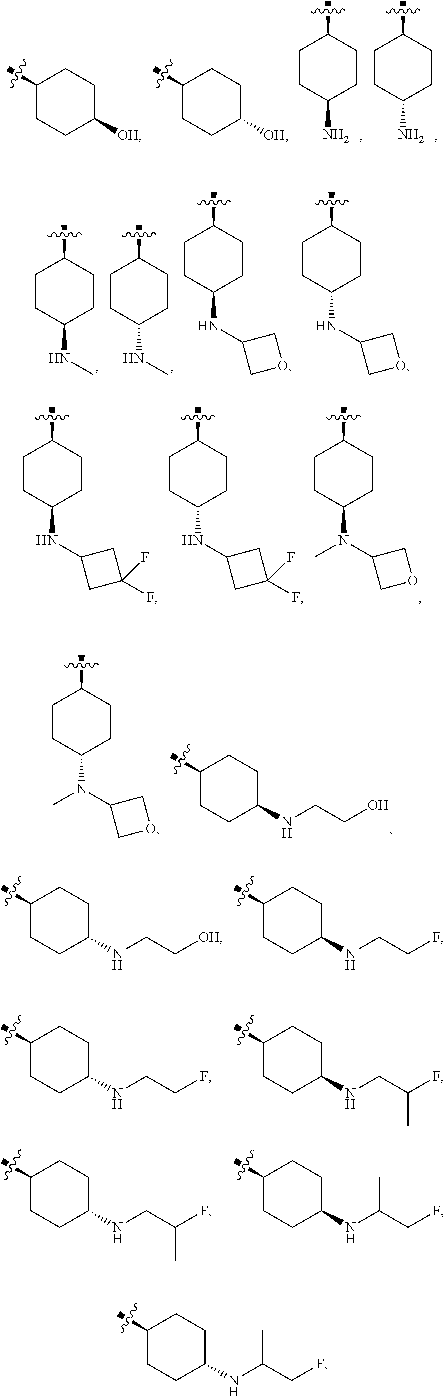

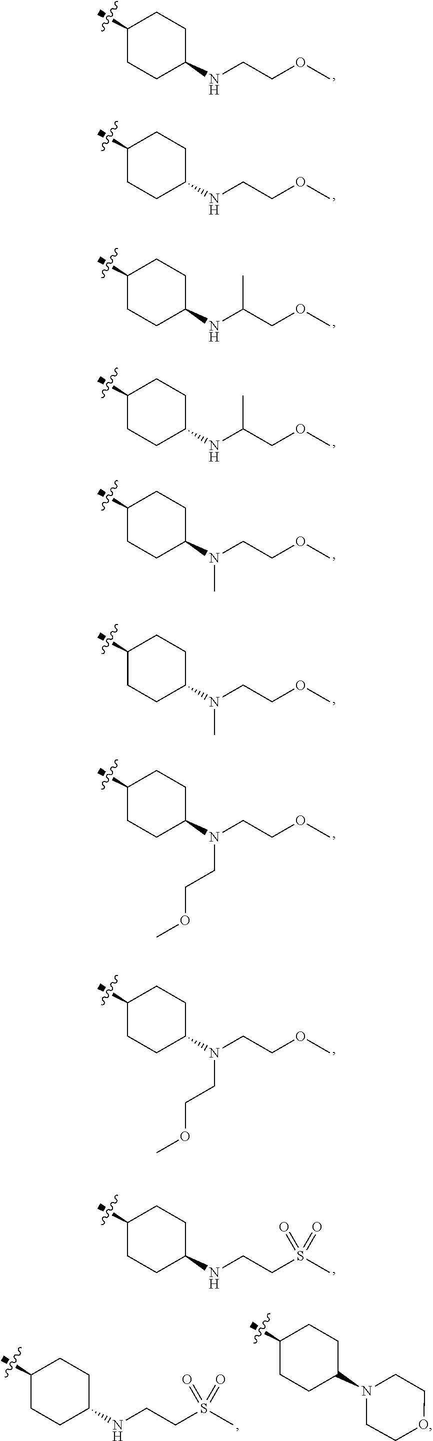

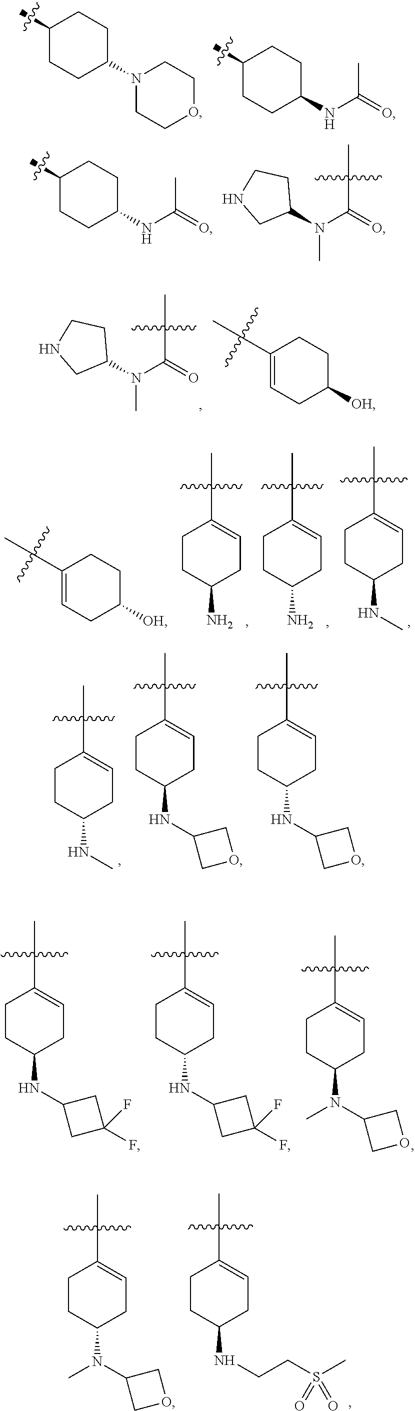

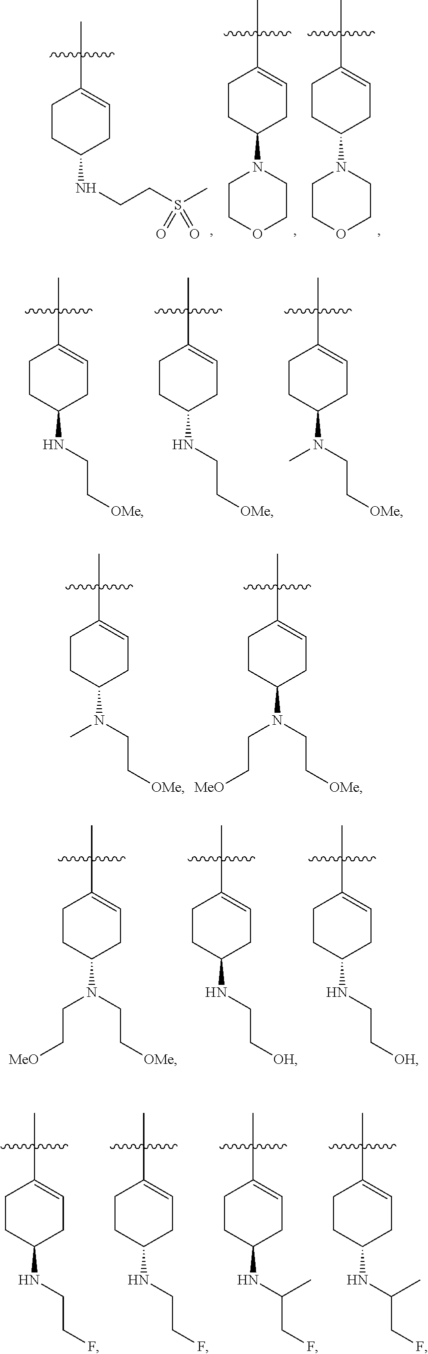

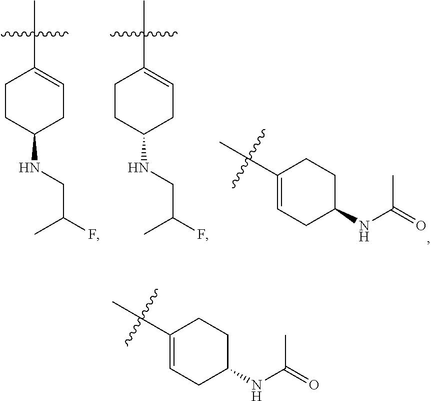 [SMILES: *C1=CC[C@@H](NCC(C)F)CC1 ; *C1=CC[C@H](NCC(C)F)CC1 ; *C1=CC[C@H](NC(C)=O)CC1 ; *C1=CC[C@@H](NC(C)=O)CC1]

In certain embodiments, R⁴ is —NH₂.

In certain embodiments, R⁵, if present, is a halogen (such as but not limited to F). In other embodiments, q=1 and R⁵ is F.

In certain embodiments, p is 0. In certain embodiments, p is 1. In certain embodiments, p is 2. In certain embodiments, p is 3. In certain embodiments, p is 4. In certain embodiments, p is 5.

In certain embodiments, the compound is

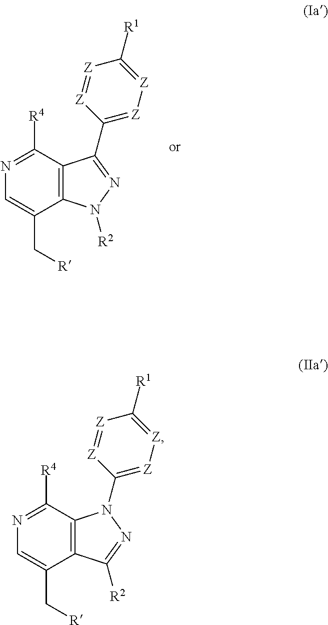

wherein R′ is R³ as defined elsewhere herein.

In certain embodiments, R′ is optionally substituted heterocyclyl. In certain embodiments, R′ is optionally substituted —NH-(optionally substituted heterocyclyl). In certain embodiments, R′ is optionally substituted —N(C₁-C₆ alkyl)-(optionally substituted heterocyclyl). In certain embodiments, R′ is

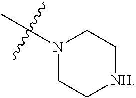 [SMILES: *N1CCNCC1]

In certain embodiments, R′ is

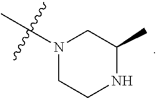 [SMILES: *N1CCN[C@H](C)C1]

In certain embodiments, R′ is

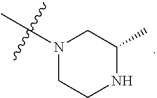 [SMILES: *N1CCN[C@@H](C)C1]

In certain embodiments, R′ is

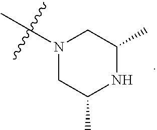 [SMILES: *N1C[C@@H](C)N[C@@H](C)C1]

In certain embodiments, R′ is

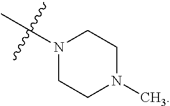 [SMILES: *N1CCN(C)CC1]

In certain embodiments, R′ is

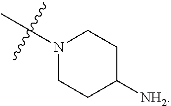 [SMILES: *N1CCC(N)CC1]

In certain embodiments, R′ is

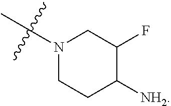 [SMILES: *N1CCC(N)C(F)C1]

In certain embodiments, R′ is

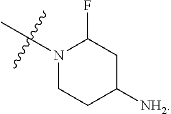 [SMILES: *N1CCC(N)CC1F]

In certain embodiments, R′ is

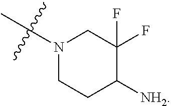 [SMILES: *N1CCC(N)C(F)(F)C1]

In certain embodiments, R′ is

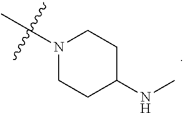 [SMILES: *N1CCC(NC)CC1]

In certain embodiments, R′ is

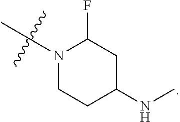 [SMILES: *N1CCC(NC)CC1F]

In certain embodiments, R′ is

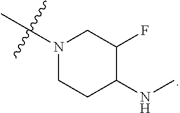 [SMILES: *N1CCC(NC)C(F)C1]

In certain embodiments, R′ is

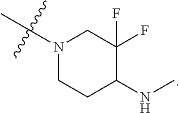 [SMILES: *N1CCC(NC)C(F)(F)C1]

In certain embodiments, R′ is

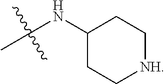 [SMILES: *NC1CCNCC1]

In certain embodiments, R′ is

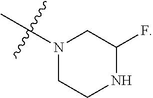 [SMILES: *N1CCNC(F)C1]

In certain embodiments, R′ is

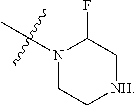 [SMILES: *N1CCNCC1F]

In certain embodiments, R′ is

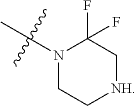 [SMILES: *N1CCNCC1(F)F]

In certain embodiments, R′ is

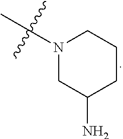 [SMILES: *N1CCCC(N)C1]

In certain embodiments, R′ is

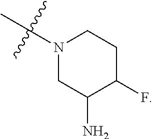 [SMILES: *N1CCC(F)C(N)C1]

In certain embodiments, R′ is

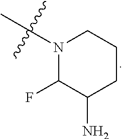 [SMILES: *N1CCCC(N)C1F]

In certain embodiments, R′ is

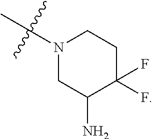 [SMILES: *N1CCC(F)(F)C(N)C1]

In certain embodiments, R′ is

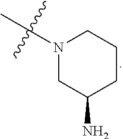 [SMILES: *N1CCC[C@@H](N)C1]

In certain embodiments, R′ is

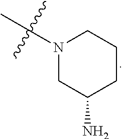 [SMILES: *N1CCC[C@H](N)C1]

In certain embodiments, R′ is

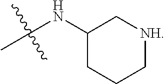 [SMILES: *NC1CCCNC1]

In certain embodiments, R′ is

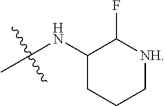 [SMILES: *NC1CCCNC1F]

In certain embodiments, R′ is

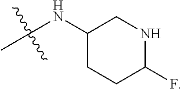 [SMILES: *NC1CCC(F)NC1]

In certain embodiments, R′ is

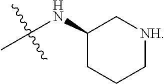 [SMILES: *N[C@@H]1CCCNC1]

In certain embodiments, R′ is

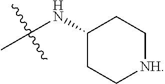

In certain embodiments, R′ is

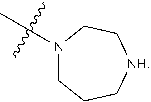 [SMILES: *N1CCCNCC1]

In certain embodiments, R′ is

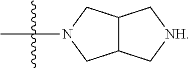 [SMILES: *N1CC2CNCC2C1]

In certain embodiments, the compound is

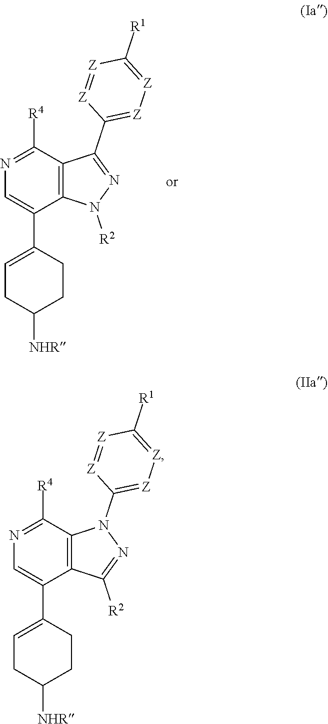

wherein R″ is H, optionally substituted C₁-C₆ alkyl, optionally substituted C₃-C₅ cycloalkyl, or optionally substituted heterocyclyl.

In certain embodiments, R″ is H. In certain embodiments, R″ is optionally substituted C₁-C₆ alkyl. In certain embodiments, R″ is optionally substituted C₃-C₅ cycloalkyl. In certain embodiments, R″ is optionally substituted heterocyclyl.

In certain embodiments, the compound is

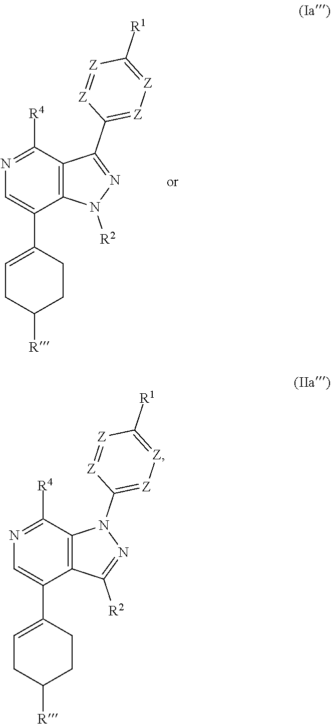

wherein R′″ in (Ia′″) or (IIa′) is selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl), wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, C₁-C₆ alkoxy, and C₁-C₆ sulfonylalkyl.

In certain embodiments, R′″ is H. In certain embodiments, R′″ is —OH. In certain embodiments, R′″ is —NH₂. In certain embodiments, R′″ is —NHCH₃. In certain embodiments, R′″ is —N(CH₃)₂. In certain embodiments, R′″ is —NHCH₂CH₂F. In certain embodiments, R′″ is —N(Me)CH₂CH₂F. In certain embodiments, R′″ is —NHCH₂CHF₂. In certain embodiments, R′″ is —N(Me)CH₂CHF₂. In certain embodiments, R′″ is —NHCH₂CF₃. In certain embodiments, R′″ is —N(Me)CH₂CF₃. In certain embodiments, R′″ is —NHCH₂CH₂CF₃. In certain embodiments, R′″ is —N(Me)CH₂CH₂CF₃. In certain embodiments, R′″ is —NHCH₂CH₂C(═O)NMe₂. In certain embodiments, R′″ is —N(Me)CH₂CH₂C(═O)NMe₂. In certain embodiments, R′″ is —NHCH₂CH₂C(═O)NH₂. In certain embodiments, R′″ is —N(Me)CH₂CH₂C(═O)NH₂. In certain embodiments, R′″ is —NHCH₂CH₂C(═O)NHMe. In certain embodiments, R′″ is —SO₂(C₁-C₆ alkyl). In certain embodiments, R′″ is —N(Me)CH₂CH₂C(═O)NHMe₂. In certain embodiments, R′″ is

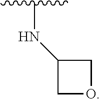 [SMILES: *NC1COC1]

In certain embodiments, the compound is

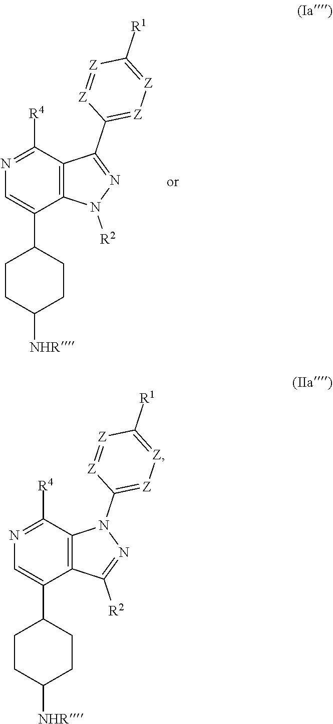

wherein R″″ is H or optionally substituted C₁-C₆ alkyl.

In certain embodiments, R″″ is H. In certain embodiments, R″″ is optionally substituted C₁-C₆ alkyl.

In certain embodiments, the compound is

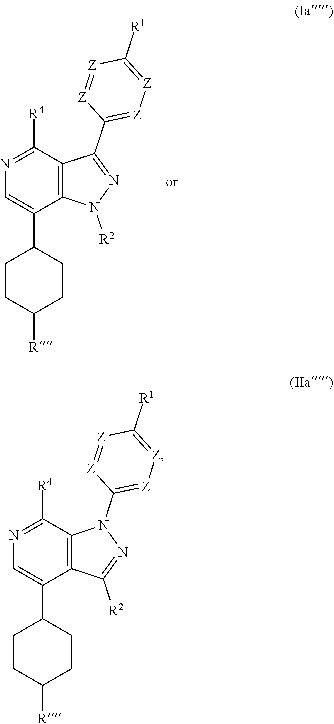

wherein R″″ in (Ia′″″) or (IIa′″″) is selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl), wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, C₁-C₆ alkoxy, and C₁-C₆ sulfonylalkyl.

In certain embodiments, R″″ is H. In certain embodiments, R″″ is —OH. In certain embodiments, R″″ is —NH₂. In certain embodiments, R″″ is —NHCH₃. In certain embodiments, R″″ is —N(CH₃)₂. In certain embodiments, R″″ is —NHCH₂CH₂F. In certain embodiments, R″″ is —N(Me)CH₂CH₂F. In certain embodiments, R″″ is —NHCH₂CHF₂. In certain embodiments, R″″ is —N(Me)CH₂CHF₂. In certain embodiments, R″″ is —NHCH₂CF₃. In certain embodiments, R″″ is —N(Me)CH₂CF₃. In certain embodiments, R″″ is —NHCH₂CH₂CF₃. In certain embodiments, R″″ is —N(Me)CH₂CH₂CF₃. In certain embodiments, R″″ is —NHCH₂CH₂C(═O)NMe₂. In certain embodiments, R″″ is —N(Me)CH₂CH₂C(═O)NMe₂. In certain embodiments, R″″ is —NHCH₂CH₂C(═O)NH₂. In certain embodiments, R″″ is —N(Me)CH₂CH₂C(═O)NH₂. In certain embodiments, R″″ is —NHCH₂CH₂C(═O)NHMe. In certain embodiments, R″″ is —SO₂(C₁-C₆ alkyl). In certain embodiments, R″″ is —N(Me)CH₂CH₂C(═O)NHMe₂. In certain embodiments, R″″ is

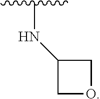 [SMILES: *NC1COC1]

In certain embodiments, the compound contemplated within the invention is a compound listed in any of Tables enclosed herein, such as but not limited to Table 1, Table 2, Table 3, Table 4, Table 5, Table 6, Table 7, Table 8, Table 9, Table 10, Table 11, Table 12, Table 13, Table 14, Table 15, Table 16, Table 17, Table 18, Table 19, Table 20, Table 21, Table 22, Table 23, Table 24, Table 25, Table 26, or a salt, solvate, enantiomer, diastereoisomer, isotopologue or tautomer thereof.

In certain embodiments, the compound is at least one of the following:
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-chloro-5-methoxy benzenesulfonamide;
N-(4-(4-amino-1-isopropyl-7-((1r,4r)-4-morpholinocyclohexyl)-1H-pyrazolo[4,3-c]pyridin-3-yl)-2,5-difluorobenzenesulphonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-chloro-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-(difluoromethoxy)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-fluoro-3-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-ethoxy-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-3-methoxybenzenesulfonamide;
N-(4-(4-amino-1-isopropyl-7-((1s,4s)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-fluoro-3-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-chlorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-chloro-4-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-chloro-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(4-fluorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-3-fluorophenyl)-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-5-cyano-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,6-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,4-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,3-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(2-fluorophenyl)methanesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluoro-5-methoxybenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-fluoro-5-methoxybenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,5-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-4-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-3-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-((1r,4r)-4-((2-methoxy ethyl)(methyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-2-fluorobenzenesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2-fluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-isopropyl-7-((1r,4r)-4-((1-methoxypropan-2-yl)amino)cyclohexyl)-1H-pyrazolo[4,3-c]pyridin-3-yl)-2-fluorophenyl)-1-(2-chlorophenyl)methanesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-fluoro-5-(trifluoromethoxy)benzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-fluoro-5-propoxybenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,5-dichlorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-chloro-5-ethylbenzenesulfonamide;
N-(4-(4-amino-7-((1r,4r)-4-((2-fluoropropyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-isopropyl-7-(4-((l-methoxy propan-2-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((l-methoxy propan-2-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)phenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-7-(4(R)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((1-fluoropropan-2-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4-((3,3-difluorocyclobutyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((3,3-difluorocyclobutyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-1-isopropyl-7-(4-((2-methoxy ethyl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((2-methoxy ethyl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-7-(4-((2-fluoro ethyl)amino)cyclohex-1-en-1-yl)-1-isopropyl-1H-pyrazolo[4,3-c]pyridin-3-yl)-2-fluorophenyl)-2-chlorobenzenesulfonamide;
   or a salt, solvate, enantiomer, diastereoisomer, isotopologue, or tautomer thereof.

In certain embodiments, the compound is at least one of the following:
N-(4-(4-amino-1-isopropyl-7-((1r,4r)-4-((l-methoxy propan-2(R)-yl)amino)cyclohexyl)-1H-pyrazolo[4,3-c]pyridin-3-yl)-2-fluorophenyl)-1-(2-chlorophenyl)methanesulfonamide;
N-(4-(4-amino-1-isopropyl-7-((1r,4r)-4-((l-methoxy propan-2(S)-yl)amino)cyclohexyl)-1H-pyrazolo[4,3-c]pyridin-3-yl)-2-fluorophenyl)-1-(2-chlorophenyl)methanesulfonamide;
N-(4-(4-amino-7-((1r,4r)-4-42(R)-fluoropropyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-7-41r,4r)-4-42(S)-fluoropropyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-isopropyl-7-(4(R)-((1-methoxypropan-2(R)-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(R)-((1-methoxypropan-2(S)-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(S)-((1-methoxy propan-2(R)-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(S)-((1-methoxypropan-2(S)-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(R)-((1-methoxypropan-2(R)-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(R)-((1-methoxypropan-2(S)-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(S)-((1-methoxypropan-2(R)-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(S)-((1-methoxy propan-2(S)-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-cyclopropyl-7-(4(R)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-cyclopropyl-7-(4(S)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-cyclopropyl-7-(4(R)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-cyclopropyl-7-(4(S)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-cyclopropyl-7-(4(R)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-cyclopropyl-7-(4(S)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4(R)-(oxetan-3-ylamino)cyclohex-1-en-1-yl)-1H-pyrazolo[4,3-c]pyridin-3-yl)phenyl)-1-(2-chlorophenyl)methanesulfonamide;
N-(4-(4-amino-1-ethyl-7-(4(S)-(oxetan-3-ylamino)cyclohex-1-en-1-yl)-1H-pyrazolo[4,3-c]pyridin-3-yl)phenyl)-1-(2-chlorophenyl)methanesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-2-chloro-5-methoxybenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-2-chloro-5-methoxybenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-7-(4(R)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((2(R)-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(R)-((2(S)-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(S)-((2(R)-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(S)-((2(S)-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(R)-((1-fluoropropan-2(R)-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(R)-((1-fluoropropan-2(S)-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(S)-((1-fluoropropan-2(R)-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(S)-((1-fluoropropan-2(S)-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(R)-((2(R)-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((2(S)-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((2(R)-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((2(S)-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((3,3-difluorocyclobutyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(S)-((3,3-difluorocyclobutyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4(R)-((3,3-difluorocyclobutyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((3,3-difluorocyclobutyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-1-isopropyl-7-(4(R)-((2-methoxy ethyl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(S)-((2-methoxy ethyl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(R)-((2-methoxy ethyl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(S)-((2-methoxy ethyl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-isopropyl-7-(4(R)-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-isopropyl-7-(4(S)-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4(R)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-ethyl-7-(4(S)-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-7-(4 (R)-((2-fluoroethyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((2-fluoro ethyl)amino)cyclohex-1-en-1-yl)-1-isopropyl-1H-pyrazolo[4,3-c]pyridin-3-yl)-2-fluorophenyl)-2-chlorobenzenesulfonamide;
   or a salt, solvate, enantiomer, diastereoisomer, isotopologue, or tautomer thereof.

In certain embodiments, the compound is an inhibitor of IRE1. In other embodiments, the compound is an inhibitor of IRE1α. In yet other embodiments, the compound is an inhibitor of IRE1α kinase activity. In yet other embodiments, the compound is an inhibitor of IRE1α RNase activity. In yet other embodiments, the compound binds the ATP binding site of IRE1α. In yet other embodiments, the compound binds IRE1α in the DFG-out conformation. In yet other embodiments, the compound binds IRE1α in the DFG-in conformation. In yet other embodiments, the compound induces the DFG-out conformation of IRE1α. In yet other embodiments, the compound is an inhibitor of IRE1α oligomerization. In yet other embodiments, the compound is an inhibitor of IRE1α dimerization. In yet other embodiments, the compound is an inhibitor of IRE1α phosphorylation. In yet other embodiments, the compound is an inhibitor of IRE1α autophosphorylation. In yet other embodiments, the compound is an inhibitor of apoptosis. In yet other embodiments, the compound is an inhibitor of IRE1α induced apoptosis. In yet other embodiments, the compound is an inhibitor of cell death. In yet other embodiments, the compound is an inhibitor of IRE1α induced cell death. In yet other embodiments, the compound is an inhibitor of a pathway induced by IRE1α phosphorylation. In yet other embodiments, the compound is an inhibitor of a pathway induced by IRE1α kinase activity. In yet other embodiments, the compound is an inhibitor of a pathway induced by IRE1α RNase activity. In yet other embodiments, the compound is an inhibitor of neuronal cell death. In yet other embodiments, the compound is a cytotoxic agent. In yet other embodiments, the compound is an anticancer agent. In yet other embodiments, the compound is an inhibitor of demyelination. In yet other embodiments, the compound is an antidiabetic agent. In yet other embodiments, the compound is a neuroprotective agent. In yet other embodiments, the compound protects against loss of photoreceptor cells. In yet other embodiments, the compound is an inhibitor of fibrosis. In yet other embodiments, the compound decreases apoptosis in cells under ER stress. In yet other embodiments, the compound decreases apoptosis in cells under ER stress, but not cells that are under the same conditions but not under ER stress. In yet other embodiments, the compound decreases apoptosis in cells under ER stress more than in cells that are under the same conditions but not under ER stress. In yet other embodiments, the compound decreases cleavage of miR-17. In yet other embodiments, the compound decreases IRE1α associated cleavage of miR-17. In yet other embodiments, the compound decreases cleavage of miR-34a. In yet other embodiments, the compound decreases IRE1α associated cleavage of miR-34a. In yet other embodiments, the compound decreases cleavage of miR-96. In yet other embodiments, the compound decreases IRE1α associated cleavage of miR-96. In yet other embodiments, the compound decreases cleavage of miR-125b. In yet other embodiments, the compound decreases IRE1α associated cleavage of miR-125b. In yet other embodiments, the compound decreases XBP 1 mRNA splicing. In yet other embodiments, the compound decreases IRE1α associated XBP1 mRNA splicing. In yet other embodiments, the compound decreases UPR signaling. In yet other embodiments, the compound decreases IRE1α associated UPR signaling. In yet other embodiments, the compound decreases terminal UPR signaling. In other embodiments, the compound decreases IRE1α associated terminal UPR signaling.

The compounds described herein may form salts with acids and/or bases, and such salts are included in the present invention. In certain other embodiments, the salts are pharmaceutically acceptable salts. The term “salts” embraces addition salts of free acids and/or bases that are useful within the methods of the invention. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hemisulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, ammonium, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound. Salts may be comprised of a fraction of less than one, one, or more than one molar equivalent of acid or base with respect to any compound of the invention.

In certain other embodiments, the at least one compound of the invention is a component of a pharmaceutical composition further including at least one pharmaceutically acceptable carrier.

### Synthesis

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups).

In the procedures that follow, some of the starting materials are identified through a “Step” or “Example” number. This is provided merely for assistance to the skilled chemist. The starting material may not necessarily have been prepared from the batch referred to.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

In this section, as in all other sections unless the context indicates otherwise, references to Formula (Ia) or (IIa) also include all other sub-groups and examples thereof as defined herein. The general preparation of some typical examples of the compounds of Formula (Ia) or (IIa) is described hereunder and in the specific examples, and are generally prepared from starting materials which are either commercially available or prepared by standard synthetic processes commonly used by those skilled in the art. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art of organic chemistry.

The skilled person will realize that in the reactions described in the Schemes, although this is not always explicitly shown, it may be necessary to protect reactive functional groups where these are desired in the final product, to avoid their unwanted participation in the reactions. In general, conventional protecting groups can be used in accordance with standard practice. The protecting groups may be removed at a convenient subsequent stage using methods known from the art. This is illustrated in the specific examples.

The skilled person will realize that in the reactions described in the Schemes, it may be advisable or necessary to perform the reaction under an inert atmosphere, such as for example under N₂ gas atmosphere.

The skilled person will realize that another sequence of the chemical reactions shown in the Schemes below, may also result in the desired compound of Formula (Ia) or (IIa).

The skilled person will realize that intermediates and final compounds shown in the schemes below may be further functionalized according to methods well-known by the person skilled in the art.

In general, intermediates of formula (II), where R² is R^{2a} being methyl, ethyl, propyl, CHF₂, cyclopropyl, 1-methylcyclopropyl, isopropyl, tert-butyl and C₃-C₈cycloalkyl, can be prepared according to the following reactions in Scheme 1. In Scheme 1, halogen is defined as Cl, Br or I. For compounds wherein R^{a} is c-Pr, step 1 is preferred over steps 2 or 3.

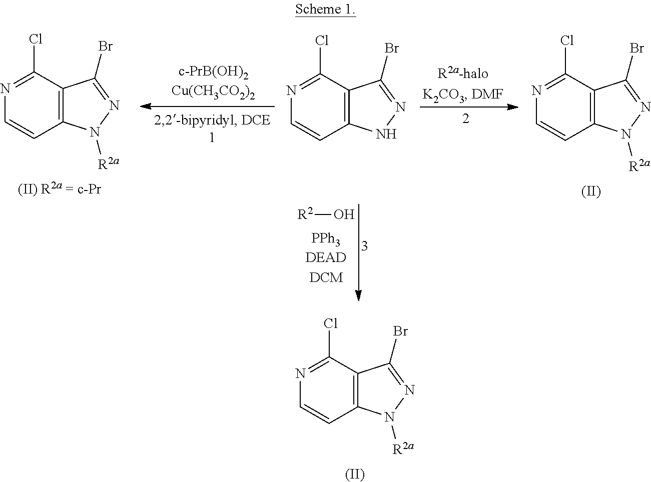

In Scheme 1, the following reaction conditions apply:

Step 1: at a suitable temperature such as 50° C., in the presence of a suitable catalyst such as copper(II) acetate, a suitable ligand such as 2,2′-bipyridyl and a suitable solvent such as 1,2-dichloroethane;

Step 2: at a suitable temperature such as room temperature, in the presence of a suitable base such as potassium carbonate, and a suitable solvent such as dimethylformamide;

Step 3: at a suitable temperature such as room temperature, in the presence of suitable coupling reagents such as diethyl azodicarboxylate (DEAD) and triphenylphosphine, and a suitable solvent such as dichloromethane.

In general, intermediates of formula (III) to (V), where R² is defined according to the scope of the present invention and PG′ represents a suitable protecting group, such as tert-(butoxycarbonyl), can be prepared according to the following reactions in Scheme 2.

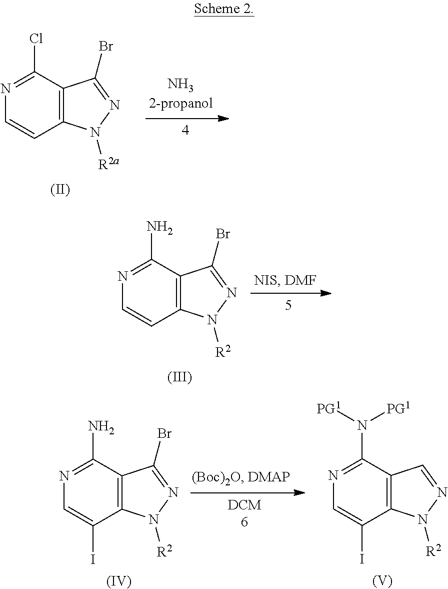

In Scheme 2, the following reaction conditions apply:

Step 4: at a suitable temperature and pressure such as 145° C. and 12.5 bar, and a suitable solvent such as 2-propanol;

Step 5: at a suitable temperature such as room temperature, in the presence of a suitable iodinating agent such as N-iodosuccinimide and a suitable solvent such as dimethylformamide;

Step 6: at a suitable temperature such as room temperature, in the presence of a suitable catalyst such as N-(dimethylamino)pyridine and a suitable solvent such as dichloromethane.

In general, intermediates of formula (VI) and (VII), where R², R⁵, Z and q are defined according to the scope of the present invention, and PG¹ and PG² represent suitable protecting groups, such as tert-(butoxycarbonyl), can be prepared according to the following reactions in Scheme 3.

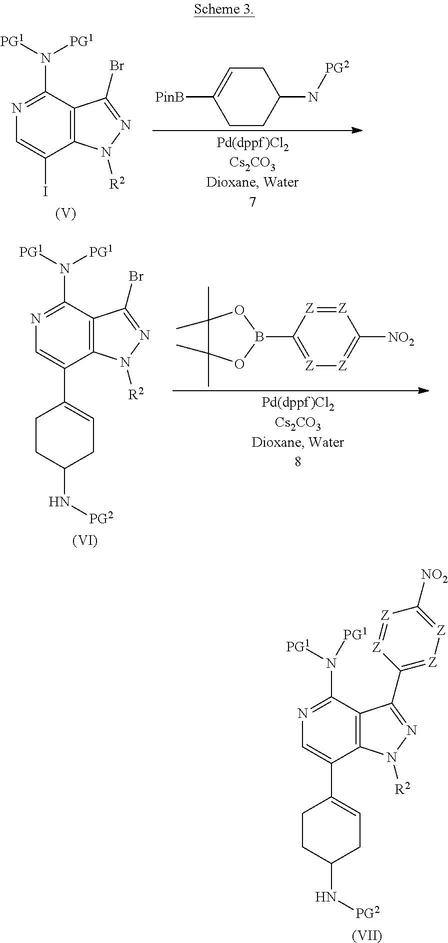

In Scheme 3, the following reaction conditions apply:

Step 7: at a suitable temperature such as 82° C., in the presence of a suitable catalyst such as Pd(dppf)Cl₂, a suitable base such as cesium carbonate and a suitable solvent such as a mixture of 1,4-dioxane and water;

Step 8: at a suitable temperature such as ranged between 62° C. and 82° C., in the presence of a suitable catalyst such as Pd(dppf)Cl₂, a suitable base such as cesium carbonate and a suitable solvent such as a mixture of 1,4-dioxane and water.

In general, intermediates of formula (VIII) and (IX), where R¹, R², R⁵, Z and q are defined according to the scope of the present invention, and PG¹ and PG² represent suitable protecting groups, such as tert-(butoxycarbonyl), can be prepared according to the following reactions in Scheme 4.

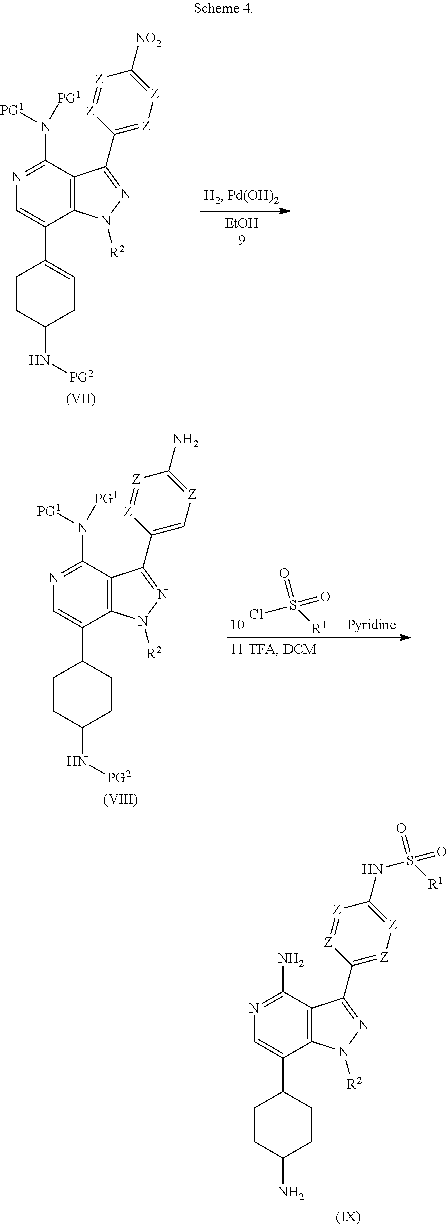

In Scheme 4, the following reaction conditions apply:

Step 9: at a suitable temperature and pressure such as 35° C. and 4 bar, in the presence of a suitable catalyst such as palladium hydroxide on carbon paste and a suitable solvent such as ethyl acetate;

Step 10: at a suitable temperature such as 40° C., in the presence of a suitable base such as pyridine and a suitable solvent such as DCM;

Step 11: at a suitable temperature such as room temperature, in the presence of a suitable acid such as trifluoroacetic acid and a suitable solvent such as DCM.

In general, final compounds of formula (Ia) or (IIa), where L is a bond and R³ is a N-substituted 4-aminocyclohexane, and R¹, R², R⁵, Z and q are defined according to the scope of the present invention, can be prepared according to the following reactions in Scheme 5. The 4-aminocyclohexane N-substituents, represented by R in Scheme 5, are either alkyl, heteroalkyl, cycloalkyl or heterocycloalkyl.

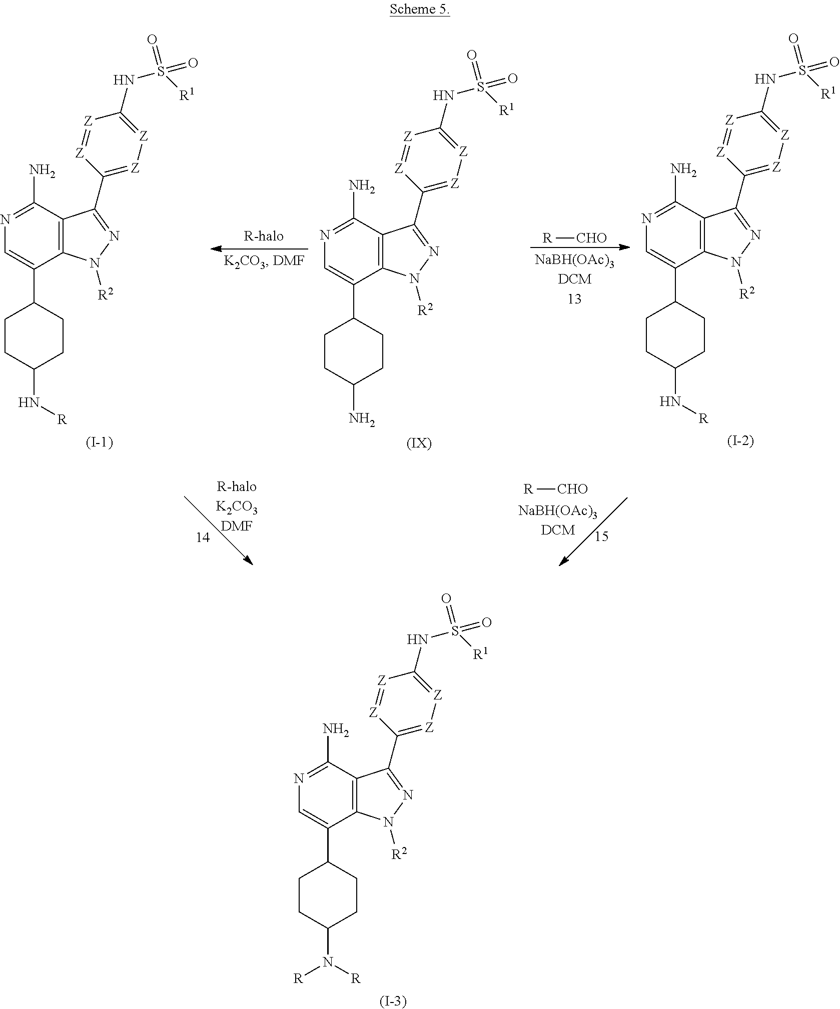

In Scheme 5, the following reaction conditions apply:

Steps 12 and 14: at a suitable temperature such as room temperature, in the presence of a suitable base such as potassium carbonate, and a suitable solvent such as dimethylformamide;

Step 13 and 15: at a suitable temperature such as room temperature, in the presence of a suitable acid such as acetic acid, a suitable reducing agent such as sodium triacetoxyborohydride and a suitable solvent such as dichloromethane.

The skilled person will realize that it is possible to convert primary amines (IX) to tertiary amines (I-3) in one step, instead of a two step process, by increasing the molar ratio of reagents to primary amine (IX) used in the reaction.

In general, final compounds of formula (Ia) or (Ib), where L is a bond and R³ is a N-substituted 4-aminocyclohexene, and R¹, R², R⁵, Z and q are defined according to the scope of the present invention, can be prepared according to the following reactions in Scheme 6. In Scheme 6, PG¹ and PG² represent suitable protecting groups, such as tert-(butoxycarbonyl), X is Cl, Br, I or triflate and the 4-aminocyclohexane N-substituent, represented by R, is alkyl, heteroalkyl, cycloalkyl or heterocycloalkyl.

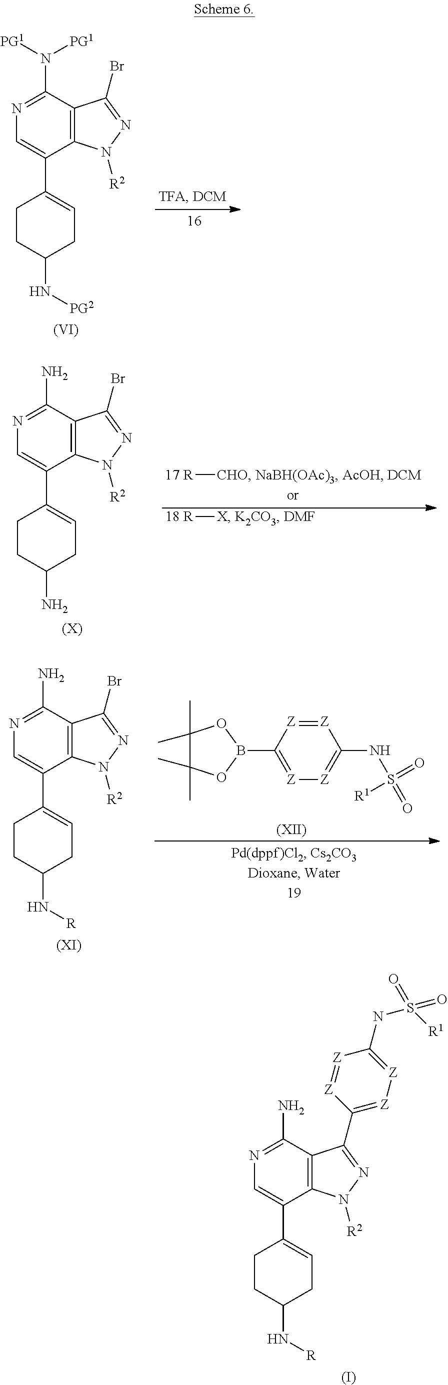

In Scheme 6, the following reaction conditions apply:

Step 16: at a suitable temperature such as room temperature, in the presence of a suitable acid such as trifluoroacetic acid and a suitable solvent such as DCM;

Step 17: at a suitable temperature such as room temperature, in the presence of a suitable acid such as acetic acid, a suitable reducing agent such as sodium triacetoxyborohydride and a suitable solvent such as dichloromethane;

Step 18: at a suitable temperature such as room temperature, in the presence of a suitable base such as potassium carbonate, and a suitable solvent such as dimethylformamide;

Step 19: at a suitable temperature such as 80° C., in the presence of a suitable catalyst such as Pd(dppf)Cl₂, a suitable base such as cesium carbonate and a suitable solvent such as a mixture of 1,4-dioxane and water.

In general, final compounds of formula (Ia) or (IIa), where L is a bond and R³ is a N-substituted 4-aminocyclohexene, and R¹, R², R⁵, Z and q are defined according to the scope of the present invention, can be prepared according to the following reactions in Scheme 6. In Scheme 6, PG² represent suitable protecting groups, such as tert-(butoxycarbonyl), X is Cl, Br, I or triflate and the 4-aminocyclohexane N-substituent, represented by R, is alkyl, heteroalkyl, cycloalkyl or heterocycloalkyl.

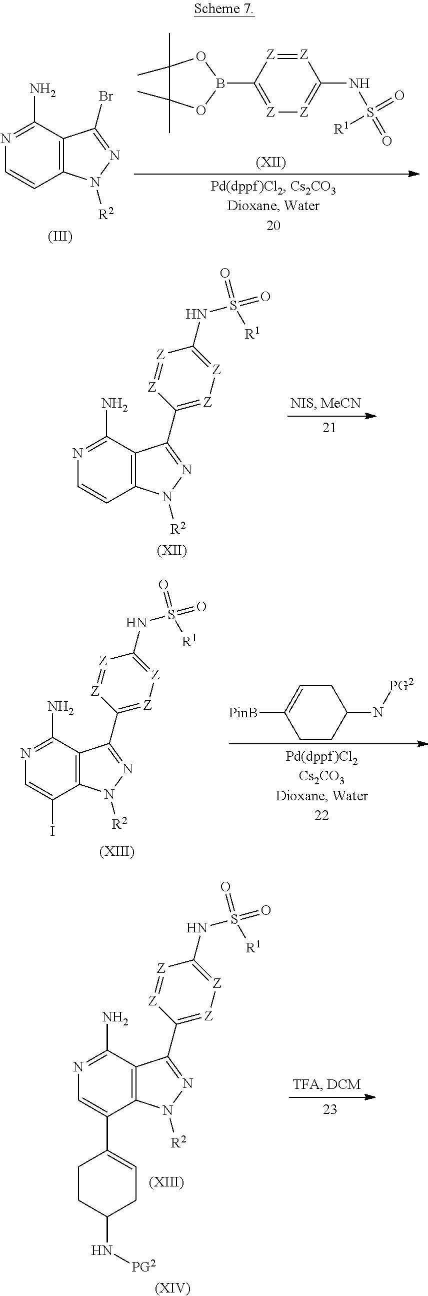

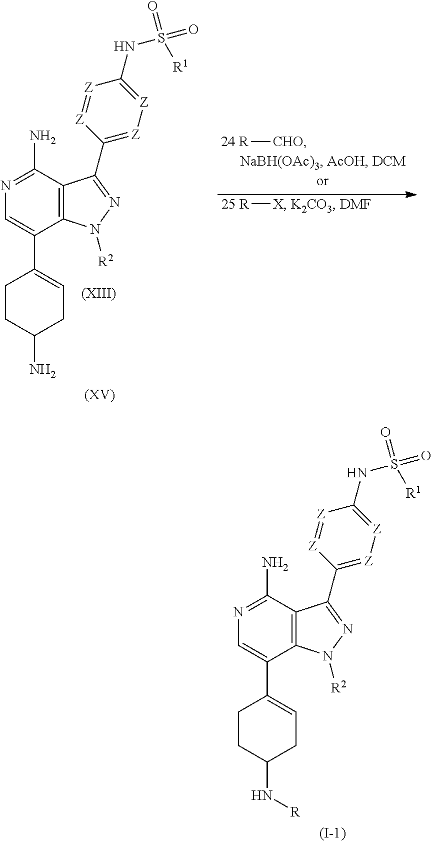

In Scheme 7, the following reaction conditions apply:

Step 20: at a suitable temperature such as 70° C., in the presence of a suitable catalyst such as Pd(dppf)Cl₂, a suitable base such as cesium carbonate and a suitable solvent such as a mixture of 1,4-dioxane and water;

Step 21: at a suitable temperature such as room temperature, in the presence of a suitable iodinating agent such as N-iodosuccinimide and a suitable solvent such as acetonitrile;

Step 22: at a suitable temperature such as 90° C., in the presence of a suitable catalyst such as Pd(dppf)Cl₂, a suitable base such as cesium carbonate and a suitable solvent such as a mixture of 1,4-dioxane and water;

Step 23: at a suitable temperature such as room temperature, in the presence of a suitable acid such as trifluoroacetic acid and a suitable solvent such as DCM;

Step 24: at a suitable temperature such as room temperature, in the presence of a suitable acid such as acetic acid, a suitable reducing agent such as sodium triacetoxyborohydride and a suitable solvent such as dichloromethane;

Step 25: at a suitable temperature such as room temperature, in the presence of a suitable base such as potassium carbonate, and a suitable solvent such as dimethylformamide.

The compounds of the invention may possess one or more stereocenters, and each stereocenter may exist independently in either the (R) or (S) configuration. In certain other embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain other embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain other embodiments, the compounds described herein exist in solvated forms with pharmaceutically acceptable solvents such as water, and ethanol. In other embodiments, the compounds described herein exist in unsolvated form.

In certain other embodiments, the compounds of the invention exist as tautomers. All tautomers are included within the scope of the compounds recited herein.

In certain other embodiments, compounds described herein are prepared as prodrugs. A “prodrug” is an agent converted into the parent drug in vivo. In certain other embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain other embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain other embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²F, and ³⁵S. In certain other embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain other embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and in the art. General methods for the preparation of compound as described herein are modified by the use of appropriate reagents and conditions, for the introduction of the various moieties found in the formula as provided herein.

### Methods

The invention includes methods of treating disorders associated with ER stress. In certain embodiments, the invention provides methods of treating a disease or disorder in a subject, the method comprising administering to the subject a therapeutically effective amount of one or more compounds of the invention, or pharmaceutically acceptable salts, solvates, enantiomers, diastereoisomers, or tautomers thereof. In other embodiments, the subject is in need of the treatment.

In certain embodiments, the disease or disorder is selected from the group consisting of a neurodegenerative disease, a demyelinating disease, cancer, an eye disease, a fibrotic disease, and diabetes.

In certain embodiments, the disease is a neurodegenerative disease selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru.

In certain embodiments, the disease is a demyelinating disease selected from the group consisting of Wolfram Syndrome, Pelizaeus-Merzbacher Disease, Transverse Myelitis, Charcot-Marie-Tooth Disease, and Multiple Sclerosis.

In certain embodiments, the disease is cancer. In other embodiments, the disease is multiple myeloma.

In certain embodiments, the disease is diabetes. In other embodiments, the disease is selected from the group consisting of type I diabetes and type II diabetes.

In certain embodiments, the disease is an eye disease selected from the group consisting of retinitis pigmentosa, retinal degeneration, macular degeneration, and Wolfram Syndrome.

In certain embodiments, the disease is a fibrotic disease selected from the group consisting of idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), and hepatic fibrosis.

Without being limited to any single theory, the compounds of the invention treat the aforementioned diseases and disorders by modulating the activity of an IRE1 protein. In certain embodiments, the compounds inhibit the activity of an IRE1 protein.

In certain embodiments, the compounds of the invention modulate kinase activity of an IRE1 protein. In other embodiments, the compounds of the invention modulate autophosphorylation activity of an IRE1 protein. In yet other embodiments, the compounds of the invention modulate oligomerization activity of an IRE1 protein. In yet other embodiments, the compounds of the invention modulate dimerization activity of an IRE1 protein.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain other embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating or preventing diseases and disorders related to IRE1) in the composition. In yet other embodiments, the compound of the invention is the only biologically active agent (i.e., capable of treating or preventing diseases and disorders related to IRE1) in therapeutically effective amounts in the composition.

In certain other embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 lag to about 10,000 mg, about 20 μg to about 9,500 mg, about 40 μg to about 9,000 mg, about 75 lag to about 8,500 mg, about 150 μg to about 7,500 mg, about 200 μg to about 7,000 mg, about 300 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain other embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include intravitreal, oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravitreal, intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intravitreal, intraperitoneal, intramuscular, intrasternal injection, and kidney dialytic infusion techniques.

### Intravitreal Administration

As used herein, “intravitreal administration” of a pharmaceutical composition includes administration into the vitreous fluid within the eye of a subject. Intravitreal administration includes, but is not limited to, administration of a pharmaceutical composition into the eye of a subject by injection of the composition. In some embodiments, the pharmaceutical composition can be administered through the use of a hypodermic needle or through a surgical incision. Preferably, administration takes place through the sclera of the eye, avoiding damage to the cornea or lens.

In certain embodiments, the pharmaceutical composition of the invention can be formulated for administration to the eye of the subject with sustained release over a period of 3-12 months.

### Controlled Release Formulations and Drug Delivery Systems

In certain other embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. In certain embodiments, the compounds of the invention can be formulated for sustained release over a period of 3-12 months.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain other embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### General Experimental Details

Reactions were not carried out under an inert atmosphere unless specified, and all solvents and commercial reagents were used as received.

Purification by chromatography refers to purification using the COMBIFLASH® Companion purification system or the Biotage SP1 purification system. Where products were purified using an ISOLUTE® SPE Si II cartridge, ‘Isolute SPE Si cartridge’ refers to a pre-packed polypropylene column containing unbonded activated silica with irregular particles with average size of 50 μm and nominal 60 Å porosity. Fractions containing the required product (identified by TLC and/or LCMS analysis) were pooled and the solvent removed by evaporation to give the desired product. Where thin layer chromatography (TLC) has been used, it refers to silica-gel TLC using plates, typically 3×6 cm silica-gel on aluminum foil plates (e.g. Fluka 60778) with a fluorescent indicator (254 nm). Microwave experiments were carried out using a Biotage Initiator 60 which uses a single-mode resonator and dynamic field tuning. Temperature from 40-250° C. can be achieved, and pressures of up to 30 bar can be reached.

NMR spectra were obtained on a Bruker Avance 400 MHz, 5 mm QNP probe H, C, F, P, single Z gradient, two channel instrument running TopSpin 2.1 or on a Bruker Avance III 400 MHz, 5 mm BBFO Plus probe, single Z gradient, two channel instrument running TopSpin 3.0.

Compound names were standardly generated using the Convert Structure to Name function in ChemDraw Professional 17.1.

Unless indicated otherwise herein, when a stereocenter is indicated with ‘RS’ this means that a mixture of the two enantiomers are present. Unless indicated otherwise herein, when a stereocenter is indicated with ‘R or S’ this means that only one of the two enantiomers are present.

### Analytical LC-MS Conditions

Method 1: Waters QDA, single quadrapole UPLC-MS with PDA detector. Column: Acquity UPLC HSS C18 (1.8 μm, 50×2.1 mm), maintained at 40° C. Conditions: 0.1% aqueous formic acid [eluent A]; MeCN (containing 0.1% formic acid) [eluent B]. Gradient: 3 to 97% B over 1.5 min at 1 mL/min.

Method 2: UPLC+Waters DAD+Waters SQD2, single quadrapole UPLC-MS. Column: Acquity UPLC BEH Shield RP18 (1.7 μm 100×2.1 mm), maintained at 40° C. Conditions: 10 mM aqueous ammonium bicarbonate [eluent A]; MeCN [eluent B]. Gradient: isocratic at 5% B for 1.2 min then 5 to 100% B over 2.3 min at 0.5 mL/min.

Method 3: Acquity H-Class UPLC with DAD detector and QDa Mass Spectrometer. Column: Acquity UPLC BEH Shield RP18 (1.7 μm 50×2.1 mm), maintained at 40° C. Conditions: 7.66 mM ammonia in water [eluent A]; 7.66 mM ammonia in MeCN [eluent B]. Gradient: 3 to 97% B over 4.4 min at 0.8 mL/min.

Method 4: Acquity H-Class UPLC with quaternary pump/PDA detector and QDa Mass Spectrometer. Column: Acquity UPLC CSH C18 (1.7 μm 50×2.1 mm), maintained at 40° C. Conditions: 0.1% aqueous formic acid [eluent A]; MeCN (containing 0.1% formic acid) [eluent B]. Gradient: 3 to 99% B over 1.5 min, then isocratic for 0.4 mins at 1 mL/min.

### QC LC-MS Conditions

QC Method 1: Acquity i-Class (quarternary pump/PDA detector)+Quattro Micro Mass Spectrometer. Column: Acquity UPLC BEH C18 (1.7 μm, 100×2.1 mm), maintained at 40° C. Conditions: 0.1% aqueous formic acid [eluent A]; MeCN (containing 0.1% formic acid) [eluent B]. Gradient: isocratic at 5% B for 0.4 min then 5 to 95% B over 5.6 min at 0.4 mL/min.

QC Method 2: Acquity UPLC (binary pump/PDA detector)+ZQ Mass Spectrometer. Column: Acquity UPLC BEH C18 (1.7 μm, 100×2.1 mm), maintained at 40° C. Conditions: 0.1% aqueous formic acid [eluent A]; MeCN (containing 0.1% formic acid) [eluent B]. Gradient: isocratic at 5% B for 0.4 min then 5 to 95% B over 5.6 min at 0.4 mL/min.

### SFC Methods

Preparative SFC: Waters Thar Prep100 preparative SFC system (P200 CO₂ pump, 2545 modifier pump, 2998 UV/VIS detector, 2767 liquid handler with Stacked Injection Module). Column: Phenomenex Lux Cellulose-4 or YMC Cellulose-SC (5 μm, 10-21.2×250 mm), maintained at 40° C. Conditions: supercritical fluid CO₂ and eluents chosen from MeOH, EtOH, IPA, MeCN, EtOAc, THF with modifiers chosen from Et₂NH or formic acid as specified. Gradient/isocratic as specified at 100 mL/min, 120 bar (or as appropriate).

Analytical SFC was carried out on a similar system using smaller columns and lower flow rates.

### Preparative HPLC:

Intershim PuriFlash XS420 system, C18 80 g cartridge (PF_C18HP, 15 μm from Intershim), using 5-98% MeCN in water (+0.1% FA) at 34 mL/min over 7 column volumes run, UV detection 220 nm.

### Preparation of Selected Intermediates:

### Preparation of intermediate 1: 3-bromo-4-chloro-1-isopropyl-1H-pyrazolo[4,3-c]pyridine

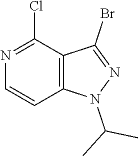

2-Iodopropane (129 mL, 219.4 g, 1.291 mol) was added dropwise at RT to a mechanically stirred suspension of 3-bromo-4-chloro-1H-pyrazolo[4,3-c]pyridine (CAS: 1246349-99-4) (100 g, 0.43 mol) and anhydrous potassium carbonate (89.2 g, 0.645 mol) in dry DMF (1 L) and the resulting suspension stirred at RT for 16 h. Water (5 L) and EtOAc (2 L) were added with vigorous stirring to give a 2-phase solution. The aqueous layer was separated and further extracted with EtOAc (3×1 L) then the combined organic extracts were washed with water (2×500 mL), 5 wt % aqueous lithium chloride solution (500 mL), saturated brine (500 mL) then dried (Na₂SO₄) and concentrated in vacuo to give the crude product as an approximately 3:1 mixture of the 1-isopropyl and 2-isopropyl alkylation products as a viscous syrup that solidified on standing. The products were separated by column chromatography on SiO₂, using a gradient eluent of 0-50% TBME in cyclohexane. The unwanted 3-bromo-4-chloro-2-isopropyl-2H-pyrazolo[4,3-c]pyridine by-product was the first eluting component, which was discarded. Fractions containing the more polar, later eluting component were combined and evaporated to give the title compound (86.3 g, 70%) as a colourless syrup that solidified on standing to a colourless solid. ¹H NMR (400 MHz, CDCl₃) δ: 8.16 (1H, d, J=6 Hz), 7.27 (1H, d, J=6 Hz), 4.77 (1H, septet, J=6.7 Hz), 1.59 (6H, d, J=6.7 Hz).

Intermediate 2 (Table 1) was prepared by using an analogous reaction protocol as described for intermediate 1 from 3-bromo-4-chloro-1H-pyrazolo[4,3-c]pyridine (CAS: 1246349-99-4).

TABLE 1

| | | LCMS | HPLC Rₜ |
|---|---|---|---|
| Intermediate | Structure | m/z (M + H) | (min)/Method |
| 2 | 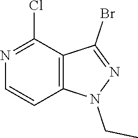 | 260/262 | 1.26/Method D |

### Preparation of intermediate 3: 3-bromo-4-chloro-1-cyclpropyl-1H-pyrazolo[4,3-c]pyridine

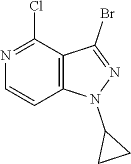 [SMILES: Clc1nccc2c1c(Br)nn2C1CC1]

A mixture of cyclopropyl boronic acid (9.24 g, 107.54 mmol), 3-bromo-4-chloro-1H-pyrazolo[4,3-c]pyridine (CAS: 1246349-99-4) (10 g, 43.02 mmol), copper(II) acetate (7.82 g, 43.02 mmol) and 2,2′-bipyridyl (6.72 g, 43.02 mmol) in 1,2-dichloroethane (200 mL) was stirred for 2 days at 50° C. then cooled to RT. The mixture was diluted with DCM and washed with saturated aqueous ammonium chloride solution. The aqueous layer was extracted with DCM (×3) and the combined organic extracts were washed with saturated brine, dried (Na₂SO₄) and concentrated in vacuo. The residue was purified by chromatography on SiO₂ (220 g), eluting with DCM/EtOAc (0 to 10%), to give the title compound as a white solid (7.4 g, 63%). LCMS (Method E): Rt=1.53 min, m/z [M+H]⁺=272/274/276

### Preparation of intermediate 4: 3-bromo-1-isopropyl-1H-pyrazolo[4,3-c]pyridin-4-amine

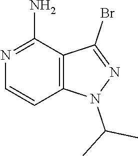

Ammonia gas was bubbled for 45 minutes through 33 wt % aqueous ammonium hydroxide (200 mL, 1.66 mol) solution chilled to −15° C. to −5° C. internal temperature, resulting in the formation of a super-saturated solution of ammonia (56 g, 3.29 mol) in 33 wt % aqueous ammonium hydroxide. The ammonia solution was charged to a pre-chilled steel pressure vessel containing a suspension of 3-bromo-4-chloro-1-isopropyl-1H-pyrazolo[4,3-c]pyridine (intermediate 1) (41.50 g, 0.151 mol) in 2-propanol (200 mL) and the pressure vessel sealed. The vessel was heated to 145° C. resulting in a pressure rise to 12.5 bar, and the mixture stirred at this temperature for 48 h then cooled to RT. Remaining excess pressure was released, the vessel unsealed and the resulting suspended white solid collected by filtration. The solid was rinsed with 2-propanol (20 mL) then vacuum dried to give the title compound (24.80 g, 66%) as an off-white solid. ¹H NMR (400 MHz, CDCl₃) δ: 7.81 (1H, d, J=6.2 Hz), 6.67 (1H, d, J=6.2 Hz), 5.45 (2H, bs), 4.66 (1H, septet, J=6.7 Hz), 1.55 (6H, d, J=6.7 Hz).

Intermediates 5-6 (Table 2) were prepared by using an analogous reaction protocol as described for intermediate 4 from the appropriate starting material.

TABLE 2

| | | Starting | LCMS | HPLC Rₜ |
|---|---|---|---|---|
| Intermediate | Structure | material | m/z (M + H) | (min)/Method |
| 5 | 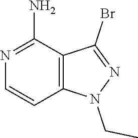 | Intermediate 2 | 241/240 | 0.61/Method D |
| 6 | 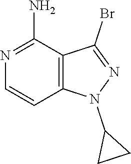 [SMILES: Nc1nccc2c1c(Br)nn2C1CC1] | Intermediate 3 | 253/255 | 0.58/Method D |

### Preparation of intermediate 7: 3-bromo-7-iodo-1-isopropyl-1H-pyrazolo[4,3-c]pyridin-4-amine

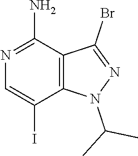

NIS (33.33 g, 0.148 mol) was added to a stirred solution of 3-bromo-1-isopropyl-1H-pyrazolo[4,3-c]pyridin-4-amine (intermediate 4) (25.20 g, 98.8 mmol) in dry DMF (125 mL) to give a dark orange-brown solution, which was stirred at RT for 16 h, resulting in the formation of a dark tan coloured suspension. The suspended solids were collected by filtration then the filter cake was washed sequentially with water (100 mL) and EtOAc (50 mL) then vacuum dried to give the title compound (24.15 g, 64% yield) as an off-white solid. The filtrates were diluted with water (400 mL) and EtOAc (400 mL) then 10 wt % aqueous sodium metabisulfite (200 mL) was added to destroy most of the dark orange colour. The resulting aqueous phase was separated, basified to pH 11 by addition of 1M sodium hydroxide then further extracted with EtOAc (2×200 mL). The combined organic extracts were washed with 10 wt % aqueous sodium metabisulfite (100 mL), water (100 mL), 5 wt % aqueous lithium chloride (100 mL) and saturated brine (100 mL) then dried (Na₂SO₄) and evaporated to give a second crop of slightly impure title compound (13.6 g, 35%) as a dark tan coloured solid. ¹H NMR (400 MHz, CDCl₃) δ: 8.08 (1H, s), 5.82 (1H, septet, J=6.6 Hz), 5.52 (2H, bs), 1.55 (6H, d, J=6.6 Hz).

Intermediates 8-9 (Table 3) were prepared by using an analogous reaction protocol as described for intermediate 7 from the appropriate starting material.

TABLE 3

| | | Starting | LCMS | HPLC Rₜ |
|---|---|---|---|---|
| Intermediate | Structure | material | m/z (M + H) | (min)/Method |
| 8 | 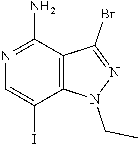 | Intermediate 5 | 367/369 | 0.80/Method D |
| 9 | 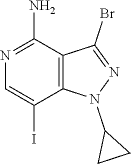 [SMILES: Nc1ncc(I)c2c1c(Br)nn2C1CC1] | Intermediate 6 | 379/380 | 0.82/Method D |

### Preparation of intermediate 10: tert-butyl (3-bromo-7-iodo-1-isopropyl-1H-pyrazolo[4,3-c]pyridin-4-yl)(tert-butoxycarbonyl)carbamate

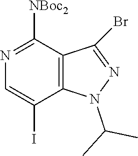

Di-tert-butyldicarbonate (20.62 g, 94.5 mmol) was added to a stirred solution of 3-bromo-7-iodo-1-isopropyl-1H-pyrazolo[4,3-c]pyridin-4-amine (intermediate 7) (24.0 g, 62.99 mmol) and 4-(dimethylamino)pyridine (0.19 g, 1.57 mmol) in dry DCM (400 mL) and the resulting suspension stirred at RT for 72 h. A second portion of di-tert-butyldicarbonate (11.33 g, 51.91 mmol) was added and stirring continued for a further 24 h. The resulting mixture was washed sequentially with saturated aqueous NaHCO₃ (100 mL), 10 wt % aqueous citric acid (100 mL), saturated brine (100 mL), dried (Na₂SO₄) and concentrated in vacuo to give the title compound (36.6 g, quantitative yield) as a tan coloured solid, which was used without further purification. ¹H NMR (400 MHz, CDCl₃) δ: 8.63 (1H, s), 5.89 (1H, septet, J=6.6 Hz), 1.59 (6H, d, J=6.6 Hz), 1.42 (18H, s).

Intermediates 11-12 (Table 4) were prepared by using an analogous reaction protocol as described for intermediate 10 from the appropriate starting material.

TABLE 4

| | | Starting | LCMS | HPLC Rₜ |
|---|---|---|---|---|
| Intermediate | Structure | material | m/z (M + H) | (min)/Method |
| 11 | 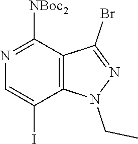 | Intermediate 8 | 567/569 | 1.65/Method D |
| 12 | 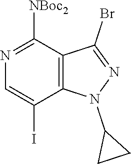 | Intermediate 9 | 579/580 | 1.66/Method D |

### Preparation of intermediate 13: tert-butyl (3-bromo-7-(4-((tert-butoxycarbonyl)amino) cyclohex-1-en-1-yl)-1-isopropyl-1H-pyrazolo[4,3-c]pyridin-4-yl)(tert-butoxycarbonyl)carbamate

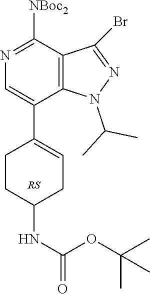

A cloudy solution of tert-butyl (3-bromo-7-iodo-1-isopropyl-1H-pyrazolo[4,3-c]pyridin-4-yl)(tert-butoxycarbonyl)carbamate (intermediate 10) (27.28 g, 39.8 mmol), cesium carbonate (38.88 g, 119.3 mmol) and tert-butyl (4-(4,4,5,5-tetramethyl-1,3,2-dioxaborolan-2-yl)cyclohex-3-en-1-yl)carbamate (CAS: 1251732-64-5; 12.86 g, 39.87 mmol) in a mixture of 1,4-dioxane (130 mL) and water (35 mL) was de-oxygenated by means of evacuation and argon refill, then the mixture was treated with Pd(dppf)Cl₂.DCM (3.25 g, 3.98 mmol) and heated to 82° C. with mechanical stirring for 16 h. The resulting black suspension was diluted with water (200 mL) and products extracted into EtOAc (1×200 mL and 3×100 mL). The combined extracts were washed with saturated brine, dried (Na₂SO₄), filtered through CELITE® and concentrated in vacuo to give the crude product as a dark brown foam. The product was purified by SiO₂-pad column chromatography eluting with 0-30% EtOAc in cyclohexane gradient solvent to give the title compound (18.98 g, 69%) as a pale-yellow foam. ¹H NMR (400 MHz, CDCl₃) δ: 7.98 (1H, s), 5.82 (1H, m), 4.90 (1H, m), 4.58 (1H, bs), 3.93 (1H, m), 2.70-2.60 (1H, m), 2.52-2.30 (2H, m), 2.18-2.06 (2H, m), 1.85-1.67 (1H, m), 1.65-1.35 (6H. m), 1.48 (9H, s), 1.45 (18H, s).

Intermediates 14-15 (Table 5) were prepared by using an analogous reaction protocol as described for intermediate 13 from the appropriate starting material.

TABLE 5

| | | | LCMS | HPLC Rₜ |
|---|---|---|---|---|
| Intermediate | Structure | Starting material | m/z (M + H) | (min)/Method |
| 14 | 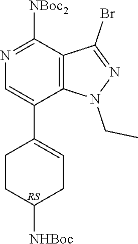 | Intermediate 11: tert-Butyl (4-(4,4,5,5- tetramethyl-1,3,2- dioxaborolan-2- yl)cyclohex-3-en-1- yl)carbamate CAS: 1251732-64-5 | 636/638 | 3.65/Method F |
| 15 | 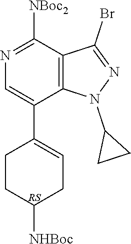 | Intermediate 12: tert-Butyl (4-(4,4,5,5- tetramethyl-1,3,2- dioxaborolan-2- yl)cyclohex-3-en-1- yl)carbamate CAS: 1251732-64-5 | 648/650 | 1.70/Method D |

### Preparation of intermediate 16: tert-butyl (tert-butoxycarbonyl)(7-(4-((tert-butoxycarbonyl) amino)-cyclohex-1-en-1-yl)-3-(3-fluoro-4-nitrophenyl)-1-isopropyl-1H-pyrazolo[4,3-c]pyridin-4-yl)carbamate

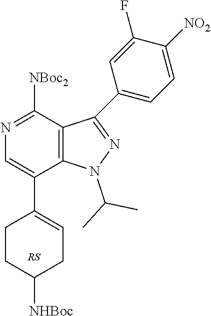

A suspension of ter t-butyl (3-bromo-7-(4-((tert-butoxycarbonyl)amino)cyclohex-1-en-1-yl)-1-isopropyl-1H-pyrazolo[4,3-c]pyridin-4-yl)(tert-butoxycarbonyl)carbamate (intermediate 13) (20.0 g, 30.74 mmol), cesium carbonate (30.05 g, 92.2 mmol) and 2-(3-fluoro-4-nitrophenyl)-4,4,5,5-tetramethyl-1,3,2-dioxaborolane (CAS: 939968-60-2; 9.85 g, 36.89 mmol) in a mixture of 1,4-dioxane (100 mL) and water (10 mL) was degassed by sonication, evacuation and argon refill to give a cloudy solution. Pd(dppf)Cl₂.DCM (2.51 g, 32.07 mmol) was added and the stirred mixture heated to 62° C. (internal temperature) for 18 h. The mixture was cooled to RT, second portions of 2-(3-fluoro-4-nitrophenyl)-4,4,5,5-tetramethyl-1,3,2-dioxaborolane (0.66 g, 2.47 mmol) and Pd(dppf)Cl₂.DCM (0.65 g, 0.80 mmol) added and re-heated to 75° C. for a further 22 h. The resulting black suspension was cooled to RT, filtered to remove suspended solids, diluted with EtOAc (100 mL) and water (100 mL) and the layers separated. The aqueous layer was further extracted with EtOAc (2×100 mL) then the combined organic layers were dried (Na₂SO₄) and concentrated in vacuo to give a dark orange gum. The product was partially purified by column chromatography on SiO₂ using a 0-50% EtOAc in cyclohexane gradient to give the desired impure product (19.3 g) as a yellow foam. The impure product was then re-dissolved in a mixture of 1,4-dioxane (100 mL) and water (10 mL), cesium carbonate (29.0 g, 88.99 mmol) and fresh 2-(3-fluoro-4-nitrophenyl)-4,4,5,5-tetramethyl-1,3,2-dioxaborolane (3.96 g, 14.83 mmol) added, and the stirred mixture degassed by evacuation and argon refill. Pd(dppf)Cl₂.DCM (1.21 g, 1.48 mmol) was added and the stirred mixture heated to 80° C. for 4 h. The resulting black suspension was cooled to RT and diluted with EtOAc (400 mL) and water (100 mL). The aqueous layer was separated and further extracted with EtOAc (2×200 mL) then the combined organic layers were washed with saturated brine, dried (Na₂SO₄), filtered through glass-fibre paper and concentrated in vacuo to give a dark brown foam. The crude product was purified by column chromatography on SiO₂ using a 5-30% EtOAc in cyclohexane gradient eluent to give the title compound (15.33 g, 70% yield) as a yellow foam. The product was then recrystallised from hot EtOH (100 mL) and the crystallised solid collected by filtration, rinsed with EtOH (50 mL) and vacuum dried at 40° C./7 mbar in a desiccator to give the pure title compound (12.59 g, 57%) as an off-white solid. ¹H NMR (400 MHz, CDCl₃) δ: 8.12 (1H, dd, J=8.7 and 7.7 Hz), 8.03 (1H, s), 7.71-7.63 (2H, m), 5.86 (1H, m), 5.12-4.95 (1H, m), 4.72-4.48 (1H, m), 4.02-3.85 (1H, m), 2.72-2.62 (1H, m), 2.58-2.32 (2H, m), 2.27-2.06 (2H. m), 1.88-1.70 (1H, m), 1.70-1.40 (6H, m), 1.49 (9H, s), 1.37 (18H, s).

### Preparation of intermediate 17: tert-butyl (3-(4-amino-2,5-difluorophenyl)-7-4-((tert-butoxycarbonyl)amino)cyclohex-1-en-1-isopropyl-1H-pyrazolo[4,3-c]pyridin-4-yl)carbamate

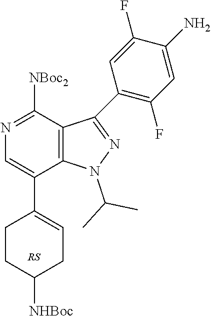

A suspension of ter t-butyl (3-bromo-7-(4-((tert-butoxycarbonyl)amino)cyclohex-1-en-1-yl)-1-isopropyl-1H-pyrazolo[4,3-c]pyridin-4-yl)(tert-butoxycarbonyl)carbamate (Intermediate 13) (5.0 g, 7.85 mmol), cesium carbonate (7.80 g, 23.94 mmol) and 2,5-difluoro-4-(4,4,5,5-tetramethyl-1,3,2-dioxaborolan-2-yl)aniline (CAS: 939807-75-7; 3.02 g, 11.84 mmol) in a mixture of 1,4-dioxane (100 mL) and water (10 mL) was degassed by sonication, evacuation and argon refill to give a cloudy solution. Pd(PPh₃)₄ (0.91 g, 0.789 mmol) was added and the stirred mixture heated to 90° C. (internal temperature) for 24 h. The mixture was cooled to RT, diluted with EtOAc (250 mL) and water (100 mL) and the layers separated. The aqueous layer was further extracted with EtOAc (2×100 mL) then the combined organic layers were dried (Na₂SO₄) and concentrated in vacuo to give a dark orange gum. The product was partially purified by column chromatography on SiO₂ using a 0-60% EtOAc in cyclohexane gradient eluent system to give the desired product (4.74 g, 88%) as an off-white solid. LCMS (Method G): Rt=1.76 min, m/z [M+H]⁺=685

Intermediates 18-21 (Table 6) were prepared by using an analogous reaction protocol as described for intermediate 17 from the appropriate starting material.

TABLE 6

| | | | LCMS | HPLC Rₜ |
|---|---|---|---|---|
| Intermediate | Structure | Starting materials | m/z (M + H) | (min)/Method |
| 18 | 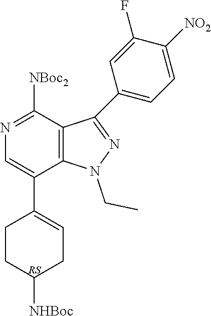 | Intermediate 14: 2-(3-Fluoro-4-nitrophenyl)- 4,4,5,5-tetramethyl-1,3,2- dioxaborolane (CAS: 939968-60-2) | 697 | 1.76/Method D |
| 19 | 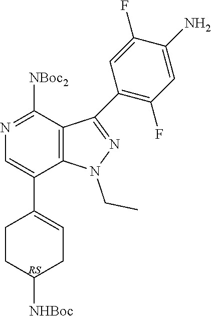 | Intermediate 14: 2,5-Difluoro-4-(4,4,5,5- tetramethyl- 1,3,dioxaborolane-2- yl)aniline (CAS: 939807- 75-7) | 685 | 1.76/Method G |
| 20 | 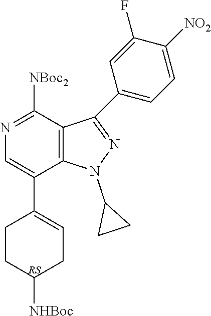 | Intermediate 15: 2-(3-Fluoro-4-nitrophenyl)- 4,4,5,5-tetramethyl-1,3,2- dioxaborolane (CAS: 939968-60-2) | 709 | 3.78/Method G |
| 21 | 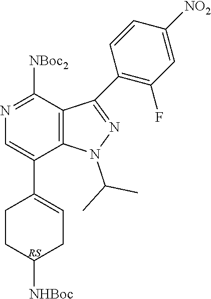 | Intermediate 13: 2-(2-Fluoro-4-nitrophenyl)- 4,4,5,5-tetramethyl-1,3,2- dioxaborolane (CAS: 939807-75-7) | 681 | 1.75/Method G |

### Preparation of intermediate 22: cis/trans-tert-butyl (3-(4-amino-3-fluorophenyl)-7-(4-((tert-butoxy-carbonyl)amino)cyclohexyl)-1-isopropyl-1H-pyrazolo[4,3-c]pyridin-4-yl)(tert-butoxycarbonyl)carbamate

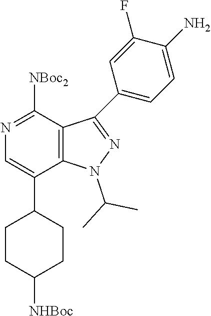

A solution of tert-butyl (tert-butoxycarbonyl)(7-(4-((tert-butoxycarbonyl)amino)-cyclohex-1-en-1-yl)-3-(3-fluoro-4-nitrophenyl)-1-isopropyl-1H-pyrazolo[4,3-c]pyridin-4-yl)carbamate (intermediate 16) (12.59 g, 17.7 mmol), in EtOAc (300 mL) was charged to a reaction flask containing a large magnetic stirrer bar and palladium hydroxide on carbon paste (10 wt % Pd, 50 wt % water, 12.50 g, 4.45 mmol) under a nitrogen atmosphere. The vessel was evacuated by application of vacuum then refilled with hydrogen and the resulting suspension stirred under a balloon of hydrogen for 16 h. The hydrogen atmosphere was purged by evacuation and N₂ refill, then the catalyst was removed by filtration through CELITE® with the filter cake being rinsed with EtOH (200 mL) then DCM (300 mL) until the filtrate was colourless to fully wash the aniline cyclohexene intermediate off the catalyst. The filtrates were concentrated in vacuo to give the partially reduced aniline cyclohexene intermediate as an orange glass, which was re-dissolved in EtOH (150 mL) and the resulting solution charged to a 1 L stainless steel pressure hydrogenation vessel containing a large magnetic stirrer bar and a fresh portion of palladium hydroxide on carbon paste (10 wt % Pd, 50 wt % water, 12.50 g, 4.45 mmol). The vessel was sealed, flushed with hydrogen, and the reaction mixture hydrogenated at 35° C. and 4 bar hydrogen pressure for 4 days. The vessel was purged with nitrogen then the catalyst was removed by filtration through CELITE® and the vessel and filter cake washed with EtOH (200 mL) then DCM (800 mL). The solution was concentrated in vacuo to give the title compound as a mixture of cis/trans isomers (12.7 g, 100%) as a pale-yellow foam. ¹H NMR (400 MHz, CDCl₃) δ: 8.20-7.10 (1H, m, cis/trans isomers), 7.35-7.27 (1H, m), 7.25-7.20 (1H, m), 6.81 (1H, m), 5.00-4.87 (1H, m), 4.86-4.44 (1H, m, cis/trans isomers), 4.06-3.94 (1H, m), 3.66-3.50 (1H, m), 3.21-3.03 (1H, m, cis/trans isomers), 2.72-2.36 (1H, m), 2.31-2.22 (1H, m), 2.18-1.69 (7H, m), 1.63 (6H, d, J=6.5 Hz), 1.54-1.43 (9H, m, cis/trans isomers), 1.39-1.32 (18H, m, cis/trans isomers).

Intermediates 23-27 (Table 7) were prepared by using an analogous reaction protocol as described for intermediate 22 from the appropriate starting material.

TABLE 7

| | | Starting | LCMS | HPLC Rₜ |
|---|---|---|---|---|
| Intermediate | Structure | material | m/z (M + H) | (min)/Method |
| 23 | 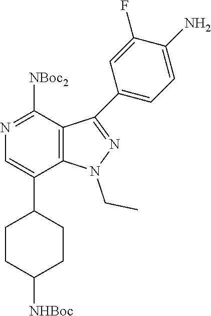 | Intermediate 18 | 669 | 3.31 (trans)/3.35 (cis)/Method H |
| 24 | 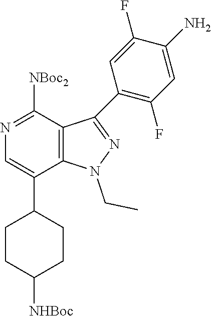 | Intermediate 19 | 687 | 3.31 (trans)/3.35 (cis)/Method H |
| 25 | 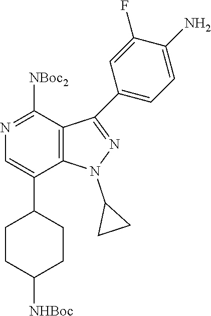 | Intermediate 20 | 681 | 4.10 (trans)/4.19 (cis)/Method I |
| 26 | 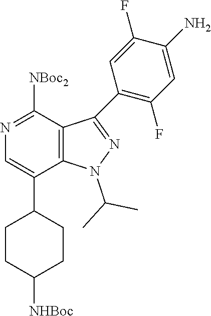 | Intermediate 17 | 701 | 1.78 (trans)/1.80 (cis)/Method G |
| 27 | 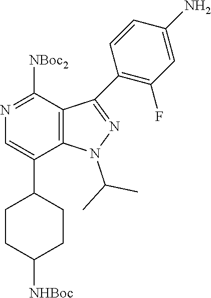 | Intermediate 21 | 683 | 3.27 (trans)/3.31 (cis)/Method H |

### Preparation of intermediate 28 and intermediate 29: tert-butyl (tert-butoxycarbonyl)(741s,4s)-4-((tert-butoxy-carbonyl)amino)cyclohexyl)-3-(3-fluoro-4-(2-fluorophenyl)sulfonamido)phenyl)-1-isopropyl-1H-pyrazolo[4,3-c]pyridin-4-yl)carbamate (intermediate 28) and tert-butyl (tert-butoxycarbonyl)(7-((1r,4r)-4-((tert-butoxy-carbonyl)amino)cyclohexyl)-3-(3-fluoro-4-(2-fluorophenyl)sulfonamido)phenyl)-1-isopropyl-1H-pyrazolo[4,3-c]pyridin-4-yl)carbamate (intermediate 29)

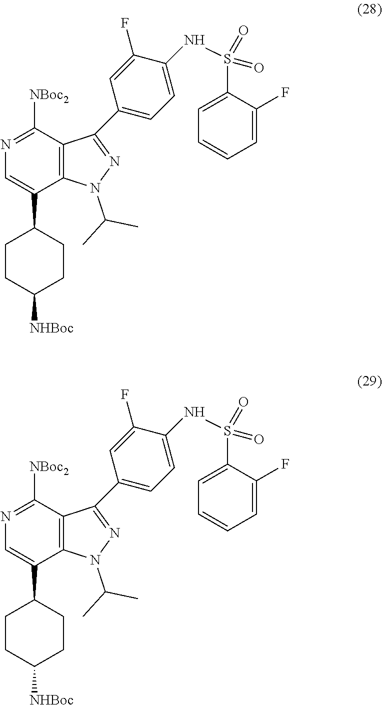

2-Fluorobenzenesulfonyl chloride (CAS: 2905-21-7; 3.0 mL, 4.34 g, 22.32 mmol) was added to a stirred solution of cis/trans-tert-butyl (3-(4-amino-3-fluorophenyl)-7-(4-((tert-butoxy-carbonyl)amino)-cyclohexyl)-1-isopropyl-1H-pyrazolo[4,3-c]pyridin-4-yl)(tert-butoxycarbonyl)carbamate (intermediate 22) (12.70 g, 18.60 mmol) and pyridine (4.5 mL, 4.41 g, 55.8 mmol) in DCM (223 mL) and the resulting mixture heated to 40° C. for 18 h. Further portions of pyridine (4.5 mL, 4.41 g, 55.8 mmol) and 2-fluorobenzenesulfonyl chloride (3.0 mL, 4.34 g, 22.32 mmol) were added and heating continued for a further 4 h before a final portion of 2-fluorobenzenesulfonyl chloride (1.10 g, 5.65 mmol) was added and the mixture heated for 3 h. Water (15 mL) was added and the mixture filtered through a hydrophobic filter paper. The filtrate was concentrated in vacuo to give a crude product that was partially purified by column chromatography on 800 g, 50 μm SiO₂ column, using a 0-50% EtOAc in cyclohexane gradient to generate a batch of approximately 1:1 cis/trans sulfonamide product (9.5 g, 60% yield) plus a batch of mostly cis isomer (intermediate 28) (4.6 g, 29%). LCMS (Method H): Rt=2.47 min; m/z [M+H]⁺=841.

A 7.6 g portion of the cis/trans mixed product was further purified by column chromatography on a 330 g, 15 μm SiO₂ column, using a 0-50% EtOAc in cyclohexane gradient to generate a batch of pure title compound (intermediate 29) (1.87 g, 11%). ¹H NMR (400 MHz, CDCl₃) δ: 8.18 (1H, s), 7.92 (1H, tm, J=7.5 Hz), 7.63-7.55 (2H, m), 7.39-7.32 (2H, m), 7.30-7.18 (2H, m), 7.17 (1H, bs), 4.93 (1H, septet, J=6.6 Hz), 4.52-4.38 (1H, m), 3.65-3.48 (1H, m), 3.06 (1H, tm, J=11.8 Hz), 2.30-2.20 (2H, m), 2.14-2.04 (2H, m), 1.86-1.71 (2H, m), 1.60 (6H, d, J=6.6 Hz), 1.60-1.56 (2H, m), 1.46 (9H, s), 1.30 (18H, s). LCMS (Method H): Rt=2.45 min; m/z [M+H]⁺=841

Intermediates 30-70 and 167-170 (Table 8) were prepared by using an analogous reaction protocol as described for intermediate 29 from the appropriate starting material.

TABLE 8

| | | Starting | LCMS | HPLC Rₜ |
|---|---|---|---|---|
| Intermediate | Structure | materials | m/z (M + H) | (min)/Method |
| 30 | 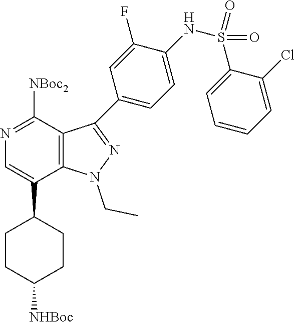 | Intermediate 23 2-Chlorobenzene sulfonyl chloride (CAS: 2905-23-9) | 843 | 2.62/Method H |
| 31 | 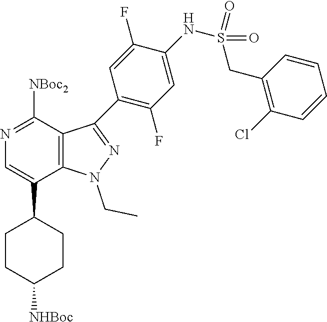 | Intermediate 24 (2-Chlorophenyl) methanesulfonyl chloride (CAS: 77421- 13-7) | 875 | 3.62/Method H |
| 32 | 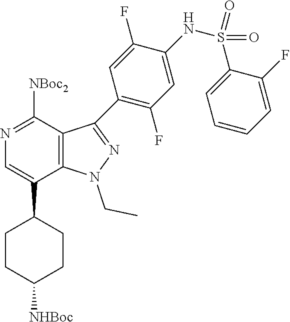 | Intermediate 24 2-Fluorobenzene sulfonyl chloride (CAS: 2905-21-7) | 845 | 2.19/Method H |
| 33 | 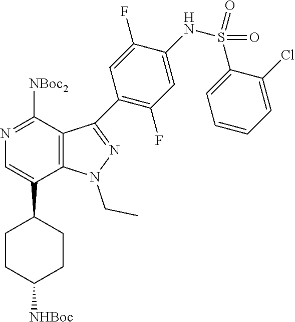 | Intermediate 24 2-Chlorobenzene sulfonyl chloride (CAS: 2905-23-9) | 861 | 1.69/Method D |
| 34 | 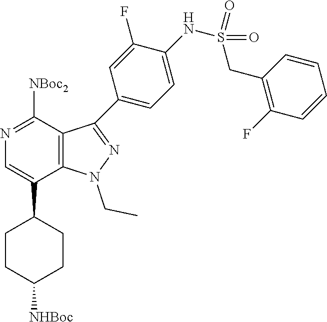 | Intermediate 23 (2-Fluorophenyl) methanesulfonyl chloride (CAS: 24974- 71-8) | 841 | 2.80/Method H |
| 35 | 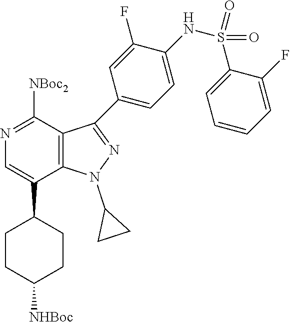 | Intermediate 25 2-Fluorobenzene sulfonyl chloride (CAS: 2905-21-7) | 839 | 2.53/Method H |
| 36 | 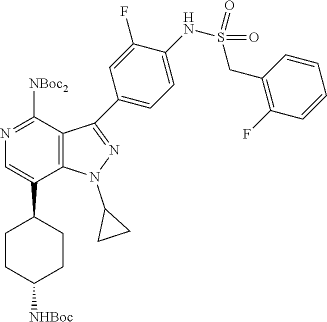 | Intermediate 25 (2-Fluorophenyl) methanesulfonyl chloride (CAS: 24974- 71-8) | 853 | 1.68/Method D |
| 37 | 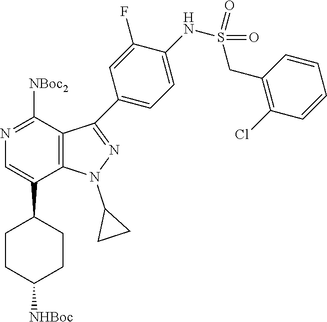 | Intermediate 25 (2-Chlorophenyl) methanesulfonyl chloride (CAS: 77421- 13-7) | 869 | 1.72/Method D |
| 38 | 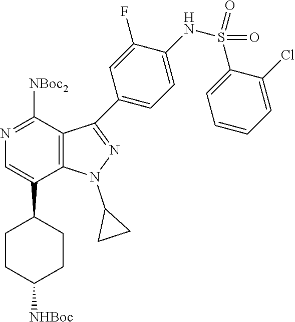 | Intermediate 25 2-Chlorobenzene sulfonyl chloride (CAS: 2905-23-9) | 855 | 1.71/Method D |
| 39 | 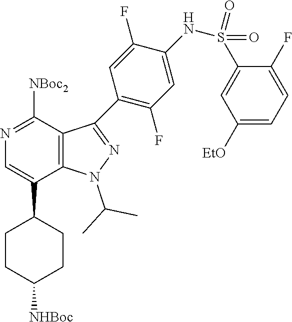 | Intermediate 26 Intermediate 119 | 903 | 1.85/Method D |
| 40 | 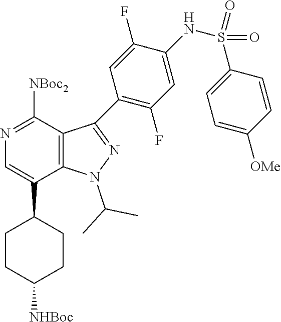 | Intermediate 26 4-Methoxy benzene sulfonyl chloride (CAS: 98-68-0) | 872 | 2.29/Method H |
| 41 | 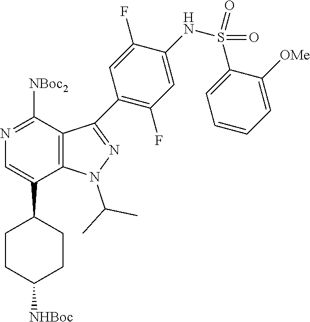 | Intermediate 26 2-Methoxy benzene sulfonyl chloride (CAS: 10130-87-7) | 872 | 2.37/Method H |
| 42 | 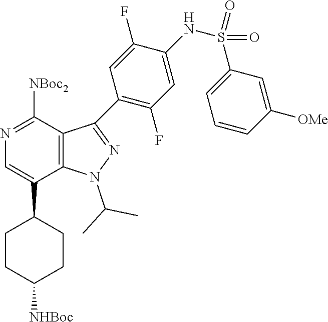 | Intermediate 26 3-Methoxy benzene sulfonyl chloride (CAS: 10130-74-2) | 872 | 2.25/Method H |
| 43 | 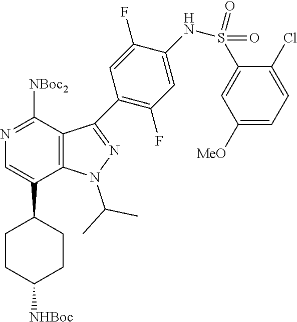 | Intermediate 26 2-Chloro-5- methoxybenzene sulfonyl chloride (CAS: 201935-41-3) | 905/907 | 2.28/Method H |
| 44 | 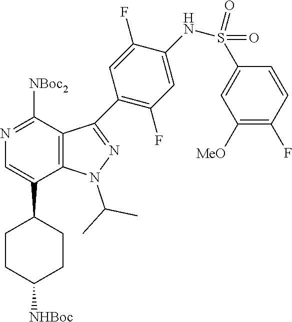 | Intermediate 26 4-Fluoro-3- methoxybenzene sulfonyl chloride (CAS: 887266-97-9) | 889 | 1.87/Method D |
| 45 | 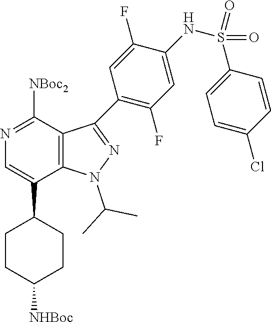 | Intermediate 26 4-Chlorobenzene sulfonyl chloride (CAS: 98-60-2) | 875/877 | 2.35/Method H |
| 46 | 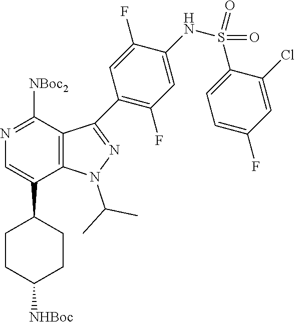 | Intermediate 26 2-Chloro-4- fluorobenzene sulfonyl chloride (CAS: 85958- 57-2) | 893/895 | 1.26/Method G |
| 47 | 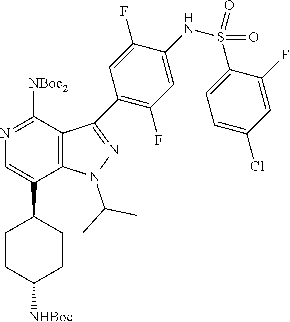 | Intermediate 26 4-Chloro-2-fluoro benzenesulfonyl chloride (CAS: 141337- 26-0) | 893/895 | 1.40/Method G |
| 48 | 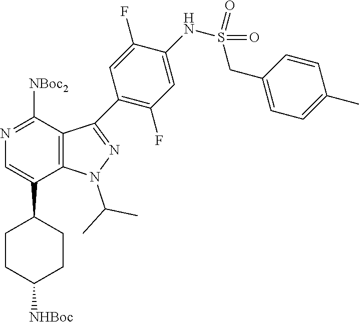 | Intermediate 26 (4-Fluorophenyl) methanesulfonyl chloride (CAS: 103360- 04-9) | 874 | 1.42/Method G |
| 49 | 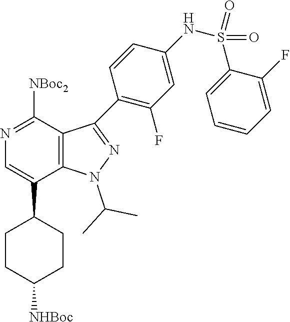 | Intermediate 27 2-Fluorobenzene sulfonyl chloride (CAS: 2905-21-7) | 841 | 2.42/Method H |
| 50 | 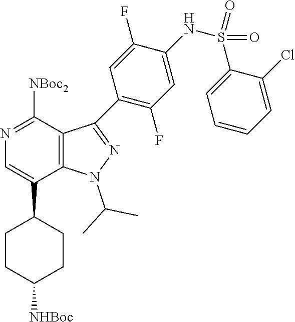 | Intermediate 26 2-Chlorobenzene sulfonyl chloride (CAS: 2905-23-9) | 875/877 | 1.37/Method G |
| 51 | 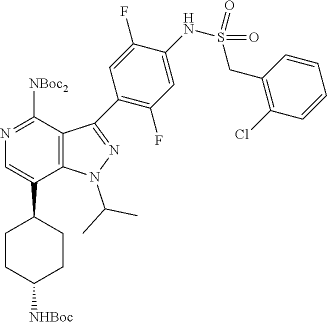 | Intermediate 26 (2-Chlorophenyl) methanesulfonyl chloride (CAS: 77421- 13-7) | 889/891 | 1.36/Method G |
| 52 | 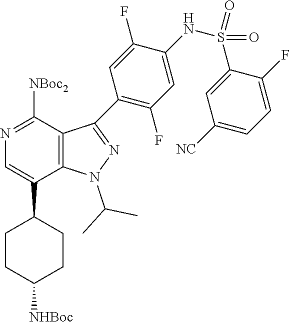 | Intermediate 26 5-Cyano-2-fluoro benzenesulfonyl chloride (CAS: 1101120-80-2) | 884 | 1.32/Method G |
| 53 | 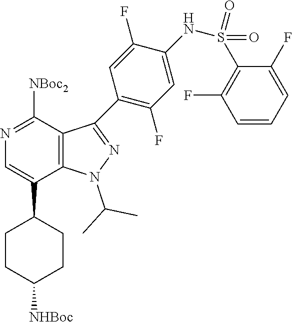 | Intermediate 26 2,6-Difluorobenzene sulfonyl chloride (CAS: 60230-36-6) | 877 | 1.31/Method G |
| 54 | 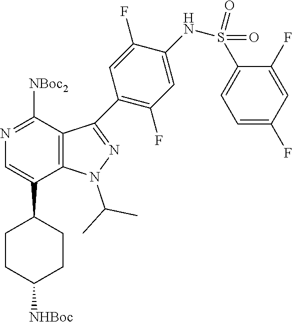 | Intermediate 26 2,4-Difluorobenzene sulfonyl chloride (CAS: 13918-92-8) | 877 | 1.34/Method G |
| 55 | 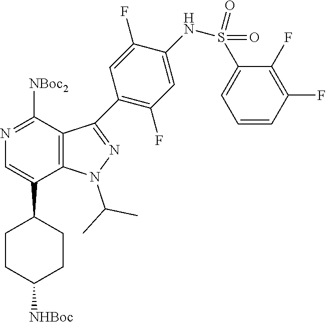 | Intermediate 26 2,3-Difluorobenzene sulfonyl chloride (CAS: 210532-24-4) | 877 | 1.34/Method G |
| 56 | 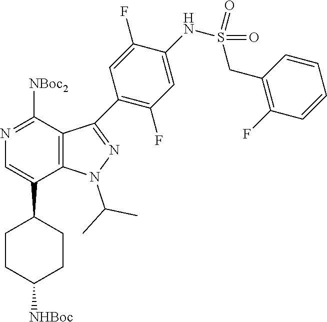 | Intermediate 26 (2-Fluorophenyl) methanesulfonyl chloride (CAS: 24974- 71-8) | 873 | 1.80/Method D |
| 57 | 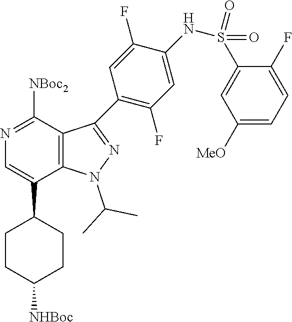 | Intermediate 26 2-Fluoro-5- methoxybenzene sulfonyl chloride (CAS: 1214334-01-6) | 890 | 2.23/Method H |
| 58 | 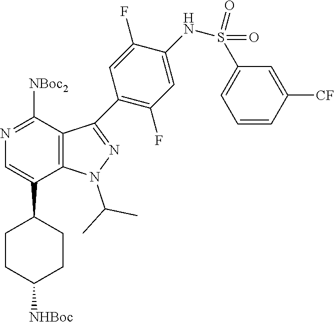 | Intermediate 26 3-(Trifluoromethyl) benzene sulfonyl chloride (CAS: 777-44- 6) | 909 | 2.67/Method H |
| 59 | 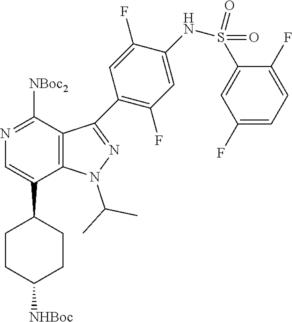 | Intermediate 26 2,5-Difluorobenzene sulfonyl chloride (CAS: 26120-86-5) | 877 | 1.33/Method G |
| 60 | 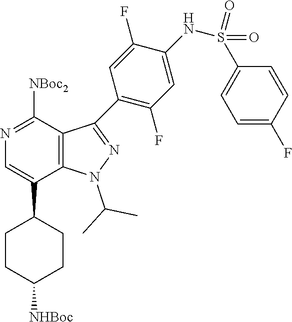 | Intermediate 26 4-Fluorobenzene sulfonyl chloride (CAS: 349-88-2) | 859 | 2.12/Method E |
| 61 | 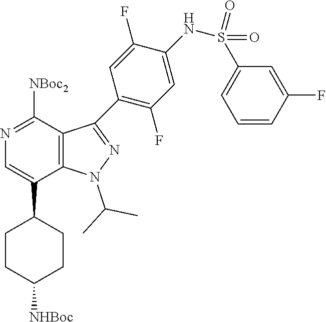 | Intermediate 26 3-Fluorobenzene sulfonyl chloride (CAS: 701-27-9) | 859 | 2.13/Method E |
| 62 | 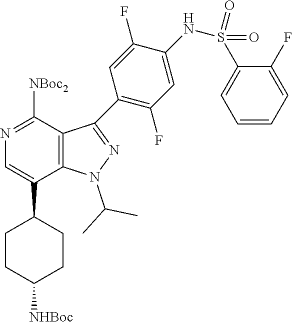 | Intermediate 26 2-Fluorobenzene sulfonyl chloride (CAS: 2905-21-7) | 859 | 2.21/Method H |
| 63 | 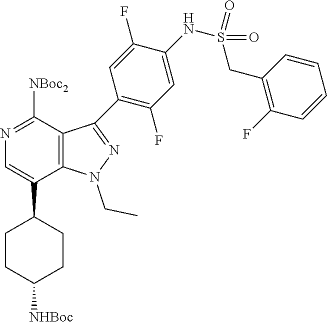 | Intermediate 24 (2-Fluorophenyl) methanesulfonyl chloride (CAS: 24974- 71-8) | 859 | 3.53/Method F |
| 64 | 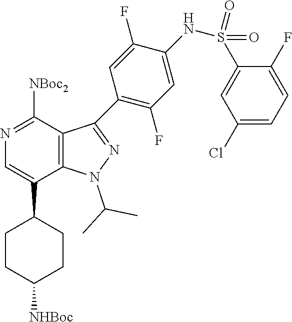 | Intermediate 26 5-Chloro-2- fluorobenzene sulfonyl chloride (CAS: 351003- 49-1) | 893/895 | 2.65/Method H |
| 65 | 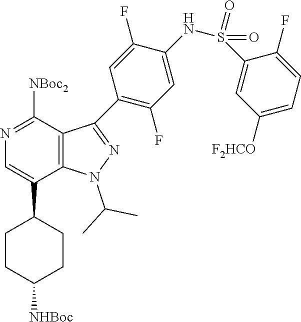 | Intermediate 26 (5-Difluoro methoxy)- 2-fluorobenzene sulfonyl chloride (CAS: 1261746-78-4) | 925 | 1.87/Method D |
| 66 | 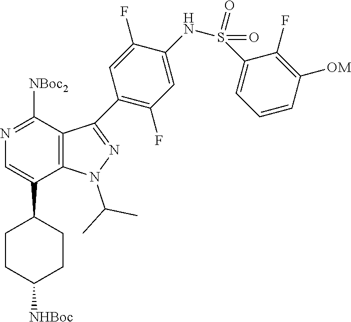 | Intermediate 26 2-Fluoro-3- methoxybenzene sulfonyl chloride (CAS: 1214350-99-8) | 889 | 2.20/Method H |
| 67 | 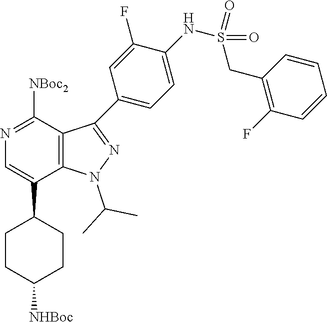 | Intermediate 22 (2-Fluorophenyl) methanesulfonyl chloride (CAS: 24974- 71-8) | 855 | 2.80/Method H |
| 68 | 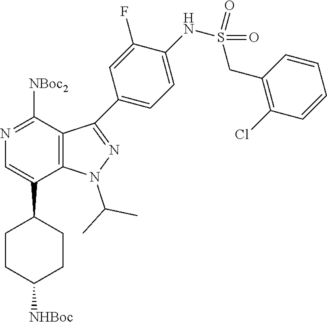 | Intermediate 22 (2-Chlorophenyl) methanesulfonyl chloride (CAS: 77421- 13-7) | 871/873 | 1.74/Method D |
| 69 | 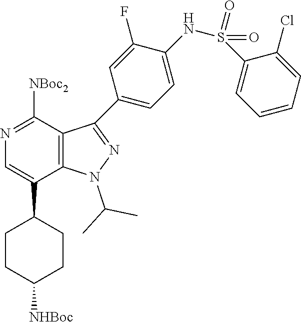 | Intermediate 22 2-Chlorobenzene sulfonyl chloride (CAS: 2905-23-9) | 857/859 | 1.56/Method G |
| 70 | 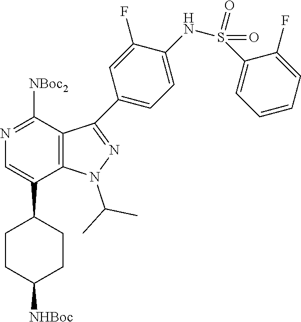 | Intermediate 22 2-Fluorobenzene sulfonyl chloride (CAS: 2905-21-7) | 841 | 1.89/Method D |
| 167 | 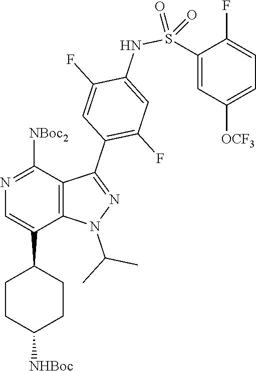 | Intermediate 26 2-Fluoro-5- (trifluoromethoxy) benzene sulfonyl chloride (CAS: 1446478-70-1) | 943 | 1.86/Method D |
| 168 | 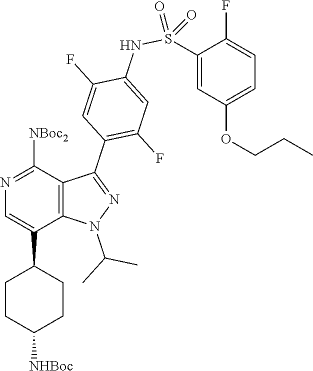 | Intermediate 26 Intermediate 121 | 917 | 1.88/Method D |
| 169 | 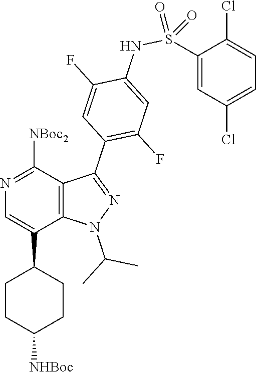 | Intermediate 26 2,5-Dichlorobenzene sulfonyl chloride (CAS: 5402-73-3) | 909/911/913 | 1.89/Method D |
| 170 | 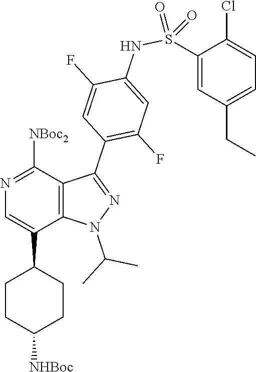 | Intermediate 26 2-Chloro-5- ethylbenzene sulfonyl chloride (CAS: 1254302-08-3) | 903/905 | 1.86/Method D |

### Preparation of intermediate 71: N-(4-(4-amino-7-((1r,4r)-4-aminocyclohexyl)-1-isopropyl-1H-pyrazolo[4,3-c]pyridin-3-yl)-2-fluorophenyl)-2-fluorobenzenesulfonamide

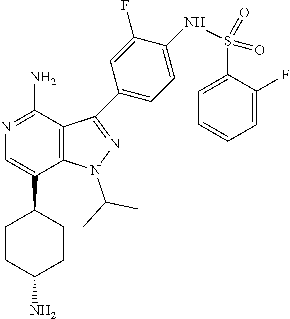

Trifluoroacetic acid (8.0 mL, 11.99 g, 105.11 mmol) was added to a stirred solution of tert-butyl (tert-butoxycarbonyl)(7-41r,4r)-4-((tert-butoxy-carbonyl)amino)cyclohexyl)-3-(3-fluoro-4-((2-fluoro-phenyl)sulfonamido)phenyl)-1-isopropyl-1H-pyrazolo[4,3-c]pyridin-4-yl)carbamate (intermediate 29) (3.40 g, 4.04 mmol) in DCM (30 mL) and the solution stirred at RT for 18 h. The solution was concentrated in vacuo, the residue dissolved in MeOH (5 mL) and the product solution charged to a 50 g ISOLUTE® SCX-2 cartridge pre-wetted with MeOH. The cartridge was eluted with MeOH to wash off the trifluoroacetic acid then the cartridge was washed with a solution of ammonia in MeOH (100 mL, 2N) to release the target compound from the SCX-2 solid. The eluted solution was concentrated in vacuo to give the title compound (2.07 g, 89%) as a white solid. ¹H NMR (400 MHz, DMSO-d₆) δ: 7.78 (1H, td, J=7.8 and 1.8 Hz), 7.57 (1H, s), 7.45-7.38 (1H, m), 7.24 (1H, dd, J=9 and 8.5 Hz), 7.21-7.13 (2H, m), 7.07 (1H, dd, J=12.1 and 2.1 Hz), 6.95 (1H, dd, J=8.3 and 1.8 Hz), 5.51 (2H, bs), 4.82 (1H, septet, J=6.5 Hz), 4.10 (1.1H, bs, NH₂), 3.19-3.08 (1H, m), 2.95-2.84 (1H, m), 2.13-2.03 (2H, m), 2.02-1.92 (2H, m), 1.72-1.43 (4H, m), 1.50 (6H, d, J=6.5 Hz).

Intermediates 72-116 (Table 9) were prepared by using an analogous reaction protocol as described for intermediate 71 from the appropriate starting material.

TABLE 9

| | | Starting | LCMS m/z | HPLC Rₜ |
|---|---|---|---|---|
| Intermediate | Structure | material | (M + H) | (min)/Method |
| 72 | 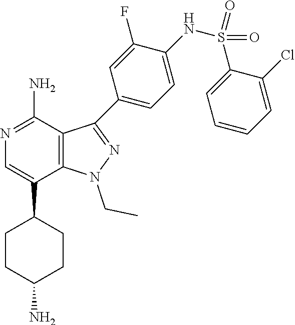 [SMILES: CCn1nc(-c2ccc(NS(=O)(=O)c3ccccc3Cl)c(F)c2)c2c(N)ncc([C@H]3CC[C@H](N)CC3)c21] | Intermediate 30 | 543 | 1.53/Method H |
| 73 | 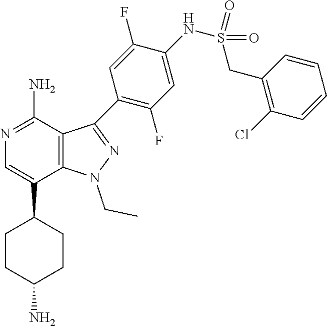 [SMILES: CCn1nc(-c2cc(F)c(NS(=O)(=O)Cc3ccccc3Cl)cc2F)c2c(N)ncc([C@H]3CC[C@H](N)CC3)c21] | Intermediate 31 | 575 | 1.63/Method H |
| 74 | 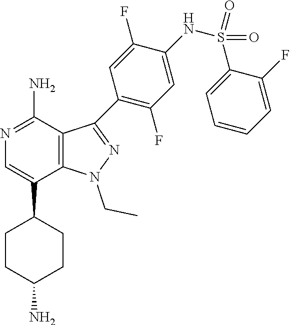 [SMILES: CCn1nc(-c2cc(F)c(NS(=O)(=O)c3ccccc3F)cc2F)c2c(N)ncc([C@H]3CC[C@H](N)CC3)c21] | Intermediate 32 | 545 | 1.96/Method I |
| 75 | 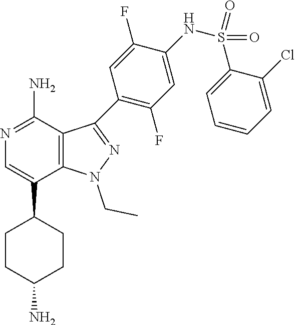 [SMILES: CCn1nc(-c2cc(F)c(NS(=O)(=O)c3ccccc3Cl)cc2F)c2c(N)ncc([C@H]3CC[C@H](N)CC3)c21] | Intermediate 33 | 561 | 1.54/Method H |
| 76 | 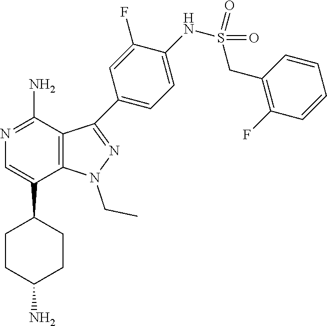 [SMILES: CCn1nc(-c2ccc(NS(=O)(=O)Cc3ccccc3F)c(F)c2)c2c(N)ncc([C@H]3CC[C@H](N)CC3)c21] | Intermediate 34 | 541 | 1.56/Method H |
| 77 | 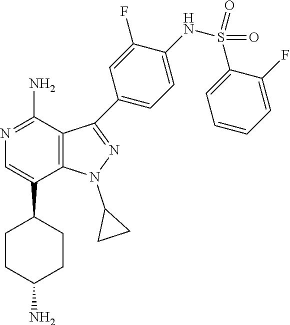 [SMILES: Nc1ncc([C@H]2CC[C@H](N)CC2)c2c1c(-c1ccc(NS(=O)(=O)c3ccccc3F)c(F)c1)nn2C1CC1] | Intermediate 35 | 539 | 1.50/Method H |
| 78 | 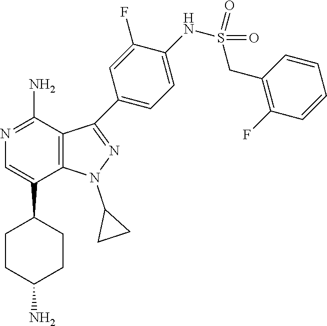 [SMILES: Nc1ncc([C@H]2CC[C@H](N)CC2)c2c1c(-c1ccc(NS(=O)(=O)Cc3ccccc3F)c(F)c1)nn2C1CC1] | Intermediate 36 | 553 | 2.05/Method I |
| 79 | 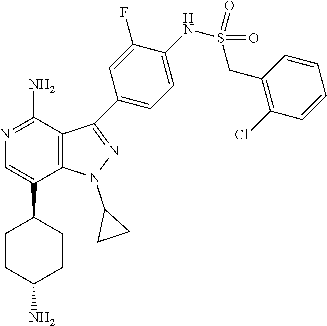 [SMILES: Nc1ncc([C@H]2CC[C@H](N)CC2)c2c1c(-c1ccc(NS(=O)(=O)Cc3ccccc3Cl)c(F)c1)nn2C1CC1] | Intermediate 37 | 569 | 2.20/Method I |
| 80 | 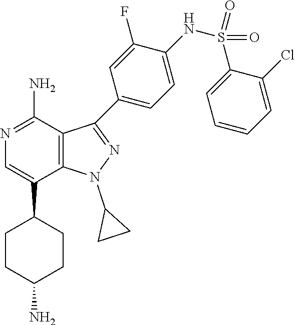 [SMILES: Nc1ncc([C@H]2CC[C@H](N)CC2)c2c1c(-c1ccc(NS(=O)(=O)c3ccccc3Cl)c(F)c1)nn2C1CC1] | Intermediate 38 | 555 | 2.04/Method I |
| 81 | 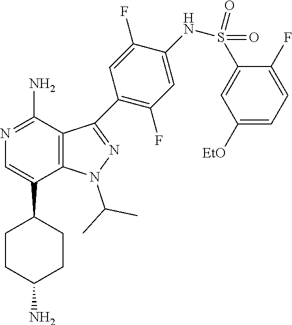 [SMILES: CCOc1ccc(F)c(S(=O)(=O)Nc2cc(F)c(-c3nn(C(C)C)c4c3c(N)ncc4[C@H]3CC[C@H](N)CC3)cc2F)c1] | Intermediate 39 | 603 | 0.91/Method D |
| 82 | 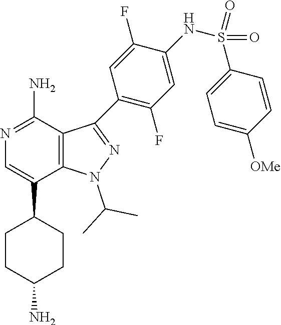 [SMILES: COc1ccc(S(=O)(=O)Nc2cc(F)c(-c3nn(C(C)C)c4c3c(N)ncc4[C@H]3CC[C@H](N)CC3)cc2F)cc1] | Intermediate 40 | 571 | 1.02/Method G |
| 83 | 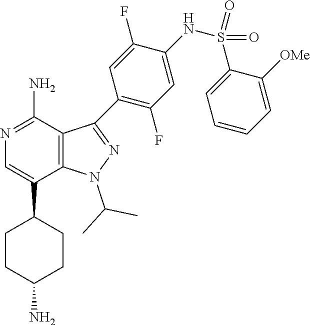 [SMILES: COc1ccccc1S(=O)(=O)Nc1cc(F)c(-c2nn(C(C)C)c3c2c(N)ncc3[C@H]2CC[C@H](N)CC2)cc1F] | Intermediate 41 | 571 | 1.49/Method H |
| 84 | 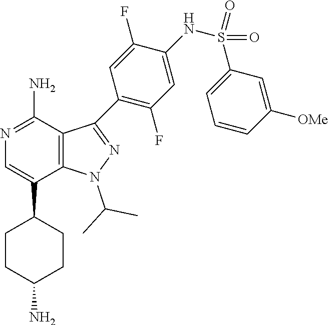 [SMILES: COc1cccc(S(=O)(=O)Nc2cc(F)c(-c3nn(C(C)C)c4c3c(N)ncc4[C@H]3CC[C@H](N)CC3)cc2F)c1] | Intermediate 42 | 571 | 1.52/Method H |
| 85 | 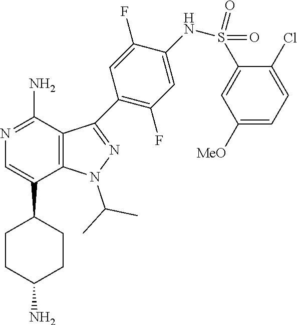 [SMILES: COc1ccc(Cl)c(S(=O)(=O)Nc2cc(F)c(-c3nn(C(C)C)c4c3c(N)ncc4[C@H]3CC[C@H](N)CC3)cc2F)c1] | Intermediate 43 | 605/607 | 1.60/Method H |
| 86 | 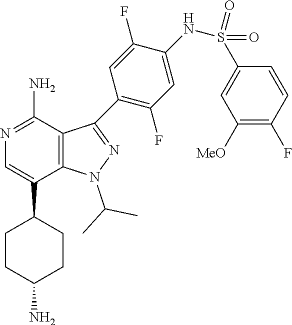 [SMILES: COc1cc(S(=O)(=O)Nc2cc(F)c(-c3nn(C(C)C)c4c3c(N)ncc4[C@H]3CC[C@H](N)CC3)cc2F)ccc1F] | Intermediate 44 | 589 | 0.92/Method D |
| 87 | 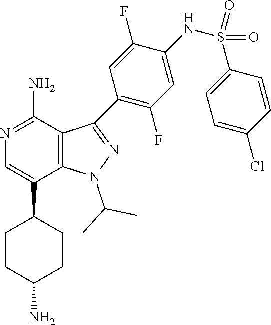 [SMILES: CC(C)n1nc(-c2cc(F)c(NS(=O)(=O)c3ccc(Cl)cc3)cc2F)c2c(N)ncc([C@H]3CC[C@H](N)CC3)c21] | Intermediate 45 | 575/578 | 1.65/Method H |
| 88 | 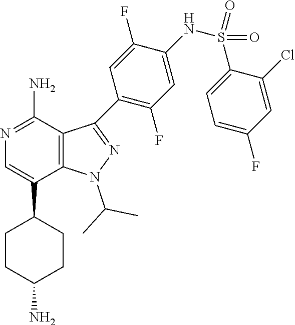 [SMILES: CC(C)n1nc(-c2cc(F)c(NS(=O)(=O)c3ccc(F)cc3Cl)cc2F)c2c(N)ncc([C@H]3CC[C@H](N)CC3)c21] | Intermediate 46 | 593/595 | 1.00/Method G |
| 89 | 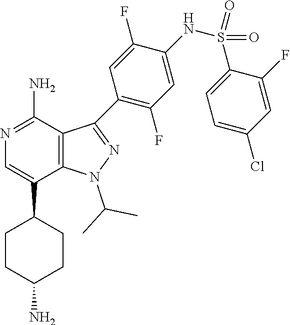 [SMILES: CC(C)n1nc(-c2cc(F)c(NS(=O)(=O)c3ccc(Cl)cc3F)cc2F)c2c(N)ncc([C@H]3CC[C@H](N)CC3)c21] | Intermediate 47 | 593/595 | 1.07/Method G |
| 90 | 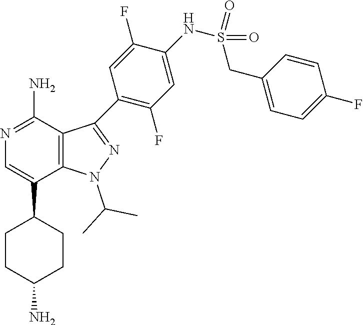 [SMILES: CC(C)n1nc(-c2cc(F)c(NS(=O)(=O)Cc3ccc(F)cc3)cc2F)c2c(N)ncc([C@H]3CC[C@H](N)CC3)c21] | Intermediate 48 | 573 | 1.03/Method G |
| 91 | 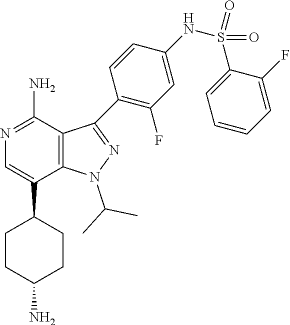 [SMILES: CC(C)n1nc(-c2ccc(NS(=O)(=O)c3ccccc3F)cc2F)c2c(N)ncc([C@H]3CC[C@H](N)CC3)c21] | Intermediate 49 | 541 | 1.00/Method G |
| 92 | 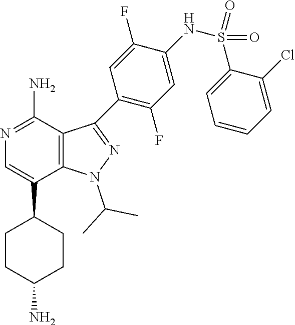 [SMILES: CC(C)n1nc(-c2cc(F)c(NS(=O)(=O)c3ccccc3Cl)cc2F)c2c(N)ncc([C@H]3CC[C@H](N)CC3)c21] | Intermediate 50 | 575/577 | 1.02/Method G |
| 93 | 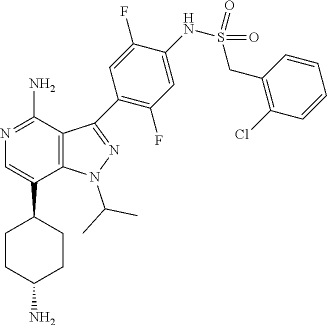 [SMILES: CC(C)n1nc(-c2cc(F)c(NS(=O)(=O)Cc3ccccc3Cl)cc2F)c2c(N)ncc([C@H]3CC[C@H](N)CC3)c21] | Intermediate 51 | 589/591 | 1.02/Method G |
| 94 | 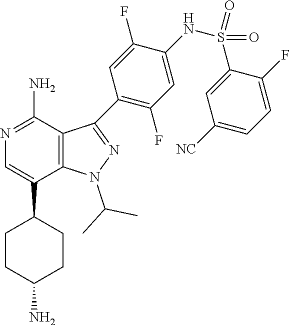 [SMILES: CC(C)n1nc(-c2cc(F)c(NS(=O)(=O)c3cc(C#N)ccc3F)cc2F)c2c(N)ncc([C@H]3CC[C@H](N)CC3)c21] | Intermediate 52 | 584 | 1.02/Method G |
| 95 | 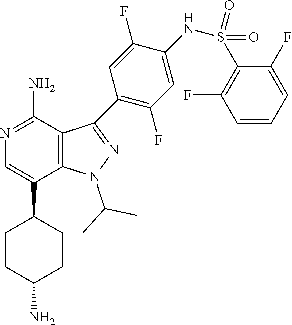 [SMILES: CC(C)n1nc(-c2cc(F)c(NS(=O)(=O)c3c(F)cccc3F)cc2F)c2c(N)ncc([C@H]3CC[C@H](N)CC3)c21] | Intermediate 53 | 577 | 0.99/Method G |
| 96 | 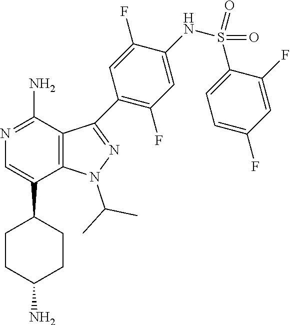 [SMILES: CC(C)n1nc(-c2cc(F)c(NS(=O)(=O)c3ccc(F)cc3F)cc2F)c2c(N)ncc([C@H]3CC[C@H](N)CC3)c21] | Intermediate 54 | 577 | 1.01/Method G |
| 97 | 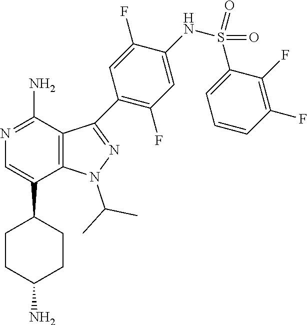 [SMILES: CC(C)n1nc(-c2cc(F)c(NS(=O)(=O)c3cccc(F)c3F)cc2F)c2c(N)ncc([C@H]3CC[C@H](N)CC3)c21] | Intermediate 55 | 577 | 1.02/Method G |
| 98 | 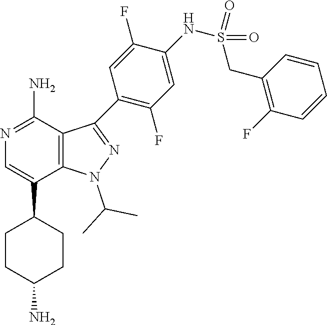 [SMILES: CC(C)n1nc(-c2cc(F)c(NS(=O)(=O)Cc3ccccc3F)cc2F)c2c(N)ncc([C@H]3CC[C@H](N)CC3)c21] | Intermediate 56 | 573 | 0.86/Method D |
| 99 | 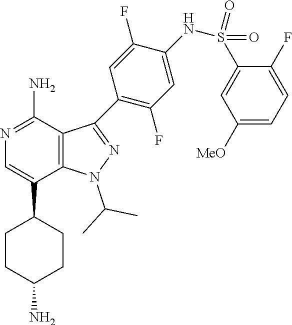 [SMILES: COc1ccc(F)c(S(=O)(=O)Nc2cc(F)c(-c3nn(C(C)C)c4c3c(N)ncc4[C@H]3CC[C@H](N)CC3)cc2F)c1] | Intermediate 57 | 589 | 0.97/Method G |
| 100 | 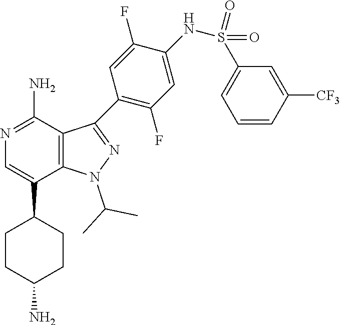 [SMILES: CC(C)n1nc(-c2cc(F)c(NS(=O)(=O)c3cccc(C(F)(F)F)c3)cc2F)c2c(N)ncc([C@H]3CC[C@H](N)CC3)c21] | Intermediate 58 | 609 | 1.87/Method H |
| 101 | 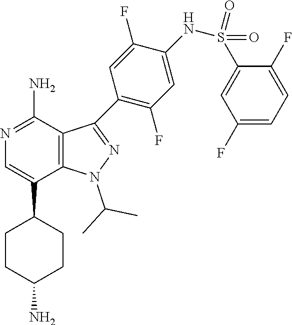 [SMILES: CC(C)n1nc(-c2cc(F)c(NS(=O)(=O)c3cc(F)ccc3F)cc2F)c2c(N)ncc([C@H]3CC[C@H](N)CC3)c21] | Intermediate 59 | 577 | 1.01/Method G |
| 102 | 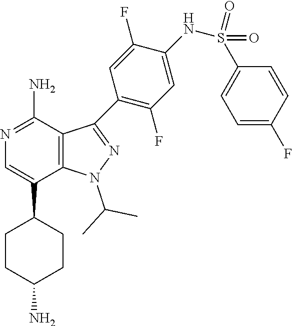 [SMILES: CC(C)n1nc(-c2cc(F)c(NS(=O)(=O)c3ccc(F)cc3)cc2F)c2c(N)ncc([C@H]3CC[C@H](N)CC3)c21] | Intermediate 60 | 559 | 0.97/Method G |
| 103 | 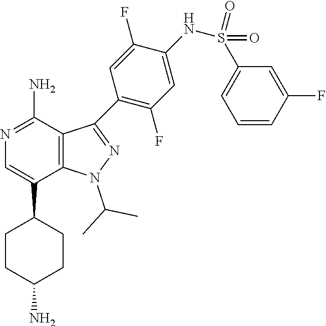 [SMILES: CC(C)n1nc(-c2cc(F)c(NS(=O)(=O)c3cccc(F)c3)cc2F)c2c(N)ncc([C@H]3CC[C@H](N)CC3)c21] | Intermediate 61 | 559 | 0.97/Method G |
| 104 | 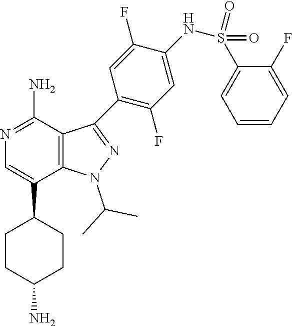 [SMILES: CC(C)n1nc(-c2cc(F)c(NS(=O)(=O)c3ccccc3F)cc2F)c2c(N)ncc([C@H]3CC[C@H](N)CC3)c21] | Intermediate 62 | 559 | 1.51/Method H |
| 105 | 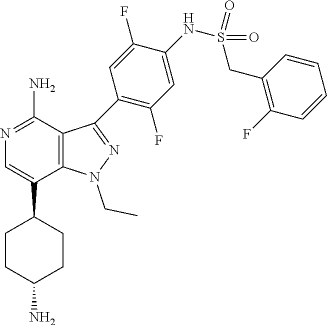 [SMILES: CCn1nc(-c2cc(F)c(NS(=O)(=O)Cc3ccccc3F)cc2F)c2c(N)ncc([C@H]3CC[C@H](N)CC3)c21] | Intermediate 63 | 559 | 1.55/Method H |
| 106 | 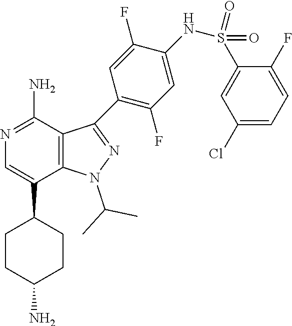 [SMILES: CC(C)n1nc(-c2cc(F)c(NS(=O)(=O)c3cc(Cl)ccc3F)cc2F)c2c(N)ncc([C@H]3CC[C@H](N)CC3)c21] | Intermediate 64 | 593/595 | 1.06/Method G |
| 107 | 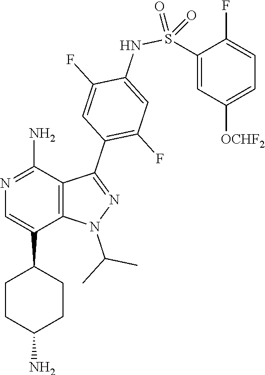 [SMILES: CC(C)n1nc(-c2cc(F)c(NS(=O)(=O)c3cc(OC(F)F)ccc3F)cc2F)c2c(N)ncc([C@H]3CC[C@H](N)CC3)c21] | Intermediate 65 | 625 | 0.93/Method D |
| 108 | 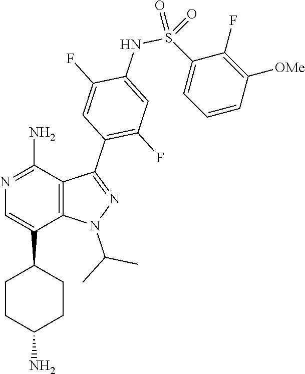 [SMILES: COc1cccc(S(=O)(=O)Nc2cc(F)c(-c3nn(C(C)C)c4c3c(N)ncc4[C@H]3CC[C@H](N)CC3)cc2F)c1F] | Intermediate 66 | 589 | 1.50/Method H |
| 109 | 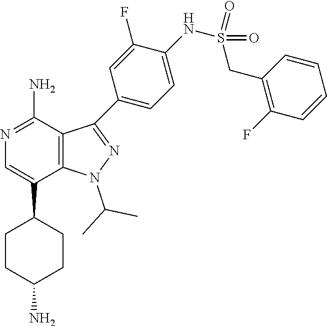 [SMILES: CC(C)n1nc(-c2ccc(NS(=O)(=O)Cc3ccccc3F)c(F)c2)c2c(N)ncc([C@H]3CC[C@H](N)CC3)c21] | Intermediate 67 | 555 | 2.17/Method I |
| 110 | 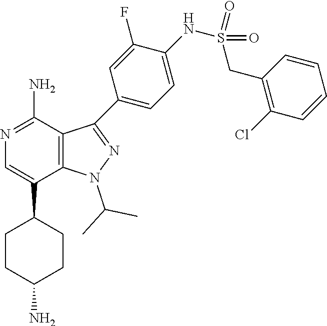 [SMILES: CC(C)n1nc(-c2ccc(NS(=O)(=O)Cc3ccccc3Cl)c(F)c2)c2c(N)ncc([C@H]3CC[C@H](N)CC3)c21] | Intermediate 68 | 571/573 | 0.77/Method D |
| 111 | 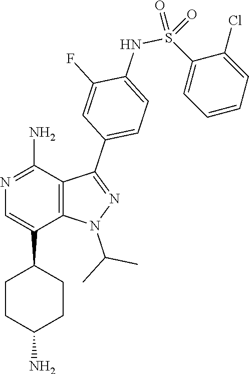 [SMILES: CC(C)n1nc(-c2ccc(NS(=O)(=O)c3ccccc3Cl)c(F)c2)c2c(N)ncc([C@H]3CC[C@H](N)CC3)c21] | Intermediate 69 | 557/559 | 1.05/Method G |
| 112 | 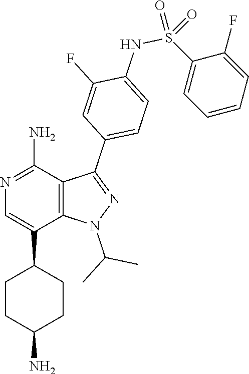 [SMILES: CC(C)n1nc(-c2ccc(NS(=O)(=O)c3ccccc3F)c(F)c2)c2c(N)ncc([C@H]3CC[C@@H](N)CC3)c21] | Intermediate 70 | 541 | 1.73/Method H |
| 113 | 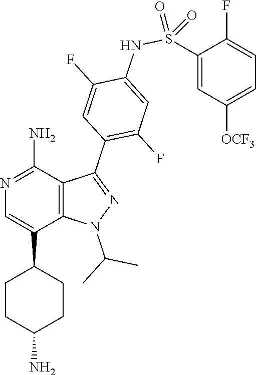 [SMILES: CC(C)n1nc(-c2cc(F)c(NS(=O)(=O)c3cc(OC(F)(F)F)ccc3F)cc2F)c2c(N)ncc([C@H]3CC[C@H](N)CC3)c21] | Intermediate 167 | 643 | 0.93/Method D |
| 114 | 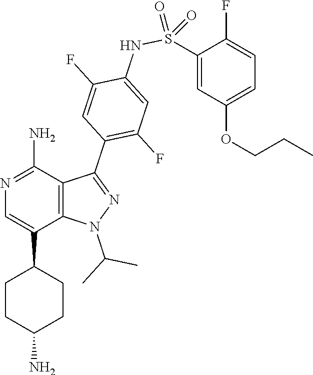 [SMILES: CCCOc1ccc(F)c(S(=O)(=O)Nc2cc(F)c(-c3nn(C(C)C)c4c3c(N)ncc4[C@H]3CC[C@H](N)CC3)cc2F)c1] | Intermediate 168 | 617 | 0.95/Method D |
| 115 | 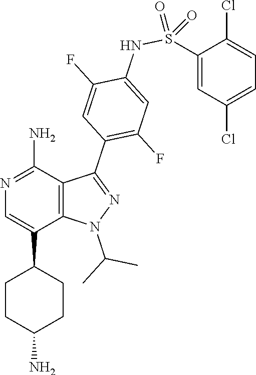 [SMILES: CC(C)n1nc(-c2cc(F)c(NS(=O)(=O)c3cc(Cl)ccc3Cl)cc2F)c2c(N)ncc([C@H]3CC[C@H](N)CC3)c21] | Intermediate 169 | 609/611/613 | 0.92/Method D |
| 116 | 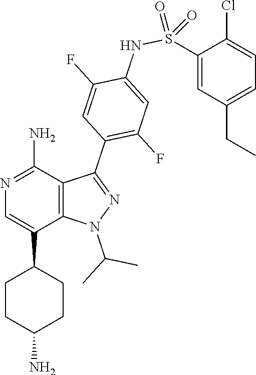 [SMILES: CCc1ccc(Cl)c(S(=O)(=O)Nc2cc(F)c(-c3nn(C(C)C)c4c3c(N)ncc4[C@H]3CC[C@H](N)CC3)cc2F)c1] | Intermediate 170 | 603/605 | 0.82/Method D |

### Preparation of intermediate 117: N-((1r,4r)-4-(4-amino-3-(2,5-difluoro-4-((2-fluorophenyl)sulfonamido)phenyl)-1-isopropyl-1H-pyrazolo[4,3-c]pyridin-7-yl)cyclohexyl)-2-fluoropropanamide

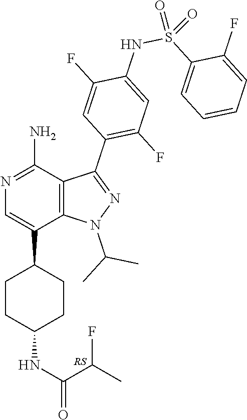

HATU (68 mg, 0.179 mmol) was added to a mixture of N-(4-(4-amino-7-((1r,4r)-4-aminocyclohexyl)-1-isopropyl-1H-pyrazolo[4,3-c]pyridin-3-yl)-2,5-difluorophenyl)-2-fluorobenzenesulfonamide (intermediate 104; 77 mg, 0.138 mmol), 2-fluoropropionic acid (CAS: 6087-13-4; 0.013 mL, 0.165 mmol) and triethylamine (0.038 mL, 0.276 mmol) in DMF (2.0 mL), and the resulting mixture was stirred at RT for 5 min. The mixture was diluted with water and extracted with ethyl acetate. The combined extracts were dried (MgSO4), concentrated in vacuo and purified by chromatography on silica, eluting with DCM/MeOH 0-5%, to give the title compound as a white solid (32 mg, 37%). LCMS (Method D): Rt=1.12 min; m/z [M+H]⁺=633

### Preparation of intermediate 118: N-(4-(4-amino-7-((1r,4r)-4-((2-(2-chloroethoxy)ethyl)amino)cyclohexyl)-1-isopropyl-1H-pyrazolo[4,3-c]pyridin-3-yl)-2,5-ifluorophenyl)-2-fluorobenzenesulfonamide

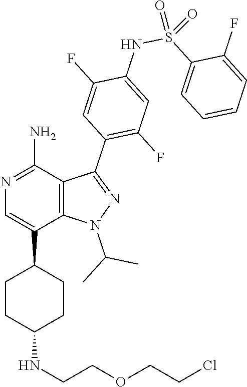

Sodium cyanoborohydride (10 mg, 0.166 mmol) was added to a mixture of 2-(2-chloroethoxy)acetaldehyde (CAS: 284021-70-1; 27 mg, 0.222 mmol), N-(4-(4-amino-7-((1r,4r)-4-aminocyclohexyl)-1-isopropyl-1H-pyrazolo[4,3-c]pyridin-3-yl)-2,5-difluorophenyl)-2-fluorobenzenesulfonamide (intermediate 104) (62 mg, 0.111 mmol) and acetic acid (2 drops) in dry MeOH (3.0 mL) and the mixture stirred at RT for 18 h. The mixture was concentrated in vacuo and then loaded as a solution in MeOH onto a 2 g ISOLUTE® SCX-2 cartridge pre-wetted with MeOH, and the cartridge washed with MeOH until all acids had eluted. The target compound was washed off the SCX-2 solid with 2N ammonia in MeOH (20 mL) and the resulting eluate concentrated in vacuo to give the title compound (74 mg, 100%) as a white solid. LCMS (Method G): Rt=1.11 min; m/z [M+H]⁺=665/667

### Preparation of intermediate 119: 5-Ethoxy-2-fluorobenzenesulfonyl chloride

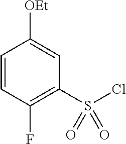 [SMILES: CCOc1ccc(F)c(S(=O)(=O)Cl)c1]

To a suspension of 5-ethoxy-2-fluoroaniline (CAS: 1190075-01-4; 1 g, 6.44 mmol) in 12N aqueous HCl (7 mL) at 0° C. was added, dropwise, a solution of sodium nitrite (0.51 g, 7.41 mmol) in water (6 mL) and the reaction mixture stirred at 0° C. for 30 min during which the solids dissolved. In a separate flask, thionyl chloride (2.2 mL, 29.6 mmol) was added dropwise, to water (14 mL) at 0° C. On complete addition, copper(I) chloride (32 mg, 0.32 mmol) was added and the first solution of diazonium salt introduced dropwise. The reaction was stirred at 0° C. for 30 minutes then extracted with DCM (3×10 mL). The combined extracts were dried (MgSO₄) and concentrated in vacuo to give the title compound as a dark brown oil (1.3 g, 84%). ¹H NMR (400 MHz CDCl₃) δ: 7.43-7.38 (1H, m), 7.27-7.20 (2H, m), 3.87 (3H, s).

### Preparation of intermediate 120: 2-Fluoro-5-propoxyaniline

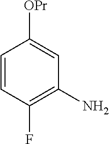

A solution of 1-fluoro-2-nitro-4-propoxybenzene (CAS: 1048368-31-5; 1.27 g, 6.38 mmol) in ethyl acetate (50 mL) was hydrogenated over palladium on carbon (10%; 300 mg) for 2 days. The catalyst was removed by filtration and the eluent concentrated in vacuo to give the title compound as a dark brown oil (1.08 g, 100%). NMR(CDCl₃ 400 MHz) δ: 6.86 (1H, dd, J=10.4, 8.8 Hz), 6.33 (1H, dd, J=7.6, 3.2 Hz), 6.20 (1H, dt, J=8.8, 3.4 Hz), 3.83 (2H, t, J=6.6 Hz), 3.68 (2H, br s), 1.76 (2H, sext, J=7.1 Hz), 1.01 (3H, t, J=7.3 Hz).

Intermediate 121 (Table 10) was prepared by using an analogous reaction protocol as described for intermediate 119 from the appropriate starting material.

TABLE 10

| | | Starting | |
|---|---|---|---|
| Intermediate | Structure | material | NMR |
| 121 | 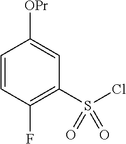 | Intermediate 120 | (CDCl₃ 400 MHz) δ: 7.45-7.37 (1H, m), 7.25-7.20 (2H, m), 3.95 (2H, t, J = 6.4 Hz), 1.83 (2H, sext, J = 6.4 Hz), 1.05 (3H, t, J = 7.4 Hz). |

### Preparation of intermediate 122: 7-(4-aminocyclohex-1-en-1-yl)-3-bromo-1-isopropyl-1H-pyrazolo[4,3-c]pyridin-4-amine

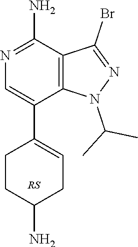

Trifluoroacetic acid (15.9 mL, 23.66 g, 207.50 mmol) was added to a stirred solution of tert-butyl (3-bromo-7-(4-((tert-butoxycarbonyl)amino)cyclohex-1-en-1-yl)-1-isopropyl-1H-pyrazolo[4,3-c]pyridin-4-yl)(tert-butoxycarbonyl)carbamate (intermediate 13) (4.50 g, 6.92 mmol) in DCM (50 mL) and the solution stirred at RT for 18 h. The solution was concentrated in vacuo, azeotrope twice with MeOH/toluene, the residue dissolved in MeOH (10 mL) and charged to a 50 g ISOLUTE® SCX-2 cartridge pre-wetted with MeOH. The cartridge was eluted with MeOH, then the cartridge was washed with a solution of ammonia in MeOH (100 mL, 2N) to release the target compound from the SCX-2 solid. The eluted solution was concentrated in vacuo to give the title compound (2.42 g, 85%) as a pale orange foam. LCMS (Method G): Rt=1.22 min; m/z [M+H]⁺=350/352

Intermediates 123 and 124 (Table 11) were prepared by using an analogous reaction protocol as described for intermediate 122 from the appropriate starting material.

TABLE 11

| | | | LCMS | HPLC Rₜ |
|---|---|---|---|---|
| Inter- | | Starting | m/z | (min)/ |
| mediate | Structure | material | (M + H) | Method |
| 123 | 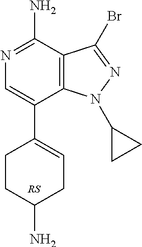 [SMILES: Nc1ncc(C2=CCC(N)CC2)c2c1c(Br)nn2C1CC1] | Intermediate 15 | 348/350 | 0.18/ Method D |
| 124 | 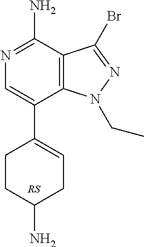 [SMILES: CCn1nc(Br)c2c(N)ncc(C3=CCC(N)CC3)c21] | Intermediate 14 | 336/338 | 1.76/ Method H |

### Preparation of intermediate 125: 3-bromo-1-isopropyl-7-(4-(oxetan-3-ylamino)cyclohex-1-en-1-yl)-1H-pyrazolo[4,3-c]pyridin-4-amine

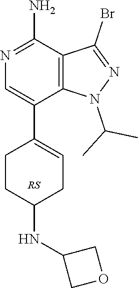

3-Oxetanone (CAS: 6704-31-0; 0.33 mL, 0.37 g, 5.14 mmol) was added to a stirred suspension of 7-(4-aminocyclohex-1-en-1-yl)-3-bromo-1-isopropyl-1H-pyrazolo[4,3-c]pyridin-4-amine (intermediate 122) (1.50 g, 4.28 mmol) and acetic acid (0.74 mL, 0.77 g, 12.85 mmol) in DCM (40 mL) at RT under an argon atmosphere. Sodium triacetoxyborohydride (1.27 g, 6.00 mmol) was added and the mixture stirred under an argon atmosphere for 18 h. Second portions of 3-oxetanone (CAS: 6704-31-0; 0.11 mL, 0.123 g, 1.71 mmol) and sodium triacetoxyborohydride (0.40 g, 1.89 mmol) were added and stirring continued for a further 4 h. The mixture was treated with saturated aqueous NaHCO₃ solution and extracted with DCM. The aqueous layer was further extracted with DCM and the combined extracts were washed with saturated brine and passed through a phase separator cartridge. The eluate was loaded onto a 20 g ISOLUTE® SCX-2 cartridge pre-wetted with MeOH, and the cartridge washed with DCM, MeOH and DCM. The crude product was washed off the SCX-2 with 7N ammonia in MeOH solution and the resulting eluate concentrated in vacuo to give the title compound as an off-white solid (1.69 g, 97%). LCMS (Method H): Rt=2.04 min; m/z [M+H]⁺=406/408.

Intermediates 126-128 (Table 12) were prepared by using an analogous reaction protocol as described for intermediate 125 from the appropriate starting material.

TABLE 12

| | | | LCMS | HPLC Rₜ |
|---|---|---|---|---|
| Inter- | | Starting | m/z | (min)/ |
| mediate | Structure | material | (M + H) | Method |
| 126 | 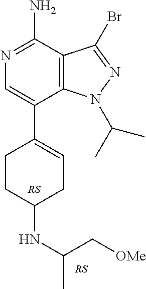 [SMILES: COCC(C)NC1CC=C(c2cnc(N)c3c(Br)nn(C(C)C)c23)CC1] | Intermediate 122 Methoxy acetone (Cas: 5878-19-3) | 422/424 | 0.60/ Method D |
| 127 | 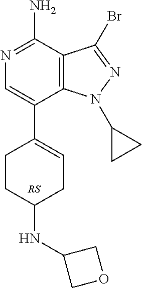 [SMILES: Nc1ncc(C2=CCC(NC3COC3)CC2)c2c1c(Br)nn2C1CC1] | Intermediate 123 3- Oxetanone (CAS: 6704-31-0) | 404/406 | 1.89/ Method H |
| 128 | 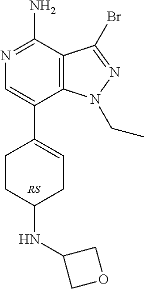 [SMILES: CCn1nc(Br)c2c(N)ncc(C3=CCC(NC4COC4)CC3)c21] | Intermediate 124 3- Oxetanone (CAS: 6704-31-0) | 392/394 | 1.83/ Method H |

### Preparation of intermediate 129: 1-fluoropropan-2-yl trifluoromethanesulfonate

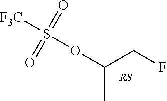

A solution of 1-fluoropropan-2-ol (CAS 430-50-2; 200 mg, 2.56 mmol) in dry DCM (3.0 mL) was cooled to 0° C., treated with 2,6-lutidine (325 mg, 3.03 mmol), stirred for 10 minutes and then trifluoromethanesulfonic anhydride (CAS 358-23-6; 856 mg, 3.04 mmol) added dropwise. After 20 minutes, the reaction was quenched with saturated aqueous NH₄Cl and extracted with DCM. The combined extracts were washed with saturated aqueous NaHCO₃ and dried (Mg₂SO₄). The resultant solution was passed through a SCX-2 cartridge and the eluent concentrated in vacuo to give the title compound (367 mg, 68%) as a yellow residue.

### Preparation of intermediate 130: 3-bromo-7-(4-((1-fluoropropan-2-yl)amino)cyclohex-1-en-1-yl)-1-isopropyl-1H-pyrazolo[4,3-c]pyridin-4-amine

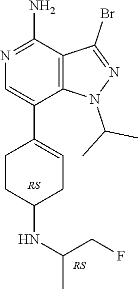

1-fluoropropan-2-yl trifluoromethanesulfonate (intermediate 129) (0.12 g, 0.57 mmol) dissolved in 1,4-dioxane was added slowly to a solution of 7-(4-aminocyclohex-1-en-1-yl)-3-bromo-1-isopropyl-1H-pyrazolo[4,3-c]pyridin-4-amine (intermediate 122) (0.20 g, 0.57 mmol) and DIPEA (0.20 mL, 0.15 g, 1.15 mmol) in 1,4-dioxane (4 mL) at RT under an argon atmosphere. The resulting mixture was stirred at RT for 24 h. A second portion of 1-fluoropropan-2-yl trifluoromethanesulfonate (intermediate 122) (0.05 g, 0.24 mmol) was added and stirring continued for a further 2 h. The reaction mixture was diluted with DCM and water, the organic layer passed through a phase separator cartridge and concentrated in vacuo. The product was purified by column chromatography on SiO₂, eluting with a gradient of 0-10% ammonia in MeOH (2N)/DCM to give the title compound (0.18 g, 75%) as a beige solid. LCMS (Method D): Rt=0.55 min; m/z [M+H]⁺=410/412

Intermediates 131-133 (Table 13) were prepared by using an analogous reaction protocol as described for intermediate 130 from the appropriate starting material.

TABLE 13

| | | | LCMS m/z | HPLC Rₜ |
|---|---|---|---|---|
| Intermediate | Structure | Starting materials | (M + H) | (min)/Method |
| 131 | 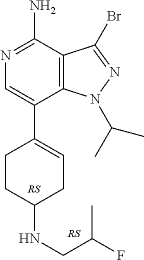 [SMILES: CC(F)CNC1CC=C(c2cnc(N)c3c(Br)nn(C(C)C)c23)CC1] | Intermediate 122 Intermediate 128 | 410/412 | 0.57/Method D |
| 132 | 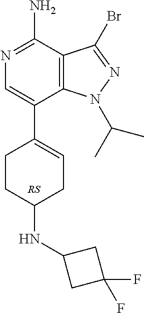 [SMILES: CC(C)n1nc(Br)c2c(N)ncc(C3=CCC(NC4CC(F)(F)C4)CC3)c21] | Intermediate 122 Intermediate 127 | 440/442 | 0.61/Method D |
| 133 | 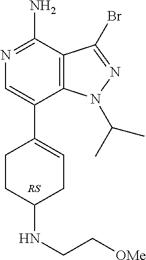 [SMILES: COCCNC1CC=C(c2cnc(N)c3c(Br)nn(C(C)C)c23)CC1] | Intermediate 122 2-Methoxyethyl triflate (CAS: 112981-50-7) | 408/410 | 1.37/Method G |

### Preparation of intermediate 134: 3,3-difluorocyclobutyl trifluoromethanesulfonate

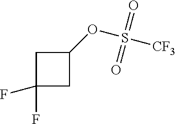 [SMILES: O=S(=O)(OC1CC(F)(F)C1)C(F)(F)F]

A solution of 3,3-difluorocyclobutanol (CAS: 637031-88-2; 200 mg, 1.85 mmol), 2,6 lutidine (236 mg, 2.20 mmol) and DCM (5 mL) was cooled in an ice-bath and trifluoromethanesulfonic anhydride (620 mg, 2.20 mmol) added dropwise over 10 minutes. The mixture was diluted with saturated aqueous NH₄Cl and, after 45 minutes, the phases were separated. The organic phase was washed with saturated aqueous NaHCO₃, dried (Na₂SO₄) and passed through an SCX-2 cartridge. The eluent was concentrated in vacuo to give the title compound as a pale brown oil (250 mg, 56%). ¹H NMR (400 MHz, CDCl₃) δ: 5.27-5.17 (1H, m), 3.25-2.96 (4H, m).

Intermediate 135 (Table 14) was prepared by using an analogous reaction protocol as described for intermediate 134 from the appropriate starting material.

TABLE 14

| Intermediate | Structure | Starting material |
|---|---|---|
| 135 (CAS: 1630196-24-5) | 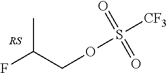 | 2-Fluoropropan-1-ol (CAS: 3824-87-1) |

### Preparation of intermediate 136: N-(2-fluoro-4-(4,4,5,5-tetramethyl-1,3,2-dioxaborolan-2-yl)phenyl)-1-(2-fluorophenyl)methanesulfonamide

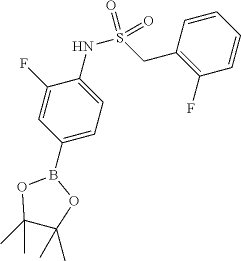 [SMILES: CC1(C)OB(c2ccc(NS(=O)(=O)Cc3ccccc3F)c(F)c2)OC1(C)C]

A solution of 2-fluoro-4-(4,4,5,5-tetramethyl-1,3,2-dioxaborolan-2-yl)aniline (CAS: 819058-34-9; 1.00 g, 4.22 mmol) and pyridine (1.0 mL, 12.65 mmol) in DCM (20 mL) was cooled in an ice-bath and (2-fluorophenyl)methanesulfonyl chloride (CAS: 24974-71-8; 5.18 g, 26.63 mmol) in DCM (10 mL) added dropwise over 5 minutes. The mixture was warmed to RT overnight and then partitioned between an aqueous NH₄Cl and DCM. The organic layer was dried (Na₂SO₄) and concentrated in vacuo. The resultant residue was re-dissolved in DCM and purified by chromatography on SiO₂, eluting with a gradient of EtOAc and DCM (0-8%). Fractions were concentrated in vacuo and the residue triturated with a mixture of Et₂O and cyclohexane to give the title compound as a white solid (725 mg, 42%). LCMS (Method G): Rt=0.62 min; m/z [M−H]⁻=408

Intermediates 137-142 (Table 15) were prepared by using an analogous reaction protocol as described for intermediate 136 from the appropriate starting materials.

TABLE 15

| | | | LCMS | |
|---|---|---|---|---|
| | | | m/z | HPLC Rₜ |
| Intermediate | Structure | Starting materials | (M + H) | (min)/Method |
| 137 | 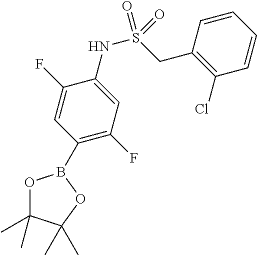 [SMILES: CC1(C)OB(c2cc(F)c(NS(=O)(=O)Cc3ccccc3Cl)cc2F)OC1(C)C] | 2-Chlorobenzyl sulfonyl chloride (CAS: 77421-13-7) 2,5-Difluoro-4-(4,4,5,5- tetramethyl-1,3,2- dioxaborolan-2-yl) aniline (CAS: 939807- 75-7) | Appears as boronic acid 360 [M − H]⁻ | 1.11/Method D |
| 138 | 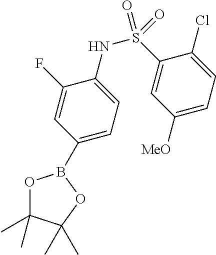 [SMILES: COc1ccc(Cl)c(S(=O)(=O)Nc2ccc(B3OC(C)(C)C(C)(C)O3)cc2F)c1] | 2-Chloro-5- methoxybenzene sulfonyl chloride (CAS: 201935-41-3) 2-Fluoro-4-(4,4,5,5- tetramethyl-1,3,2- dioxaborolan-2- yl)aniline (CAS: 819058-34-9) | 442 | 1.60/Method D |
| 139 | 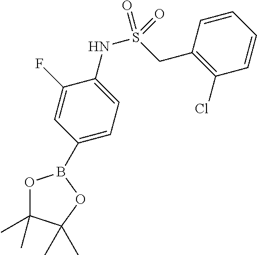 [SMILES: CC1(C)OB(c2ccc(NS(=O)(=O)Cc3ccccc3Cl)c(F)c2)OC1(C)C] | 2-Chlorobenzyl sulfonyl chloride (CAS: 77421-13-7) 2-Fluoro-4-(4,4,5,5- tetramethyl-1,3,2- dioxaborolan-2- yl)aniline(CAS: 819058-34-9) | [M − H]⁻ 424/426 | 1.58/Method D |
| 140 | 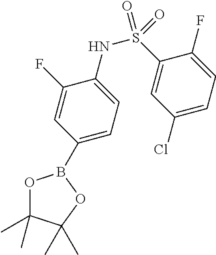 [SMILES: CC1(C)OB(c2ccc(NS(=O)(=O)c3cc(Cl)ccc3F)c(F)c2)OC1(C)C] | 5-Chloro-2- fluorobenzenesulfonyl chloride (CAS: 351003-49-1) 2-Fluoro-4-(4,4,5,5- tetramethyl-1,3,2- dioxaborolan-2- yl)aniline (CAS: 819058-34-9) | 430 | 1.62/Method D |
| 141 | 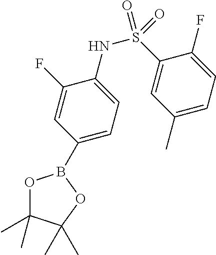 [SMILES: Cc1ccc(F)c(S(=O)(=O)Nc2ccc(B3OC(C)(C)C(C)(C)O3)cc2F)c1] | 2-Fluoro-5- methylbenzenesulfonyl chloride (CAS: 870704-14-6) 2-Fluoro-4-(4,4,5,5- tetramethyl-1,3,2- dioxaborolan-2- yl)aniline (CAS: 819058-34-9) | 410 | 1.59/Method D |
| 142 | 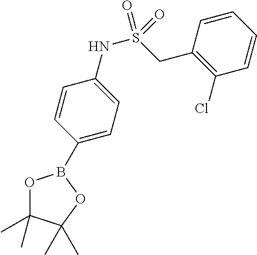 [SMILES: CC1(C)OB(c2ccc(NS(=O)(=O)Cc3ccccc3Cl)cc2)OC1(C)C] | 2-Chlorobenzyl sulfonyl chloride (CAS: 77421-13-7) 4-Aminophenyl boronic acid pinacol ester (CAS: 214360-73-3) | 406 | 1.54/Method D |

### Preparation of intermediate 143: N-(4-bromo-2-fluorophenyl)-2-fluorobenzenesulfonamide

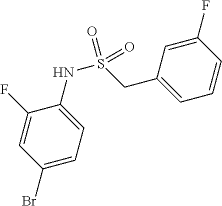 [SMILES: O=S(=O)(Cc1cccc(F)c1)Nc1ccc(Br)cc1F]

A solution of 4-bromo-2-fluoroaniline (CAS 367-24-8; 4.60 g, 24.21 mmol), 2-fluorobenzenesulfonyl chloride (CAS 2905-21-7; 5.18 g, 26.63 mmol) and pyridine (5.9 mL, 72.63 mmol) in DCM (35 mL) was stirred at RT for 20 hr. The solution was washed with 1N HCl, dried (Na₂SO₄) and concentrated in vacuo. The residue was triturated with 10% Et₂O in petroleum ether (bp 40-60° C.) to give the title compound as a white solid (8.05 g, 95%). LCMS (Method D): Rt=1.26 min; m/z [M+H]⁺=348/350

Intermediates 144-146 (Table 16) were prepared by using an analogous reaction protocol as described for intermediate 143 from the appropriate starting materials.

TABLE 16

| | | Starting | LCMS m/z | HPLC Rₜ |
|---|---|---|---|---|
| Intermediate | Structure | materials | (M + H) | (min)/Method |
| 144 | 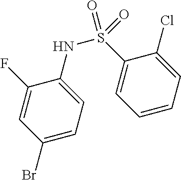 [SMILES: O=S(=O)(Nc1ccc(Br)cc1F)c1ccccc1Cl] | 4-Bromo-2- fluoroaniline (CAS: 367-24-8) 2-Chloro benzene sulfonyl chloride (CAS: 2905-23-9) | 362/364/366 [M − H]⁻ | 1.70/Method E |
| 145 | 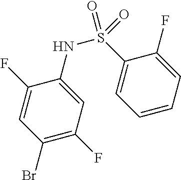 [SMILES: O=S(=O)(Nc1cc(F)c(Br)cc1F)c1ccccc1F] | 4-Bromo-2,5- difluoroaniline (CAS: 112279-60- 4) 2-Fluorobenzene sulfonyl chloride (CAS: 2905-21-7) | 366 | 1.03/Method G |
| 146 | 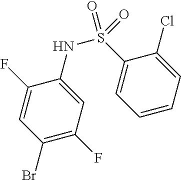 [SMILES: O=S(=O)(Nc1cc(F)c(Br)cc1F)c1ccccc1Cl] | 4-Bromo-2,5- difluoroaniline (CAS: 112279-60- 4) 2- Chlorobenzene- sulfonyl chloride (CAS: 2905-23-9) | 380 [M − H]⁻ | 1.04/Method G |

### Preparation of intermediate 147: 2-chloro-N-(2,5-difluoro-4-(4,4,5,5-tetramethyl-1,3,2-dioxaborolan-2-yl)phenyl)benzenesulfonamide

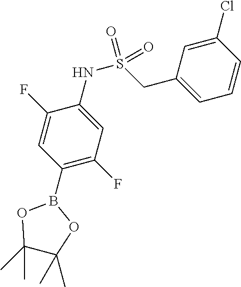 [SMILES: CC1(C)OB(c2cc(F)c(NS(=O)(=O)Cc3cccc(Cl)c3)cc2F)OC1(C)C]

A mixture of intermediate 146 (5.02 g, 13.12 mmol), bis(pinacolato)diboron (CAS 73183-34-3; 4.00 g, 15.74 mmol) and potassium acetate (3.22 g, 32.80 mmol) in 1,4-dioxane (40 mL) was degassed before [1,1′-bis(diphenylphosphino)ferrocene]palladium(II)chloride complex with DCM (CAS 95464-05-4; 540 mg, 0.656 mmol) was added. It was degassed again and then heated at 100° C. overnight. The conversion was incomplete so further bis(pinacolato)diboron (CAS 73183-34-3; 4.00 g, 15.74 mmol) and potassium acetate (3.22 g, 32.80 mmol) in 1,4-dioxane (10 mL) were added, again degassed before [1,1′-bis(diphenylphosphino)ferrocene]palladium(II)chloride complex with DCM (CAS 95464-05-4; 540 mg, 0.656 mmol) added and the mixture heated at 100° C. for 2 hours. The cooled mixture was diluted with EtOAc, filtered via CELITE® and the filtrate concentrated in vacuo. Water was added to the resultant residue and extracted into EtOAc. The combined extracts were washed with saturated brine, dried (MgSO₄) and concentrated in vacuo. This residue was re-dissolved in DCM, passed through a pad of silica washing through with 10% EtOAc/cyclohexane and the filtrate concentrated in vacuo. The orange residue was triturated with 10% Et₂O/cyclohexane, solid materials removed by filtration and the filtrate again concentrated in vacuo. Crude product was purified by chromatography on SiO₂, eluting with a gradient of EtOAc/cyclohexane (0-15%) and fractions concentrated in vacuo to give the title compound as an orange oil (5.1 g, 90%). LCMS (Method E): Rt=1.24 min; m/z [M−H]−=346/348 (boronic acid)

Intermediates 148-150 (Table 17) were prepared by using an analogous reaction protocol as described for intermediate 147 from the appropriate starting materials.

TABLE 17

| | | | LCMS m/z | HPLC Rₜ | ¹H NMR (300 |
|---|---|---|---|---|---|
| Intermediate | Structure | Starting materials | (M + H) | (min)/Method | MHz, CDCl₃) |
| 148 | 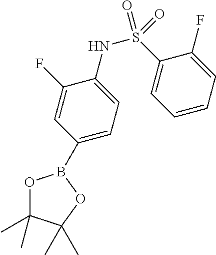 [SMILES: CC1(C)OB(c2ccc(NS(=O)(=O)c3ccccc3F)c(F)c2)OC1(C)C] | Intermediate 143 Bis(pinacolato) diboron (CAS 73183- 34-3) | 394 [M − H]⁻ | 1.55/Method H | |
| 149 | 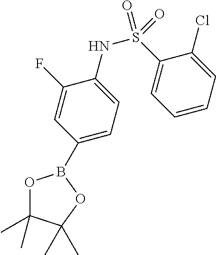 [SMILES: CC1(C)OB(c2ccc(NS(=O)(=O)c3ccccc3Cl)c(F)c2)OC1(C)C] | Intermediate 144 Bis(pinacolato) diboron (CAS 73183- 34-3) | | | 8.09-8.04 (1H, m), 7.53-7.30 (7H, m), 1.29 (12H, s) |
| 150 | 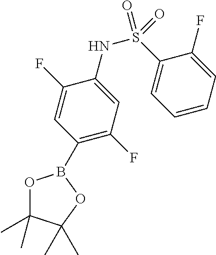 [SMILES: CC1(C)OB(c2cc(F)c(NS(=O)(=O)c3ccccc3F)cc2F)OC1(C)C] | Intermediate 145 Bis(pinacolato) diboron (CAS 73183- 34-3) | Boronic acid 330 [M − H]⁻ | 1.15/Method E | |

### Preparation of intermediate 151: N-(4-(4-amino-1-isopropyl-1H-pyrazolo[4,3-c]pyridin-3-yl)-2-fluorophenyl)-2-chlorobenzenesulfonamide

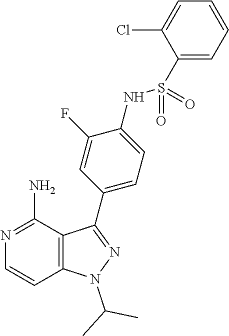 [SMILES: CC(C)n1nc(-c2ccc(NS(=O)(=O)c3ccccc3Cl)c(F)c2)c2c(N)nccc21]

3-Bromo-1-isopropyl-1H-pyrazolo[4,3-c]pyridin-4-amine (intermediate 4) (287 mg, 1.12 mmol), cesium carbonate (1.10 g, 3.37 mmol) and 2-chloro-N-(2-fluoro-4-(4,4,5,5-tetramethyl-1,3,2-dioxaborolan-2-yl)phenyl)benzenesulfonamide (intermediate 149) (648 mg, 1.57 mmol) in a mixture of 1,4-dioxane (5 mL) and water (0.5 mL) was degassed with nitrogen, treated with Pd(dppf)Cl₂.DCM (92 mg, 0.11 mmol) and heated to 90° C. under nitrogen for 24 h. The mixture was cooled to RT, diluted with EtOAc and washed with water. The aqueous layer was extracted with EtOAc and the combined organic layers were washed with water, saturated brine, dried (Na₂SO₄), filtered through CELITE® and concentrated in vacuo to give the crude product. This crude product was purified by column chromatography using a gradient of MeOH EtOAc (0-10%) and relevant fractions concentrated in vacuo to give the title compound (426 mg, 82%) as an off-white solid. LCMS (Method G): Rt=1.02 min; m/z [M+H]⁺=460

Intermediates 152-154 (Table 18) were prepared by using an analogous reaction protocol as described for intermediate 151 from the appropriate starting material.

TABLE 18

| | | Starting | LCMS m/z | HPLC Rₜ |
|---|---|---|---|---|
| Intermediate | Structure | material | (M + H) | (min)/Method |
| 152 | 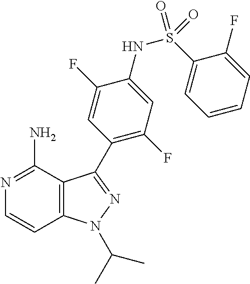 [SMILES: CC(C)n1nc(-c2cc(F)c(NS(=O)(=O)c3ccccc3F)cc2F)c2c(N)nccc21] | Intermediate 4 Intermediate 150 | 462 | 1.10/Method E |
| 153 | 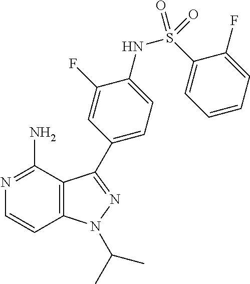 [SMILES: CC(C)n1nc(-c2ccc(NS(=O)(=O)c3ccccc3F)c(F)c2)c2c(N)nccc21] | Intermediate 4 Intermediate 148 | 444 | 0.95/Method D |
| 154 | 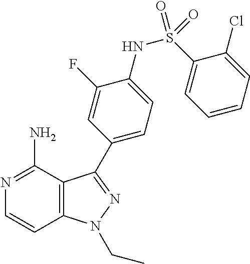 [SMILES: CCn1nc(-c2ccc(NS(=O)(=O)c3ccccc3Cl)c(F)c2)c2c(N)nccc21] | Intermediate 5 Intermediate 149 | 446 | 0.98/Method G |

### Preparation of intermediate 155: N-(4-(4-amino-7-iodo-1-isopropyl-1H-pyrazolo[4,3-c]pyridin-3-yl)-2-fluorophenyl)-2-chlorobenzenesulfonamide

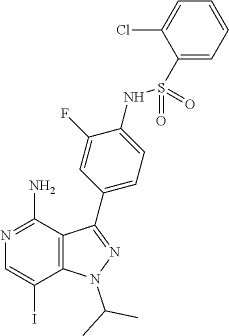 [SMILES: CC(C)n1nc(-c2ccc(NS(=O)(=O)c3ccccc3Cl)c(F)c2)c2c(N)ncc(I)c21]

N-(4-(4-amino-1-isopropyl-1H-pyrazolo[4,3-c]pyridin-3-yl)-2-fluorophenyl)-2-chlorobenzenesulfonamide (intermediate 151) (0.51 g, 1.10 mmol) and NIS (0.50 g, 2.20 mmol) in acetonitrile (38 mL) was heated to 70° C. for 4 h. A further portion of NIS (247 mg, 1.1 mmol) was added and the resulting mixture heated to 70° C. for a further 0.5 h. The mixture was cooled to RT, diluted with EtOAc and washed with 1N Na₂S₂O₅. The aqueous layer was extracted with EtOAc and the combined organic layers were washed with saturated brine, dried (Mg₂SO₄), filtered through CELITE® and concentrated in vacuo to give the crude product. Purification by column chromatography on SiO₂, eluting with a gradient of 0-80% EtOAc and cyclohexane, gave the title compound (0.22 g, 33%) as a beige solid. LCMS (Method E): Rt=1.30 min; m/z [M+H]⁺=586

Intermediates 156-158 (Table 19) were prepared by using an analogous reaction protocol as described for intermediate 155 from the appropriate starting material.

TABLE 19

| | | Starting | LCMS m/z | HPLC Rₜ |
|---|---|---|---|---|
| Intermediate | Structure | material | (M + H) | (min)/Method |
| 156 | 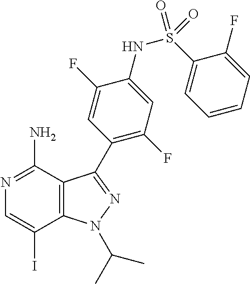 [SMILES: CC(C)n1nc(-c2cc(F)c(NS(=O)(=O)c3ccccc3F)cc2F)c2c(N)ncc(I)c21] | Intermediate 152 | 588 | 1.12/Method D |
| 157 | 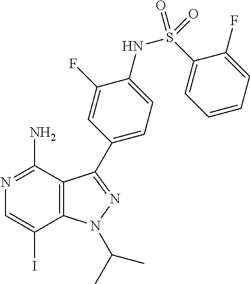 [SMILES: CC(C)n1nc(-c2ccc(NS(=O)(=O)c3ccccc3F)c(F)c2)c2c(N)ncc(I)c21] | Intermediate 153 | 570 | 1.12/Method D |
| 158 | 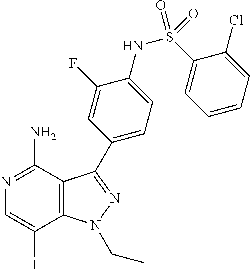 [SMILES: CCn1nc(-c2ccc(NS(=O)(=O)c3ccccc3Cl)c(F)c2)c2c(N)ncc(I)c21] | Intermediate 154 | 572 | 1.13/Method G |

### Preparation of intermediate 159: tert-butyl (4-(4-amino-3-(4-((2-chlorophenyl)sulfonamido)-3-fluorophenyl)-1-isopropyl-1H-pyrazolo[4,3-c]pyridin-7-yl)cyclohex-3-en-1-yl)carbamate

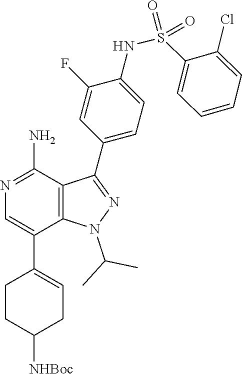 [SMILES: CC(C)n1nc(-c2ccc(NS(=O)(=O)c3ccccc3Cl)c(F)c2)c2c(N)ncc(C3=CCC(NC(=O)OC(C)(C)C)CC3)c21]

Split between two microwave vials: N-(4-(4-amino-7-iodo-1-isopropyl-1H-pyrazolo[4,3-c]pyridin-3-yl)-2-fluorophenyl)-2-chlorobenzenesulfonamide (intermediate 155) (1.22 g, 2.08 mmol), cesium carbonate (2.03 g, 6.24 mmol) and tert-butyl (4-(4,4,5,5-tetramethyl-1,3,2-dioxaborolan-2-yl)cyclohex-3-en-1-yl)carbamate (CAS: 1251732-64-5; 0.81 g, 2.50 mmol) in a mixture of 1,4-dioxane (29 mL) and water (2.9 mL) was degassed with nitrogen, treated with Pd(dppf)Cl₂.DCM (0.17 g, 0.21 mmol) and heated under microwave irradiation at 80° C. for 2 h. The mixture was cooled to RT, diluted with ethyl acetate and washed with water. The aqueous layer was extracted with EtOAc and the combined organic layers dried (Na₂SO₄), filtered through CELITE® and concentrated in vacuo. The residue was loaded onto a 25 g ISOLUTE® SCX-2 cartridge pre-wetted with MeOH, and the cartridge washed with MeOH. The crude product was washed off the SCX-2 with 2N ammonia in MeOH solution and the resulting eluate concentrated in vacuo, to give the title compound as beige solid (0.31 g, 23%). LCMS (Method E): Rt=1.59 min; m/z [M+H]⁺=460

Intermediates 160-162 (Table 20) were prepared by using an analogous reaction protocol as described for intermediate 159 from the appropriate starting material

TABLE 20

| | | | LCMS m/z | HPLC Rₜ |
|---|---|---|---|---|
| Intermediate | Structure | Starting material | (M + H) | (min)/Method |
| 160 | 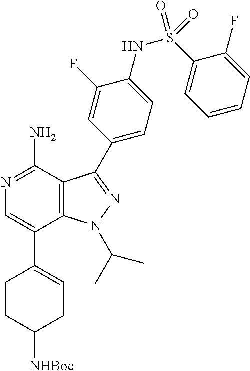 [SMILES: CC(C)n1nc(-c2ccc(NS(=O)(=O)c3ccccc3F)c(F)c2)c2c(N)ncc(C3=CCC(NC(=O)OC(C)(C)C)CC3)c21] | Intermediate 157 tert-Butyl (4-(4,4,5,5- tetramethyl-1,3,2- dioxaborolan-2-yl)cyclohex- 3-en-1-yl)carbamate (CAS: 1251732-64-5) | 639 | 1.05 & 1.15/Method D |
| 161 | 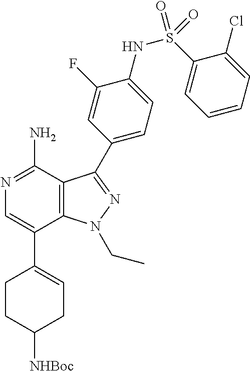 [SMILES: CCn1nc(-c2ccc(NS(=O)(=O)c3ccccc3Cl)c(F)c2)c2c(N)ncc(C3=CCC(NC(=O)OC(C)(C)C)CC3)c21] | Intermediate 158 tert-Butyl (4-(4,4,5,5- tetramethyl-1,3,2- dioxaborolan-2-yl)cyclohex- 3-en-1-yl)carbamate (CAS: 1251732-64-5) | 641/643 | 1.99/Method H |
| 162 | 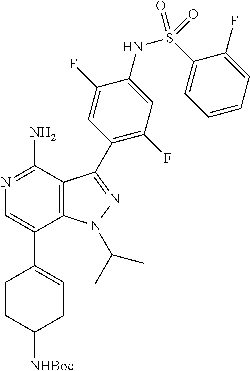 [SMILES: CC(C)n1nc(-c2cc(F)c(NS(=O)(=O)c3ccccc3F)cc2F)c2c(N)ncc(C3=CCC(NC(=O)OC(C)(C)C)CC3)c21] | Intermediate 156 tert-Butyl (4-(4,4,5,5- tetramethyl-1,3,2- dioxaborolan-2-yl)cyclohex- 3-en-1-yl)carbamate (CAS: 1251732-64-5) | 657 | 1.19/Method D |

### Preparation of intermediate 163: N-(4-(4-amino-7-(4-aminocyclohex-1-en-1-yl)-1-isopropyl-1H-pyrazolo[4,3-c]pyridin-3-yl)-2-fluorophenyl)-2-chlorobenzenesulfonamide

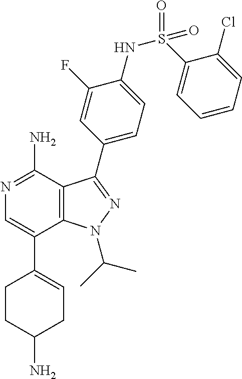 [SMILES: CC(C)n1nc(-c2ccc(NS(=O)(=O)c3ccccc3Cl)c(F)c2)c2c(N)ncc(C3=CCC(N)CC3)c21]

4M hydrochloric acid in 1,4-dioxane (4.35 mL, 17.40 mmol) was added to a stirred solution of tert-butyl(4-(4-amino-3-(4-((2-chlorophenyl)sulfonamido)-3-fluorophenyl)-1-isopropyl-1H-pyrazolo[4,3-c]pyridin-7-yl)cyclohex-3-en-1-yl)carbamate (intermediate 159) (0.20 g, 0.30 mmol) in dioxane (6 mL) and the solution stirred at RT for 3 h. The solution was concentrated in vacuo, the residue dissolved in MeOH and charged to a 5 g ISOLUTE® SCX-2 cartridge pre-wetted with MeOH. The cartridge was eluted with MeOH, then with a solution of ammonia in MeOH (2N) to release the title compound from the SCX-2 solid. The eluted solution was concentrated in vacuo to give the title compound as an off-white solid (0.16 g, 82%). LCMS (Method D): Rt=0.78 min; m/z [M+H]⁺=555

Intermediates 164-166 (Table 21) were prepared by using an analogous reaction protocol as described for intermediate 163 from the appropriate starting material.

TABLE 21

| | | Starting | LCMS m/z | HPLC Rₜ |
|---|---|---|---|---|
| Intermediate | Structure | material | (M + H) | (min)/Method |
| 164 | 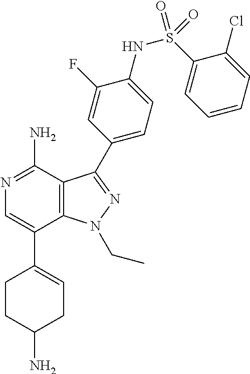 [SMILES: CCn1nc(-c2ccc(NS(=O)(=O)c3ccccc3Cl)c(F)c2)c2c(N)ncc(C3=CCC(N)CC3)c21] | Intermediate 161 | 541 | 0.75/Method D |
| 165 | 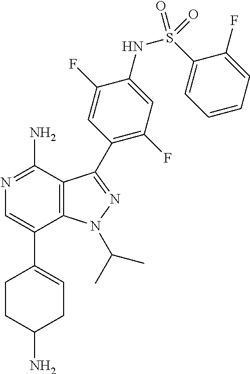 [SMILES: CC(C)n1nc(-c2cc(F)c(NS(=O)(=O)c3ccccc3F)cc2F)c2c(N)ncc(C3=CCC(N)CC3)c21] | Intermediate 162 | 657 | 1.19/Method D |
| 166 | 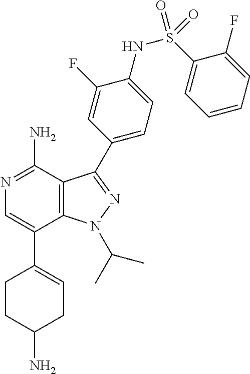 [SMILES: CC(C)n1nc(-c2ccc(NS(=O)(=O)c3ccccc3F)c(F)c2)c2c(N)ncc(C3=CCC(N)CC3)c21] | Intermediate 160 | 539 | 1.01/Method G |

Non-limiting examples of compounds of the invention include:
N-(4-(4-Amino-7-(4-aminocyclohex-1-en-1-yl)-1-isopropyl-1H-pyrazolo[4,3-c]pyridin-3-yl)-2-fluorophenyl)-2-chlorobenzenesulfonamide;
N-(4-(4-Amino-7-(4-((2-fluoroethyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-Amino-7-(4-aminocyclohex-1-en-1-yl)-1-isopropyl-1H-pyrazolo[4,3-c]pyridin-3-yl)-2-fluorophenyl)-2-fluorobenzenesulfonamide:

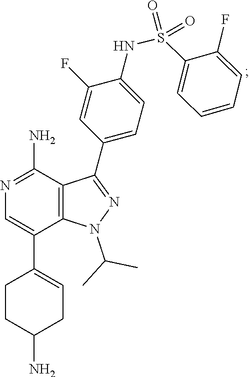 [SMILES: CC(C)n1nc(-c2ccc(NS(=O)(=O)c3ccccc3F)c(F)c2)c2c(N)ncc(C3=CCC(N)CC3)c21]

N-(4-(4-Amino-1-isopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide:

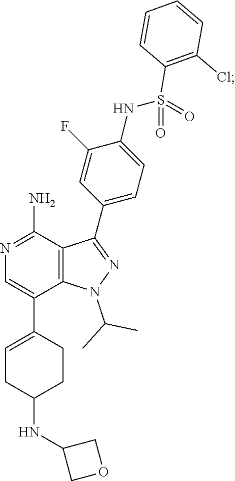 [SMILES: CC(C)n1nc(-c2ccc(NS(=O)(=O)c3ccccc3Cl)c(F)c2)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide:

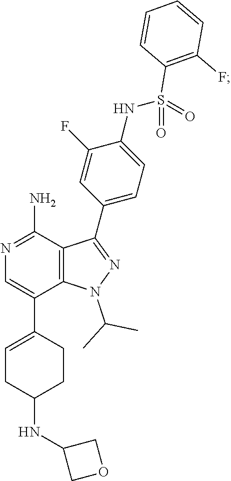 [SMILES: CC(C)n1nc(-c2ccc(NS(=O)(=O)c3ccccc3F)c(F)c2)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide:

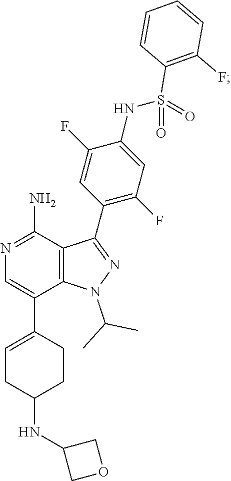 [SMILES: CC(C)n1nc(-c2cc(F)c(NS(=O)(=O)c3ccccc3F)cc2F)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide:

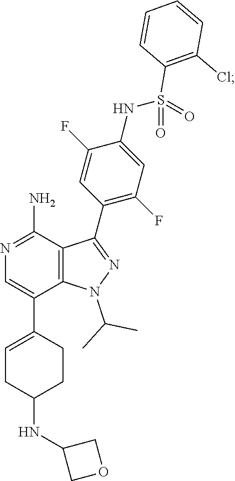 [SMILES: CC(C)n1nc(-c2cc(F)c(NS(=O)(=O)c3ccccc3Cl)cc2F)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chloro-5-fluorobenzenesulfonamide:

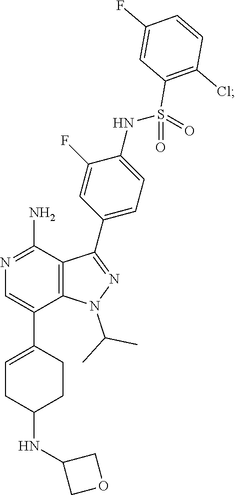 [SMILES: CC(C)n1nc(-c2ccc(NS(=O)(=O)c3cc(F)ccc3Cl)c(F)c2)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2,5-dichlorobenzenesulfonamide:

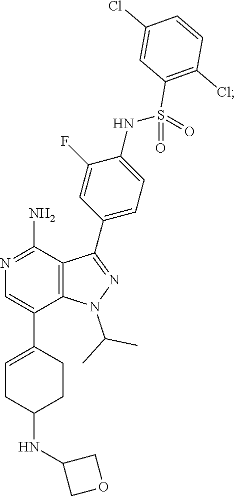 [SMILES: CC(C)n1nc(-c2ccc(NS(=O)(=O)c3cc(Cl)ccc3Cl)c(F)c2)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chloro-5-methylbenzenesulfonamide:

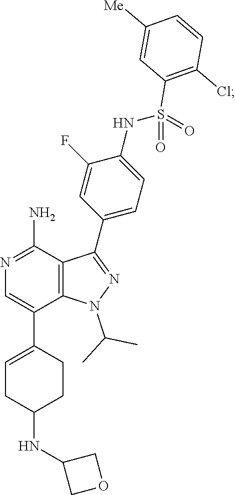 [SMILES: Cc1ccc(Cl)c(S(=O)(=O)Nc2ccc(-c3nn(C(C)C)c4c(C5=CCC(NC6COC6)CC5)cnc(N)c34)cc2F)c1]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2,5-difluorobenzenesulfonamide:

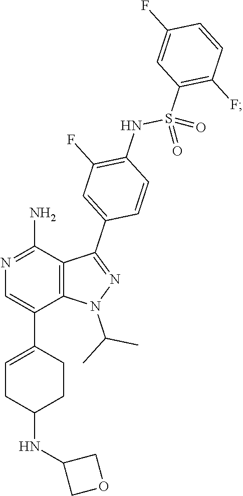 [SMILES: CC(C)n1nc(-c2ccc(NS(=O)(=O)c3cc(F)ccc3F)c(F)c2)c2c(N)ncc(C3=CCC(NC4COC4)CC3)c21]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluoro-5-methoxy benzenesulfonamide:

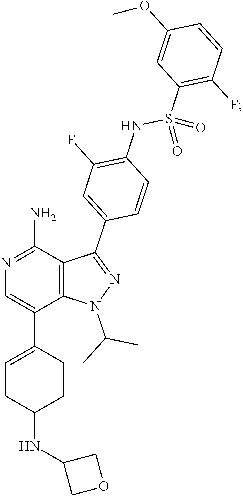 [SMILES: COc1ccc(F)c(S(=O)(=O)Nc2ccc(-c3nn(C(C)C)c4c(C5=CCC(NC6COC6)CC5)cnc(N)c34)cc2F)c1]

N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-methylthiazole-4-sulfonamide:

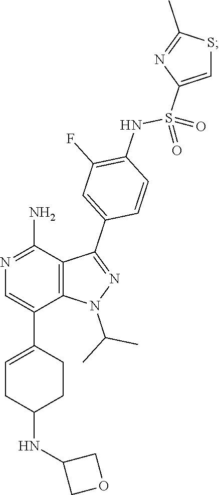 [SMILES: Cc1nc(S(=O)(=O)Nc2ccc(-c3nn(C(C)C)c4c(C5=CCC(NC6COC6)CC5)cnc(N)c34)cc2F)cs1]

N-(4-(4-amino-1-isopropyl-7-(4-(oxetan-3-ylamino)cyclohex-1-en-1-yl)-1H-pyrazolo[4,3-c]pyridin-3-yl)-3-chloro-2-fluorophenyl)-2-fluorobenzenesulfonamide:

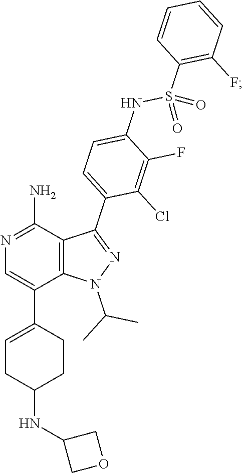 [SMILES: CC(C)n1nc(-c2ccc(NS(=O)(=O)c3ccccc3F)c(F)c2Cl)c2c(N)ncc(C3=CCC(NC4COC4)CC3)c21]

N-(4-(4-amino-1-isopropyl-7-(4-(oxetan-3-ylamino)cyclohexyl)-1H-pyrazolo[4,3-c]pyridin-3-yl)-2-fluorophenyl)-2-chlorobenzenesulfonamide:

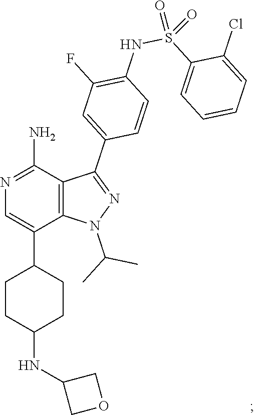 [SMILES: CC(C)n1nc(-c2ccc(NS(=O)(=O)c3ccccc3Cl)c(F)c2)c2c(N)ncc(C3CCC(NC4COC4)CC3)c21]

N-(4-(4-amino-1-isopropyl-7-(4-(oxetan-3-ylamino)cyclohexyl)-1H-pyrazolo[4,3-c]pyridin-3-yl)-2-fluorophenyl)-2-fluorobenzenesulfonamide:

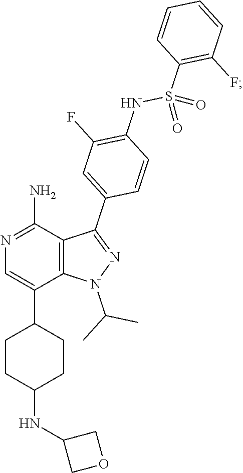 [SMILES: CC(C)n1nc(-c2ccc(NS(=O)(=O)c3ccccc3F)c(F)c2)c2c(N)ncc(C3CCC(NC4COC4)CC3)c21]

N-(4-(4-amino-7-(3,3-difluoro-4-(methylamino)cyclohexyl)-1-isopropyl-1H-pyrazolo[4,3-c]pyridin-3-yl)-2-fluorophenyl)-2-chlorobenzenesulfonamide:

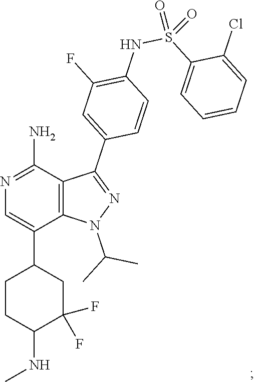 [SMILES: CNC1CCC(c2cnc(N)c3c(-c4ccc(NS(=O)(=O)c5ccccc5Cl)c(F)c4)nn(C(C)C)c23)CC1(F)F]

N-(4-(4-amino-7-(3,5-difluoro-4-(methylamino)cyclohexyl)-1-isopropyl-1H-pyrazolo[4,3-c]pyridin-3-yl)-2-fluorophenyl)-2-chlorobenzenesulfonamide:

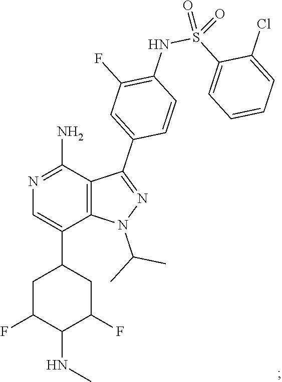 [SMILES: CNC1C(F)CC(c2cnc(N)c3c(-c4ccc(NS(=O)(=O)c5ccccc5Cl)c(F)c4)nn(C(C)C)c23)CC1F]

N-(4-(4-amino-1-isopropyl-7-(4-(oxetan-3-ylamino)piperidin-1-yl)-1H-pyrazolo[4,3-c]pyridin-3-yl)-2-fluorophenyl)-2-chlorobenzenesulfonamide:

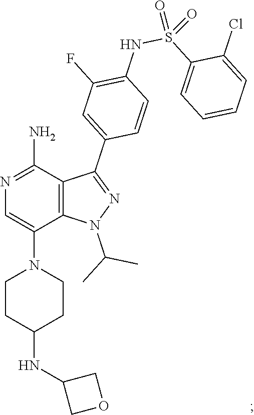 [SMILES: CC(C)n1nc(-c2ccc(NS(=O)(=O)c3ccccc3Cl)c(F)c2)c2c(N)ncc(N3CCC(NC4COC4)CC3)c21]

N-(4-(4-amino-7-(3-fluoro-4-(methylamino)piperidin-1-yl)-1-isopropyl-1H-pyrazolo[4,3-c]pyridin-3-yl)-2-fluorophenyl)-2-chlorobenzenesulfonamide:

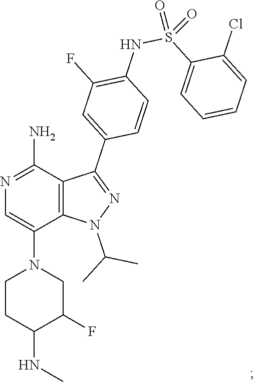 [SMILES: CNC1CCN(c2cnc(N)c3c(-c4ccc(NS(=O)(=O)c5ccccc5Cl)c(F)c4)nn(C(C)C)c23)CC1F]

N-(4-(4-amino-7-(3,3-difluoro-4-(methylamino)piperidin-1-yl)-1-isopropyl-1H-pyrazolo[4,3-c]pyridin-3-yl)-2-fluorophenyl)-2-chlorobenzenesulfonamide:

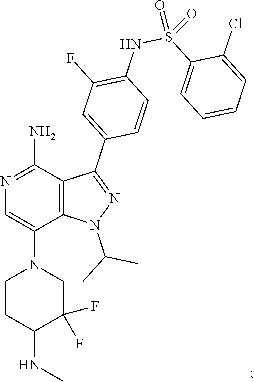 [SMILES: CNC1CCN(c2cnc(N)c3c(-c4ccc(NS(=O)(=O)c5ccccc5Cl)c(F)c4)nn(C(C)C)c23)CC1(F)F]

N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide:

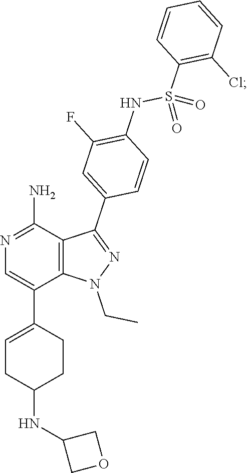 [SMILES: CCn1nc(-c2ccc(NS(=O)(=O)c3ccccc3Cl)c(F)c2)c2c(N)ncc(C3=CCC(NC4COC4)CC3)c21]

N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide:

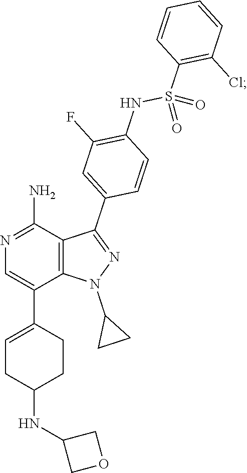 [SMILES: Nc1ncc(C2=CCC(NC3COC3)CC2)c2c1c(-c1ccc(NS(=O)(=O)c3ccccc3Cl)c(F)c1)nn2C1CC1]

N-(4-(4-amino-1-ethyl-7-(4-(oxetan-3-ylamino)cyclohexyl)-1H-pyrazolo[4,3-c]pyridin-3-yl)-2-fluorophenyl)-2-chlorobenzenesulfonamide:

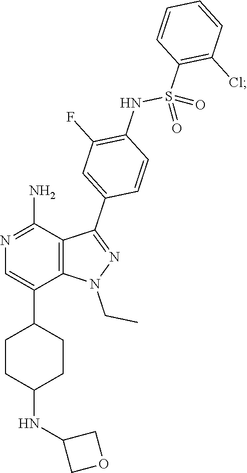 [SMILES: CCn1nc(-c2ccc(NS(=O)(=O)c3ccccc3Cl)c(F)c2)c2c(N)ncc(C3CCC(NC4COC4)CC3)c21]

N-(4-(4-amino-1-cyclopropyl-7-(4-(oxetan-3-ylamino)cyclohexyl)-1H-pyrazolo[4,3-c]pyridin-3-yl)-2-fluorophenyl)-2-chlorobenzenesulfonamide:

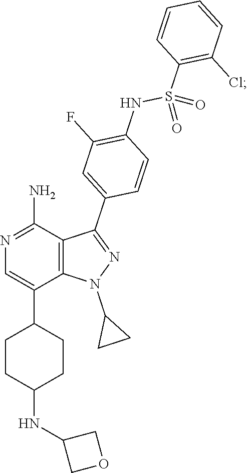 [SMILES: Nc1ncc(C2CCC(NC3COC3)CC2)c2c1c(-c1ccc(NS(=O)(=O)c3ccccc3Cl)c(F)c1)nn2C1CC1]

N-(4-(4-amino-1-cyclopropyl-7-(3-fluoro-4-(methylamino)cyclohexyl)-1H-pyrazolo[4,3-c]pyridin-3-yl)-2-fluorophenyl)-2-chlorobenzenesulfonamide:

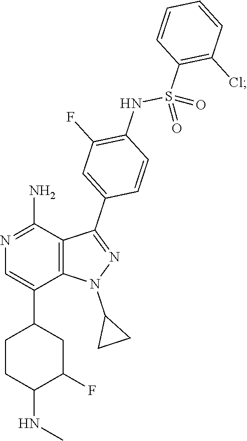 [SMILES: CNC1CCC(c2cnc(N)c3c(-c4ccc(NS(=O)(=O)c5ccccc5Cl)c(F)c4)nn(C4CC4)c23)CC1F]

N-(4-(4-amino-1-cyclopropyl-7-(3,3-difluoro-4-(methylamino)cyclohexyl)-1H-pyrazolo[4,3-c]pyridin-3-yl)-2-fluorophenyl)-2-chlorobenzenesulfonamide:

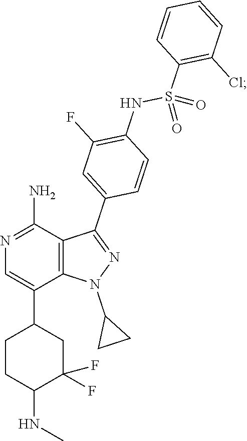 [SMILES: CNC1CCC(c2cnc(N)c3c(-c4ccc(NS(=O)(=O)c5ccccc5Cl)c(F)c4)nn(C4CC4)c23)CC1(F)F]

N-(4-(4-amino-1-cyclopropyl-7-(3,3-difluoro-4-(methylamino)piperidin-1-yl)-1H-pyrazolo[4,3-c]pyridin-3-yl)-2-fluorophenyl)-2-chlorobenzenesulfonamide:

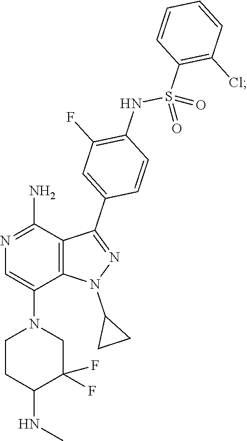 [SMILES: CNC1CCN(c2cnc(N)c3c(-c4ccc(NS(=O)(=O)c5ccccc5Cl)c(F)c4)nn(C4CC4)c23)CC1(F)F]

N-(4-(4-amino-7-(3,3-difluoro-4-(methylamino)cyclohexyl)-1-ethyl-1H-pyrazolo[4,3-c]pyridin-3-yl)-2-fluorophenyl)-2-chlorobenzenesulfonamide:

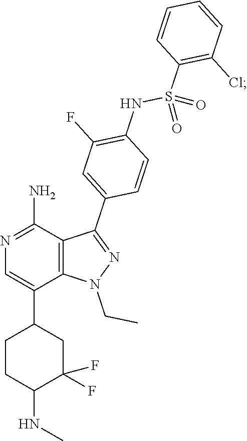 [SMILES: CCn1nc(-c2ccc(NS(=O)(=O)c3ccccc3Cl)c(F)c2)c2c(N)ncc(C3CCC(NC)C(F)(F)C3)c21]

N-(4-(4-amino-7-(3,3-difluoro-4-(methylamino)piperidin-1-yl)-1-ethyl-1H-pyrazolo[4,3-c]pyridin-3-yl)-2-fluorophenyl)-2-chlorobenzenesulfonamide:

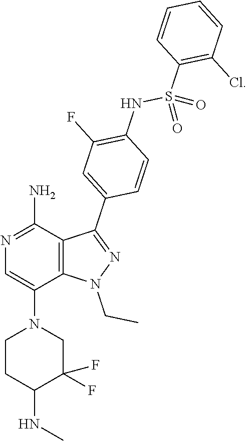 [SMILES: CCn1nc(-c2ccc(NS(=O)(=O)c3ccccc3Cl)c(F)c2)c2c(N)ncc(N3CCC(NC)C(F)(F)C3)c21]

### Preparation of Selected Examples

### Preparation of compound A1 [Method A]: N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide

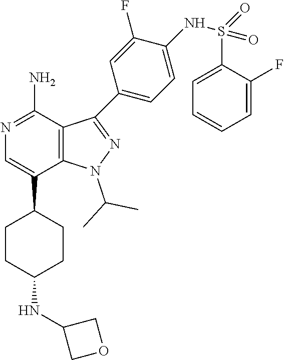 [SMILES: CC(C)n1nc(-c2ccc(NS(=O)(=O)c3ccccc3F)c(F)c2)c2c(N)ncc([C@H]3CC[C@H](NC4COC4)CC3)c21]

3-Oxetanone (CAS: 6704-31-0; 0.49 mL, 0.55 g, 7.66 mmol) was added to a stirred suspension of N-(4-(4-amino-7-((1r,4r)-4-amino cyclohexyl)-isopropyl-1H-pyrazolo[4,3-c]pyridin-3-yl)-2-fluorophenyl)-2-fluorobenzene-sulfonamide (intermediate 71) (2.07 g, 3.83 mmol) and acetic acid (0.46 g, 7.66 mmol) in DCM (60 mL) at RT to give a cloudy solution. Sodium triacetoxyborohydride (1.62 g, 7.66 mmol) was added and the mixture stirred under a nitrogen atmosphere for 18 h. Second portions of 3-oxetanone (CAS: 6704-31-0; 0.24 mL, 0.270 g, 3.74 mmol) and sodium triacetoxyborohydride (0.81 g, 3.82 mmol) were added and stirring continued for a further 4.5 h. The reaction mixture was diluted with water (1 mL) and MeOH (3 mL) then the mixture was concentrated in vacuo. The crude product residue was loaded as a solution in MeOH onto a 50 g ISOLUTE® SCX-2 cartridge pre-wetted with MeOH, and the cartridge washed with MeOH until all acids had eluted. The crude product was washed off the SCX-2 with 2N ammonia in MeOH solution (100 mL) and the resulting eluate concentrated in vacuo to give a white solid. The crude material was purified by reverse-phase column chromatography using a 10-90% acetonitrile in water gradient eluent containing 0.3% concentrated aqueous ammonia followed by freeze drying to give the title compound as a white crystalline solid (1.78 g, 38%). ¹H NMR (400 MHz, DMSO-d₆) δ: 7.78 (1H, m), 7.64 (1H, m), 7.57 (1H, s), 7.41-7.25 (5H, m), 5.53 (2H, s), 4.87 (1H, m, J=6.5 Hz), 4.66 (2H, t, J=6.6 Hz), 4.37 (2H, t, J=4.3 Hz), 4.06 (1H, m, J=6.7 Hz), 2.89 (1H, t, J=11.6 Hz), 2.56 (1H, m, J=11.2 Hz, partially incorporated into DMSO signal), 1.90 (4H, d, J=10.5 Hz), 1.54 (2H, m), 1.50 (6H, d, J=6.5 Hz), 1.28 (2H, m).

### Preparation of compound A2 [Method B]: N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-chloro-5-methoxybenzenesulfonamide

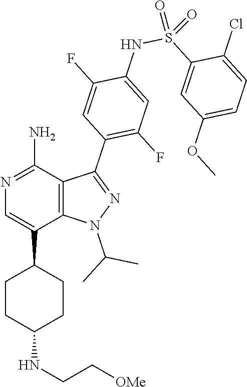 [SMILES: COCCN[C@H]1CC[C@H](c2cnc(N)c3c(-c4cc(F)c(NS(=O)(=O)c5cc(OC)ccc5Cl)cc4F)nn(C(C)C)c32)CC1]

Sodium cyanoborohydride (51 mg, 0.810 mmol) was added to a mixture of 2-methoxyacetaldehyde (CAS: 10312-83-1; 21 mg, 0.289 mmol), N-(4-(4-amino-7-((1r,4r)-4-aminocyclohexyl)-1-isopropyl-1H-pyrazolo[4,3-c]pyridin-3-yl)-2,5-difluorophenyl)-2-chloro-5-methoxybenzenesulfonamide (intermediate 85) (70 mg, 0.116 mmol) and formic acid (13 mg, 0.289 mmol) in dry MeOH (2.0 mL) and the mixture stirred at room temperature for 18 h. The mixture was concentrated in vacuo and then loaded as a solution in MeOH onto a 5 g ISOLUTE® SCX-2 cartridge pre-wetted with MeOH, and the cartridge washed with MeOH until all acids had eluted. The crude product was washed off the SCX-2 with 2N ammonia in MeOH solution (20 mL) and the resulting eluate concentrated in vacuo to give a solid which was purified by column chromatography on a 4 g, 15 μm SiO₂ column, using a 0-20% 2N methanolic ammonia in DCM gradient to give the title compound as a white crystalline solid (64 mg, 83%). ¹H NMR (400 MHz, DMSO) δ: 8.32 (1H, br. s), 7.56 (1H, s), 7.51 (1H, d, J=3.1 Hz), 7.34 (1H, d, J=8.7 Hz), 7.02 (1H, dd, J=7.5, 13.2 Hz), 6.99-6.92 (2H, m), 5.41 (2H, s), 4.83 (1H, sept, J=6.4 Hz), 3.75 (3H, s), 3.58 (2H, t, J=5.0 Hz), 3.34 (2H, s), 3.19-3.12 (4H, m), 2.92 (1H, t, J=10.9 Hz), 2.18 (2H, d, J=10.8 Hz), 2.01 (2H, d, J=12.3 Hz), 1.70-1.54 (4H, m), 1.51 (6H, d, J=6.5 Hz). LCMS (Method B): Rt=3.22 min; m/z [M+H]⁺=663/665

### Preparation of compound A3: N-(4-(4-amino-1-isopropyl-7-((1r,4r)-4-morpholinocyclohexyl)-1H-pyrazolo[4,3-c]pyridin-3-yl)-2,5-difluorobenzenesulphonamide

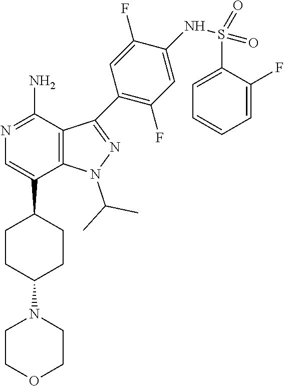 [SMILES: CC(C)n1nc(-c2cc(F)c(NS(=O)(=O)c3ccccc3F)cc2F)c2c(N)ncc([C@H]3CC[C@H](N4CCOCC4)CC3)c21]

A mixture of N-(4-(4-amino-7-((1r,4r)-4-((2-(2-chloroethoxy)ethyl)amino)cyclohexyl)-1-isopropyl-1H-pyrazolo[4,3-c]pyridin-3-yl)-2,5-difluorophenyl)-2-fluorobenzenesulfonamide (intermediate 118) (75 mg, 0.113 mmol) and potassium carbonate (47 mg, 0.339 mmol) in DMF (10 mL) was heated at 70° C. for 1.5 h. The reaction mixture was concentrated in vacuo and the residue was loaded as a solution in MeOH onto a 1 g ISOLUTE® SCX-2 cartridge pre-wetted with MeOH, and the cartridge washed with MeOH until all acids had eluted. The crude product was washed off the SCX-2 with 2N ammonia in MeOH solution (100 mL) and the resulting eluate concentrated in vacuo to give a solid which purified by column chromatography on a 4 g, 15 um SiO₂ column, using a 0-20% 2N methanolic ammonia in DCM. Further purification by MDAP gave the title compound as a white crystalline solid (17.5 mg, 25%). NMR (400 MHz, DMSO) δ: 7.83 (1H, ddd, J=7.5, 7.5, 1.7 Hz), 7.57-7.55 (2H, m), 7.32 (2H, dd, J=9.0, 16.9 Hz), 7.16 (2H, dd, J=7.2, 11.6 Hz), 5.83 (2H, s), 4.92 (1H, sept, J=6.7 Hz), 3.66 (3H, s), 2.94 (2H, t, J=9.4 Hz), 2.76-2.67 (2H, m), 2.03-2.01 (5H, m), 1.53-1.50 (12H, m). LCMS (Method C): Rt=2.78 min; m/z [M+H]⁺=629/631

The following compounds in Table 22 were prepared using an analogous method to compounds A1 or A2.

TABLE 22

| Cmpd | Structure | Starting Materials | Method |
|---|---|---|---|
| A4 | 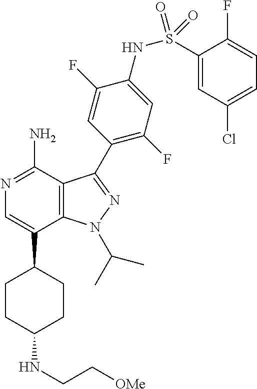 [SMILES: COCCN[C@H]1CC[C@H](c2cnc(N)c3c(-c4cc(F)c(NS(=O)(=O)c5cc(Cl)ccc5F)cc4F)nn(C(C)C)c32)CC1] | Intermediate 106 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-5-chloro-2- | | |
| | fluorobenzenesulfonamide | | |
| A5 | 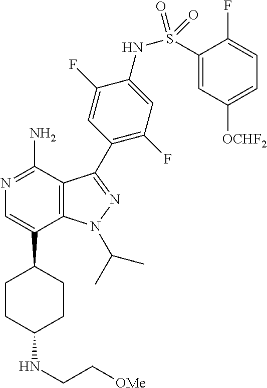 [SMILES: COCCN[C@H]1CC[C@H](c2cnc(N)c3c(-c4cc(F)c(NS(=O)(=O)c5cc(OC(F)F)ccc5F)cc4F)nn(C(C)C)c32)CC1] | Intermediate 107 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-5- | | |
| | (difluoromethoxy)-2-fluorobenzenesulfonamide | | |
| A6 | 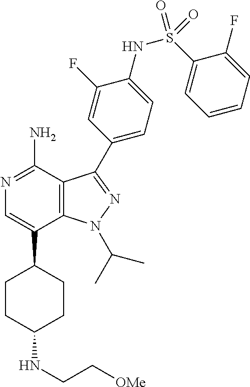 [SMILES: COCCN[C@H]1CC[C@H](c2cnc(N)c3c(-c4ccc(NS(=O)(=O)c5ccccc5F)c(F)c4)nn(C(C)C)c32)CC1] | Intermediate 71 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2-fluorophenyl)-2- | | |
| | fluorobenzenesulfonamide | | |
| A7 | 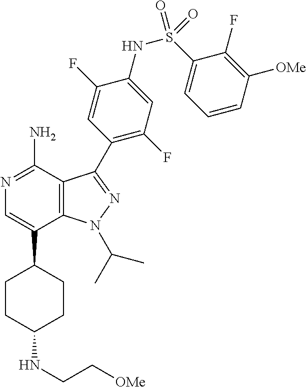 [SMILES: COCCN[C@H]1CC[C@H](c2cnc(N)c3c(-c4cc(F)c(NS(=O)(=O)c5cccc(OC)c5F)cc4F)nn(C(C)C)c32)CC1] | Intermediate 108 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2-fluoro-3- | | |
| | methoxybenzenesulfonamide | | |
| A8 | 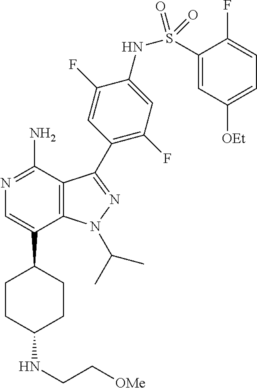 [SMILES: CCOc1ccc(F)c(S(=O)(=O)Nc2cc(F)c(-c3nn(C(C)C)c4c3c(N)ncc4[C@H]3CC[C@H](NCCOC)CC3)cc2F)c1] | Intermediate 81 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-5-ethoxy-2- | | |
| | fluorobenzenesulfonamide | | |
| A9 | 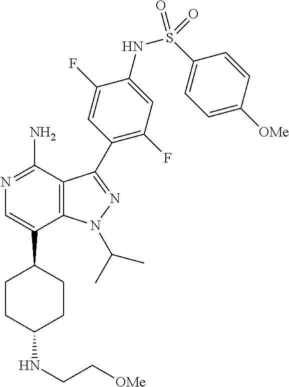 [SMILES: COCCN[C@H]1CC[C@H](c2cnc(N)c3c(-c4cc(F)c(NS(=O)(=O)c5ccc(OC)cc5)cc4F)nn(C(C)C)c32)CC1] | Intermediate 82 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4- | | |
| | methoxybenzenesulfonamide | | |
| A10 | 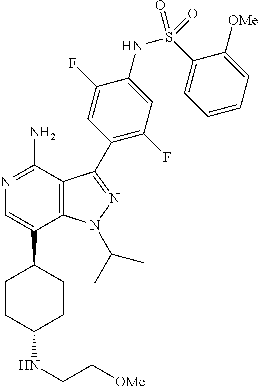 [SMILES: COCCN[C@H]1CC[C@H](c2cnc(N)c3c(-c4cc(F)c(NS(=O)(=O)c5ccccc5OC)cc4F)nn(C(C)C)c32)CC1] | Intermediate 83 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2- | | |
| | methoxybenzenesulfonamide | | |
| A11 | 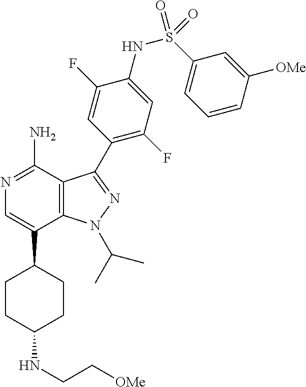 [SMILES: COCCN[C@H]1CC[C@H](c2cnc(N)c3c(-c4cc(F)c(NS(=O)(=O)c5cccc(OC)c5)cc4F)nn(C(C)C)c32)CC1] | Intermediate 84 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-3- | | |
| | methoxybenzenesulfonamide | | |
| A12 | 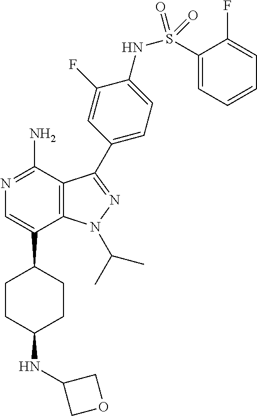 [SMILES: CC(C)n1nc(-c2ccc(NS(=O)(=O)c3ccccc3F)c(F)c2)c2c(N)ncc([C@H]3CC[C@@H](NC4COC4)CC3)c21] | Intermediate 112 3-Oxetanone (CAS: 6704-31-0) | A |
| | N-(4-(4-amino-1-isopropyl-7-((1s,4s)-4-(oxetan-3- | | |
| | ylamino)cyclohexyl)-1H-pyrazolo[4,3-c]pyridin-3-yl)- | | |
| | 2-fluorophenyl)-2-fluorobenzenesulfonamide | | |
| A13 | 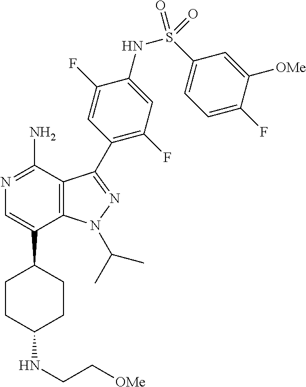 [SMILES: COCCN[C@H]1CC[C@H](c2cnc(N)c3c(-c4cc(F)c(NS(=O)(=O)c5ccc(F)c(OC)c5)cc4F)nn(C(C)C)c32)CC1] | Intermediate 86 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4-fluoro-3- | | |
| | methoxybenzenesulfonamide | | |
| A14 | 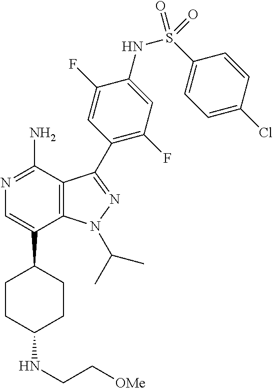 [SMILES: COCCN[C@H]1CC[C@H](c2cnc(N)c3c(-c4cc(F)c(NS(=O)(=O)c5ccc(Cl)cc5)cc4F)nn(C(C)C)c32)CC1] | Intermediate 87 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4- | | |
| | chlorobenzenesulfonamide | | |
| A15 | 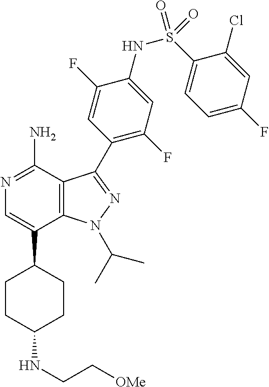 [SMILES: COCCN[C@H]1CC[C@H](c2cnc(N)c3c(-c4cc(F)c(NS(=O)(=O)c5ccc(F)cc5Cl)cc4F)nn(C(C)C)c32)CC1] | Intermediate 88 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2-chloro-4- | | |
| | fluorobenzenesulfonamide | | |
| A16 | 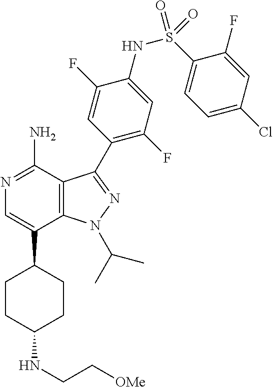 [SMILES: COCCN[C@H]1CC[C@H](c2cnc(N)c3c(-c4cc(F)c(NS(=O)(=O)c5ccc(Cl)cc5F)cc4F)nn(C(C)C)c32)CC1] | Intermediate 89 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4-chloro-2- | | |
| | fluorobenzenesulfonamide | | |
| A17 | 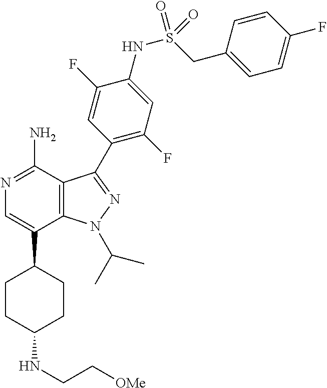 [SMILES: COCCN[C@H]1CC[C@H](c2cnc(N)c3c(-c4cc(F)c(NS(=O)(=O)Cc5ccc(F)cc5)cc4F)nn(C(C)C)c32)CC1] | Intermediate 90 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-1-(4- | | |
| | fluorophenyl)methanesulfonamide | | |
| A18 | 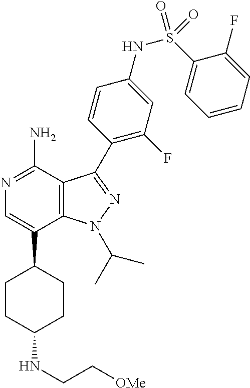 [SMILES: COCCN[C@H]1CC[C@H](c2cnc(N)c3c(-c4ccc(NS(=O)(=O)c5ccccc5F)cc4F)nn(C(C)C)c32)CC1] | Intermediate 91 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2- | | |
| | fluorobenzenesulfonamide | | |
| A19 | 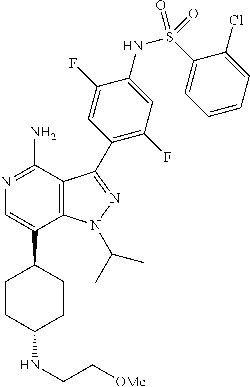 [SMILES: COCCN[C@H]1CC[C@H](c2cnc(N)c3c(-c4cc(F)c(NS(=O)(=O)c5ccccc5Cl)cc4F)nn(C(C)C)c32)CC1] | Intermediate 92 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2- | | |
| | chlorobenzenesulfonamide | | |
| A20 | 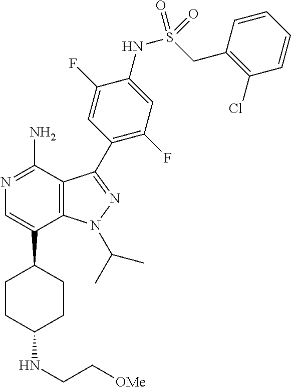 [SMILES: COCCN[C@H]1CC[C@H](c2cnc(N)c3c(-c4cc(F)c(NS(=O)(=O)Cc5ccccc5Cl)cc4F)nn(C(C)C)c32)CC1] | Intermediate 93 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-1-(2- | | |
| | chlorophenyl)methanesulfonamide | | |
| A21 | 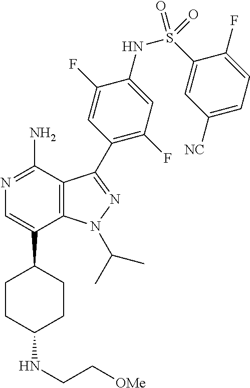 [SMILES: COCCN[C@H]1CC[C@H](c2cnc(N)c3c(-c4cc(F)c(NS(=O)(=O)c5cc(C#N)ccc5F)cc4F)nn(C(C)C)c32)CC1] | Intermediate 94 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-5-cyano-2- | | |
| | fluorobenzenesulfonamide | | |
| A22 | 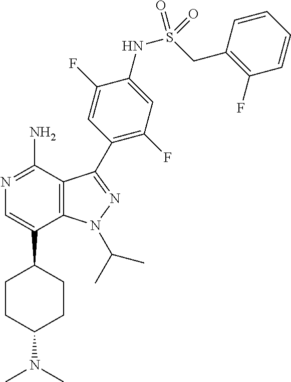 [SMILES: CC(C)n1nc(-c2cc(F)c(NS(=O)(=O)Cc3ccccc3F)cc2F)c2c(N)ncc([C@H]3CC[C@H](N(C)C)CC3)c21] | Intermediate 98 Paraformaldehyde (CAS: 30525-89-4) | B |
| | N-(4-(4-amino-7-((1r,4r)-4- | | |
| | (dimethylamino)cyclohexyl)-1-isopropyl-1H- | | |
| | pyrazolo[4,3-c]pyridin-3-yl)-2,5-difluorophenyl)-1-(2- | | |
| | fluorophenyl)methanesulfonamide | | |
| A23 | 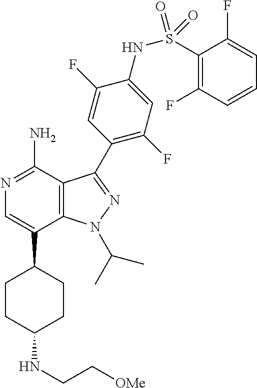 [SMILES: COCCN[C@H]1CC[C@H](c2cnc(N)c3c(-c4cc(F)c(NS(=O)(=O)c5c(F)cccc5F)cc4F)nn(C(C)C)c32)CC1] | Intermediate 95 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2,6- | | |
| | difluorobenzenesulfonamide | | |
| A24 | 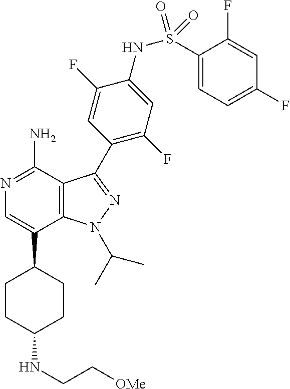 [SMILES: COCCN[C@H]1CC[C@H](c2cnc(N)c3c(-c4cc(F)c(NS(=O)(=O)c5ccc(F)cc5F)cc4F)nn(C(C)C)c32)CC1] | Intermediate 96 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2,4- | | |
| | difluorobenzenesulfonamide | | |
| A25 | 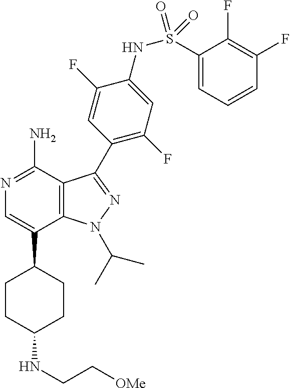 [SMILES: COCCN[C@H]1CC[C@H](c2cnc(N)c3c(-c4cc(F)c(NS(=O)(=O)c5cccc(F)c5F)cc4F)nn(C(C)C)c32)CC1] | Intermediate 97 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2,3- | | |
| | difluorobenzenesulfonamide | | |
| A26 | 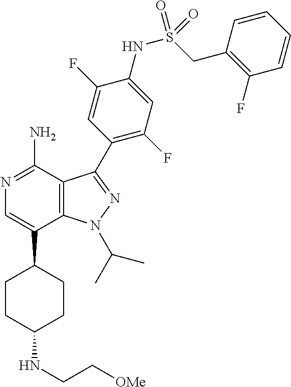 [SMILES: COCCN[C@H]1CC[C@H](c2cnc(N)c3c(-c4cc(F)c(NS(=O)(=O)Cc5ccccc5F)cc4F)nn(C(C)C)c32)CC1] | Intermediate 98 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-1-(2- | | |
| | fluorophenyl)methanesulfonamide | | |
| A27 | 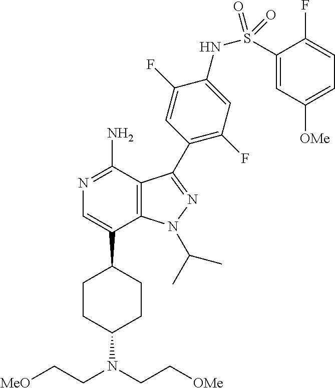 [SMILES: COCCN(CCOC)[C@H]1CC[C@H](c2cnc(N)c3c(-c4cc(F)c(NS(=O)(=O)c5cc(OC)ccc5F)cc4F)nn(C(C)C)c32)CC1] | Intermediate 99 Methoxyacetaldehyde (CAS: 10312-83-1) | A |
| | N-(4-(4-amino-7-((1r,4r)-4-(bis(2- | | |
| | methoxyethyl)amino)cyclohexyl)-1-isopropyl-1H- | | |
| | pyrazolo[4,3-c]pyridin-3-yl)-2,5-difluorophenyl)-2- | | |
| | fluoro-5-methoxybenzenesulfonamide | | |
| A28 | 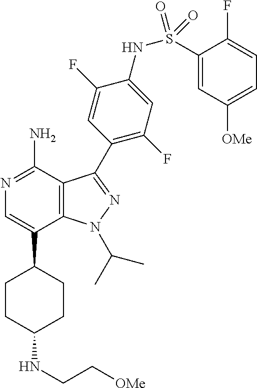 [SMILES: COCCN[C@H]1CC[C@H](c2cnc(N)c3c(-c4cc(F)c(NS(=O)(=O)c5cc(OC)ccc5F)cc4F)nn(C(C)C)c32)CC1] | Intermediate 99 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4-fluoro-5- | | |
| | methoxybenzenesulfonamide | | |
| A29 | 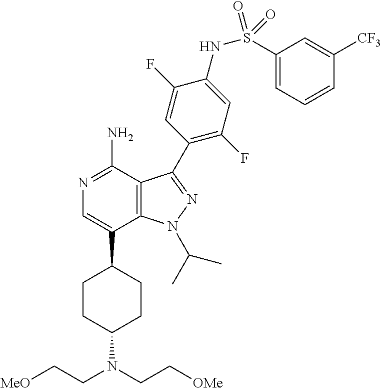 [SMILES: COCCN(CCOC)[C@H]1CC[C@H](c2cnc(N)c3c(-c4cc(F)c(NS(=O)(=O)c5cccc(C(F)(F)F)c5)cc4F)nn(C(C)C)c32)CC1] | Intermediate 100 Methoxyacetaldehyde (CAS: 10312-83-1) | A |
| | N-(4-(4-amino-7-((1r,4r)-4-(bis(2- | | |
| | methoxyethyl)amino)cyclohexyl)-1-isopropyl-1H- | | |
| | pyrazolo[4,3-c]pyridin-3-yl)-2,5-difluorophenyl)-3- | | |
| | (trifluoromethyl)benzenesulfonamide | | |
| A30 | 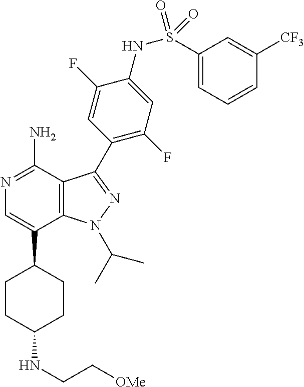 [SMILES: COCCN[C@H]1CC[C@H](c2cnc(N)c3c(-c4cc(F)c(NS(=O)(=O)c5cccc(C(F)(F)F)c5)cc4F)nn(C(C)C)c32)CC1] | Intermediate 100 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-3- | | |
| | (trifluoromethyl)benzenesulfonamide | | |
| A31 | 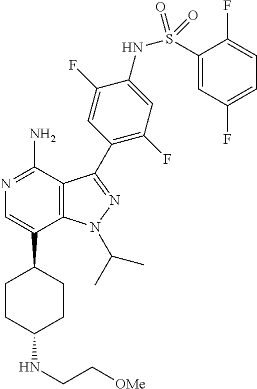 [SMILES: COCCN[C@H]1CC[C@H](c2cnc(N)c3c(-c4cc(F)c(NS(=O)(=O)c5cc(F)ccc5F)cc4F)nn(C(C)C)c32)CC1] | Intermediate 101 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2,5- | | |
| | difluorobenzenesulfonamide | | |
| A32 | 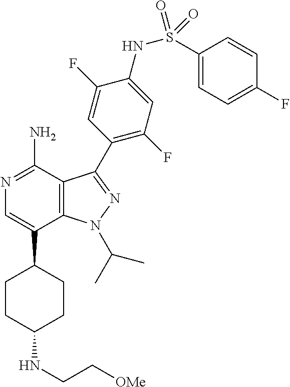 [SMILES: COCCN[C@H]1CC[C@H](c2cnc(N)c3c(-c4cc(F)c(NS(=O)(=O)c5ccc(F)cc5)cc4F)nn(C(C)C)c32)CC1] | Intermediate 102 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-4- | | |
| | fluorobenzenesulfonamide | | |
| A33 | 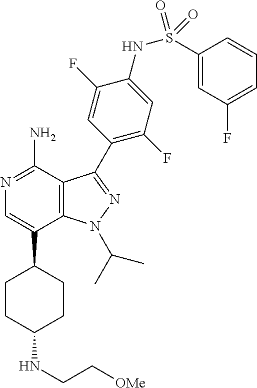 [SMILES: COCCN[C@H]1CC[C@H](c2cnc(N)c3c(-c4cc(F)c(NS(=O)(=O)c5cccc(F)c5)cc4F)nn(C(C)C)c32)CC1] | Intermediate 103 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-3- | | |
| | fluorobenzenesulfonamide | | |
| A34 | 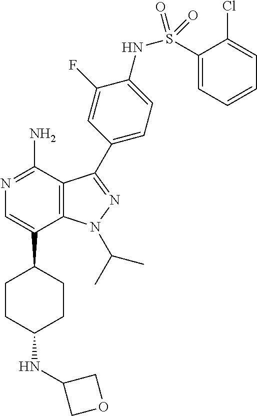 [SMILES: CC(C)n1nc(-c2ccc(NS(=O)(=O)c3ccccc3Cl)c(F)c2)c2c(N)ncc([C@H]3CC[C@H](NC4COC4)CC3)c21] | Intermediate 111 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-2-chlorobenzenesulfonamide | | |
| A35 | 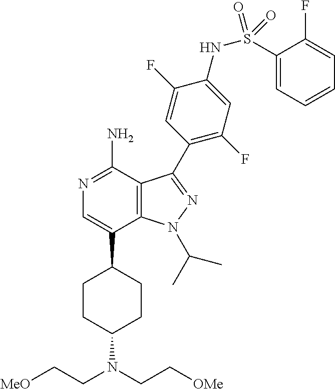 [SMILES: COCCN(CCOC)[C@H]1CC[C@H](c2cnc(N)c3c(-c4cc(F)c(NS(=O)(=O)c5ccccc5F)cc4F)nn(C(C)C)c32)CC1] | Intermediate 104 Methoxyacetaldehyde (CAS: 10312-83-1) | A |
| | N-(4-(4-amino-7-((1r,4r)-4-(bis(2- | | |
| | methoxyethyl)amino)cyclohexyl)-1-isopropyl-1H- | | |
| | pyrazolo[4,3-c]pyridin-3-yl)-2,5-difluorophenyl)-2- | | |
| | fluorobenzenesulfonamide | | |
| A36 | 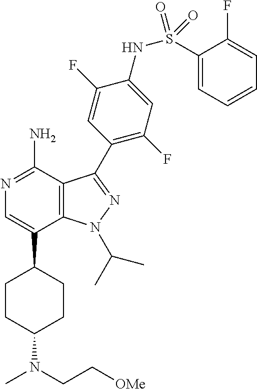 [SMILES: COCCN(C)[C@H]1CC[C@H](c2cnc(N)c3c(-c4cc(F)c(NS(=O)(=O)c5ccccc5F)cc4F)nn(C(C)C)c32)CC1] | Compound A37 Paraformaldehyde (CAS: 30525-89-4) | B |
| | N-(4-(4-amino-1-isopropyl-7-((1r,4r)-4-((2- | | |
| | methoxyethyl)(methyl)amino)cyclohexyl)-1H- | | |
| | pyrazolo[4,3-c]pyridin-3-yl)-2,5-difluorophenyl)-2- | | |
| | fluorobenzenesulfonamide | | |
| A37 | 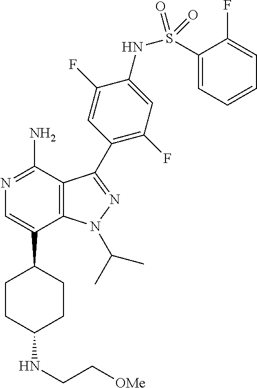 [SMILES: COCCN[C@H]1CC[C@H](c2cnc(N)c3c(-c4cc(F)c(NS(=O)(=O)c5ccccc5F)cc4F)nn(C(C)C)c32)CC1] | Intermediate 104 Methoxyacetaldehyde (CAS: 10312-83-1) | A |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2- | | |
| | fluorobenzenesulfonamide | | |
| A38 | 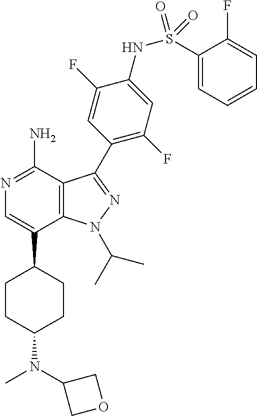 [SMILES: CC(C)n1nc(-c2cc(F)c(NS(=O)(=O)c3ccccc3F)cc2F)c2c(N)ncc([C@H]3CC[C@H](N(C)C4COC4)CC3)c21] | Compound A50 Paraformaldehyde (CAS: 30525-89-4) | B |
| | N-(4-(4-amino-1-isopropyl-7-((1r,4r)-4- | | |
| | (methyl(oxetan-3-yl)amino)cyclohexyl)-1H- | | |
| | pyrazolo[4,3-c]pyridin-3-yl)-2,5-difluorophenyl)-(2- | | |
| | fluorobenzenesulfonamide | | |
| A39 | 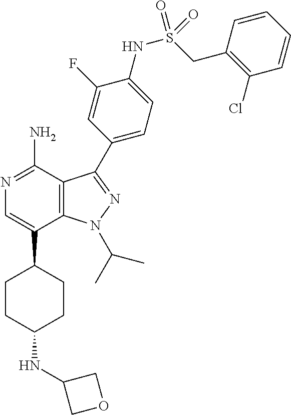 [SMILES: CC(C)n1nc(-c2ccc(NS(=O)(=O)Cc3ccccc3Cl)c(F)c2)c2c(N)ncc([C@H]3CC[C@H](NC4COC4)CC3)c21] | Intermediate 110 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-1-(2- | | |
| | chlorophenyl)methanesulfonamide | | |
| A40 | 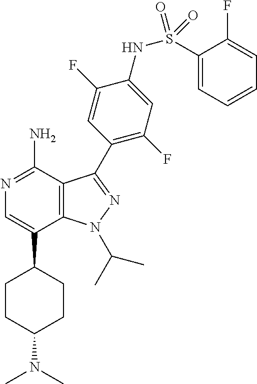 [SMILES: CC(C)n1nc(-c2cc(F)c(NS(=O)(=O)c3ccccc3F)cc2F)c2c(N)ncc([C@H]3CC[C@H](N(C)C)CC3)c21] | Intermediate 104 Paraformaldehyde (CAS: 30525-89-4) | B |
| | N-(4-(4-amino-7-((1r,4r)-4- | | |
| | (dimethylamino)cyclohexyl)-1-isopropyl-1H- | | |
| | pyrazolo[4,3-c]pyridin-3-yl)-2,5-difluorophenyl)-2- | | |
| | fluorobenzenesulfonamide | | |
| A41 | 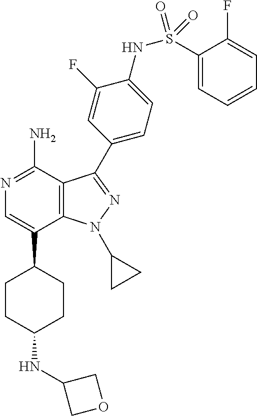 [SMILES: Nc1ncc([C@H]2CC[C@H](NC3COC3)CC2)c2c1c(-c1ccc(NS(=O)(=O)c3ccccc3F)c(F)c1)nn2C1CC1] | Intermediate 77 3-Oxetanone (CAS: 6704-31-0) | A |
| | N-(4-(4-amino-1-cyclopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-2-fluorobenzenesulfonamide | | |
| A42 | 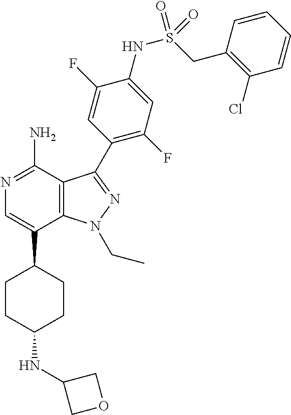 [SMILES: CCn1nc(-c2cc(F)c(NS(=O)(=O)Cc3ccccc3Cl)cc2F)c2c(N)ncc([C@H]3CC[C@H](NC4COC4)CC3)c21] | Intermediate 73 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-1-(2- | | |
| | chlorophenyl)methanesulfonamide | | |
| A43 | 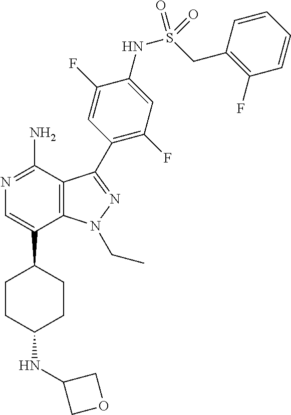 [SMILES: CCn1nc(-c2cc(F)c(NS(=O)(=O)Cc3ccccc3F)cc2F)c2c(N)ncc([C@H]3CC[C@H](NC4COC4)CC3)c21] | Intermediate 105 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-1-(2- | | |
| | fluorophenyl)methanesulfonamide | | |
| A44 | 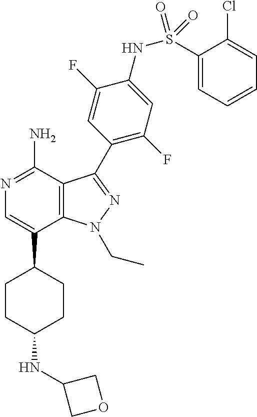 [SMILES: CCn1nc(-c2cc(F)c(NS(=O)(=O)c3ccccc3Cl)cc2F)c2c(N)ncc([C@H]3CC[C@H](NC4COC4)CC3)c21] | Intermediate 75 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-2-chlorobenzenesulfonamide | | |
| A45 | 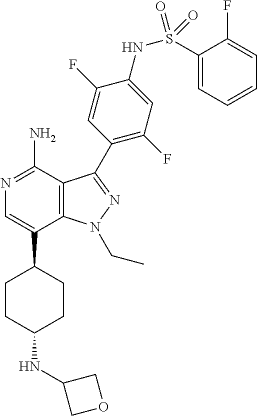 [SMILES: CCn1nc(-c2cc(F)c(NS(=O)(=O)c3ccccc3F)cc2F)c2c(N)ncc([C@H]3CC[C@H](NC4COC4)CC3)c21] | Intermediate 74 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-2-fluorobenzenesulfonamide | | |
| A46 | 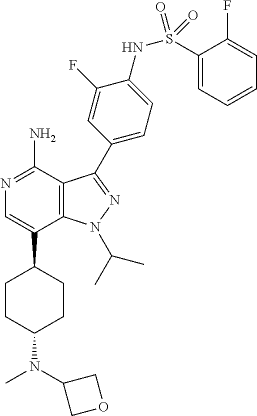 [SMILES: CC(C)n1nc(-c2ccc(NS(=O)(=O)c3ccccc3F)c(F)c2)c2c(N)ncc([C@H]3CC[C@H](N(C)C4COC4)CC3)c21] | Compound A1 Paraformaldehyde (CAS: 30525-89-4) | B |
| | N-(4-(4-amino-1-isopropyl-7-((1r,4r)-4- | | |
| | (methyl(oxetan-3-yl)amino)cyclohexyl)-1H- | | |
| | pyrazolo[4,3-c]pyridin-3-yl)-2,5-difluorophenyl)-2- | | |
| | fluorobenzenesulfonamide | | |
| A47 | 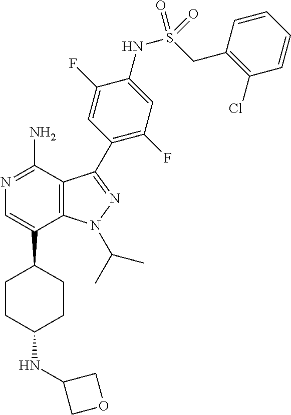 [SMILES: CC(C)n1nc(-c2cc(F)c(NS(=O)(=O)Cc3ccccc3Cl)cc2F)c2c(N)ncc([C@H]3CC[C@H](NC4COC4)CC3)c21] | Intermediate 93 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-1-(2- | | |
| | chlorophenyl)methanesulfonamide | | |
| A48 | 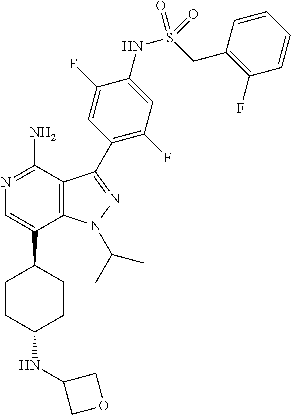 [SMILES: CC(C)n1nc(-c2cc(F)c(NS(=O)(=O)Cc3ccccc3F)cc2F)c2c(N)ncc([C@H]3CC[C@H](NC4COC4)CC3)c21] | Intermediate 98 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-1-(2- | | |
| | fluorophenyl)methanesulfonamide | | |
| A49 | 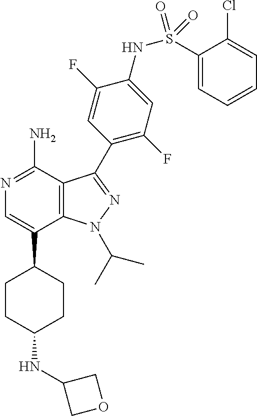 [SMILES: CC(C)n1nc(-c2cc(F)c(NS(=O)(=O)c3ccccc3Cl)cc2F)c2c(N)ncc([C@H]3CC[C@H](NC4COC4)CC3)c21] | Intermediate 92 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-2-chlorobenzenesulfonamide | | |
| A50 | 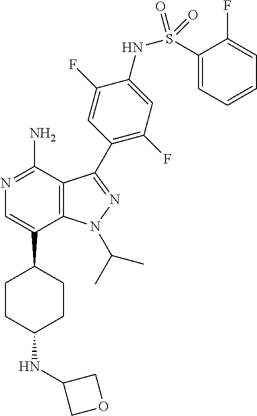 [SMILES: CC(C)n1nc(-c2cc(F)c(NS(=O)(=O)c3ccccc3F)cc2F)c2c(N)ncc([C@H]3CC[C@H](NC4COC4)CC3)c21] | Intermediate 104 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2,5-difluorophenyl)-2-fluorobenzenesulfonamide | | |
| A51 | 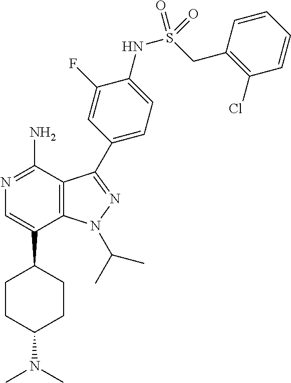 [SMILES: CC(C)n1nc(-c2ccc(NS(=O)(=O)Cc3ccccc3Cl)c(F)c2)c2c(N)ncc([C@H]3CC[C@H](N(C)C)CC3)c21] | Intermediate 93 Paraformaldehyde (CAS: 30525-89-4) | B |
| | N-(4-(4-amino-7-((1r,4r)-4- | | |
| | (dimethylamino)cyclohexyl)-1-isopropyl-1H- | | |
| | pyrazolo[4,3-c]pyridin-3-yl)-2-difluorophenyl)-1-(2- | | |
| | chlorophenyl)methanesulfonamide | | |
| A52 | 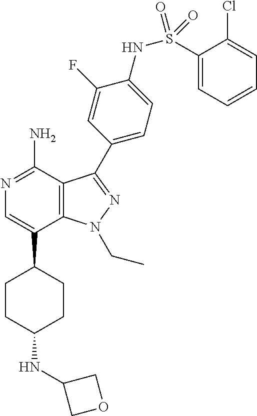 [SMILES: CCn1nc(-c2ccc(NS(=O)(=O)c3ccccc3Cl)c(F)c2)c2c(N)ncc([C@H]3CC[C@H](NC4COC4)CC3)c21] | Intermediate 72 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2-fluorophenyl)-2-chlorobenzenesulfonamide | | |
| A53 | 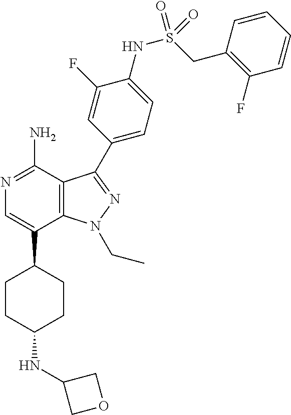 [SMILES: CCn1nc(-c2ccc(NS(=O)(=O)Cc3ccccc3F)c(F)c2)c2c(N)ncc([C@H]3CC[C@H](NC4COC4)CC3)c21] | Intermediate 76 3-Oxetanone (CAS: 6704-31-0) | A |
| | N-(4-(4-amino-1-ethyl-7-((1r,4r)-4-(oxetan-3- | | |
| | ylamino)cyclohexyl)-1H-pyrazolo[4,3-c]pyridin-3-yl)- | | |
| | 2-fluorophenyl)-1-(2- | | |
| | fluorophenyl)methanesulfonamide | | |
| A54 | 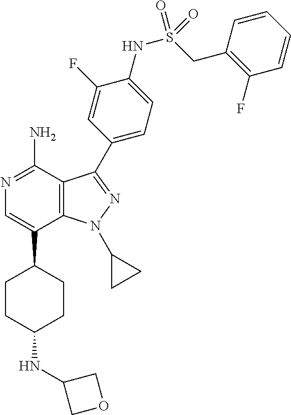 [SMILES: Nc1ncc([C@H]2CC[C@H](NC3COC3)CC2)c2c1c(-c1ccc(NS(=O)(=O)Cc3ccccc3F)c(F)c1)nn2C1CC1] | Intermediate 78 3-Oxetanone (CAS: 6704-31-0) | A |
| | N-(4-(4-amino-1-cyclopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-1-(2- | | |
| | fluorophenyl)methanesulfonamide | | |
| A55 | 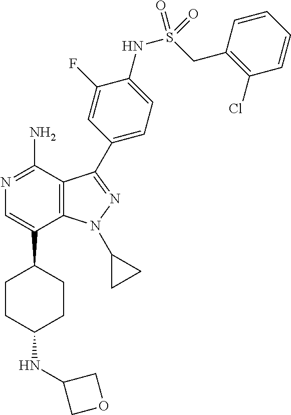 [SMILES: Nc1ncc([C@H]2CC[C@H](NC3COC3)CC2)c2c1c(-c1ccc(NS(=O)(=O)Cc3ccccc3Cl)c(F)c1)nn2C1CC1] | Intermediate 79 3-Oxetanone (CAS: 6704-31-0) | A |
| | N-(4-(4-amino-1-cyclopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-1-(2- | | |
| | chlorophenyl)methanesulfonamide | | |
| A56 | 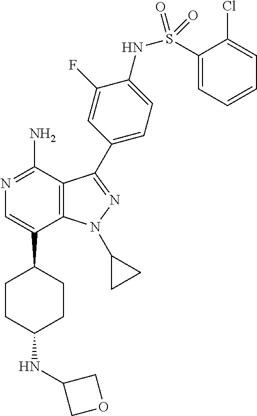 [SMILES: Nc1ncc([C@H]2CC[C@H](NC3COC3)CC2)c2c1c(-c1ccc(NS(=O)(=O)c3ccccc3Cl)c(F)c1)nn2C1CC1] | Intermediate 80 3-Oxetanone (CAS: 6704-31-0) | A |
| | N-(4-(4-amino-1-cyclopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-2-chlorobenzenesulfonamide | | |
| A57 | 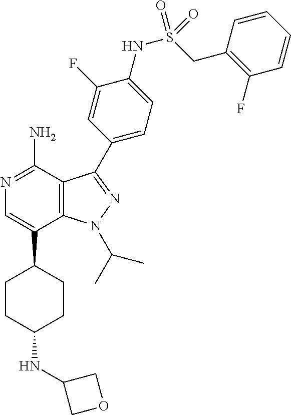 [SMILES: CC(C)n1nc(-c2ccc(NS(=O)(=O)Cc3ccccc3F)c(F)c2)c2c(N)ncc([C@H]3CC[C@H](NC4COC4)CC3)c21] | Intermediate 109 3-Oxetanone (CAS: 6704-31-0) | A |
| | N-(4-(4-amino-1-isopropyl-7-((1r,4r)-4-(oxetan-3- | | |
| | ylamino)cyclohexyl)-1H-pyrazolo[4,3-c]pyridin-3-yl)- | | |
| | 2-fluorophenyl)-1-(2- | | |
| | fluorophenyl)methanesulfonamide | | |
| A58 | 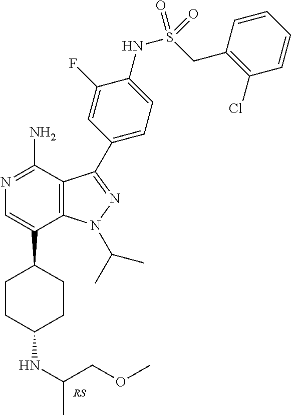 [SMILES: COCC(C)N[C@H]1CC[C@H](c2cnc(N)c3c(-c4ccc(NS(=O)(=O)Cc5ccccc5Cl)c(F)c4)nn(C(C)C)c32)CC1] | Intermediate 110 Methoxyacetone (CAS: 5878-19-3) | A |
| | N-(4-(4-amino-1-isopropyl-7-((1r,4r)-4-((1- | | |
| | methoxypropan-2-yl)amino)cyclohexyl)-1H- | | |
| | pyrazolo[4,3-c]pyridin-3-yl)-2-fluorophenyl)-1-(2- | | |
| | chlorophenyl)methanesulfonamide | | |
| A59 | 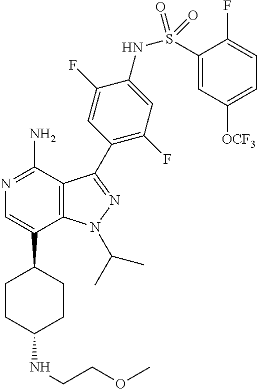 [SMILES: COCCN[C@H]1CC[C@H](c2cnc(N)c3c(-c4cc(F)c(NS(=O)(=O)c5cc(OC(F)(F)F)ccc5F)cc4F)nn(C(C)C)c32)CC1] | Intermediate 113 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2-fluoro-5- | | |
| | (trifluoromethoxy)benzenesulfonamide | | |
| A60 | 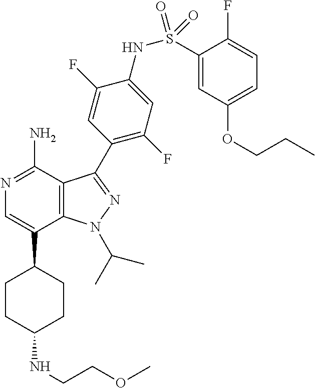 [SMILES: CCCOc1ccc(F)c(S(=O)(=O)Nc2cc(F)c(-c3nn(C(C)C)c4c3c(N)ncc4[C@H]3CC[C@H](NCCOC)CC3)cc2F)c1] | Intermediate 114 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2-fluoro-5- | | |
| | propoxybenzenesulfonamide | | |
| A61 | 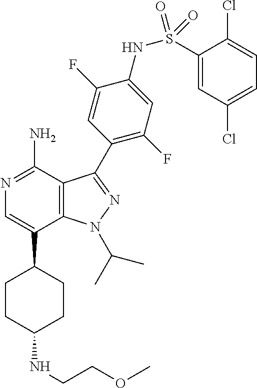 [SMILES: COCCN[C@H]1CC[C@H](c2cnc(N)c3c(-c4cc(F)c(NS(=O)(=O)c5cc(Cl)ccc5Cl)cc4F)nn(C(C)C)c32)CC1] | Intermediate 115 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2,5- | | |
| | dichlorobenzenesulfonamide | | |
| A62 | 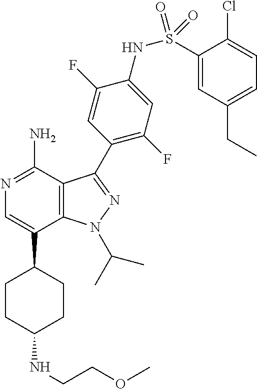 [SMILES: CCc1ccc(Cl)c(S(=O)(=O)Nc2cc(F)c(-c3nn(C(C)C)c4c3c(N)ncc4[C@H]3CC[C@H](NCCOC)CC3)cc2F)c1] | Intermediate 116 Methoxyacetaldehyde (CAS: 10312-83-1) | B |
| | N-(4-(4-amino-1-isopropyl-7-((1r,4r)-4-((2- | | |
| | methoxyethyl)amino)cyclohexyl)-1H-pyrazolo[4,3- | | |
| | c]pyridin-3-yl)-2,5-difluorophenyl)-2chloro-5- | | |
| | ethylbenzenesulfonamide | | |

### Preparation of compound A63: N-(4-(4-amino-7-((1r,4r)-4-((2-fluoropropyl)amino)cyclohexyl)-1-isopropyl-1H-pyrazolo[4,3-c]pyridin-3-yl)-2,5-difluorophenyl)-2-fluorobenzenesulfonamide

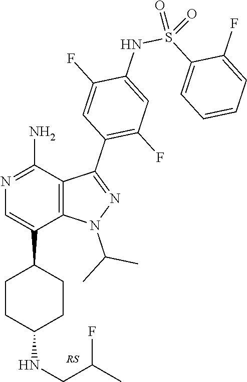 [SMILES: CC(F)CN[C@H]1CC[C@H](c2cnc(N)c3c(-c4cc(F)c(NS(=O)(=O)c5ccccc5F)cc4F)nn(C(C)C)c32)CC1]

Borane dimethyl sulphide complex solution (0.5M; 0.10 mL, 0.051 mmol) was added to a mixture of N-((1r,4r)-4-(4-amino-3-(2,5-difluoro-4-((2-fluorophenyl)sulfonamido)phenyl)-1-isopropyl-1H-pyrazolo[4,3-c]pyridin-7-yl)cyclohexyl)-2-fluoropropanamide (intermediate 117; 32 mg, 0.051 mmol) and THF (3.0 mL), and the resulting mixture was stirred at RT for 4 h. Further borane dimethyl sulphide complex solution (0.5M; 0.20 mL, 0.10 mmol) was added and stirring continued for 18 h. The mixture was loaded as a solution in MeOH onto a 2 g ISOLUTE® SCX-2 cartridge pre-wetted with MeOH, and the cartridge washed with MeOH until all acids had eluted. The crude product was washed off the SCX-2 with 2N ammonia in MeOH solution (20 mL) and the eluate concentrated in vacuo to give a solid. Further purification by chromatography (C18 cartridge), eluting with water/acetonitrile (0.1% formic acid) 0-100% gave the title compound as white solid (8.0 mg, 26%). NMR (400 MHz, DMSO) δ: 8.40 (1H, br. s), 7.69 (1H, dd, J=3.7, 3.7 Hz), 7.57 (1H, s), 7.50-7.46 (1H, m), 7.36 (1H, dd, J=8.8, 8.8 Hz), 5.43 (2H, s), 4.83 (1H, sept, J=6.3 Hz), 3.60 (2H, t, J=5.1 Hz), 3.34 (3H, s), 3.17 (3H, br. d, J=4.2 Hz), 2.91 (1H, br. t, J=12.1 Hz), 2.20 (2H, br. d, J=11.4 Hz), 2.08 (1H, s), 2.02 (2H, br. d, J=12.1 Hz), 1.69-1.53 (4H, m), 1.51 (6H, d, J=6.4 Hz). LCMS (Method C): Rt=2.96 min; m/z [M+H]⁺=619

### Preparation of compounds B1 and B2: N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide

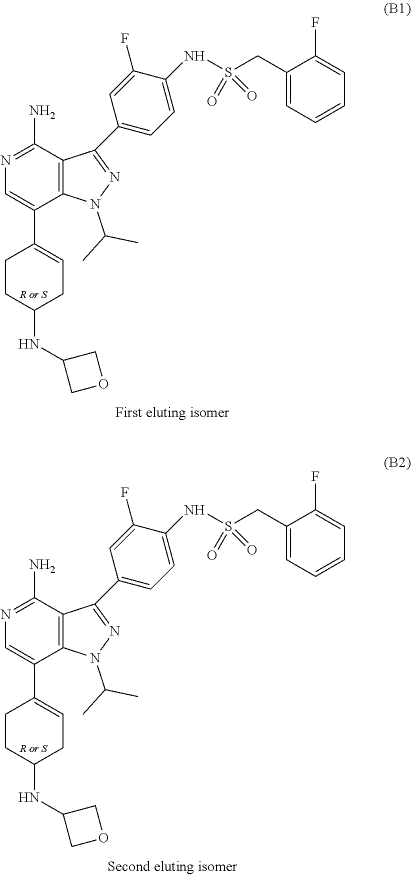 [SMILES: CC(C)n1nc(-c2ccc(NS(=O)(=O)Cc3ccccc3F)c(F)c2)c2c(N)ncc(C3=CCC(NC4COC4)CC3)c21 ; CC(C)n1nc(-c2ccc(NS(=O)(=O)Cc3ccccc3F)c(F)c2)c2c(N)ncc(C3=CCC(NC4COC4)CC3)c21]

3-bromo-1-isopropyl-7-(4-(oxetan-3-ylamino)cyclohex-1-en-1-yl)-1H-pyrazolo[4,3-c]pyridin-4-amine (intermediate 125) (0.25 g, 0.62 mmol), cesium carbonate (0.60 g, 1.85 mmol) and N-(2-fluoro-4-(4,4,5,5-tetramethyl-1,3,2-dioxaborolan-2-yl)phenyl)-1-(2-fluorophenyl)methanesulfonamide (intermediate 136) (0.30 g, 0.74 mmol) in a mixture of 1,4-dioxane (5 mL) and water (2 mL) was degassed with nitrogen, then treated with Pd(dppf)Cl₂.DCM (0.045 g, 0.062 mmol) and heated to 80° C. under nitrogen for 2 h. The mixture was cooled to RT, diluted with EtOAc (100 mL) and washed with saturated brine (15 mL), dried (Na₂SO₄), filtered through CELITE® and concentrated in vacuo to give a brown residue. Purification by SiO₂-pad column chromatography eluting with a gradient of 0-10% ammonia in MeOH (2N)/DCM gave a light brown solid. Further purification by MDAP and then SFC gave the title compounds as white solids (compound B1 40 mg, 11% and compound B2 41 mg, 11%).

### Compound B1 (First Eluting Isomer)

¹H NMR (400 MHz, DMSO) δ: 7.49 (1H, dd, J=8.7, 8.7 Hz), 7.45-7.40 (1H, m), 7.37 (1H, s), 7.36-7.30 (1H, m), 7.21 (1H, dd, J=1.7, 11.9 Hz), 7.17-7.11 (3H, m), 5.68 (3H, s), 4.95 (1H, sept, J=6.7 Hz), 4.72-4.66 (2H, m), 4.39 (2H, ddd, J=6.1, 6.1, 0.9 Hz), 4.23 (2H, s), 4.05 (1H, quint, J=6.8 Hz), 2.37-2.24 (3H, m), 1.98-1.91 (1H, m), 1.87-1.82 (1H, m), 1.57-1.48 (4H, m), 1.39-1.38 (3H, m). LCMS (Method B): Rt=4.11 min; m/z [M+H]⁺=609

### Compound B2 (Second Eluting Isomer)

¹H NMR (400 MHz, DMSO) δ: 7.49 (1H, dd, J=8.7, 8.7 Hz), 7.43 (1H, ddd, J=7.5, 7.5, 2.4 Hz), 7.37 (1H, s), 7.36-7.30 (1H, m), 7.21 (1H, dd, J=1.7, 11.9 Hz), 7.17-7.11 (3H, m), 5.68 (3H, s), 4.95 (1H, sept, J=6.7 Hz), 4.72-4.66 (2H, m), 4.41-4.36 (2H, m), 4.23 (2H, s), 4.05 (1H, quint, J=6.8 Hz), 2.87-2.80 (4H, m), 2.37-2.24 (3H, m), 1.98-1.91 (1H, m), 1.87-1.82 (1H, m), 1.57-1.48 (4H, m), 1.39-1.38 (3H, m), 1.13 (5H, t, J=7.2 Hz). LCMS (Method B): Rt=4.07 min; m/z [M+H]⁺=609

The following compounds in Table 23 were prepared using an analogous method to compounds B1 and B2.

TABLE 23

| Cmpd | Structure | Starting Materials |
|---|---|---|
| B3 | 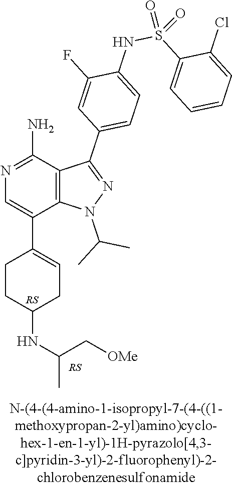 [SMILES: COCC(C)NC1CC=C(c2cnc(N)c3c(-c4ccc(NS(=O)(=O)c5ccccc5Cl)c(F)c4)nn(C(C)C)c23)CC1] | Intermediate 126 Intermediate 149 |
| B4 (Second eluting isomer) | 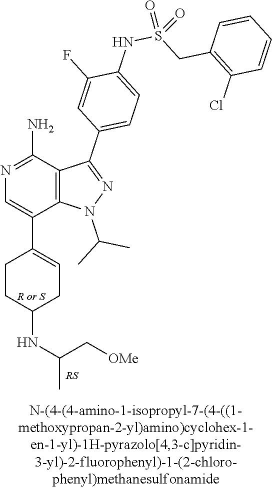 [SMILES: COCC(C)NC1CC=C(c2cnc(N)c3c(-c4ccc(NS(=O)(=O)Cc5ccccc5Cl)c(F)c4)nn(C(C)C)c23)CC1] | Intermediate 126 Intermediate 139 |
| B5 (First eluting isomer) | 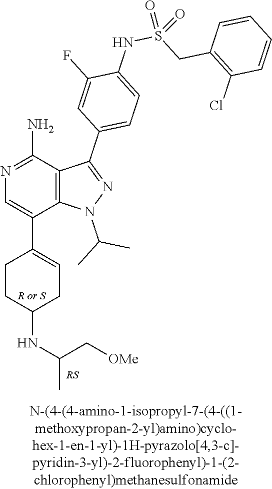 [SMILES: COCC(C)NC1CC=C(c2cnc(N)c3c(-c4ccc(NS(=O)(=O)Cc5ccccc5Cl)c(F)c4)nn(C(C)C)c23)CC1] | Intermediate 126 Intermediate 139 |
| B6 (Second eluting isomer) | 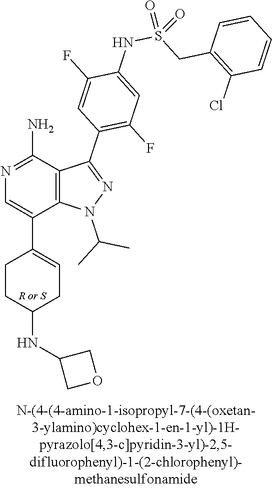 [SMILES: CC(C)n1nc(-c2cc(F)c(NS(=O)(=O)Cc3ccccc3Cl)cc2F)c2c(N)ncc(C3=CCC(NC4COC4)CC3)c21] | Intermediate 125 Intermediate 137 |
| B7 (First eluting isomer) | 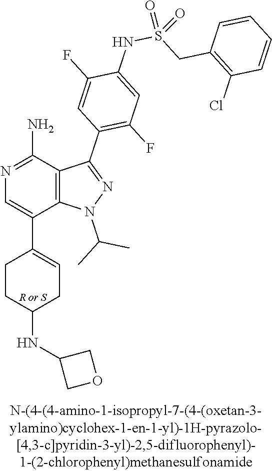 [SMILES: CC(C)n1nc(-c2cc(F)c(NS(=O)(=O)Cc3ccccc3Cl)cc2F)c2c(N)ncc(C3=CCC(NC4COC4)CC3)c21] | Intermediate 125 Intermediate 137 |
| B8 (Second eluting isomer) | 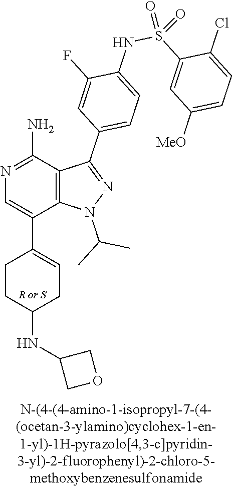 [SMILES: COc1ccc(Cl)c(S(=O)(=O)Nc2ccc(-c3nn(C(C)C)c4c(C5=CCC(NC6COC6)CC5)cnc(N)c34)cc2F)c1] | Intermediate 125 Intermediate 138 |
| B9 (First eluting isomer) | 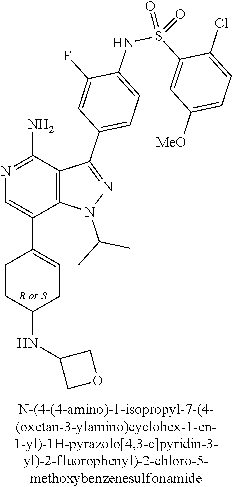 [SMILES: COc1ccc(Cl)c(S(=O)(=O)Nc2ccc(-c3nn(C(C)C)c4c(C5=CCC(NC6COC6)CC5)cnc(N)c34)cc2F)c1] | Intermediate 125 Intermediate 138 |
| B10 (Second eluting isomer) | 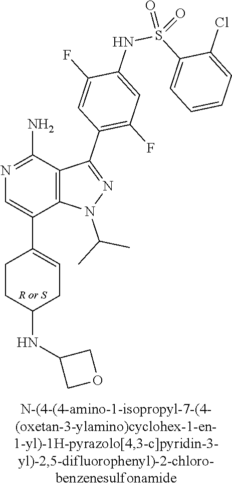 [SMILES: CC(C)n1nc(-c2cc(F)c(NS(=O)(=O)c3ccccc3Cl)cc2F)c2c(N)ncc(C3=CCC(NC4COC4)CC3)c21] | Intermediate 125 Intermediate 147 |
| B11 (First eluting isomer) | 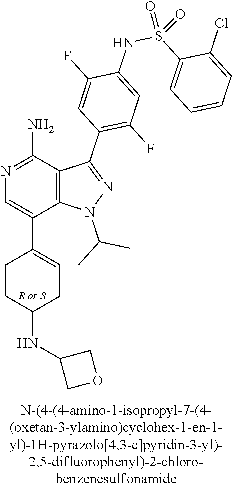 [SMILES: CC(C)n1nc(-c2cc(F)c(NS(=O)(=O)c3ccccc3Cl)cc2F)c2c(N)ncc(C3=CCC(NC4COC4)CC3)c21] | Intermediate 125 Intermediate 147 |
| B12 (Second eluting isomer) | 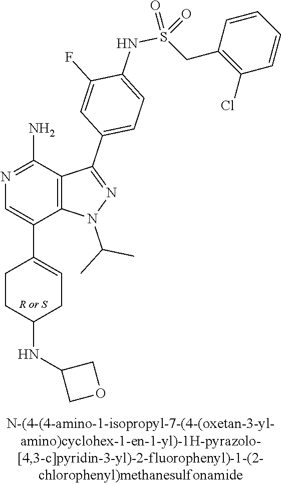 [SMILES: CC(C)n1nc(-c2ccc(NS(=O)(=O)Cc3ccccc3Cl)c(F)c2)c2c(N)ncc(C3=CCC(NC4COC4)CC3)c21] | Intermediate 125 Intermediate 139 |
| B13 (First eluting isomer) | 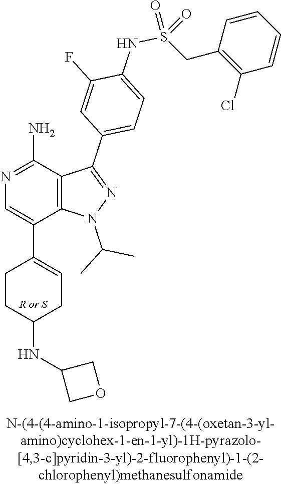 [SMILES: CC(C)n1nc(-c2ccc(NS(=O)(=O)Cc3ccccc3Cl)c(F)c2)c2c(N)ncc(C3=CCC(NC4COC4)CC3)c21] | Intermediate 125 Intermediate 139 |
| B14 (First eluting isomer) | 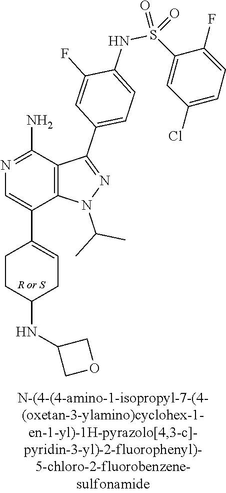 [SMILES: CC(C)n1nc(-c2ccc(NS(=O)(=O)c3cc(Cl)ccc3F)c(F)c2)c2c(N)ncc(C3=CCC(NC4COC4)CC3)c21] | Intermediate 125 Intermediate 140 |
| B15 (Second eluting isomer) | 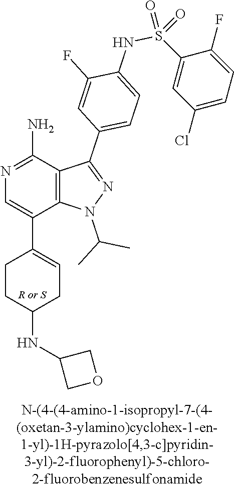 [SMILES: CC(C)n1nc(-c2ccc(NS(=O)(=O)c3cc(Cl)ccc3F)c(F)c2)c2c(N)ncc(C3=CCC(NC4COC4)CC3)c21] | Intermediate 125 Intermediate 140 |
| B16 (First eluting isomer) | 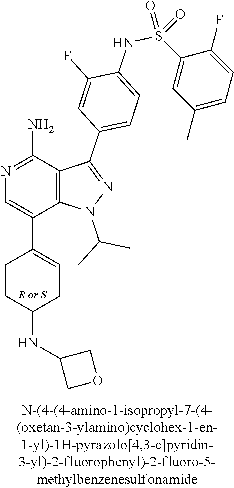 [SMILES: Cc1ccc(F)c(S(=O)(=O)Nc2ccc(-c3nn(C(C)C)c4c(C5=CCC(NC6COC6)CC5)cnc(N)c34)cc2F)c1] | Intermediate 125 Intermediate 141 |
| B17 (Second eluting isomer) | 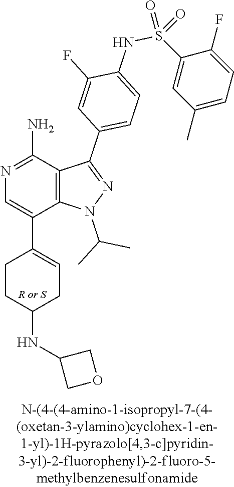 [SMILES: Cc1ccc(F)c(S(=O)(=O)Nc2ccc(-c3nn(C(C)C)c4c(C5=CCC(NC6COC6)CC5)cnc(N)c34)cc2F)c1] | Intermediate 125 Intermediate 141 |
| B18 (First eluting isomer) | 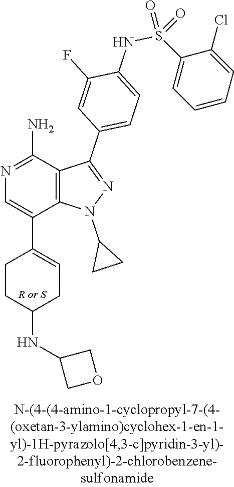 [SMILES: Nc1ncc(C2=CCC(NC3COC3)CC2)c2c1c(-c1ccc(NS(=O)(=O)c3ccccc3Cl)c(F)c1)nn2C1CC1] | Intermediate 127 Intermediate 149 |
| B19 (Second eluting isomer) | 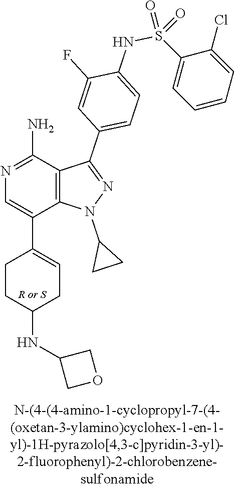 [SMILES: Nc1ncc(C2=CCC(NC3COC3)CC2)c2c1c(-c1ccc(NS(=O)(=O)c3ccccc3Cl)c(F)c1)nn2C1CC1] | Intermediate 127 Intermediate 149 |
| B20 (First eluting isomer) | 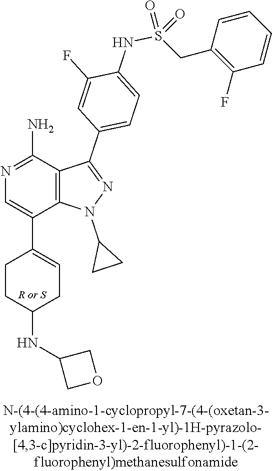 [SMILES: Nc1ncc(C2=CCC(NC3COC3)CC2)c2c1c(-c1ccc(NS(=O)(=O)Cc3ccccc3F)c(F)c1)nn2C1CC1] | Intermediate 127 Intermediate 136 |
| B21 (Second eluting isomer) | 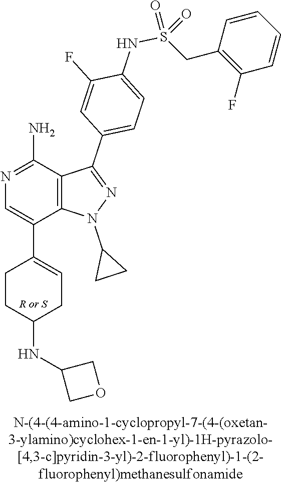 [SMILES: Nc1ncc(C2=CCC(NC3COC3)CC2)c2c1c(-c1ccc(NS(=O)(=O)Cc3ccccc3F)c(F)c1)nn2C1CC1] | Intermediate 127 Intermediate 136 |
| B22 (Second eluting isomer) | 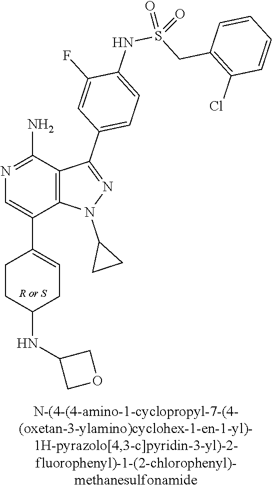 [SMILES: Nc1ncc(C2=CCC(NC3COC3)CC2)c2c1c(-c1ccc(NS(=O)(=O)Cc3ccccc3Cl)c(F)c1)nn2C1CC1] | Intermediate 127 Intermediate 139 |
| B23 (First eluting isomer) | 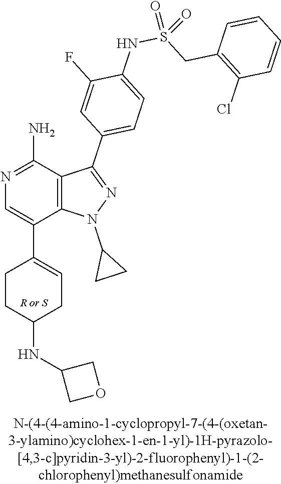 [SMILES: Nc1ncc(C2=CCC(NC3COC3)CC2)c2c1c(-c1ccc(NS(=O)(=O)Cc3ccccc3Cl)c(F)c1)nn2C1CC1] | Intermediate 127 Intermediate 139 |
| B24 (First eluting isomer) | 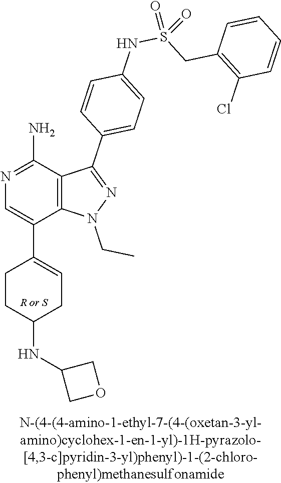 [SMILES: CCn1nc(-c2ccc(NS(=O)(=O)Cc3ccccc3Cl)cc2)c2c(N)ncc(C3=CCC(NC4COC4)CC3)c21] | Intermediate 128 Intermediate 142 |
| B25 (Second eluting isomer) | 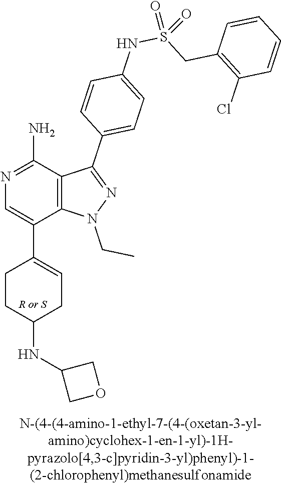 [SMILES: CCn1nc(-c2ccc(NS(=O)(=O)Cc3ccccc3Cl)cc2)c2c(N)ncc(C3=CCC(NC4COC4)CC3)c21] | Intermediate 128 Intermediate 142 |
| B26 (Second eluting isomer) | 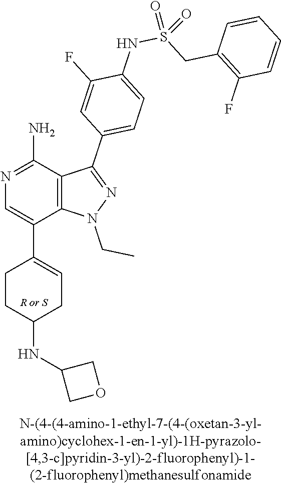 [SMILES: CCn1nc(-c2ccc(NS(=O)(=O)Cc3ccccc3F)c(F)c2)c2c(N)ncc(C3=CCC(NC4COC4)CC3)c21] | Intermediate 128 Intermediate 136 |
| B27 (First eluting isomer) | 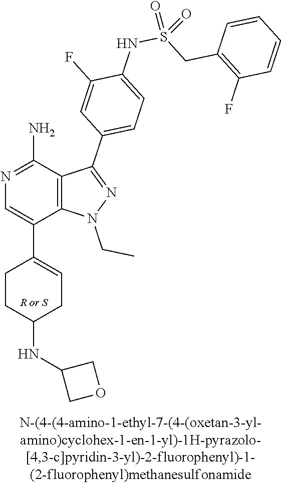 [SMILES: CCn1nc(-c2ccc(NS(=O)(=O)Cc3ccccc3F)c(F)c2)c2c(N)ncc(C3=CCC(NC4COC4)CC3)c21] | Intermediate 128 Intermediate 136 |
| B28 (Second eluting isomer) | 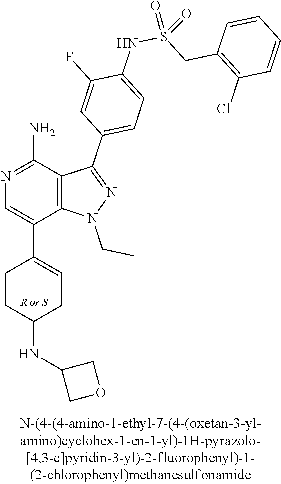 [SMILES: CCn1nc(-c2ccc(NS(=O)(=O)Cc3ccccc3Cl)c(F)c2)c2c(N)ncc(C3=CCC(NC4COC4)CC3)c21] | Intermediate 128 Intermediate 139 |
| B29 (First eluting isomer) | 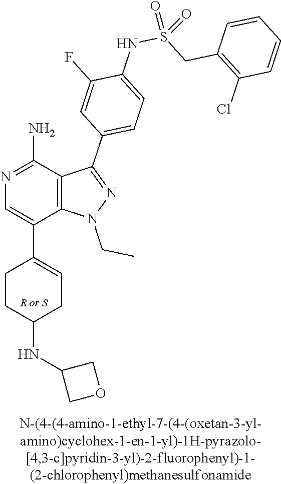 [SMILES: CCn1nc(-c2ccc(NS(=O)(=O)Cc3ccccc3Cl)c(F)c2)c2c(N)ncc(C3=CCC(NC4COC4)CC3)c21] | Intermediate 128 Intermediate 139 |
| B30 | 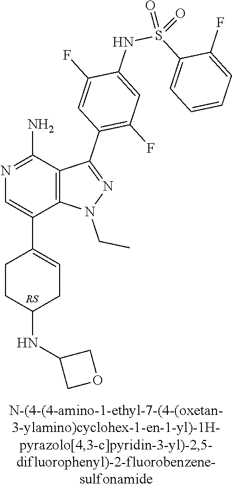 [SMILES: CCn1nc(-c2cc(F)c(NS(=O)(=O)c3ccccc3F)cc2F)c2c(N)ncc(C3=CCC(NC4COC4)CC3)c21] | Intermediate 128 Intermediate 150 |
| B31 | 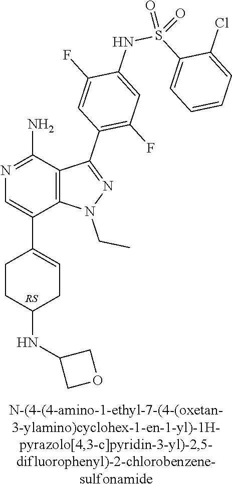 [SMILES: CCn1nc(-c2cc(F)c(NS(=O)(=O)c3ccccc3Cl)cc2F)c2c(N)ncc(C3=CCC(NC4COC4)CC3)c21] | Intermediate 128 Intermediate 147 |
| B32 | 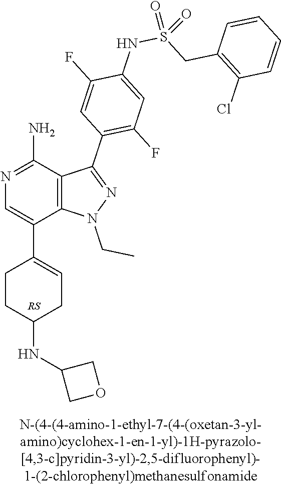 [SMILES: CCn1nc(-c2cc(F)c(NS(=O)(=O)Cc3ccccc3Cl)cc2F)c2c(N)ncc(C3=CCC(NC4COC4)CC3)c21] | Intermediate 128 Intermediate 137 |
| B33 (Second eluting isomer) | 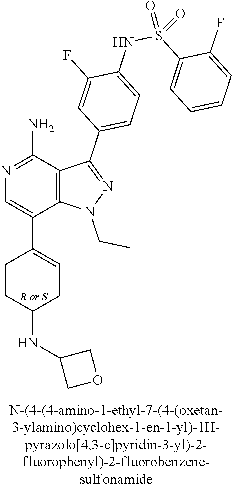 [SMILES: CCn1nc(-c2ccc(NS(=O)(=O)c3ccccc3F)c(F)c2)c2c(N)ncc(C3=CCC(NC4COC4)CC3)c21] | Intermediate 128 Intermediate 148 |
| B34 (First eluting isomer) | 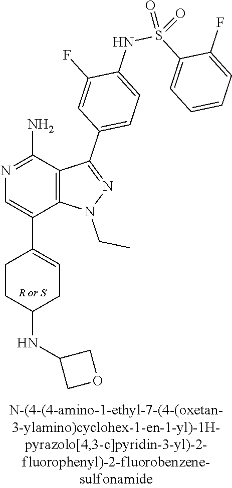 [SMILES: CCn1nc(-c2ccc(NS(=O)(=O)c3ccccc3F)c(F)c2)c2c(N)ncc(C3=CCC(NC4COC4)CC3)c21] | Intermediate 128 Intermediate 148 |
| B35 (Second eluting isomer) | 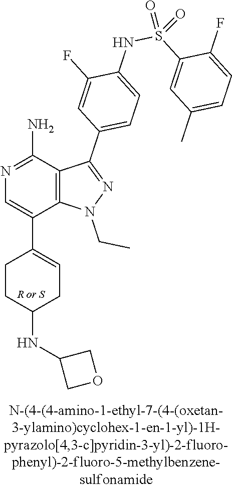 [SMILES: CCn1nc(-c2ccc(NS(=O)(=O)c3cc(C)ccc3F)c(F)c2)c2c(N)ncc(C3=CCC(NC4COC4)CC3)c21] | Intermediate 128 Intermediate 141 |
| B36 (First eluting isomer) | 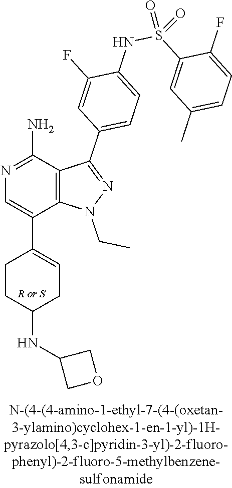 [SMILES: CCn1nc(-c2ccc(NS(=O)(=O)c3cc(C)ccc3F)c(F)c2)c2c(N)ncc(C3=CCC(NC4COC4)CC3)c21] | Intermediate 128 Intermediate 141 |
| B37 | 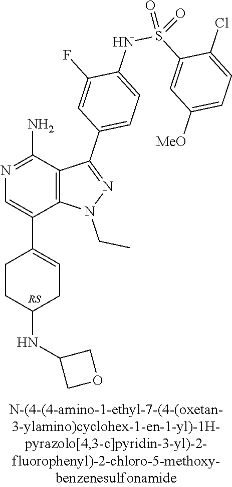 [SMILES: CCn1nc(-c2ccc(NS(=O)(=O)c3cc(OC)ccc3Cl)c(F)c2)c2c(N)ncc(C3=CCC(NC4COC4)CC3)c21] | Intermediate 128 Intermediate 138 |
| B38 (Second eluting isomer) | 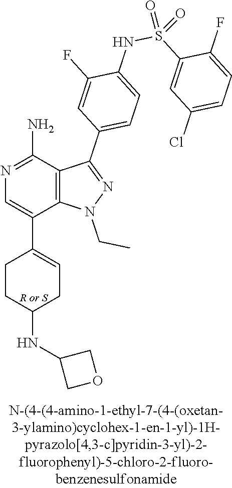 [SMILES: CCn1nc(-c2ccc(NS(=O)(=O)c3cc(Cl)ccc3F)c(F)c2)c2c(N)ncc(C3=CCC(NC4COC4)CC3)c21] | Intermediate 128 Intermediate 140 |
| B39 (First eluting isomer) | 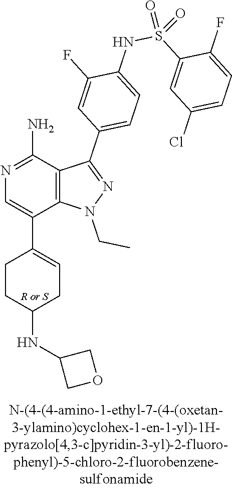 [SMILES: CCn1nc(-c2ccc(NS(=O)(=O)c3cc(Cl)ccc3F)c(F)c2)c2c(N)ncc(C3=CCC(NC4COC4)CC3)c21] | Intermediate 128 Intermediate 140 |

### Preparation of compounds B40 and B41: N-(4-(4-amino-7-(4-((1-fluoropropan-2-yl)amino)cyclohex-1-en-1-yl)-1-isopropyl-1H-pyrazolo[4,3-c]pyridin-3-yl)-2-fluorophenyl)-2-chlorobenzenesulfonamide

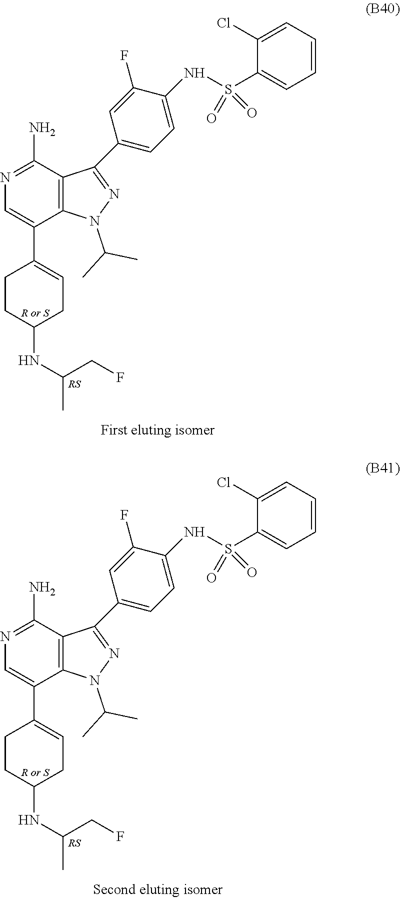 [SMILES: CC(CF)NC1CC=C(c2cnc(N)c3c(-c4ccc(NS(=O)(=O)c5ccccc5Cl)c(F)c4)nn(C(C)C)c23)CC1 ; CC(CF)NC1CC=C(c2cnc(N)c3c(-c4ccc(NS(=O)(=O)c5ccccc5Cl)c(F)c4)nn(C(C)C)c23)CC1]

3-bromo-7-(4-((1-fluoropropan-2-yl)amino)cyclohex-1-en-1-yl)-1-isopropyl-1H-pyrazolo[4,3-c]pyridin-4-amine (intermediate 130) (0.08 g, 0.18 mmol), cesium carbonate (0.15 g, 46 mmol) and 2-chloro-N-(2-fluoro-4-(4,4,5,5-tetramethyl-1,3,2-dioxaborolan-2-yl)phenyl)benzenesulfonamide (intermediate 149) (0.08 g, 0.18 mmol) in a mixture of 1,4-dioxane (4.5 mL) and water (1.1 mL) was degassed with argon, then treated with Pd(dppf)Cl₂.DCM (0.012 g, 0.015 mmol) and irradiated in a microwave at 70° C. for 0.5 h. The resulting reaction mixture was diluted with EtOAc, organic layer separated and washed with saturated brine, dried (MgSO₄), filtered through CELITE® and concentrated in vacuo to give a brown oil. Purification by SFC, provided compound B40 as a white solid (17 mg, 11%) and compound B41 as a white solid (17 mg, 11%).

### Compound B40 (First Eluting Isomer)

¹H NMR (400 MHz, DMSO) δ: 7.99 (1H, dd, J=1.6, 7.8 Hz), 7.58 (1H, d, J=8.0 Hz), 7.53 (1H, dd, J=7.4, 7.4 Hz), 7.45 (1H, ddd, J=7.4, 7.4, 1.4 Hz), 7.38 (1H, s), 7.29 (2H, dd, J=9.8, 18.2 Hz), 7.19 (1H, d, J=8.0 Hz), 5.69 (1H, s), 5.65 (2H, s), 4.92-4.85 (1H, m), 4.42 (1H, d, J=4.5 Hz), 4.30 (1H, d, J=4.8 Hz), 3.19 (1H, s), 3.09 (1H, s), 2.30 (2H, s), 2.00 (2H, s), 1.59 (1H, s), 1.48 (3H, d, J=5.0 Hz), 1.36 (3H, d, J=5.4 Hz), 1.12-1.09 (3H, m). LCMS (Method B): Rt=4.48 min; m/z [M+H]⁺=615.

### Compound B41 (Second Eluting Isomer)

¹H NMR (400 MHz, DMSO) δ: 7.99 (1H, dd, J=1.6, 7.8 Hz), 7.57-7.48 (2H, m), 7.43 (1H, dd, J=7.2, 7.2 Hz), 7.37 (1H, s), 7.27 (2H, dd, J=11.1, 19.8 Hz), 7.14 (1H, d, J=7.1 Hz), 5.69 (1H, s), 5.64 (2H, s), 4.90-4.85 (1H, m), 4.44-4.37 (1H, m), 4.30-4.27 (1H, m), 3.18 (1H, s), 3.07 (1H, s), 2.30 (2H, s), 1.99 (2H, s), 1.47 (3H, s), 1.37 (3H, s), 1.25 (1H, s), 1.17 (1H, t, J=5.3 Hz), 1.09 (3H, d, J=6.9 Hz), 0.91-0.84 (1H, m). LCMS (Method C): Rt=2.90 min; m/z [M+4]⁺=615.

The following compounds in Table 24 were prepared using an analogous method to compounds B40 and B41.

TABLE 24

| Compound | Structure | Starting Materials |
|---|---|---|
| B42 (Second eluting isomer) | 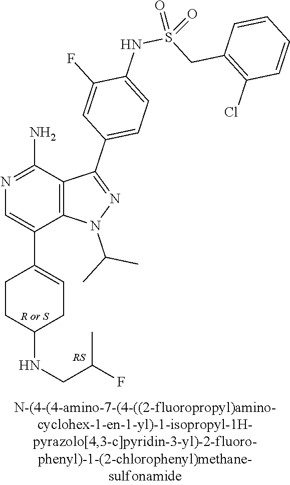 [SMILES: CC(F)CNC1CC=C(c2cnc(N)c3c(-c4ccc(NS(=O)(=O)Cc5ccccc5Cl)c(F)c4)nn(C(C)C)c23)CC1] | Intermediate 131 Intermediate 139 |
| B43 (First eluting isomer) | 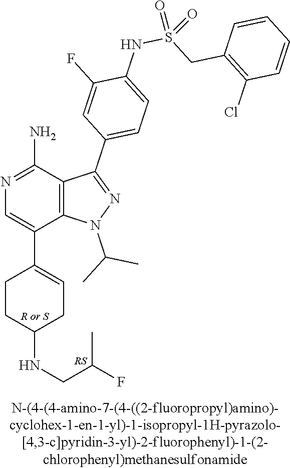 [SMILES: CC(F)CNC1CC=C(c2cnc(N)c3c(-c4ccc(NS(=O)(=O)Cc5ccccc5Cl)c(F)c4)nn(C(C)C)c23)CC1] | Intermediate 131 Intermediate 139 |
| B44 (Second eluting isomer) | 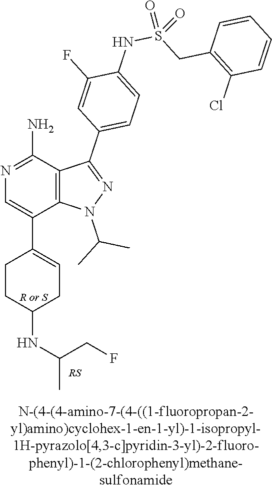 [SMILES: CC(CF)NC1CC=C(c2cnc(N)c3c(-c4ccc(NS(=O)(=O)Cc5ccccc5Cl)c(F)c4)nn(C(C)C)c23)CC1] | Intermediate 130 Intermediate 139 |
| B45 (First eluting isomer) | 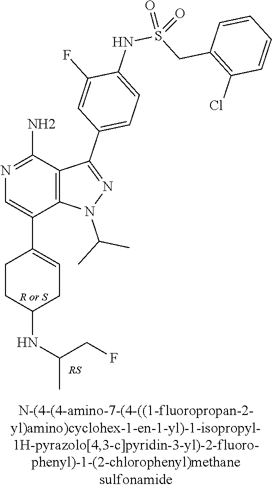 [SMILES: CC(CF)NC1CC=C(c2cnc(N)c3c(-c4ccc(NS(=O)(=O)Cc5ccccc5Cl)c(F)c4)nn(C(C)C)c23)CC1] | Intermediate 130 Intermediate 139 |
| B46 (Second eluting isomer) | 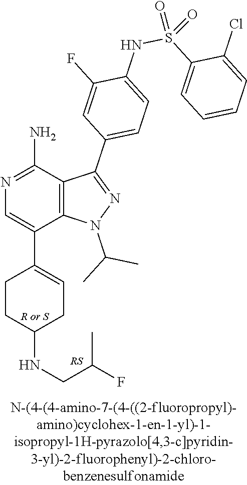 [SMILES: CC(F)CNC1CC=C(c2cnc(N)c3c(-c4ccc(NS(=O)(=O)c5ccccc5Cl)c(F)c4)nn(C(C)C)c23)CC1] | Intermediate 131 Intermediate 149 |
| B47 (First eluting isomer) | 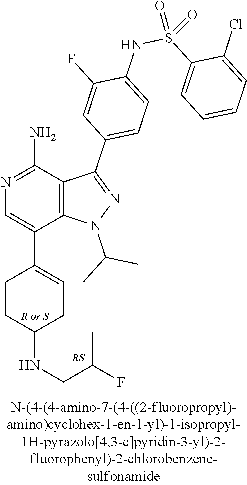 [SMILES: CC(F)CNC1CC=C(c2cnc(N)c3c(-c4ccc(NS(=O)(=O)c5ccccc5Cl)c(F)c4)nn(C(C)C)c23)CC1] | Intermediate 131 Intermediate 149 |
| B48 (Second eluting isomer) | 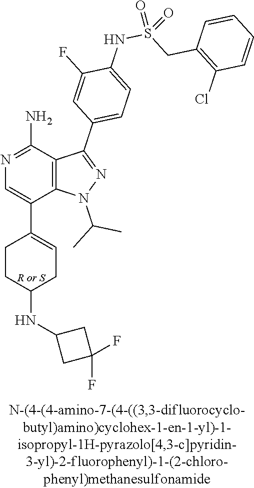 [SMILES: CC(C)n1nc(-c2ccc(NS(=O)(=O)Cc3ccccc3Cl)c(F)c2)c2c(N)ncc(C3=CCC(NC4CC(F)(F)C4)CC3)c21] | Intermediate 132 Intermediate 139 |
| B49 (First eluting isomer) | 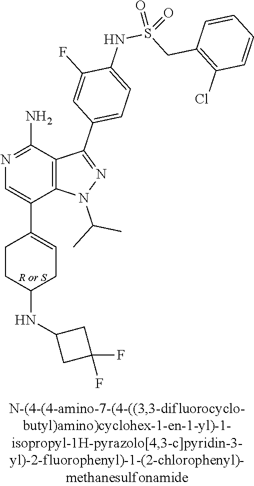 [SMILES: CC(C)n1nc(-c2ccc(NS(=O)(=O)Cc3ccccc3Cl)c(F)c2)c2c(N)ncc(C3=CCC(NC4CC(F)(F)C4)CC3)c21] | Intermediate 132 Intermediate 139 |
| B50 (Second eluting isomer) | 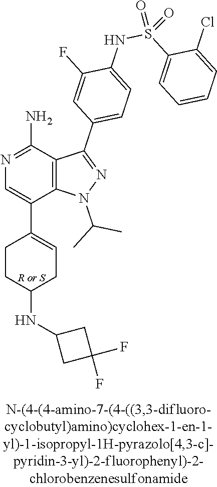 [SMILES: CC(C)n1nc(-c2ccc(NS(=O)(=O)c3ccccc3Cl)c(F)c2)c2c(N)ncc(C3=CCC(NC4CC(F)(F)C4)CC3)c21] | Intermediate 132 Intermediate 149 |
| B51 (First eluting isomer) | 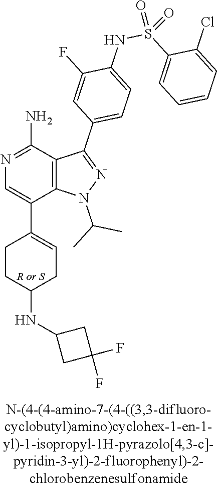 [SMILES: CC(C)n1nc(-c2ccc(NS(=O)(=O)c3ccccc3Cl)c(F)c2)c2c(N)ncc(C3=CCC(NC4CC(F)(F)C4)CC3)c21] | Intermediate 132 Intermediate 149 |
| B52 (Second eluting isomer) | 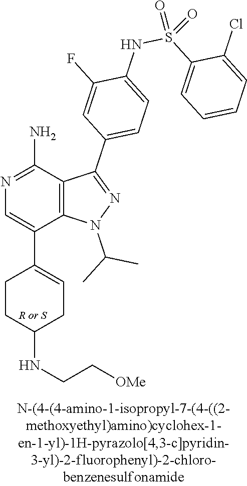 [SMILES: COCCNC1CC=C(c2cnc(N)c3c(-c4ccc(NS(=O)(=O)c5ccccc5Cl)c(F)c4)nn(C(C)C)c23)CC1] | Intermediate 133 Intermediate 149 |
| B53 (First eluting isomer) | 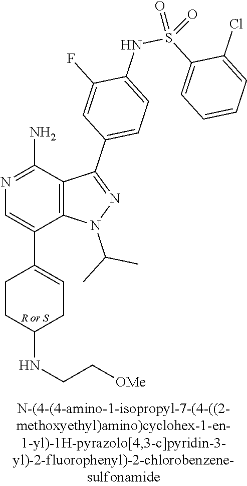 [SMILES: COCCNC1CC=C(c2cnc(N)c3c(-c4ccc(NS(=O)(=O)c5ccccc5Cl)c(F)c4)nn(C(C)C)c23)CC1] | Intermediate 133 Intermediate 149 |
| B54 (First eluting isomer) | 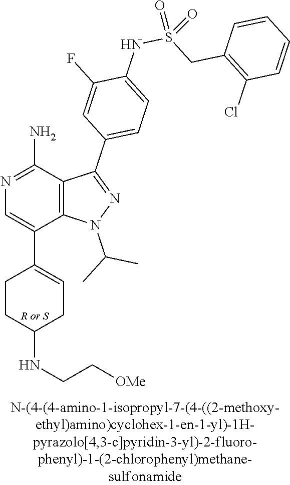 [SMILES: COCCNC1CC=C(c2cnc(N)c3c(-c4ccc(NS(=O)(=O)Cc5ccccc5Cl)c(F)c4)nn(C(C)C)c23)CC1] | Intermediate 133 Intermediate 139 |
| B55 (Second eluting isomer) | 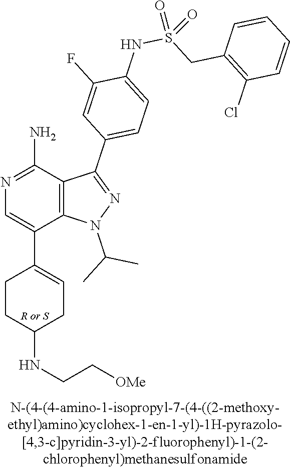 [SMILES: COCCNC1CC=C(c2cnc(N)c3c(-c4ccc(NS(=O)(=O)Cc5ccccc5Cl)c(F)c4)nn(C(C)C)c23)CC1] | Intermediate 133 Intermediate 139 |

### Preparation of compounds B56 and B57: N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide

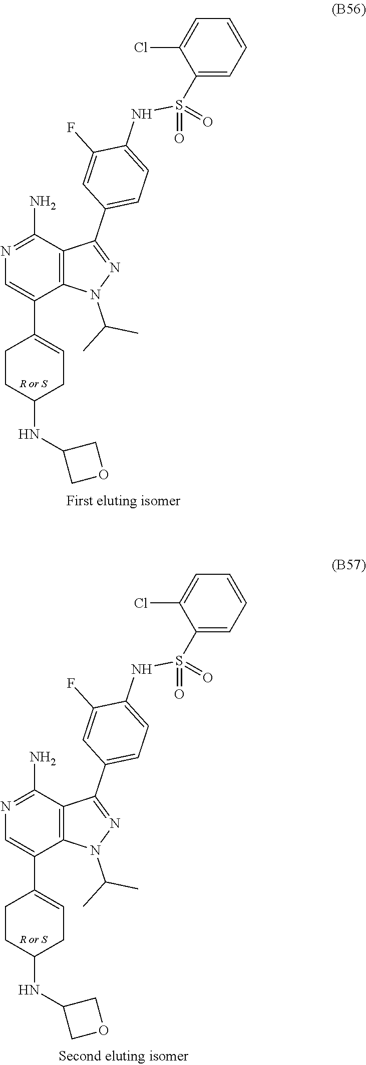 [SMILES: CC(C)n1nc(-c2ccc(NS(=O)(=O)c3ccccc3Cl)c(F)c2)c2c(N)ncc(C3=CCC(NC4COC4)CC3)c21 ; CC(C)n1nc(-c2ccc(NS(=O)(=O)c3ccccc3Cl)c(F)c2)c2c(N)ncc(C3=CCC(NC4COC4)CC3)c21]

3-Oxetanone (CAS: 6704-31-0; 0.022 mL, 0.025 g, 0.342 mmol) was added to a stirred suspension of N-(4-(4-amino-7-(4-aminocyclohex-1-en-1-yl)-1-isopropyl-1H-pyrazolo[4,3-c]pyridin-3-yl)-2-fluorophenyl)-2-chlorobenzenesulfonamide (intermediate 163) (0.16 g, 0.29 mmol) and acetic acid (0.05 mL, 0.05 g, 0.86 mmol) in DCM (6 mL). Sodium triacetoxyborohydride (0.085 g, 0.40 mmol) was added and the mixture stirred at RT for 1 h. Second portions of 3-oxetanone (CAS: 6704-31-0; 0.022 mL, 0.025 g, 0.342 mmol) and sodium triacetoxyborohydride (0.085 g, 0.40 mmol) were added and stirred for a further 2.5 h. The mixture was treated with saturated aqueous NaHCO₃ and extracted with DCM. The combined organic extracts were washed with brine, dried (Na₂SO₄) and concentrated in vacuo. The crude product was purified by prep-HPLC and then charged to an ISOLUTE® SCX-2 cartridge pre-wetted with MeOH. The cartridge was eluted with MeOH, then washed with a solution of ammonia in MeOH (2N) to release the title compounds from the SCX-2 solid. Further purification by SFC gave compound B56 as a white solid (24.3 mg, 14%) and compound B57 as a white solid (24.7 mg, 14%).

### Compound B56 (First Eluting Isomer)

¹H NMR (400 MHz, DMSO) δ: 8.00-7.97 (1H, m), 7.44-7.40 (1H, m), 7.38-7.31 (3H, m), 7.18 (1H, dd, J=8.8, 8.8 Hz), 7.08 (1H, dd, J=2.1, 12.2 Hz), 6.94 (1H, dd, J=1.9, 8.3 Hz), 5.66 (1H, s), 5.60 (2H, s), 4.91 (1H, sept, J=6.6 Hz), 4.68 (2H, dd, J=6.1, 11.0 Hz), 4.37 (2H, t, J=5.6 Hz), 4.04 (1H, quint, J=6.5 Hz), 2.78-2.68 (1H, m), 2.35-2.25 (3H, m), 1.94 (1H, s), 1.83 (1H, s), 1.53 (1H, s), 1.45 (3H, s), 1.35 (3H, s). LCMS (Method B): Rt=3.72 min; m/z [M+H]⁺=611.

Compound B57 (Second Eluting Isomer)

¹H NMR (400 MHz, DMSO) δ: 8.00-7.97 (1H, m), 7.45-7.42 (1H, m), 7.39-7.34 (2H, m), 7.33 (1H, s), 7.19 (1H, dd, J=8.8, 8.8 Hz), 7.10 (1H, dd, J=2.1, 12.0 Hz), 6.96 (1H, dd, J=1.9, 8.4 Hz), 5.65 (1H, s), 5.61 (2H, s), 4.91 (1H, quint, J=6.5 Hz), 4.71-4.65 (2H, m), 4.38 (2H, t, J=5.6 Hz), 4.04 (1H, quint, J=6.8 Hz), 2.79 (1H, s), 2.35-2.25 (3H, m), 1.96-1.90 (1H, m), 1.86-1.79 (1H, m), 1.53-1.51 (1H, m), 1.46-1.44 (3H, m), 1.35 (3H, s). LCMS (Method C): Rt=2.80 min; m/z [M+H]⁺=611.

The following compounds in Table 25 were prepared using an analogous method to compounds B56 and B57

TABLE 25

| Cmpd | Structure | Starting Materials |
|---|---|---|
| B58 (First eluting isomer) | 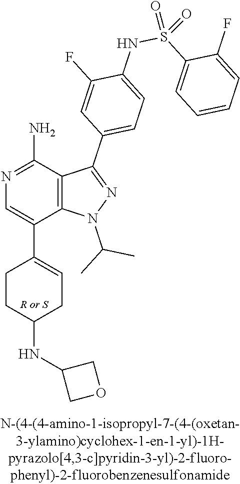 [SMILES: CC(C)n1nc(-c2ccc(NS(=O)(=O)c3ccccc3F)c(F)c2)c2c(N)ncc(C3=CCC(NC4COC4)CC3)c21] | Intermediate 166 3-Oxetanone (CAS: 6704-31-0) |
| B59 (Second eluting isomer) | 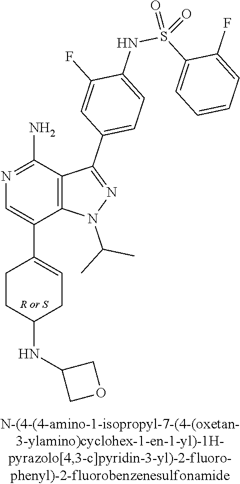 [SMILES: CC(C)n1nc(-c2ccc(NS(=O)(=O)c3ccccc3F)c(F)c2)c2c(N)ncc(C3=CCC(NC4COC4)CC3)c21] | Intermediate 166 3-Oxetanone (CAS: 6704-31-0) |
| B60 | 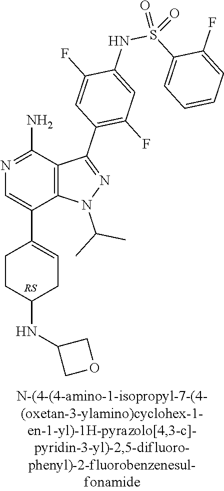 [SMILES: CC(C)n1nc(-c2cc(F)c(NS(=O)(=O)c3ccccc3F)cc2F)c2c(N)ncc(C3=CCC(NC4COC4)CC3)c21] | Intermediate 165 3-Oxetanone (CAS: 6704-31-0) |
| B61 (Second eluting isomer) | 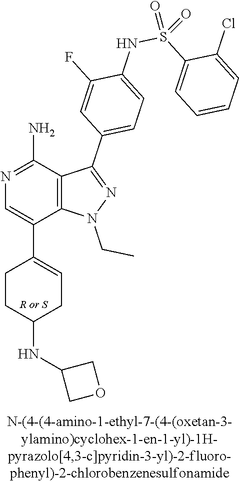 [SMILES: CCn1nc(-c2ccc(NS(=O)(=O)c3ccccc3Cl)c(F)c2)c2c(N)ncc(C3=CCC(NC4COC4)CC3)c21] | Intermediate 164 3-Oxetanone (CAS: 6704-31-0) |
| B62 (First eluting isomer) | 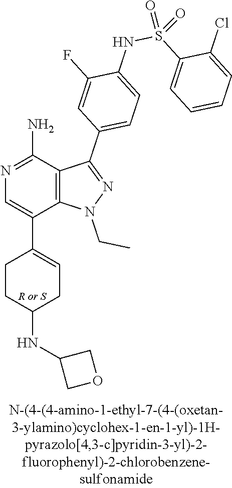 [SMILES: CCn1nc(-c2ccc(NS(=O)(=O)c3ccccc3Cl)c(F)c2)c2c(N)ncc(C3=CCC(NC4COC4)CC3)c21] | Intermediate 164 3-Oxetanone (CAS: 6704-31-0) |

### Preparation of compound B63: N-(4-(4-amino-7-(4-((2-fluoroethyl)amino)cyclohex-1-en-1-yl)-1-isopropyl-1H-pyrazolo[4,3-c]pyridin-3-yl)-2-fluorophenyl)-2-chlorobenzenesulfonamide

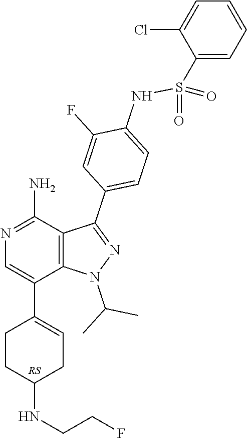 [SMILES: CC(C)n1nc(-c2ccc(NS(=O)(=O)c3ccccc3Cl)c(F)c2)c2c(N)ncc(C3=CCC(NCCF)CC3)c21]

2-Fluoroethyl trifluoromethanesulfonate (CAS: 95353-04-1; 0.020 mL, 0.031 g, 0.16 mmol) was added to a stirred solution of N-(4-(4-amino-7-(4-aminocyclohex-1-en-1-yl)-1-isopropyl-1H-pyrazolo[4,3-c]pyridin-3-yl)-2-fluorophenyl)-2-chlorobenzenesulfonamide (intermediate 163) (0.088 g, 0.16 mmol) and diisopropylamine (0.055 mL, 0.04 g, 0.32 mmol) in 1,4-dioxane (2 mL) and the resulting mixture heated to 90° C. for 24 h. The mixture was cooled to RT, diluted with water (20 mL) and extracted with EtOAc (3×20 mL). The combined extracts were dried (MgSO₄), filtered and concentrated in vacuo. Purification by column chromatography on SiO₂ eluting with a gradient of 0-10% MeOH in DCM gave the title compound as a white solid (0.035 g, 37%). ¹H NMR (400 MHz, DMSO) δ: 7.98 (1H, dd, J=1.5, 7.9 Hz), 7.66-7.56 (2H, m), 7.48 (1H, ddd, J=7.5, 7.5, 1.2 Hz), 7.39 (1H, s), 7.37-7.31 (2H, m), 7.27 (1H, dd, J=1.7, 8.4 Hz), 5.70 (1H, s), 5.67 (2H, s), 4.85 (1H, sept, J=6.3 Hz), 4.63 (2H, td, J=4.5, 47.9 Hz), 3.61-3.51 (1H, m), 3.20-3.12 (3H, m), 2.32 (2H, dd, J=1.7, 3.6 Hz), 2.17-2.06 (2H, m), 1.66 (1H, t, J=18.0 Hz), 1.46 (3H, d, J=3.4 Hz), 1.35 (3H, d, J=5.4 Hz), 1.23 (5H, d, J=6.4 Hz). LCMS (Method A): Rt=2.78 min; m/z [M+H]⁺=601

Compound A4: ¹H NMR (400 MHz, DMSO) δ: 8.38 (1H, br. s), 7.72 (1H, dd, J=2.8, 6.0 Hz), 7.56 (1H, s), 7.52-7.49 (1H, m), 7.27 (1H, dd, J=9.0, 9.0 Hz), 7.09 (1H, dd, J=7.5, 13.0 Hz), 6.98 (1H, dd, J=7.4, 11.1 Hz), 5.46 (2H, s), 4.83 (1H, 6.5 Hz), 3.60 (2H, t, J=5.0 Hz), 3.34 (3H, s), 3.17 (3H, t, J=4.9 Hz), 2.91 (1H, t, J=10.3 Hz), 2.19 (2H, d, J=10.4 Hz), 2.02 (2H, d, J=11.9 Hz), 1.70-1.54 (4H, m), 1.51 (6H, d, J=6.5 Hz). LCMS (Method B): Rt=2.93 min; m/z [M+H]⁺=651/653.

Compound A5: ¹H NMR (400 MHz, DMSO) δ: 8.41 (1H, br. s), 7.56 (1H, s), 7.56-7.53 (1H, m), 7.29-7.25 (2H, m), 7.20 (1H, t, J=73.7 Hz), 7.12-7.06 (1H, m), 6.96 (1H, dd, J=7.4, 11.2 Hz), 5.45 (2H, s), 4.83 (1H, sept, J=6.4 Hz), 3.62-3.57 (2H, m), 3.34 (4H, s), 3.17 (3H, t, J=4.3 Hz), 2.91 (1H, t, J=10.2 Hz), 2.20 (2H, d, J=10.3 Hz), 2.03 (2H, d, J=12.4 Hz), 1.71-1.54 (3H, m), 1.53-1.49 (6H, m). LCMS (Method A): Rt=3.24 min; m/z [M+H]⁺=683.

Compound A6: ¹H NMR (400 MHz, DMSO) δ: 7.78 (1H, ddd, J=7.6, 7.6, 1.8 Hz), 7.57 (1H, s), 7.51-7.44 (1H, m), 7.31-7.19 (3H, m), 7.13 (1H, dd, J=1.8, 11.9 Hz), 7.03 (1H, dd, J=1.4, 8.5 Hz), 5.51 (2H, s), 4.83 (1H, sept, J=6.4 Hz), 3.57-3.52 (2H, m), 3.06 (2H, t, J=5.0 Hz), 3.02-2.96 (1H, m), 2.91 (1H, t, J=11.5 Hz), 2.15 (2H, d, J=10.6 Hz), 2.00 (2H, d, J=12.6 Hz), 1.68-1.55 (2H, m), 1.51 (6H, d, J=6.4 Hz), 1.47-1.44 (1H, m). LCMS (Method C): Rt=2.69 min; m/z [M+H]⁺=599.

Compound A7: ¹H NMR (400 MHz, DMSO) δ: 8.60 (1H, br. s), 7.57 (1H, s), 7.41 (1H, dd, J=7.6, 7.6 Hz), 7.35 (1H, ddd, J=1.7, 6.1, 7.9 Hz), 7.29-7.16 (3H, m), 6.11 (2H, br. s), 4.87 (1H, sept, J=6.4 Hz), 4.70-4.68 (3H, m), 3.87 (3H, s), 3.61 (2H, t, J=5.0 Hz), 3.34 (1H, s), 3.17 (3H, s), 2.96-2.89 (1H, m), 2.21 (2H, s), 2.02 (2H, d, J=10.2 Hz), 1.68-1.56 (3H, m), 1.52 (6H, d, J=6.4 Hz). LCMS (Method C): Rt=2.82 min; m/z [M+H]⁺=647.

Compound A8: ¹H NMR (400 MHz, DMSO) δ: 8.37 (1H, br. s), 7.56 (1H, s), 7.25 (1H, dd, J=3.2, 5.7 Hz), 7.13-7.04 (2H, m), 6.98-6.93 (2H, m), 5.41 (2H, s), 4.87-4.78 (1H, m), 3.99 (2H, q, J=7.0 Hz), 3.58 (2H, t, J=5.0 Hz), 3.34 (3H, s), 3.18-3.13 (4H, m), 2.91 (1H, t, J=10.7 Hz), 2.18 (2H, d, J=10.9 Hz), 2.01 (2H, d, J=12.8 Hz), 1.68-1.54 (3H, m), 1.50 (6H, d, J=6.5 Hz), 1.30 (3H, t, J=7.0 Hz). LCMS (Method C): Rt=3.04 min; m/z [M+H]⁺=661.

Compound A9: ¹H NMR (400 MHz, DMSO) δ: 7.68 (2H, ddd, J=2.5, 2.5, 9.3 Hz), 7.56 (1H, s), 7.04 (1H, dd, J=10.2, 10.2 Hz), 7.00-6.93 (3H, m), 5.38 (2H, s), 4.83 (1H, sept, J=6.4 Hz), 3.78 (3H, s), 3.55 (2H, t, J=5.1 Hz), 3.08-3.03 (3H, m), 2.91 (1H, t, J=11.4 Hz), 2.15 (2H, d, J=10.3 Hz), 1.98 (2H, d, J=12.3 Hz), 1.67-1.43 (10H, m). LCMS (Method B): Rt=3.12 min; m/z [M+H]⁺=629.

Compound A10: ¹H NMR (400 MHz, DMSO) δ: 7.76 (1H, dd, J=1.7, 7.8 Hz), 7.57 (1H, s), 7.45 (1H, ddd, J=4.2, 4.2, 11.4 Hz), 7.14 (1H, dd, J=7.3, 12.7 Hz), 7.09 (1H, d, J=8.3 Hz), 7.03 (1H, dd, J=7.4, 10.9 Hz), 6.97 (1H, ddd, J=7.5, 7.5, 0.9 Hz), 5.39 (2H, s), 4.84 (1H, sept, J=6.4 Hz), 3.77 (3H, s), 3.52 (2H, t, J=5.2 Hz), 3.01 (2H, t, J=4.9 Hz), 2.91 (2H, tt, J=2.9, 11.6 Hz), 2.14 (2H, d, J=10.3 Hz), 1.99 (2H, d, J=12.4 Hz), 1.61 (2H, q, J=12.0 Hz), 1.51-1.48 (6H, m), 1.47-1.40 (2H, m). LCMS (Method B): Rt=3.24 min; m/z [M+H]⁺=629.

Compound A11: ¹H NMR (400 MHz, DMSO) δ: 8.17 (1H, br. s), 7.56 (1H, s), 7.37-7.30 (2H, m), 7.27-7.25 (1H, m), 7.04 (1H, dd, J=7.5, 13.1 Hz), 7.01-6.92 (2H, m), 5.39 (2H, s), 4.83 (1H, sept, J=6.4 Hz), 3.77 (3H, s), 3.57 (2H, t, J=5.1 Hz), 3.13 (3H, t, J=4.8 Hz), 2.90 (1H, t, J=11.4 Hz), 2.18 (2H, d, J=10.1 Hz), 2.00 (2H, d, J=12.4 Hz), 1.67-1.48 (10H, m). LCMS (Method B): Rt=3.11 min; m/z [M+H]⁺=629.

Compound A12: ¹H NMR (400 MHz, DMSO) δ: 7.78 (1H, ddd, J=7.6, 7.6, 1.7 Hz), 7.69-7.66 (2H, m), 7.44-7.28 (5H, m), 5.76 (1H, s), 5.58 (2H, br. s), 4.90 (1H, br. s), 4.67 (2H, t, J=6.7 Hz), 4.56 (1H, s), 4.44 (2H, t, J=6.3 Hz), 4.03 (1H, quint, J=6.6 Hz), 2.97 (1H, t, J=11.1 Hz), 2.90 (1H, s), 1.92-1.86 (2H, m), 1.70-1.62 (6H, m), 1.49 (6H, d, J=6.4 Hz). LCMS (Method A): Rt=2.69 min; m/z [M+H]⁺=597.

Compound A13: ¹H NMR (400 MHz, CDCl₃) δ: 7.61 (1H, s), 7.53-7.44 (3H, m), 7.22-7.15 (2H, m), 5.08 (2H, s), 4.91 (1H, sept, J=6.5 Hz), 3.91 (3H, s), 3.54 (2H, t, J=5.1 Hz), 3.39 (3H, s), 2.94 (1H, tt, J=2.8, 11.8 Hz), 2.88 (2H, t, J=5.1 Hz), 2.68-2.60 (1H, m), 2.18 (2H, dd, J=3.1, 13.2 Hz), 2.04 (2H, d, J=13.4 Hz), 1.68-1.61 (2H, m), 1.58 (6H, d, J=6.5 Hz), 1.41-1.25 (2H, m). LCMS (Method B): Rt=3.20 min; m/z [M+H]⁺=647.

Compound A14: ¹H NMR (400 MHz, DMSO) δ: 8.15 (1H, s), 7.74 (2H, ddd, J=2.1, 2.1, 8.8 Hz), 7.57 (1H, s), 7.49 (2H, ddd, J=2.1, 2.1, 8.8 Hz), 6.99 (2H, ddd, J=7.4, 12.1, 35.1 Hz), 5.42-5.38 (2H, m), 4.83 (1H, sept, J=6.3 Hz), 4.56 (2H, s), 3.58 (2H, t, J=5.1 Hz), 3.15 (3H, t, J=5.5 Hz), 2.91 (1H, t, J=11.5 Hz), 2.18 (2H, d, J=11.2 Hz), 2.00 (2H, d, J=13.3 Hz), 1.68-1.53 (5H, m), 1.51 (6H, d, J=6.5 Hz). LCMS (Method B): Rt=3.33 min; m/z [M+H]⁺=633.

Compound A15: ¹H NMR (400 MHz, DMSO) δ: 8.43 (1H, br. s), 8.02 (1H, dd, J=6.4, 8.8 Hz), 7.57 (1H, s), 7.44 (1H, dd, J=2.5, 8.8 Hz), 7.25 (1H, ddd, J=8.5, 8.5, 2.6 Hz), 7.03-6.94 (2H, m), 5.45 (2H, s), 4.83 (1H, sept, J=6.6 Hz), 4.57 (1H, s), 3.60 (2H, t, J=5.0 Hz), 2.92 (1H, t, J=10.9 Hz), 2.19 (3H, d, J=10.4 Hz), 2.01 (2H, d, J=12.2 Hz), 1.69-1.55 (4H, m), 1.51 (6H, d, J=6.5 Hz), 1.24 (1H, s). LCMS (Method B): Rt=3.30 min; m/z [M+H]⁺=651/653.

Compound A16: ¹H NMR (400 MHz, DMSO) δ: 8.47 (1H, br. s), 7.79 (1H, dd, J=8.1, 8.1 Hz), 7.57 (1H, s), 7.47 (1H, dd, J=1.8, 9.6 Hz), 7.33 (1H, dd, J=1.8, 8.4 Hz), 7.08 (1H, dd, J=7.4, 12.8 Hz), 7.01 (1H, dd, J=7.4, 10.9 Hz), 5.60 (2H, s), 4.84 (1H, sept, J=6.4 Hz), 3.61 (2H, t, J=5.0 Hz), 3.18 (3H, t, J=5.0 Hz), 2.92 (1H, t, J=10.5 Hz), 2.20 (2H, d, J=9.9 Hz), 2.02 (2H, d, J=11.3 Hz), 1.69-1.55 (4H, m), 1.52 (6H, d, J=6.4 Hz). LCMS (Method B): Rt=3.45 min; m/z [M+H]⁺=651.

Compound A17: ¹H NMR (400 MHz, DMSO) δ: 7.59 (1H, s), 7.37-7.32 (2H, m), 7.21 (1H, dd, J=7.5, 12.8 Hz), 7.15-7.10 (2H, m), 7.04 (1H, dd, J=7.0, 11.5 Hz), 5.45 (2H, s), 4.87 (1H, sept, J=6.6 Hz), 4.18 (2H, s), 3.55 (2H, t, J=5.2 Hz), 3.11-3.03 (4H, m), 2.93 (1H, t, J=11.6 Hz), 2.17 (2H, d, J=10.3 Hz), 2.01 (2H, d, J=12.7 Hz), 1.63 (2H, q, J=12.2 Hz), 1.55-1.46 (9H, m). LCMS (Method B): Rt=3.30 min; m/z [M+H]⁺=631.

Compound A18: ¹H NMR (400 MHz, DMSO) δ: 7.84 (1H, ddd, J=7.6, 7.6, 1.3 Hz), 7.55-7.49 (2H, m), 7.27 (2H, dd, J=7.9, 7.9 Hz), 7.12 (1H, dd, J=8.7, 8.7 Hz), 6.87-6.79 (2H, m), 5.32 (2H, s), 4.84 (1H, sept, J=6.1 Hz), 3.52 (2H, t, J=5.3 Hz), 2.99 (2H, t, J=4.9 Hz), 2.91 (2H, t, J=11.2 Hz), 2.14 (2H, d, J=10.5 Hz), 1.99 (2H, d, J=12.9 Hz), 1.61 (2H, dd, J=11.7, 24.7 Hz), 1.51-1.39 (8H, m), 1.09 (1H, t, J=7.0 Hz). LCMS (Method B): Rt=3.12 min; m/z [M+H]⁺=599.

Compound A19: ¹H NMR (400 MHz, DMSO) δ: 8.27 (1H, br. s), 7.99-7.96 (1H, m), 7.56 (1H, s), 7.46-7.43 (1H, m), 7.41-7.36 (2H, m), 7.01-6.91 (2H, m), 5.39 (2H, s), 4.82 (1H, sept, J=6.4 Hz), 3.58 (2H, t, J=5.1 Hz), 3.34 (3H, s), 3.14 (3H, t, J=4.8 Hz), 2.91 (1H, t, J=11.1 Hz), 2.18 (2H, d, J=11.1 Hz), 2.02 (2H, d, J=12.7 Hz), 1.68-1.53 (4H, m), 1.50 (6H, d, J=6.5 Hz). LCMS (Method B): Rt=3.24 min; m/z [M+H]⁺=633.

Compound A20: ¹H NMR (400 MHz, DMSO) δ: 8.06 (1H, br. s), 7.58 (1H, s), 7.53-7.49 (1H, m), 7.40-7.37 (1H, m), 7.30-7.22 (3H, m), 6.99 (1H, dd, J=7.4, 11.1 Hz), 5.45 (2H, s), 4.85 (1H, sept, J=6.3 Hz), 4.31 (2H, s), 3.56 (2H, t, J=5.1 Hz), 3.14-3.05 (3H, m), 2.92 (1H, t, J=11.6 Hz), 2.18 (2H, d, J=9.8 Hz), 2.01 (2H, d, J=12.2 Hz), 1.69-1.48 (10H, m). LCMS (Method B): Rt=3.32 min; m/z [M+H]⁺=647/649.

Compound A21: ¹H NMR (400 MHz, DMSO) δ: 8.34 (1H, br. s), 8.13 (1H, dd, J=2.2, 6.4 Hz), 7.97 (1H, ddd, J=2.3, 4.3, 8.5 Hz), 7.57 (1H, s), 7.46 (1H, dd, J=9.0, 9.0 Hz), 7.09 (1H, dd, J=7.5, 13.0 Hz), 6.97 (1H, dd, J=7.4, 11.1 Hz), 5.40 (2H, s), 4.83 (1H, sept, J=6.4 Hz), 3.58 (2H, t, J=5.0 Hz), 3.19-3.11 (3H, m), 2.92 (1H, t, J=11.2 Hz), 2.18 (2H, d, J=11.6 Hz), 2.00 (2H, d, J=12.5 Hz), 1.68-1.56 (4H, m), 1.51 (6H, d, J=6.5 Hz). LCMS (Method A): Rt=2.91 min; m/z [M+H]⁺=642.

Compound A22: ¹H NMR (400 MHz, DMSO) δ: 9.55 (1H, s), 7.58 (1H, s), 7.42 (1H, ddd, J=7.7, 7.7, 1.4 Hz), 7.36-7.30 (1H, m), 7.23 (1H, dd, J=7.3, 12.8 Hz), 7.17-7.10 (2H, m), 7.05 (1H, dd, J=7.4, 10.7 Hz), 5.47 (2H, s), 4.91 (1H, sept, J=6.5 Hz), 4.25 (2H, s), 4.09 (1H, d, J=3.5 Hz), 3.16 (3H, s), 2.97 (2H, t, J=9.7 Hz), 2.60 (6H, s), 2.04 (4H, d, J=7.1 Hz), 1.67-1.57 (4H, m), 1.52 (6H, d, J=6.4 Hz). LCMS (Method B): Rt=3.26 min; m/z [M+H]⁺=601.

Compound A23: ¹H NMR (400 MHz, DMSO) δ: 8.33 (1H, br. s), 7.56 (1H, s), 7.45-7.37 (1H, m), 7.12 (1H, dd, J=7.5, 13.2 Hz), 7.07-7.02 (2H, m), 6.94 (1H, dd, J=7.5, 11.2 Hz), 5.40 (2H, s), 4.82 (1H, sept, J=6.4 Hz), 3.58 (2H, t, J=5.0 Hz), 3.34 (3H, s), 3.19-3.15 (3H, m), 2.91 (1H, t, J=11.0 Hz), 2.17 (2H, d, J=11.0 Hz), 2.01 (2H, d, J=12.3 Hz), 1.52-1.49 (6H, m). LCMS (Method B): Rt=3.06 min; m/z [M+H]⁺=635.

Compound A24: ¹H NMR (400 MHz, DMSO) δ: 8.32 (1H, br. s), 7.82 (1H, ddd, J=8.5, 8.5, 6.8 Hz), 7.56 (1H, s), 7.24 (1H, ddd, J=9.7, 9.7, 2.5 Hz), 7.10 (1H, dd, J=2.2, 8.5 Hz), 7.04 (1H, dd, J=7.4, 13.1 Hz), 6.95 (1H, dd, J=7.4, 11.2 Hz), 5.40 (2H, s), 4.82 (1H, sept, J=6.3 Hz), 3.58 (2H, t, J=5.0 Hz), 3.20-3.12 (3H, m), 2.90 (1H, t, J=11.1 Hz), 2.17 (2H, d, J=11.0 Hz), 2.00 (2H, d, J=12.1 Hz), 1.68-1.53 (4H, m), 1.50 (6H, d, J=6.4 Hz). LCMS (Method B): Rt=3.16 min; m/z [M+H]⁺=635.

Compound A25: ¹H NMR (400 MHz, DMSO) δ: 8.34 (1H, br. s), 7.60-7.55 (2H, m), 7.50-7.42 (1H, m), 7.25-7.19 (1H, m), 7.06 (1H, dd, J=7.5, 13.1 Hz), 6.96 (1H, dd, J=7.4, 11.2 Hz), 5.40 (2H, s), 4.82 (1H, sept, J=6.5 Hz), 3.58 (2H, t, J=5.0 Hz), 3.34 (3H, s), 3.16 (3H, t, J=5.3 Hz), 2.91 (1H, t, J=11.1 Hz), 2.17 (2H, d, J=11.2 Hz), 2.01 (2H, d, J=12.5 Hz), 1.70-1.54 (4H, m), 1.50 (6H, d, J=6.4 Hz). LCMS (Method B): Rt=3.17 min; m/z [M+H]⁺=635.

Compound A26: ¹H NMR (400 MHz, DMSO) δ: 7.58 (1H, s), 7.41 (1H, ddd, J=7.7, 7.7, 1.7 Hz), 7.34-7.28 (1H, m), 7.22 (1H, dd, J=7.5, 13.1 Hz), 7.15-7.10 (2H, m), 6.99 (1H, dd, J=7.3, 11.1 Hz), 5.45 (2H, s), 4.85 (1H, sept, J=6.4 Hz), 4.17 (2H, s), 3.56 (2H, t, J=5.1 Hz), 3.33 (3H, s), 3.09-3.06 (3H, m), 2.92 (1H, t, J=11.6 Hz), 2.17 (2H, d, J=11.8 Hz), 2.01 (2H, d, J=12.4 Hz), 1.68-1.47 (10H, m). LCMS (Method A): Rt=2.94 min; m/z [M+H]⁺=631.

Compound A27: ¹H NMR (400 MHz, DMSO) δ: 7.55 (1H, s), 7.28 (1H, dd, J=3.2, 5.6 Hz), 7.26-7.04 (4H, m), 5.85 (2H, s), 4.93 (1H, sept, J=6.4 Hz), 3.76-3.75 (3H, m), 3.47 (4H, s), 3.29 (7H, s), 2.96-2.96 (6H, m), 2.03-1.89 (4H, m), 1.66-1.56 (4H, m), 1.51 (6H, d, J=6.4 Hz). LCMS (Method B): Rt=3.51 min; m/z [M+H]⁺=705.

Compound A28: ¹H NMR (400 MHz, DMSO) δ:8.39 (1H, br. s), 7.56 (1H, s), 7.27 (1H, dd, J=3.3, 5.5 Hz), 7.16-7.05 (2H, m), 7.00-6.92 (2H, m), 5.42 (2H, s), 4.83 (1H, sept, J=6.1 Hz), 3.74 (3H, s), 3.59 (2H, t, J=4.9 Hz), 3.34 (3H, s), 3.16 (3H, t, J=4.6 Hz), 2.91 (1H, t, J=9.9 Hz), 2.20 (2H, d, J=8.1 Hz), 2.00 (2H, d, J=12.0 Hz), 1.69-1.50 (9H, m), 1.24 (1H, s). LCMS (Method B): Rt=3.04 min; m/z [M+H]⁺=647.

Compound A29: ¹H NMR (400 MHz, DMSO) δ: 8.06 (2H, d, J=8.4 Hz), 7.87 (1H, d, J=7.6 Hz), 7.74 (1H, dd, J=7.5, 7.5 Hz), 7.54 (1H, s), 7.17-7.05 (2H, m), 5.89 (2H, s), 4.92 (1H, quint, J=6.1 Hz), 3.45 (4H, s), 3.29 (7H, s), 2.97 (5H, s), 2.00-1.93 (4H, m), 1.59-1.47 (10H, m). LCMS (Method B): Rt=3.87 min; m/z [M+H]⁺=725.

Compound A30: ¹H NMR (400 MHz, DMSO) δ: 8.33 (1H, s), 8.02 (1H, d, J=7.8 Hz), 7.99 (1H, s), 7.78 (1H, d, J=7.7 Hz), 7.67 (1H, dd, J=7.8, 7.8 Hz), 7.56 (1H, s), 7.05 (1H, dd, J=7.5, 13.0 Hz), 6.95 (1H, dd, J=7.5, 11.2 Hz), 5.39 (2H, s), 4.82 (1H, sept, J=6.1 Hz), 3.58 (2H, t, J=4.9 Hz), 3.15 (3H, t, J=4.4 Hz), 2.91 (1H, t, J=11.1 Hz), 2.18 (2H, d, J=10.4 Hz), 2.02 (2H, d, J=12.5 Hz), 1.66 (4H, sept, J=11 Hz), 1.52-1.48 (5H, m), 1.24 (1H, s). LCMS (Method A): Rt=3.34 min; m/z [M+H]⁺=667.

Compound A31: ¹H NMR (400 MHz, DMSO) δ: 8.30 (1H, br. s), 7.56 (1H, s), 7.52-7.47 (1H, m), 7.33-7.22 (2H, m), 7.08 (1H, dd, J=7.5, 13.1 Hz), 6.96 (1H, dd, J=7.4, 11.2 Hz), 5.40 (2H, s), 4.78 (1H, sept, J=6.5 Hz), 3.58 (2H, t, J=4.9 Hz), 3.17 (3H, s), 2.92 (1H, t, J=11.1 Hz), 2.18 (2H, d, J=10.8 Hz), 2.02 (2H, d, J=12.7 Hz), 1.53-1.49 (10H, m). LCMS (Method B): Rt=3.09 min; m/z [M+H]⁺=635.

Compound A32: ¹H NMR (400 MHz, DMSO) δ: 8.21 (1H, s), 7.80-7.76 (2H, m), 7.56 (1H, s), 7.27-7.21 (2H, m), 7.03 (1H, dd, J=7.5, 13.1 Hz), 6.94 (1H, dd, J=7.4, 11.2 Hz), 5.39 (2H, s), 4.82 (1H, sept, J=6.5 Hz), 3.57 (2H, t, J=5.1 Hz), 3.13 (3H, t, J=5.0 Hz), 2.90 (1H, t, J=11.3 Hz), 2.17 (2H, d, J=10.3 Hz), 2.00 (2H, d, J=12.0 Hz), 1.68-1.48 (10H, m). LCMS (Method B): Rt=3.08 min; m/z [M+H]⁺=617.

Compound A33: ¹H NMR (400 MHz, DMSO) δ: 8.34 (1H, s), 7.59-7.55 (2H, m), 7.51-7.43 (2H, m), 7.28-7.23 (1H, m), 7.04 (1H, dd, J=7.5, 13.1 Hz), 6.95 (1H, dd, J=7.4, 11.2 Hz), 5.40 (2H, s), 4.82 (1H, sept, J=6.4 Hz), 3.58 (2H, t, J=5.1 Hz), 3.34 (2H, s), 3.16 (3H, t, J=5.0 Hz), 2.91 (1H, t, J=11.3 Hz), 2.18 (2H, d, J=11.1 Hz), 2.01 (2H, d, J=12.7 Hz), 1.68-1.49 (10H, m), 1.23 (1H, s). LCMS (Method B): Rt=3.07 min; m/z [M+H]⁺=617.

Compound A34: ¹H NMR (400 MHz, DMSO) δ: 7.97 (1H, dd, J=1.5, 7.9 Hz), 7.62 (1H, ddd, J=8.0, 8.0, 1.3 Hz), 7.59-7.55 (2H, m), 7.47 (1H, ddd, J=4.1, 4.1, 11.0 Hz), 7.36-7.28 (2H, m), 7.25 (1H, dd, J=1.7, 8.3 Hz), 5.53 (2H, br. s), 4.87 (1H, sept, J=6.4 Hz), 4.66 (2H, t, J=6.7 Hz), 4.37 (2H, t, J=6.3 Hz), 4.07 (1H, quint, J=6.8 Hz), 2.88 (1H, t, J=11.6 Hz), 1.94-1.86 (4H, m), 1.52-1.48 (8H, m), 1.29 (2H, q, J=12.2 Hz). LCMS (Method A): Rt=2.76 min; m/z [M+H]⁺=613/615.

Compound A35: ¹H NMR (400 MHz, DMSO) δ: 7.81 (1H, ddd, J=7.7, 7.7, 1.5 Hz), 7.61-7.51 (2H, m), 7.30 (2H, dd, J=8.6, 16.4 Hz), 7.18-7.08 (2H, m), 5.79 (2H, s), 4.93 (1H, sept, J=6.3 Hz), 3.49-3.42 (5H, m), 3.28 (7H, s), 2.93 (5H, br. s), 2.01-1.95 (2H, m), 1.93-1.89 (2H, m), 1.59-1.54 (4H, m), 1.50 (6H, d, J=6.4 Hz). LCMS (Method A): Rt=2.82 min; m/z [M+H]⁺=675.

Compound A36: ¹H NMR (400 MHz, DMSO) δ: 7.79 (1H, dd, J=7.2, 7.2 Hz), 7.56 (1H, s), 7.51 (1H, dd, J=6.4, 12.0 Hz), 7.24 (2H, dd, J=7.6, 7.6 Hz), 7.10 (1H, dd, J=7.3, 12.6 Hz), 7.03 (1H, dd, J=7.5, 10.5 Hz), 5.53 (2H, s), 4.9 (1H, sept, J=6.5 Hz), 3.62 (2H, s), 3.22-3.14 (3H, m), 2.99 (1H, t, J=10.5 Hz), 2.69 (3H, s), 2.04 (4H, d, J=9.2 Hz), 1.75-1.61 (4H, m), 1.50 (6H, d, J=6.3 Hz), 1.12-1.06 (1H, m). LCMS (Method A): Rt=2.73 min; m/z [M+H]⁺=631.

Compound A37: ¹H NMR (400 MHz, DMSO) δ: 7.78 (1H, ddd, J=7.6, 7.6, 1.8 Hz), 7.56 (1H, s), 7.47-7.41 (1H, m), 7.23-7.17 (2H, m), 7.05 (1H, dd, J=7.5, 13.2 Hz), 6.94 (1H, dd, J=7.5, 11.2 Hz), 5.39 (2H, s), 4.82 (1H, sept, J=6.4 Hz), 3.58 (2H, t, J=5.0 Hz), 3.14 (3H, t, J=4.5 Hz), 2.91 (1H, t, J=10.9 Hz), 2.18 (2H, d, J=11.0 Hz), 2.00 (2H, d, J=12.5 Hz), 1.69-1.52 (4H, m), 1.50 (6H, d, J=6.4 Hz), 1.24 (1H, s). LCMS (Method B): Rt=3.08 min; m/z [M+H]⁺=617.

Compound A38: ¹H NMR (400 MHz, DMSO) δ: 7.82 (1H, ddd, J=7.6, 7.6, 1.7 Hz), 7.59 (1H, dd, J=5.7, 13.0 Hz), 7.53 (1H, s), 7.38-7.29 (2H, m), 7.22-7.14 (2H, m), 5.94 (2H, s), 4.91 (1H, sept, J=6.4 Hz), 4.54 (4H, d, J=6.9 Hz), 4.06 (1H, quint, J=7.1 Hz), 2.92 (1H, t, J=9.2 Hz), 2.26 (3H, s), 1.97 (2H, d, J=9.3 Hz), 1.77 (2H, d, J=8.7 Hz), 1.61-1.49 (10H, m). LCMS (Method A): Rt=2.64 min; m/z [M+H]⁺=629.

Compound A39: ¹H NMR (400 MHz, DMSO) δ: 7.60 (1H, s), 7.56-7.30 (7H, m), 5.57 (2H, br. s), 4.90 (1H, sept, J=6.4 Hz), 4.69 (2H, s), 4.65 (2H, t, J=6.6 Hz), 4.34 (2H, t, J=6.3 Hz), 4.01 (1H, quint, J=6.8 Hz), 3.17 (1H, d, J=3.1 Hz), 2.91 (1H, t, J=11.6 Hz), 1.93-1.85 (4H, m), 1.55-1.51 (8H, m), 1.27 (2H, q, J=11.0 Hz). LCMS (Method A): Rt=2.85 min; m/z [M+H]⁺=627/629/Compound

A40: ¹H NMR (400 MHz, DMSO) δ: 7.79 (1H, ddd, J=7.6, 7.6, 1.7 Hz), 7.56 (1H, s), 7.46 (1H, q, J=6.8 Hz), 7.25-7.18 (2H, m), 7.07 (1H, dd, J=7.5, 13.0 Hz), 6.97 (1H, dd, J=7.4, 11.1 Hz), 5.45 (2H, s), 4.88 (1H, sept, J=6.4 Hz), 3.21-3.13 (1H, m), 2.98 (1H, t, J=10.2 Hz), 2.72 (6H, s), 2.08 (4H, t, J=10.0 Hz), 1.75-1.57 (4H, m), 1.49 (6H, d, J=6.4 Hz). LCMS (Method B): Rt=3.07 min; m/z [M+H]⁺=587.

Compound A41: ¹H NMR (400 MHz, DMSO) δ: 7.77 (1H, ddd, J=7.3, 7.3, 2.2 Hz), 7.68-7.62 (2H, m), 7.42-7.22 (5H, m), 5.51 (2H, s), 4.67 (2H, t, J=6.8 Hz), 4.38 (2H, t, J=6.3 Hz), 4.10 (1H, quint, J=6.8 Hz), 3.87 (1H, sept, J=3.6 Hz), 3.61-3.52 (1H, m), 2.61 (1H, t, J=11.7 Hz), 1.92 (4H, t, J=13.5 Hz), 1.61-1.50 (2H, m), 1.31-1.25 (4H, m), 1.12 (2H, ddd, J=6.1, 6.1, 8.3 Hz). LCMS (Method A): Rt=2.53 min; m/z [M+H]⁺=595

Compound A42: ¹H NMR (400 MHz, DMSO) δ: 7.59 (1H, s), 7.55-7.52 (1H, m), 7.46-7.43 (1H, m), 7.36-7.32 (2H, m), 7.28-7.20 (2H, m), 5.54 (2H, s), 4.69 (2H, t, J=6.8 Hz), 4.61 (2H, s), 4.47-4.39 (4H, m), 4.14 (1H, quint, J=6.7 Hz), 2.90 (1H, t, J=11.8 Hz), 2.67 (1H, t, J=11.5 Hz), 1.94 (4H, d, J=10.1 Hz), 1.58 (2H, q, J=11.9 Hz), 1.42 (3H, t, J=7.2 Hz), 1.38-1.23 (2H, m). LCMS (Method A): Rt=2.70 min; m/z [M+H]⁺=631/633

Compound A43: ¹H NMR (400 MHz, DMSO) δ: 7.60 (1H, s), 7.45 (1H, ddd, J=7.4, 7.4, 1.8 Hz), 7.41-7.34 (1H, m), 7.29-7.15 (4H, m), 5.53 (2H, s), 4.68 (2H, t, J=6.8 Hz), 4.49 (2H, s), 4.45 (2H, d, J=7.2 Hz), 4.41 (2H, t, J=6.4 Hz), 4.12 (1H, quint, J=6.8 Hz), 2.90 (1H, t, J=11.8 Hz), 2.68-2.61 (1H, m), 1.94 (4H, d, J=10.0 Hz), 1.57 (2H, q, J=12.0 Hz), 1.42 (3H, t, J=7.2 Hz), 1.36-1.22 (2H, m). LCMS (Method A): Rt=2.57 min; m/z [M+H]⁺=615.

Compound A44: ¹H NMR (400 MHz, DMSO) δ: 8.00 (1H, dd, J=1.7, 7.7 Hz), 7.56 (1H, s), 7.55-7.42 (3H, m), 7.13-7.03 (2H, m), 5.67 (2H, s), 4.73 (2H, t, J=7.1 Hz), 4.53 (2H, t, J=6.5 Hz), 4.43 (2H, q, J=7.2 Hz), 4.33 (1H, quint, J=6.8 Hz), 2.89 (3H, t, J=11.1 Hz), 1.97 (4H, d, J=11.5 Hz), 1.58 (2H, q, J=11.9 Hz), 1.45-1.37 (5H, m). LCMS (Method A): Rt=2.64 min; m/z [M+H]⁺=617/619.

Compound A45: ¹H NMR (400 MHz, DMSO) δ: 7.80 (1H, ddd, J=7.6, 7.6, 1.8 Hz), 7.56 (1H, s), 7.55-7.50 (1H, m), 7.31-7.25 (2H, m), 7.15-7.05 (2H, m), 5.63 (2H, s), 4.72 (2H, t, J=7.0 Hz), 4.50 (2H, t, J=6.4 Hz), 4.43 (2H, q, J=7.2 Hz), 4.31 (1H, t, J=6.6 Hz), 2.89 (2H, t, J=11.4 Hz), 1.96 (4H, d, J=11.2 Hz), 1.58 (2H, q, J=12.0 Hz), 1.44-1.37 (5H, m). LCMS (Method A): Rt=2.42 min; m/z [M+H]⁺=601.

Compound A46: ¹H NMR (400 MHz, DMSO) δ: 7.78 (1H, ddd, J=7.6, 7.6, 1.7 Hz), 7.72-7.65 (1H, m), 7.59 (1H, s), 7.47-7.31 (5H, m), 5.57 (2H, s), 4.90 (1H, sept, J=6.4 Hz), 4.51 (4H, d, J=6.9 Hz), 3.95 (1H, quint, J=6.8 Hz), 2.90 (1H, t, J=11.0 Hz), 2.44 (1H, dt, J=2.9, 11.2 Hz), 2.19 (3H, s), 1.95 (2H, d, J=12.2 Hz), 1.74 (2H, d, J=11.1 Hz), 1.53-1.39 (10H, m). LCMS (Method B): Rt=3.17 min; m/z [M+H]⁺=611.

Compound A47: ¹H NMR (400 MHz, DMSO) δ: 7.58 (1H, s), 7.55-7.52 (1H, m), 7.46-7.43 (1H, m), 7.37-7.32 (2H, m), 7.28-7.21 (2H, m), 5.53 (2H, s), 4.89 (1H, sept, J=6.5 Hz), 4.68 (2H, t, J=6.8 Hz), 4.61 (2H, s), 4.41 (2H, t, J=6.4 Hz), 4.13 (1H, quint, J=6.8 Hz), 2.92 (1H, t, J=11.6 Hz), 2.68-2.60 (1H, m), 1.97-1.89 (4H, m), 1.54-1.50 (8H, m), 1.33 (2H, q, J=11.4 Hz). LCMS (Method B): Rt=3.16 min; m/z [M+H]⁺=645/647.

Compound A48: ¹H NMR (400 MHz, DMSO) δ: 7.58 (1H, s), 7.46 (1H, ddd, J=7.5, 7.5, 1.6 Hz), 7.42-7.35 (1H, m), 7.29-7.22 (2H, m), 7.19 (2H, dd, J=8.3, 8.3 Hz), 5.53 (2H, s), 4.89 (1H, sept, J=6.3 Hz), 4.68 (2H, t, J=6.8 Hz), 4.51 (2H, s), 4.40 (2H, t, J=6.4 Hz), 4.12 (1H, quint, J=6.8 Hz), 2.92 (1H, t, J=11.7 Hz), 2.68-2.60 (1H, m), 1.93 (4H, dd, J=3.5, 10.3 Hz), 1.54-1.51 (8H, m), 1.39-1.24 (2H, m). LCMS (Method B): Rt=3.04 min; m/z [M+H]⁺=629.

Compound A49: ¹H NMR (400 MHz, DMSO) δ: 8.00 (1H, dd, J=1.8, 7.6 Hz), 7.54 (1H, s), 7.52-7.41 (3H, m), 7.11-7.02 (2H, m), 5.65 (2H, br. s), 4.85 (1H, sept, J=7.0 Hz), 4.72 (2H, t, J=7.0 Hz), 4.52-4.46 (2H, m), 4.29 (1H, quint, J=6.5 Hz), 2.95-2.84 (2H, m), 1.96 (4H, d, J=10.4 Hz), 1.57 (2H, q, J=12.3 Hz), 1.50 (6H, d, J=6.4 Hz), 1.40 (2H, q, J=12.3 Hz). LCMS (Method A): Rt=2.78 min; m/z [M+H]⁺=631/633.

Compound A50: ¹H NMR (400 MHz, DMSO) δ: 7.80 (1H, ddd, J=7.6, 7.6, 1.9 Hz), 7.56-7.51 (2H, m), 7.31-7.24 (2H, m), 7.15-7.06 (2H, m), 5.65 (2H, br. s), 4.86 (1H, sept, J=6.5 Hz), 4.72 (2H, t, J=7.2 Hz), 4.49 (2H, t, J=6.1 Hz), 4.29 (1H, br. s), 2.94-2.83 (2H, m), 1.96 (4H, d, J=11.4 Hz), 1.50 (6H, d, J=5.7 Hz), 1.62-1.38 (4H, m). LCMS (Method A): Rt=2.67 min; m/z [M+H]⁺=615.

Compound A51: ¹H NMR (400 MHz, DMSO) δ: 7.62 (1H, s), 7.54 (1H, dd, J=2.4, 7.0 Hz), 7.50 (1H, d, J=8.7 Hz), 7.48-7.44 (1H, m), 7.37-7.30 (3H, m), 7.24 (1H, dd, J=1.6, 8.3 Hz), 5.58 (2H, s), 4.93 (1H, sept, J=6.3 Hz), 4.54 (2H, s), 2.96 (1H, t, J=10.4 Hz), 2.56 (1H, s), 2.37 (6H, s), 2.01 (4H, t, J=9.4 Hz), 1.66-1.46 (10H, m). LCMS (Method C): Rt=2.93 min; m/z [M+H]⁺=599/601.

Compound A52: ¹H NMR (400 MHz, DMSO) δ: 7.97 (1H, dd, J=1.6, 7.9 Hz), 7.65-7.56 (3H, m), 7.47 (1H, ddd, J=7.5, 7.5, 1.2 Hz), 7.36-7.30 (2H, m), 7.25 (1H, dd, J=1.7, 8.3 Hz), 5.55 (2H, s), 4.67 (2H, t, J=6.8 Hz), 4.44-4.36 (4H, m), 4.09 (1H, quint, J=6.7 Hz), 3.17 (1H, s), 2.88 (1H, t, J=11.9 Hz), 2.59 (1H, t, J=11.3 Hz), 1.90 (4H, d, J=11.7 Hz), 1.55 (2H, q, J=11.9 Hz), 1.42-1.37 (3H, m). LCMS (Method A): Rt=2.59 min; m/z [M+H]⁺=599/601.

Compound A53: ¹H NMR (400 MHz, DMSO) δ: 7.62 (1H, s), 7.51-7.37 (4H, m), 7.32 (1H, dd, J=1.6, 8.3 Hz), 7.24-7.18 (2H, m), 5.59 (2H, s), 4.65 (2H, t, J=6.6 Hz), 4.55 (2H, s), 4.46 (2H, q, J=7.1 Hz), 4.34 (2H, t, J=6.3 Hz), 4.00 (1H, quint, J=6.9 Hz), 2.89 (1H, t, J=11.2 Hz), 1.90 (4H, t, J=10.9 Hz), 1.62-1.50 (2H, m), 1.42 (3H, t, J=7.1 Hz), 1.30-1.21 (2H, m). LCMS (Method A): Rt=2.54 min; m/z [M+H]⁺=597.

Compound A54: ¹H NMR (400 MHz, DMSO) δ: 7.68 (1H, s), 7.51-7.45 (2H, m), 7.44-7.39 (2H, m), 7.31 (1H, dd, J=1.6, 8.4 Hz), 7.25-7.19 (2H, m), 5.57 (2H, s), 4.66 (2H, t, J=6.7 Hz), 4.55 (2H, s), 4.35 (2H, t, J=6.3 Hz), 4.02 (1H, quint, J=6.5 Hz), 3.91 (1H, sept, J=3.6 Hz), 3.59 (1H, tt, J=2.8, 11.9 Hz), 1.96-1.87 (4H, m), 1.57 (2H, q, J=12.0 Hz), 1.35-1.30 (2H, m), 1.27 (2H, d, J=11.2 Hz), 1.20-1.14 (2H, m), 1.05 (1H, d, J=6.3 Hz). LCMS (Method C): Rt=2.61 min; m/z [M+H]⁺=609.

Compound A55: LCMS (Method C): Rt=2.75 min; m/z [M+H]⁺=625/627.

Compound A56: ¹H NMR (400 MHz, DMSO) δ: 7.96 (1H, dd, J=1.5, 7.9 Hz), 7.64-7.56 (3H, m), 7.46 (1H, ddd, J=7.5, 7.5, 1.2 Hz), 7.34-7.26 (2H, m), 7.21 (1H, dd, J=1.6, 8.3 Hz), 5.51 (2H, s), 4.67 (2H, t, J=6.7 Hz), 4.38 (2H, t, J=6.4 Hz), 4.09 (1H, quint, J=6.8 Hz), 3.87 (1H, sept, J=3.6 Hz), 3.62-3.53 (2H, m), 2.60 (1H, t, J=11.7 Hz), 1.91 (4H, t, J=13.2 Hz), 1.55 (2H, q, J=11.8 Hz), 1.32-1.22 (4H, m), 1.16-1.09 (2H, m). LCMS (Method A): Rt=2.71 min; m/z [M+H]⁺=611/613.

Compound A57: ¹H NMR (400 MHz, DMSO) δ: 7.61 (1H, s), 7.54-7.37 (4H, m), 7.34 (1H, d, J=8.2 Hz), 7.26-7.17 (2H, m), 5.58 (2H, s), 4.90 (1H, sept, J=6.5 Hz), 4.66 (2H, t, J=6.4 Hz), 4.58 (2H, s), 4.35 (2H, t, J=6.2 Hz), 4.02 (1H, quint, J=7.0 Hz), 2.93 (1H, t, J=11.9 Hz), 1.91 (4H, t, J=10.3 Hz), 1.56-1.50 (8H, m), 1.27 (2H, q, J=11.1 Hz). LCMS (Method A): Rt=2.74 min; m/z [M+H]⁺=611.

Compound A58: ¹H NMR (400 MHz, DMSO) δ: 7.60 (1H, s), 7.54-7.42 (3H, m), 7.35-7.27 (3H, m), 7.21 (1H, dd, J=1.6, 8.3 Hz), 5.58 (2H, br. s), 4.89 (1H, sept. J=6.7 Hz), 4.51 (2H, s), 3.90 (4H, s), 2.92 (2H, q, J=11.2 Hz), 2.09 (2H, t, J=14.6 Hz), 1.96 (2H, d, J=15.2 Hz), 1.63 (2H, quint, J=11.4 Hz), 1.53 (6H, d, J=5.6 Hz), 1.48-1.29 (2H, m), 1.08 (3H, d, J=6.4 Hz). LCMS (Method A): Rt=2.94 min; m/z [M+H]⁺=643/645.

Compound A59: H NMR (400 MHz, DMSO) δ: 8.41 (1H, br. s), 7.69 (1H, dd, J=3.4, 4.6 Hz), 7.56 (1H, s), 7.50-7.45 (1H, m), 7.36 (1H, dd, J=8.8, 8.8 Hz), 7.07 (1H, dd, J=7.4, 13.4 Hz), 6.97 (1H, dd, J=7.6, 11.0 Hz), 5.42 (2H, s), 4.83 (1H, sept, J=6.3 Hz), 3.60 (2H, t, J=5.1 Hz), 3.35 (3H, s), 3.20-3.16 (4H, m), 2.91 (1H, br. t, J=11.1 Hz), 2.20 (2H, br. d, J=11.6 Hz), 2.01 (2H, br. d, J=13.0 Hz), 1.68-1.53 (4H, m), 1.51 (6H, d, J=6.3 Hz). LCMS (Method A): Rt=3.40 min; m/z [M+H]⁺=701.

Compound A60: ¹H NMR (400 MHz, DMSO) δ: 8.42 (1H, br. s), 7.58 (1H, s), 7.28 (1H, dd, J=3.4, 5.8 Hz), 7.15-7.06 (2H, m), 6.99-6.94 (2H, m), 5.44 (2H, s), 4.84 (1H, sept, J=6.6 Hz), 3.91 (2H, t, J=6.5 Hz), 3.61 (2H, t, J=4.5 Hz), 3.36 (3H, s), 3.19-3.14 (3H, m), 2.93 (1H, br. t, J=11.8 Hz), 2.20 (2H, br. d, J=10.5 Hz), 2.02 (2H, br. d, J=12.3 Hz), 1.76-1.55 (6H, m), 1.52 (6H, d, J=6.4 Hz), 0.96 (3H, t, J=7.5 Hz). LCMS (Method C): Rt=3.25 min; m/z [M+H]⁺=676.

Compound A61: ¹H NMR (400 MHz, DMSO) δ: 8.41 (1H, br. s), 7.95 (1H, dd, J=0.8, 2.0 Hz), 7.58 (1H, s), 7.50-7.48 (2H, m), 7.05-6.95 (2H, m), 5.42 (2H, s), 4.84 (1H, sept, J=6.3 Hz), 3.59 (2H, t, J=5.2 Hz), 3.35 (3H, s), 3.15 (3H, s), 2.92 (1H, t, J=10.7 Hz), 2.19 (2H, d, J=12.7 Hz), 2.03 (2H, d, J=12.0 Hz), 1.70-1.54 (4H, m), 1.52 (6H, d, J=6.3 Hz). LCMS (Method C): Rt=3.12 min; m/z [M+H]⁺=669.

Compound A62: ¹H NMR (400 MHz, DMSO) δ: 7.84 (1H, d, J=2.2 Hz), 7.56 (1H, s), 7.33 (1H, d, J=8.0 Hz), 7.23 (1H, dd, J=2.0, 8.0 Hz), 7.02-6.91 (2H, m), 5.38 (2H, s), 4.82 (1H, sept, J=6.4 Hz), 3.57 (2H, t, J=5.6 Hz), 3.14 (3H, t, J=5.4 Hz), 2.91 (1H, t, J=10.5 Hz), 2.61 (2H, q, J=7.9 Hz), 2.18 (2H, d, J=12.9 Hz), 2.02 (2H, d, J=12.3 Hz), 1.68-1.53 (4H, m), 1.51 (6H, d, J=6.4 Hz), 1.14 (3H, t, J=8.0 Hz). LCMS (Method B): Rt=3.53 min; m/z [M+H]⁺=661/663.

Compound B3: ¹H NMR (400 MHz, DMSO) δ: 8.16 (1H, s), 7.97 (1H, dd, J=1.7, 7.8 Hz), 7.52 (1H, d, J=7.6 Hz), 7.46 (1H, ddd, J=7.6, 7.6, 1.6 Hz), 7.40 (1H, ddd, J=7.5, 7.5, 1.3 Hz), 7.36 (1H, s), 7.26 (1H, dd, J=8.6, 8.6 Hz), 7.20 (1H, dd, J=1.3, 11.7 Hz), 7.10 (1H, d, J=8.3 Hz), 5.69 (1H, s), 5.65 (2H, s), 4.81 (1H, quint, J=6.6 Hz), 2.33-2.29 (3H, m), 2.19-2.05 (2H, m), 1.75-1.60 (1H, m), 1.45 (3H, d, J=5.0 Hz), 1.35 (3H, d, J=5.4 Hz), 1.15 (3H, dd, J=4.6, 5.9 Hz). LCMS (Method C): Rt=2.86 min; m/z [M+H]⁺=627.

Compound B4 (Second eluting isomer): NMR (400 MHz, DMSO) δ: 8.20 (1H, s), 7.55-7.50 (1H, m), 7.49-7.45 (1H, m), 7.40 (1H, s), 7.38-7.33 (2H, m), 7.29 (1H, dd, J=1.6, 8.3 Hz), 5.74 (2H, s), 5.70 (1H, s), 4.88 (1H, quint, J=6.1 Hz), 4.61 (2H, s), 3.34 (2H, d, J=6.9 Hz), 3.23-3.16 (3H, m), 2.32 (2H, s), 2.13-2.02 (2H, m), 1.71-1.60 (1H, m), 1.50 (2H, d, J=4.2 Hz), 1.38 (2H, d, J=5.5 Hz), 1.12-1.07 (3H, m). LCMS (Method C): Rt=2.93 min; m/z [M+H]⁺=641.

Compound B5 (First eluting isomer): NMR (400 MHz, DMSO) δ: 8.19 (1H, s), 7.55-7.49 (2H, m), 7.49-7.45 (1H, m), 7.40-7.33 (4H, m), 7.29 (1H, dd, J=1.5, 8.3 Hz), 5.73 (2H, s), 5.70 (1H, s), 4.88 (1H, QUINT, j=6.1 Hz), 4.63-4.60 (2H, m), 3.35 (3H, d, J=5.3 Hz), 3.21-3.16 (4H, m), 2.33 (2H, s), 2.13-2.03 (2H, m), 1.72-1.59 (1H, m), 1.48 (3H, d, J=4.0 Hz), 1.40 (3H, d, J=5.4 Hz), 1.12-1.08 (3H, m). LCMS (Method C): Rt=3.01 min; m/z [M+H]⁺=641.

Compound B6 (Second eluting isomer): NMR (400 MHz, DMSO) δ: 7.53-7.48 (1H, m), 7.40-7.35 (1H, m), 7.30-7.25 (2H, m), 7.25-7.20 (1H, m), 6.92 (1H, dd, J=7.6, 11.5 Hz), 6.61 (1H, d, J=29.1 Hz), 5.94 (1H, s), 5.76 (2H, s), 4.71-4.64 (2H, m), 4.37 (2H, t, J=6.1 Hz), 4.23 (2H, s), 4.04 (1H, quint, J=7.1 Hz), 2.89-2.81 (4H, m), 2.40-2.26 (3H, m), 2.15-2.08 (1H, m), 1.95-1.76 (2H, m), 1.42-1.35 (4H, m), 1.17-1.10 (9H, m). LCMS (Method C): Rt=2.77 min; m/z [M+H]⁺=643.

Compound B7 (First eluting isomer): NMR (400 MHz, DMSO) δ: 7.53-7.49 (1H, m), 7.40-7.35 (1H, m), 7.29-7.20 (3H, m), 6.92 (1H, dd, J=7.6, 11.5 Hz), 6.65-6.56 (1H, m), 5.93 (1H, s), 5.75 (2H, s), 4.70-4.64 (2H, m), 4.37 (2H, t, J=6.1 Hz), 4.23 (2H, s), 4.04 (1H, quint, J=6.8 Hz), 3.48-3.39 (1H, m), 2.76 (1H, s), 2.40-2.31 (2H, m), 2.27 (1H, s), 2.15-2.08 (1H, m), 1.95-1.77 (2H, m), 1.43-1.35 (3H, m), 1.17-1.11 (3H, m). LCMS (Method C): Rt=2.77 min; m/z [M+H]⁺=643.

Compound B8 (Second eluting isomer): NMR (400 MHz, DMSO) δ: 7.53 (1H, d, J=8.8 Hz), 7.49 (1H, d, J=3.0 Hz), 7.39-7.32 (3H, m), 7.27 (1H, d, J=7.7 Hz), 7.16 (1H, dd, J=2.3, 8.3 Hz), 5.67-5.65 (3H, m), 4.94 (1H, quint, J=6.6 Hz), 4.69 (2H, dd, J=6.6, 10.2 Hz), 4.40 (2H, t, J=5.8 Hz), 4.07 (1H, sept, J=7.3 Hz), 3.79 (3H, s), 2.84 (1H, s), 2.36-2.28 (3H, m), 1.99-1.93 (1H, m), 1.86-1.82 (1H, m), 1.47-1.46 (3H, m), 1.36-1.33 (3H, m). LCMS (Method B): Rt=3.68 min; m/z [M+H]⁺=641.

Compound B9 (First eluting isomer): NMR (400 MHz, DMSO) δ: 8.18 (1H, s), 7.52 (1H, d, J=3.0 Hz), 7.39 (1H, d, J=8.7 Hz), 7.35 (1H, s), 7.27 (1H, dd, J=8.7, 8.7 Hz), 7.18 (1H, d, J=11.8 Hz), 7.07 (1H, d, J=8.0 Hz), 7.01 (1H, dd, J=2.8, 8.6 Hz), 5.66 (1H, s), 5.63 (2H, s), 4.93 (1H, quint, J=6.6 Hz), 4.71-4.66 (2H, m), 4.40-4.36 (2H, m), 4.05 (1H, quint, J=6.8 Hz), 3.77-3.76 (3H, m), 2.79 (1H, s), 2.35-2.22 (3H, m), 1.98-1.92 (1H, m), 1.85-1.76 (1H, m), 1.54-1.44 (4H, m), 1.37-1.35 (3H, m). LCMS (Method B): Rt=3.68 min; m/z [M+H]⁺=641.

Compound B10 (Second eluting isomer): ¹H NMR (400 MHz, DMSO) δ: 8.27 (1H, s), 8.00-7.97 (1H, m), 7.46-7.43 (1H, m), 7.41-7.37 (2H, m), 7.33 (1H, s), 7.02-6.93 (2H, m), 5.67 (1H, s), 5.46 (2H, s), 4.92 (1H, quint, J=6.5 Hz), 4.71-4.66 (2H, m), 4.38 (2H, dt, J=1.2, 6.1 Hz), 4.04 (1H, quint, J=6.8 Hz), 2.89-2.77 (1H, m), 2.36-2.22 (3H, m), 1.97-1.90 (1H, m), 1.85-1.80 (1H, m), 1.52 (1H, s), 1.44 (3H, d, J=4.5 Hz), 1.35 (3H, d, J=6.8 Hz). LCMS (Method B): Rt=3.33 min; m/z [M+H]⁺=629.

Compound B11 (First eluting isomer): ¹H NMR (400 MHz, DMSO) δ: 8.01 (1H, dd, J=1.6, 7.7 Hz), 7.52 (1H, dd, J=7.9, 7.9 Hz), 7.49-7.41 (2H, m), 7.35 (1H, s), 7.11-7.04 (2H, m), 5.69 (1H, s), 5.64 (2H, s), 4.90 (1H, quint, J=6.8 Hz), 4.74-4.69 (2H, m), 4.44 (2H, t, J=6.0 Hz), 4.17 (1H, s), 2.98-2.92 (2H, m), 2.29 (2H, s), 2.06-2.01 (1H, m), 1.92 (1H, s), 1.45 (3H, d, J=6.1 Hz), 1.36 (3H, d, J=5.4 Hz), 1.17 (2H, t, J=7.3 Hz). LCMS (Method B): Rt=3.33 min; m/z [M+H]⁺=629.

Compound B12 (Second eluting isomer): ¹H NMR (400 MHz, DMSO) δ: 7.56-7.50 (2H, m), 7.42-7.38 (1H, m), 7.36 (1H, s), 7.30-7.27 (2H, m), 7.17 (1H, dd, J=2.0, 12.0 Hz), 7.11 (1H, dd, J=1.8, 8.3 Hz), 5.69 (2H, s), 5.67 (1H, s), 4.94 (1H, sept, J=6.5 Hz), 4.72-4.66 (2H, m), 4.40-4.36 (2H, m), 4.31 (2H, s), 4.05 (1H, quint, J=6.8 Hz), 2.82 (1H, q, J=7.2 Hz), 2.36-2.24 (3H, m), 1.98-1.91 (1H, m), 1.88-1.80 (1H, m), 1.56-1.48 (4H, m), 1.38 (3H, s). LCMS (Method C): Rt=2.85 min; m/z [M+H]⁺=625.

Compound B13 (First eluting isomer): ¹H NMR (400 MHz, DMSO) δ: 7.56-7.50 (2H, m), 7.42-7.39 (1H, m), 7.36 (1H, s), 7.31-7.27 (2H, m), 7.18 (1H, dd, J=1.9, 12.1 Hz), 7.11 (1H, dd, J=1.8, 8.3 Hz), 5.69 (2H, s), 5.68 (1H, s), 4.94 (1H, quint, J=6.5 Hz), 4.72-4.66 (2H, m), 4.41-4.36 (2H, m), 4.32 (2H, s), 4.06 (1H, quint, J=6.9 Hz), 2.86-2.79 (1H, m), 2.35-2.20 (3H, m), 1.99-1.91 (1H, m), 1.86-1.77 (1H, m), 1.49 (4H, s), 1.38 (3H, s), 1.12 (5H, t, J=7.2 Hz). LCMS (Method C): Rt=2.84 min; m/z [M+H]⁺=625.

Compound B14 (First eluting isomer): ¹H NMR (400 MHz, DMSO) δ 7.75 (1H, dd, J=2.6, 5.9 Hz), 7.68-7.64 (1H, m), 7.44-7.32 (3H, m), 7.28 (1H, dd, J=1.6, 11.4 Hz), 7.22 (1H, d, J=8.2 Hz), 5.74 (2H, s), 5.69 (1H, s), 4.92 (1H, quint, J=6.3 Hz), 4.71 (2H, dd, J=6.5, 10.4 Hz), 4.43 (2H, t, J=6.1 Hz), 4.19-4.09 (1H, m), 2.91 (1H, s), 2.40-2.34 (2H, m), 2.28 (2H, s), 2.03-1.84 (2H, m), 1.57 (1H, s), 1.47 (3H, s), 1.37 (3H, s). LCMS (Method B): Rt=3.50 min; m/z [M+H]⁺=629.

Compound B15 (Second eluting isomer): ¹H NMR (400 MHz, DMSO) δ: 7.76 (1H, dd, J=2.8, 6.0 Hz), 7.74-7.69 (1H, m), 7.47 (1H, dd, J=9.2, 9.2 Hz), 7.41-7.28 (4H, m), 5.80 (2H, s), 5.72-5.67 (1H, m), 4.91 (1H, sept, J=6.5 Hz), 4.75-4.69 (2H, m), 4.46 (2H, t, J=6.2 Hz), 4.20 (1H, quint, J=6.6 Hz), 2.99 (1H, s), 2.42-2.27 (3H, m), 2.10-2.00 (1H, m), 1.94-1.89 (1H, m), 1.59 (1H, s), 1.47 (3H, s), 1.38 (3H, s). LCMS (Method B): Rt=3.49 min; m/z [M+H]⁺=629.

Compound B16 (First eluting isomer): ¹H NMR (400 MHz, DMSO) δ 7.60 (1H, dd, J=2.0, 6.9 Hz), 7.33 (1H, s), 7.28-7.19 (2H, m), 7.13-7.03 (2H, m), 7.00 (1H, dd, J=1.8, 8.3 Hz), 5.65 (1H, s), 5.62 (2H, s), 4.91 (1H, quint, J=6.5 Hz), 4.68 (2H, q, J=5.8 Hz), 4.38 (2H, t, J=5.6 Hz), 4.04 (1H, quint, J=6.8 Hz), 2.80-2.76 (1H, m), 2.31-2.28 (6H, m), 1.98-1.90 (1H, m), 1.84-1.74 (1H, m), 1.54-1.45 (4H, m), 1.35 (3H, s). LCMS (Method C): Rt=2.81 min; m/z [M+H]⁺=609.

Compound B17 (Second eluting isomer): ¹H NMR (400 MHz, DMSO) δ: 7.60 (1H, dd, J=1.9, 7.0 Hz), 7.33 (1H, s), 7.28-7.20 (2H, m), 7.13-7.04 (2H, m), 7.03-6.99 (1H, m), 5.65 (1H, s), 5.63 (2H, s), 4.92 (1H, sept, J=6.5 Hz), 4.71-4.65 (2H, m), 4.38 (2H, t, J=5.6 Hz), 4.04 (1H, quint, J=6.8 Hz), 2.90 (4H, q, J=7.3 Hz), 2.81-2.76 (1H, m), 2.31-2.29 (6H, m), 1.98-1.90 (1H, m), 1.84-1.81 (1H, m), 1.53-1.49 (1H, m), 1.45 (3H, s), 1.35 (3H, s), 1.16 (6H, t, J=7.2 Hz). LCMS (Method B): Rt=4.07 min; m/z [M+H]⁺=609.

Compound B18 (First eluting isomer): ¹H NMR (400 MHz, DMSO) δ 7.98 (1H, dd, J=1.8, 7.6 Hz), 7.50 (1H, dd, J=1.4, 8.0 Hz), 7.47-7.37 (3H, m), 7.23 (1H, dd, J=8.7, 8.7 Hz), 7.17 (1H, dd, J=1.8, 11.8 Hz), 7.04 (1H, d, J=7.7 Hz), 5.65-5.61 (3H, m), 4.70-4.64 (2H, m), 4.36 (2H, t, J=6.0 Hz), 4.04 (1H, quint, J=6.8 Hz), 3.78 (1H, sept, J=3.7 Hz), 2.82-2.76 (1H, m), 2.45-2.31 (3H, m), 1.98-1.90 (1H, m), 1.85-1.80 (1H, m), 1.57-1.47 (1H, m), 1.14-1.07 (2H, m), 1.01-0.97 (2H, m). LCMS (Method C): Rt=2.69 min; m/z [M+H]⁺=609.

Compound B19 (Second eluting isomer): ¹H NMR (400 MHz, DMSO) δ: 7.98 (1H, dd, J=1.8, 7.7 Hz), 7.49 (1H, dd, J=1.6, 7.4 Hz), 7.43 (2H, s), 7.38 (1H, ddd, J=7.4, 7.4, 1.9 Hz), 7.21 (1H, dd, J=8.7, 8.7 Hz), 7.14 (1H, dd, J=1.8, 12.0 Hz), 7.04-6.99 (1H, m), 5.65-5.61 (3H, m), 4.70-4.64 (2H, m), 4.36 (2H, t, J=5.9 Hz), 4.03 (1H, quint, J=6.8 Hz), 3.77 (1H, sept, J=3.7 Hz), 2.81-2.74 (1H, m), 2.45-2.30 (3H, m), 1.97-1.90 (1H, m), 1.85-1.82 (1H, m), 1.56-1.47 (1H, m), 1.14-1.07 (2H, m), 1.02-0.97 (2H, m). LCMS (Method C): Rt=2.69 min; m/z [M+H]⁺=609.

Compound B20 (First eluting isomer): ¹H NMR (400 MHz, DMSO) δ 7.51-7.32 (6H, m), 7.24-7.18 (2H, m), 5.72 (2H, s), 5.63 (1H, s), 4.69-4.65 (2H, m), 4.57 (2H, s), 4.36 (2H, t, J=5.9 Hz), 4.04 (1H, quint, J=6.8 Hz), 3.81 (1H, sept, 3.6 Hz), 2.82-2.75 (1H, m), 2.41-2.29 (3H, m), 1.99-1.90 (1H, m), 1.85-1.82 (1H, m), 1.57-1.47 (1H, m), 1.17-1.09 (2H, m), 1.04-1.00 (2H, m). LCMS (Method A): Rt=2.59 min; m/z [M+H]⁺=607.

Compound B21 (Second eluting isomer): NMR (400 MHz, DMSO) δ: 7.49-7.40 (3H, m), 7.35-7.27 (1H, m), 7.22 (1H, d, J=13.3 Hz), 7.17-7.12 (3H, m), 5.69 (2H, s), 5.63 (1H, s), 4.69-4.63 (2H, m), 4.35 (2H, t, J=5.9 Hz), 4.27 (2H, s), 4.02 (1H, quint, J=6.8 Hz), 3.79 (1H, sept, J=3.7 Hz), 2.86 (2H, q, J=7.3 Hz), 2.81-2.73 (1H, m), 2.41 (1H, s), 2.36-2.31 (2H, m), 1.96-1.90 (1H, m), 1.85-1.81 (1H, m), 1.57-1.46 (1H, m), 1.12 (5H, t, J=7.2 Hz), 1.03-0.98 (2H, m). LCMS (Method C): Rt=2.64 min; m/z [M+H]⁺=607.

Compound B22 (Second eluting isomer): NMR (400 MHz, DMSO) δ 7.54-7.48 (2H, m), 7.45 (1H, s), 7.43-7.38 (1H, m), 7.31-7.27 (2H, m), 7.17 (1H, dd, J=1.8, 12.0 Hz), 7.10 (1H, dd, J=1.6, 8.4 Hz), 5.69 (2H, s), 5.65 (1H, s), 4.71-4.65 (2H, m), 4.39-4.32 (4H, m), 4.04 (1H, quint, J=6.8 Hz), 3.80 (1H, sept, J=3.7), 2.80-2.77 (1H, m), 2.44-2.31 (3H, m), 1.98-1.91 (1H, m), 1.87-1.83 (1H, m), 1.58-1.48 (1H, m), 1.17-1.10 (2H, m), 1.06-0.99 (2H, m). LCMS (Method B): Rt=4.19 min; m/z [M+H]⁺=623.

Compound B23 (First eluting isomer): NMR (400 MHz, DMSO) δ: 7.53-7.48 (2H, m), 7.45 (1H, s), 7.44-7.41 (1H, m), 7.32-7.29 (2H, m), 7.21 (1H, d, J=11.5 Hz), 7.14 (1H, d, J=8.3 Hz), 5.70 (2H, s), 5.64 (1H, s), 4.71-4.65 (2H, m), 4.41-4.35 (4H, m), 4.04 (1H, quint, J=6.8 Hz), 3.80 (1H, sept, J=3.7 Hz), 2.47-2.31 (3H, m), 1.98-1.92 (1H, m), 1.87-1.82 (1H, m), 1.58-1.48 (1H, m), 1.17-1.12 (2H, m), 1.02 (2H, dd, J=1.5, 7.4 Hz). LCMS (Method B): Rt=4.19 min; m/z [M+H]⁺=623.

Compound B24 (First eluting isomer): NMR (400 MHz, DMSO) δ 7.52-7.44 (4H, m), 7.42 (1H, s), 7.39-7.35 (2H, m), 7.31 (1H, s), 7.29 (1H, s), 5.69 (1H, s), 5.64 (2H, s), 4.68 (4H, dd, J=7.5, 9.3 Hz), 4.38 (2H, t, J=6.0 Hz), 4.36-4.28 (2H, m), 4.09-4.01 (1H, m), 2.83-2.76 (1H, m), 2.37-2.32 (3H, m), 1.99-1.91 (1H, m), 1.87-1.84 (1H, m), 1.56-1.48 (1H, m), 1.34 (3H, t, J=7.2 Hz). LCMS (Method C): Rt=2.64 min; m/z [M+H]⁺=593.

Compound B25 (Second eluting isomer): NMR (400 MHz, DMSO) δ: 7.51-7.43 (4H, m), 7.41 (1H, s), 7.38-7.35 (2H, m), 7.27 (1H, s), 7.25 (1H, s), 5.67-5.63 (3H, m), 4.71-4.66 (2H, m), 4.62 (2H, s), 4.38 (2H, t, J=6.1 Hz), 4.36-4.27 (2H, m), 4.05 (1H, quint, J=6.8 Hz), 2.80-2.75 (1H, m), 2.38-2.31 (3H, m), 1.99-1.91 (1H, m), 1.87-1.84 (1H, m), 1.57-1.47 (1H, m), 1.34 (3H, t, J=7.1 Hz). LCMS (Method C): Rt=2.66 min; m/z [M+H]⁺=593.

Compound B26 (Second eluting isomer): NMR (400 MHz, DMSO) δ 7.50-7.42 (5H, m), 7.35 (2H, d, J=1.6 Hz), 7.24-7.18 (2H, m), 5.75-5.71 (2H, m), 5.69-5.66 (1H, m), 4.67 (2H, q, J=5.6 Hz), 4.56 (2H, s), 4.38 (2H, t, J=6.3 Hz), 4.34-4.25 (2H, m), 4.04 (1H, quint, J=6.8 Hz), 2.83-2.75 (1H, m), 2.39-2.30 (3H, m), 1.99-1.89 (1H, m), 1.88-1.80 (1H, m), 1.57-1.46 (1H, m), 1.33 (3H, t, J=7.1 Hz). LCMS (Method A): Rt=2.56 min; m/z [M+H]⁺=595.

Compound B27 (First eluting isomer): ¹H NMR (400 MHz, DMSO) δ: 7.49 (1H, dd, J=8.7, 8.7 Hz), 7.43 (1H, ddd, J=7.7, 7.7, 2.5 Hz), 7.40 (1H, s), 7.35-7.28 (1H, m), 7.19 (1H, dd, J=2.0, 12.0 Hz), 7.16-7.10 (3H, m), 5.71 (2H, s), 5.68 (1H, s), 4.71-4.66 (2H, m), 4.39 (2H, t, J=5.9 Hz), 4.36-4.24 (2H, m), 4.19 (2H, s), 4.05 (1H, quint, J=6.8 Hz), 2.86-2.78 (1H, m), 2.38-2.31 (3H, m), 2.00-1.91 (1H, m), 1.87-1.84 (1H, m), 1.57-1.46 (1H, m), 1.37-1.31 (3H, m), 1.15-1.09 (6H, m). LCMS (Method C): Rt=2.53 min; m/z [M+H]⁺=595.

Compound B28 (Second eluting isomer): ¹H NMR (400 MHz, DMSO) δ 7.55 (1H, dd, J=2.5, 6.9 Hz), 7.53-7.45 (3H, m), 7.44 (1H, s), 7.42-7.33 (3H, m), 5.74 (2H, s), 5.70-5.66 (1H, m), 4.72-4.66 (4H, m), 4.39 (2H, t, J=6.1 Hz), 4.36-4.29 (2H, m), 4.06 (1H, quint, J=6.8 Hz), 2.83-2.77 (1H, m), 2.38-2.26 (3H, m), 2.00-1.82 (2H, m), 1.58-1.48 (1H, m), 1.38-1.32 (3H, m). LCMS (Method B): Rt=3.71 min; m/z [M+H]⁺=611.

Compound B29 (First eluting isomer): ¹H NMR (400 MHz, DMSO) δ: 7.56-7.45 (3H, m), 7.42 (1H, s), 7.41-7.33 (4H, m), 7.28 (1H, d, J=8.5 Hz), 5.76-5.72 (2H, m), 5.69 (1H, s), 4.71-4.66 (2H, m), 4.60-4.55 (2H, m), 4.39 (2H, t, J=6.0 Hz), 4.38-4.26 (2H, m), 4.05 (1H, quint, J=6.8 Hz), 2.82-2.76 (1H, m), 2.38-2.26 (3H, m), 1.99-1.92 (1H, m), 1.89-1.84 (2H, m), 1.57-1.47 (1H, m), 1.37-1.32 (3H, m). LCMS (Method B): Rt=4.09 min; m/z [M+H]⁺=611.

Compound B30: ¹H NMR (400 MHz, DMSO) δ: 8.14 (1H, s), 7.81 (1H, ddd, J=7.5, 7.5, 1.7 Hz), 7.58 (1H, dd, J=6.6, 12.4 Hz), 7.39 (1H, s), 7.35-7.26 (2H, m), 7.18-7.11 (2H, m), 5.72 (2H, s), 5.69 (1H, s), 4.72 (2H, dt, J=3.4, 6.8 Hz), 4.49 (2H, t, J=6.4 Hz), 4.37-4.22 (3H, m), 3.05 (1H, s), 2.41 (1H, td, J=5.6, 18.2 Hz), 2.33 (2H, s), 2.09-2.02 (1H, m), 1.97-1.89 (1H, m), 1.66-1.56 (1H, m), 1.31 (3H, t, J=7.1 Hz). LCMS (Method A): Rt=2.54 min; m/z [M+H]⁺=599.

Compound B31: ¹H NMR (400 MHz, DMSO) δ: 8.14 (1H, s), 8.00 (1H, dd, J=1.7, 7.7 Hz), 7.57-7.48 (2H, m), 7.44 (1H, ddd, J=7.4, 7.4, 1.5 Hz), 7.39-7.38 (1H, m), 7.16-7.05 (2H, m), 5.70 (3H, s), 4.72 (2H, dt, J=3.5, 6.7 Hz), 4.47 (2H, t, J=6.3 Hz), 4.36-4.20 (3H, m), 3.02 (1H, s), 2.40 (1H, td, J=5.6, 17.6 Hz), 2.33-2.28 (2H, m), 2.09-2.01 (1H, m), 1.94-1.88 (1H, m), 1.64-1.53 (1H, m), 1.33-1.28 (3H, m). LCMS (Method A): Rt=2.65 min; m/z [M+H]⁺=615.

Compound B32: ¹H NMR (400 MHz, DMSO) δ 7.55 (1H, dd, J=2.3, 7.0 Hz), 7.48-7.39 (2H, m), 7.39-7.31 (3H, m), 7.24 (1H, dd, J=6.9, 11.3 Hz), 5.70-5.70 (2H, m), 4.74-4.67 (3H, m), 4.46 (1H, t, J=6.3 Hz), 4.39-4.26 (2H, m), 2.96 (1H, s), 2.42-2.16 (3H, m), 2.12-1.90 (2H, m), 1.85-1.49 (2H, m), 1.37-1.28 (3H, m). LCMS (Method A): Rt=2.76 min; m/z [M+H]⁺=629.

Compound B33 (Second eluting isomer): ¹H NMR (400 MHz, DMSO) δ: 7.79 (1H, ddd, J=7.6, 7.6, 1.8 Hz), 7.64-7.58 (1H, m), 7.40 (1H, s), 7.37-7.26 (4H, m), 7.22 (1H, d, J=8.8 Hz), 5.66 (3H, s), 4.68 (2H, dd, J=6.4, 10.4 Hz), 4.39 (2H, t, J=6.2 Hz), 4.34-4.24 (2H, m), 4.06 (1H, quint, J=6.8 Hz), 2.84-2.76 (1H, m), 2.37-2.25 (3H, m), 1.99-1.91 (1H, m), 1.90-1.81 (1H, m), 1.57-1.47 (1H, m), 1.32 (3H, t, J=7.2 Hz). LCMS (Method B): Rt=3.07 min; m/z [M+H]⁺=581.

Compound B34 (First eluting isomer): ¹H NMR (400 MHz, DMSO) δ 7.79 (1H, ddd, J=7.6, 7.6, 1.7 Hz), 7.65-7.59 (1H, m), 7.41 (1H, s), 7.39-7.27 (4H, m), 7.25 (1H, d, J=8.0 Hz), 5.68-5.64 (3H, m), 4.71-4.66 (2H, m), 4.39 (2H, t, J=6.1 Hz), 4.36-4.25 (2H, m), 4.06 (1H, sept, J=6.8 Hz), 2.84-2.77 (1H, m), 2.38-2.30 (3H, m), 1.99-1.91 (1H, m), 1.90-1.84 (1H, m), 1.57-1.47 (1H, m), 1.35-1.29 (3H, m). LCMS (Method B): Rt=3.06 min; m/z [M+H]⁺=581.

Compound B35 (Second eluting isomer): ¹H NMR (400 MHz, DMSO) δ: 7.60 (1H, dd, J=1.8, 7.0 Hz), 7.42-7.23 (6H, m), 5.66 (3H, s), 4.71-4.65 (2H, m), 4.38 (2H, t, J=6.3 Hz), 4.36-4.26 (2H, m), 4.06 (1H, quint, J=6.8 Hz), 2.84-2.77 (1H, m), 2.33-2.30 (6H, m), 1.98-1.92 (1H, m), 1.87-1.83 (1H, m), 1.57-1.48 (1H, m), 1.32 (3H, t, J=7.1 Hz). LCMS (Method C): Rt=2.69 min; m/z [M+H]⁺=595.

Compound B36 (First eluting isomer): ¹H NMR (400 MHz, DMSO) δ 7.60 (1H, dd, J=1.8, 6.9 Hz), 7.42-7.22 (6H, m), 5.68-5.64 (3H, m), 4.71-4.65 (2H, m), 4.38 (2H, t, J=6.2 Hz), 4.36-4.25 (2H, m), 4.06 (1H, quint, J=6.8 Hz), 2.83-2.76 (1H, m), 2.33-2.30 (6H, m), 1.98-1.90 (1H, m), 1.87-1.82 (1H, m), 1.57-1.49 (1H, m), 1.32 (3H, t, J=7.2 Hz). LCMS (Method C): Rt=2.69 min; m/z [M+H]⁺=595.

Compound B37: ¹H NMR (400 MHz, DMSO) δ: 8.14 (1H, s), 7.54 (1H, d, J=8.8 Hz), 7.46 (1H, d, J=3.1 Hz), 7.41 (1H, q, J=0.0 Hz), 7.40-7.34 (2H, m), 7.31 (1H, dd, J=1.7, 8.4 Hz), 7.18 (1H, dd, J=3.1, 8.8 Hz), 5.70 (2H, s), 5.65 (1H, s), 4.71-4.65 (2H, m), 4.40 (2H, t, J=6.2 Hz), 4.37-4.21 (2H, m), 4.10 (1H, quint, J=6.8 Hz), 3.78-3.77 (3H, m), 2.86-2.84 (1H, m), 2.38-2.29 (3H, m), 2.00-1.93 (1H, m), 1.91-1.84 (1H, m), 1.59-1.47 (1H, m), 1.31 (3H, t, J=7.1 Hz). LCMS (Method A): Rt=2.72 min; m/z [M+H]⁺=627.

Compound B38 (Second eluting isomer): ¹H NMR (400 MHz, DMSO) δ 7.56-7.45 (3H, m), 7.42 (1H, s), 7.41-7.33 (4H, m), 7.28 (1H, d, J=8.5 Hz), 5.76-5.72 (2H, m), 5.69 (1H, s), 4.71-4.66 (2H, m), 4.60-4.55 (2H, m), 4.39 (2H, t, J=6.0 Hz), 4.38-4.26 (2H, m), 4.05 (1H, quint, J=6.8 Hz), 2.82-2.76 (1H, m), 2.38-2.26 (3H, m), 1.99-1.92 (1H, m), 1.89-1.84 (2H, m), 1.57-1.47 (1H, m), 1.37-1.32 (3H, m). LCMS (Method C): Rt=2.76 min; m/z [M+H]⁺=615.

Compound B39 First eluting isomer): ¹H NMR (400 MHz, DMSO) δ: 8.11 (1H, s), 7.73 (1H, dd, J=2.8, 6.0 Hz), 7.51-7.46 (1H, m), 7.38 (1H, s), 7.30-7.23 (2H, m), 7.13 (1H, dd, J=2.1, 12.0 Hz), 7.05 (1H, dd, J=1.9, 8.3 Hz), 5.66 (1H, s), 5.64 (2H, s), 4.68 (2H, dd, J=6.7, 10.4 Hz), 4.38 (2H, t, J=6.1 Hz), 4.37-4.22 (2H, m), 4.05 (1H, quint, J=6.8 Hz), 2.82-2.75 (1H, m), 2.36-2.30 (3H, m), 1.98-1.90 (1H, m), 1.86-1.81 (1H, m), 1.56-1.46 (1H, m), 1.31 (3H, t, J=7.1 Hz). LCMS (Method C): Rt=2.77 min; m/z [M+H]⁺=615.

Compound B42 (Second eluting isomer): LCMS (Method C): Rt=2.91 min; m/z [M+H]⁺=629.

Compound B43 (First eluting isomer): ¹H NMR (400 MHz, DMSO) δ: 7.56-7.49 (2H, m), 7.48-7.44 (1H, m), 7.39 (1H, s), 7.37-7.31 (3H, m), 7.26 (1H, d, J=8.3 Hz), 5.71 (3H, s), 4.95 (1H, sept, J=6.7 Hz), 4.86-4.67 (1H, m), 4.53 (2H, s), 2.91-2.68 (3H, m), 2.32 (2H, s), 2.03-1.92 (2H, m), 1.58 (1H, s), 1.49 (3H, s), 1.40 (3H, s), 1.32 (4H, dd, J=6.2, 24.0 Hz). LCMS (Method C): Rt=2.97 min; m/z [M+H]⁺=629.

Compound B44 (Second eluting isomer): ¹H NMR (400 MHz, DMSO) δ: 8.65 (1H, s), 7.55-7.50 (2H, m), 7.40-7.35 (2H, m), 7.28-7.24 (2H, m), 7.06 (2H, ddd, J=2.0, 10.3, 20.7 Hz), 5.70 (1H, s), 5.67 (2H, s), 4.91 (1H, sept, J=6.4 Hz), 4.41-4.21 (2H, m), 4.20 (2H, s), 3.24-2.93 (3H, m), 2.00-1.91 (2H, m), 1.56-1.46 (5H, m), 1.39 (3H, d, J=3.5 Hz), 1.35-1.28 (1H, m), 1.25 (2H, d, J=8.2 Hz), 1.10-1.06 (3H, m). LCMS (Method C): Rt=2.93 min; m/z [M+H]⁺=629.

Compound B45 (First eluting isomer): ¹H NMR (400 MHz, DMSO) δ: 8.96 (1H, s), 8.34 (1H, s), 7.55-7.49 (2H, m), 7.41-7.37 (1H, m), 7.36 (1H, s), 7.29-7.26 (2H, m), 7.13 (1H, dd, J=1.8, 11.8 Hz), 7.08 (1H, dd, J=1.6, 8.4 Hz), 5.68 (3H, s), 4.91 (2H, sept, J=6.7 Hz), 4.41-4.30 (1H, m), 4.26 (2H, s), 4.24-4.19 (1H, m), 3.18-2.93 (3H, m), 2.78-2.70 (1H, m), 2.36-2.29 (3H, m), 2.27-2.26 (3H, m), 1.96 (2H, s), 1.56-1.49 (5H, m), 1.42-1.35 (4H, m), 1.21-1.10 (6H, m), 1.10-1.05 (8H, m). LCMS (Method C): Rt=2.92 min; m/z [M+H]⁺=629.

Compound B46 (Second eluting isomer): ¹H NMR (400 MHz, DMSO) δ: 7.99 (1H, dd, J=1.6, 7.8 Hz), 7.63-7.59 (1H, m), 7.56 (1H, ddd, J=7.6, 7.6, 1.3 Hz), 7.46 (1H, ddd, J=7.5, 7.5, 1.3 Hz), 7.39 (1H, s), 7.36-7.28 (2H, m), 7.23 (1H, d, J=8.4 Hz), 5.71 (1H, s), 5.67 (2H, s), 4.93-4.76 (2H, m), 3.06-2.93 (3H, m), 2.32 (2H, s), 2.13-2.05 (2H, m), 1.63 (1H, s), 1.47 (3H, s), 1.38 (4H, d, J=6.1 Hz), 1.31 (2H, d, J=6.3 Hz). LCMS (Method C): Rt=2.87 min; m/z [M+H]⁺=615.

Compound B47 (First eluting isomer): ¹H NMR (400 MHz, DMSO) δ: 8.01-7.97 (1H, m), 7.62 (1H, dd, J=1.2, 7.8 Hz), 7.57 (1H, ddd, J=7.6, 7.6, 1.5 Hz), 7.47 (1H, ddd, J=7.5, 7.5, 1.4 Hz), 7.39 (1H, s), 7.36-7.28 (2H, m), 7.24 (1H, dd, J=1.7, 8.4 Hz), 5.69 (1H, s), 5.67 (2H, s), 4.94-4.76 (2H, m), 3.07 (1H, s), 3.03-2.91 (2H, m), 2.33 (2H, s), 2.12-2.02 (2H, m), 1.70-1.59 (1H, m), 1.47 (3H, s), 1.37 (4H, d, J=6.3 Hz), 1.32 (2H, d, J=6.3 Hz). LCMS (Method C): Rt=2.87 min; m/z [M+H]⁺=615.

Compound B48 (Second eluting isomer): ¹H NMR (400 MHz, DMSO) δ: 8.17 (1H, s), 7.55-7.51 (1H, m), 7.50-7.45 (2H, m), 7.43-7.37 (2H, m), 7.37-7.33 (2H, m), 7.29 (1H, d, J=8.3 Hz), 6.55 (1H, br. s), 5.70 (2H, s), 5.67 (1H, s), 4.97 (1H, sept, J=6.5 Hz), 4.62 (2H, s), 2.88-2.74 (4H, m), 2.43-2.24 (6H, m), 2.00-1.88 (2H, m), 1.59-1.54 (1H, m), 1.47 (3H, d, J=4.0 Hz), 1.37 (3H, d, J=3.9 Hz). LCMS (Method C): Rt=3.10 min; m/z [M+H]⁺=659.

Compound B49 (First eluting isomer): LCMS (Method C): Rt=3.06 min; m/z [M+H]⁺=659.

Compound B50 (Second eluting isomer): ¹H NMR (400 MHz, DMSO) δ: 7.99 (1H, dd, J=1.6, 7.8 Hz), 7.57 (1H, d, J=7.5 Hz), 7.53 (1H, dd, J=7.8, 7.8 Hz), 7.44 (1H, ddd, J=7.5, 7.5, 1.1 Hz), 7.36 (1H, s), 7.33-7.24 (2H, m), 7.16 (1H, d, J=8.0 Hz), 5.68 (1H, s), 5.63 (2H, s), 4.94 (1H, sept, J=6.7 Hz), 2.87-2.76 (3H, m), 2.42-2.22 (6H, m), 2.00-1.88 (2H, m), 1.62-1.52 (1H, m), 1.46 (3H, s), 1.35 (3H, s). LCMS (Method C): Rt=2.98 min; m/z [M+H]⁺=645.

Compound B51 (First eluting isomer): ¹H NMR (400 MHz, DMSO) δ: 7.99 (1H, dd, J=1.6, 7.8 Hz), 7.57 (1H, d, J=7.9 Hz), 7.51 (1H, dd, J=7.5, 7.5 Hz), 7.43 (1H, ddd, J=7.5, 7.5, 1.4 Hz), 7.36 (1H, s), 7.32-7.22 (2H, m), 7.15 (1H, d, J=7.9 Hz), 5.67 (1H, s), 5.63 (2H, s), 4.94 (1H, sept, J=6.3 Hz), 2.89-2.75 (3H, m), 2.42-2.23 (5H, m), 2.00-1.87 (2H, m), 1.61-1.53 (1H, m), 1.46 (3H, s), 1.36 (2H, s). LCMS (Method C): Rt=2.99 min; m/z [M+H]⁺=645.

Compound B52 (Second eluting isomer): ¹H NMR (400 MHz, DMSO) δ: 7.99 (1H, dd, J=1.8, 7.7 Hz), 7.52 (1H, d, J=7.5 Hz), 7.47 (1H, dd, J=6.9, 6.9 Hz), 7.41 (1H, dd, J=1.3, 7.5 Hz), 7.38 (1H, s), 7.26 (1H, dd, J=8.6, 8.6 Hz), 7.19 (1H, d, J=11.5 Hz), 7.09 (1H, d, J=8.5 Hz), 5.72 (1H, s), 5.67 (2H, s), 4.83 (1H, sept, J=6.0 Hz), 3.56 (2H, t, J=5.2 Hz), 3.07 (2H, s), 2.34 (2H, s), 2.20-2.12 (2H, m), 1.74-1.66 (1H, m), 1.46 (3H, d, J=4.3 Hz), 1.38 (3H, d, J=5.9 Hz). LCMS (Method C): Rt=2.87 min; m/z [M+H]⁺=613.

Compound B53 (First eluting isomer): ¹H NMR (400 MHz, DMSO) δ: 7.90 (1H, dd, J=1.7, 7.7 Hz), 7.44 (1H, d, J=7.7 Hz), 7.38 (1H, ddd, J=7.5, 7.5, 1.3 Hz), 7.33 (1H, dd, J=1.3, 7.6 Hz), 7.29 (1H, s), 7.17 (1H, dd, J=8.6, 8.6 Hz), 7.11 (1H, dd, J=1.8, 11.7 Hz), 7.00 (1H, d, J=8.0 Hz), 5.63 (1H, s), 5.58 (2H, s), 4.79-4.71 (1H, m), 3.47 (2H, t, J=5.1 Hz), 3.13 (1H, s), 3.01-2.95 (2H, m), 2.25 (2H, s), 2.11-2.03 (2H, m), 1.63-1.56 (1H, m), 1.39 (3H, d, J=4.5 Hz), 1.28 (3H, d, J=5.3 Hz). LCMS (Method C): Rt=2.85 min; m/z [M+H]⁺=613.

Compound B54 (First eluting isomer): ¹H NMR (400 MHz, DMSO) δ: 7.47-7.41 (2H, m), 7.38-7.34 (1H, m), 7.30 (1H, s), 7.27-7.22 (3H, m), 7.13 (1H, t, J=4.8 Hz), 5.62 (3H, s), 4.85 (1H, sept, J=6.7 Hz), 4.40 (2H, s), 3.40 (2H, t, J=5.4 Hz), 3.22 (3H, s), 2.92-2.84 (1H, m), 2.84-2.73 (2H, m), 2.37 (1H, s), 2.27-2.18 (2H, m), 1.98-1.89 (2H, m), 1.53 (1H, s), 1.41 (1H, s), 1.31 (1H, s). LCMS (Method C): Rt=2.92 min; m/z [M+H]⁺=627.

Compound B55 (Second eluting isomer): ¹H NMR (400 MHz, DMSO) δ: 7.55-7.49 (2H, m), 7.46-7.42 (1H, m), 7.39 (1H, s), 7.33-7.30 (2H, m), 7.27 (1H, d, J=12.3 Hz), 7.20 (1H, d, J=8.3 Hz), 5.70 (3H, s), 4.94 (1H, sept, J=6.6 Hz), 4.45 (2H, s), 3.48 (2H, t, J=5.3 Hz), 3.30 (3H, s), 3.25-3.28 (1H, m), 2.95 (1H, s), 2.90-2.81 (1H, m), 2.45 (1H, s), 2.39-2.29 (2H, m), 2.05-1.92 (2H, m), 1.61 (1H, s), 1.50 (2H, s), 1.40 (2H, s). LCMS (Method C): Rt=2.89 min; m/z [M+H]⁺=627.

Compound B58 (First eluting isomer): ¹H NMR (400 MHz, DMSO) δ: 7.78 (1H, ddd, J=7.6, 7.6, 1.6 Hz), 7.49 (1H, dd, J=6.4, 12.1 Hz), 7.33 (1H, s), 7.31-7.20 (3H, m), 7.17 (1H, dd, J=1.5, 11.5 Hz), 7.08 (1H, d, J=7.9 Hz), 5.65 (1H, s), 5.60 (2H, s), 4.91 (1H, sept, J=6.4 Hz), 4.67 (2H, dd, J=6.5, 10.5 Hz), 4.36 (2H, t, J=5.7 Hz), 4.03 (1H, quint, J=6.8 Hz), 2.80-2.74 (1H, m), 2.36-2.16 (3H, m), 1.97-1.89 (1H, m), 1.83-1.80 (1H, m), 1.54-1.48 (1H, m), 1.48-1.40 (2H, m), 1.38-1.30 (2H, m), 1.30-1.22 (1H, m), 0.90-0.84 (1H, m). LCMS (Method B): Rt=3.46 min; m/z [M+H]⁺=595.

Compound B59 (Second eluting isomer): ¹H NMR (400 MHz, DMSO) δ: 7.78 (1H, ddd, J=7.6, 7.6, 1.7 Hz), 7.45-7.37 (1H, m), 7.32 (1H, s), 7.24 (1H, dd, J=8.8, 8.8 Hz), 7.21-7.14 (3H, m), 7.09 (1H, dd, J=2.0, 12.1 Hz), 6.97 (1H, dd, J=1.8, 8.3 Hz), 5.64 (1H, s), 5.59 (2H, s), 4.90 (1H, sept, J=6.5 Hz), 4.67 (2H, dd, J=6.1, 11.1 Hz), 4.36 (2H, t, J=5.7 Hz), 4.03 (1H, quint, J=6.7 Hz), 2.80-2.75 (1H, m), 2.34-2.20 (3H, m), 1.97-1.87 (1H, m), 1.85-1.78 (1H, m), 1.54-1.47 (1H, m), 1.45 (3H, t, J=2.6 Hz), 1.34-1.30 (3H, m). LCMS (Method B): Rt=3.47 min; m/z [M+H]⁺=595.

Compound B60: ¹H NMR (400 MHz, DMSO) δ: 8.14 (1H, s), 7.81 (1H, ddd, J=7.5, 7.5, 1.7 Hz), 7.61-7.55 (1H, m), 7.37-7.27 (3H, m), 7.19-7.11 (2H, m), 5.73 (3H, d, J=13.2 Hz), 4.87 (1H, sept, J=6.5 Hz), 4.75-4.69 (2H, m), 4.48 (2H, t, J=6.3 Hz), 4.24 (1H, quint, J=6.4 Hz), 3.03 (1H, s), 2.43-2.25 (3H, m), 2.07-2.02 (1H, m), 1.96-1.90 (1H, m), 1.65-1.56 (1H, m), 1.44 (3H, s), 1.35 (3H, d, J=5.3 Hz). LCMS (Method A): Rt=2.56 min; m/z [M+H]⁺=613.

Compound B61 Second eluting isomer): ¹H NMR (400 MHz, DMSO) δ: 7.97 (1H, dd, J=1.5, 7.9 Hz), 7.64-7.57 (2H, m), 7.50-7.45 (1H, m), 7.40 (1H, s), 7.35-7.30 (2H, m), 7.27 (1H, d, J=8.4 Hz), 5.66 (3H, s), 4.70-4.65 (2H, m), 4.38 (2H, t, J=6.2 Hz), 4.35-4.22 (2H, m), 4.06 (1H, quint, J=6.8 Hz), 2.85-2.79 (1H, m), 2.36-2.28 (3H, m), 1.98-1.90 (1H, m), 1.86-1.82 (1H, m), 1.56-1.46 (1H, m), 1.30 (3H, t, J=7.1 Hz). LCMS (Method C): Rt=2.63 min; m/z [M+H]⁺=597.

Compound B62 (First eluting isomer): ¹H NMR (400 MHz, DMSO) δ: 7.97 (1H, dd, J=1.6, 7.8 Hz), 7.61-7.53 (2H, m), 7.47-7.42 (1H, m), 7.40-7.39 (1H, m), 7.34-7.28 (2H, m), 7.23 (1H, d, J=8.2 Hz), 5.66-5.63 (3H, m), 4.70-4.64 (2H, m), 4.37 (2H, t, J=6.2 Hz), 4.35-4.22 (2H, m), 4.05 (1H, quint, J=6.8 Hz), 2.83-2.76 (1H, m), 2.35-2.24 (3H, m), 1.97-1.91 (1H, m), 1.85-1.81 (1H, m), 1.55-1.46 (1H, m), 1.32-1.28 (3H, m). LCMS (Method C): Rt=2.64 min; m/z [M+H]⁺=597.

### Analytical Analysis

Method A: Experiments were performed on a Waters Acquity ZQ mass spectrometer linked to a Waters Acquity UPLC binary pump/PDA detector. The spectrometer had an electrospray source operating in positive and negative ion mode. Additional detection was achieved using a Acquity UPLC BEH C18 1.7 uM, 100×2.1 mm column maintained at 40° C. and a 0.4 mL/minute flow rate. The initial solvent system was 9.5% water containing 0.1% formic acid (solvent A) and 5% MeCN containing 0.1% formic acid (solvent B) for the first 0.4 minute followed by a gradient up to 5% solvent A and 95% solvent B over the next 5.6 min. The final solvent system was held constant for a further 0.8 min.

Method B: Experiments were performed on a Waters Acquity ZQ mass spectrometer linked to a Waters Acquity UPLC binary pump/PDA detector. The spectrometer had an electrospray source operating in positive and negative ion mode. Additional detection was achieved using a Acquity UPLC BEH C18 1.7 μM, 100×2.1 mm column maintained at 40° C. and a 0.4 mL/minute flow rate. The initial solvent system was 95% water containing 0.03% aqueous ammonia (solvent A) and 5% MeCN containing 0.03% aqueous ammonia (solvent B) for the first 0.4 minute followed by a gradient up to 5% solvent A and 95% solvent B over the next 4 min. The final solvent system was held constant for a further 0.8 min.

Method C: Experiments were performed on a Waters Acquity SQD2 mass spectrometer linked to a Waters Acquity UPLC binary pump/PDA detector. The spectrometer had an electrospray source operating in positive and negative ion mode. Additional detection was achieved using a Acquity UPLC HSS C18 1.7 uM, 100×2.1 mm column maintained at 40° C. and a 0.4 nit/minute flow rate. The initial solvent system was 95% water containing 0.1% formic acid (solvent A) and 5% MeCN containing 0.1% formic acid (solvent B) for the first 0.4 minute followed by a gradient up to 5% solvent A and 95% solvent B over the next 5.6 min. The final solvent system was held constant for a further 0.8 min.

Method D: Experiments were performed on a Waters Acquity ZQ mass spectrometer linked to a Waters Acquity H-class UPLC, with DAD detector and QDa. The spectrometer had an electrospray source operating in positive and negative ion mode. Additional detection was achieved using a Acquity UPLC CSH 1.7 uM, 50×2.1 mm column maintained at 40° C. and a 1.0 mL/minute flow rate. The initial solvent system was 97% water containing 0.1% formic acid (solvent A) and 3% MeCN containing 0.1% formic acid (solvent B) for the first 0.4 minute followed by a gradient up to 1% solvent A and 99% solvent B over the next 1.4 min. The final solvent system was held constant for a further 0.5 min,

Method E: Experiments were performed on a Waters Acquity ZQ mass spectrometer linked to a Waters Acquity H-class UPLC with 996 DAD detector and Quattro Micro MS. The spectrometer had an electrospray source operating in positive and negative ion mode. Additional detection was achieved using a Acquity UPLC CSH 1.7 uM, 50×2.1 mm column maintained at 40° C. and a 1.0 mL/minute flow rate. The initial solvent system was 97% water containing 0.1% formic acid (solvent A) and 3% MeCN containing 0.1% formic acid (solvent B) for the first 0.15 minutes followed by a gradient up to 1% solvent A and 99% solvent B over the next 1.4 min. The final solvent system was held constant for a further 0.5 min.

Method F: Experiments were performed on a Waters Acquity ZQ mass spectrometer linked to a Waters Acquity H-class UPLC with 996 DAD detector and Quattro Micro MS. The spectrometer had an electrospray source operating in positive and negative ion mode. Additional detection was achieved using a Acquity UPLC CSH 1.7 uM, 50×2.1 mm column maintained at 40° C. and a 1.0 mL/minute flow rate. The initial solvent system was 97% water containing 0.1% formic acid (solvent A) and 3% MeCN containing 0.1% formic acid (solvent B) for the first 0.15 minutes followed by a gradient up to 1% solvent A and 99% solvent B over the next 4.6 min. The final solvent system was held constant for a further 0.1 min.

Method G: Experiments were performed on a Waters Acquity ZQ mass spectrometer linked to a Waters Acquity H-class UPLC with DAD detector and QDa. The spectrometer had all electrospray source operating in positive and negative ion mode. Additional detection was achieved using a Acquity BEH UPLC 1.7 uM, 50×2.1 mm column maintained at 40° C. and a 0.8 mL/minute flow rate. The initial solvent system was 97% of 7.66 mM ammonia in water (solvent A) and 3% of 7.66 mM ammonia in MeCN containing (solvent B) for the first 0.4 minutes followed by a gradient up to 3% solvent A and 97% solvent B over the next 1.6 min. The final solvent system was held constant for a further 0.5 min.

Method H: Experiments were performed on a Waters Acquity ZQ mass spectrometer linked to a Waters Acquity H-class UPLC with DAD detector and QDa. The spectrometer had an electrospray source operating in positive and negative ion mode. Additional detection was achieved using a Acquity BEH UPLC 1.704, 50×2.1 mm column maintained at 40° C. and a 0.8 mL/minute flow rate. The initial solvent system was 97% of 7.66 mM ammonia in water (solvent A) and 3% of 7.66 mM ammonia in MeCN containing (solvent B) for the first 0.4 minutes followed by a gradient up to 3% solvent A and 97% solvent B over the next 4.1 min. The final solvent system was held constant for a further 0.5 min.

Method I: Experiments were performed on a Waters Acquity ZQ mass spectrometer linked to a HPLC 1100 system with DM) detector and CTC autosampler. The spectrometer had an electrospray source operating in positive and negative ion mode. Additional detection was achieved using a Waters XBridge 3.5 uM, 50×4.6 mm column maintained at 40° C. and a 2.0 mL/minute flow rate. The initial solvent system was 95% of 7.66 mM ammonia in water (solvent A) and 5% of 7.66 mM ammonia in MeCN containing (solvent B) for the first 0.3 minutes followed by a gradient up to 5% solvent A and 95% solvent B over the next 4.0 min. The final solvent system was held constant for a. further 1.0 min

Representative MDAP conditions: Sunfire C18, 3×50 mm, 3 μm, 5-95% ACN/H₂O (10 mM (NH₄)₂CO₃), 1.7 mL/min, RT.

Representative SFC conditions: LUX Cellulose-4, 4.6×250 mm, 5 μm, 55/45% MeOH (0.1% DEA)/CO₂, 5.0 mL/min, 120 bar, 40° C.

### Biochemical Assays to Detect Inhibition of Kinase Activity of IRE1α

The kinase reactions were performed in 384 well white ProxiPlate-384 Plus plates (PERKIN Elmer 6008280) using 25 mM MOPS assay buffer with 1 mM dithiothreitol, 25 mM MgCl₂, 12.5 mM β-glycerophosphate, 5 mM EGTA, and 50 μg/mL BSA. Test compounds were prepared on the day of assay and dispensed using D300 digital dispenser as a 10-point ½ log dilution series in duplicate, normalized to a final DMSO concentration of 3%. Test compounds were pre-incubated for 30 min at room temperature with 10 nM IRE1α kinase (E31-11G from Signal Chem) in 2.5 μL of assay buffer and the reaction started by addition of 2.5 μL of ATP in assay buffer, to give a final ATP concentration of 100 μM and 5 nM IRE1α kinase. After 4 hours incubation at room temperature the reactions were stopped and the kinase activity determined using the ADP-Glo™ reagent from Promega, according to the manufacturer's instructions. Luminescence was measured on a luminometer (EnVision, PerkinElmer) and IC₅₀ values calculated by fitting a sigmoidal curve to percent inhibition of control versus Log₁₀ of compound concentration.

### Pharmacological In Vitro Assays

### Biochemical Assay: Inhibition of RNase Activity of IRE1α

The RNase reactions were performed in 384 well black ProxiPlate-384 Plus plates (PERKIN Elmer) using 50 mM Tris assay buffer with 0.5 mM MgCl₂, 10 mM KCl, 0.03% Tween, 2 mM DTT and 1% DMSO. Test compounds were prepared on the day of assay and dispensed using D300 digital dispenser as a 10-point ½ log dilution series in duplicate, normalized to a final DMSO concentration of 4%. Test compounds were pre-incubated for 30 min at room temperature with IRE1α kinase (E31-11G from Signal Chem) in 2.5 μL of assay buffer. Then 2.5 μl of assay buffer containing substrate (5′ Alexa Fluor 647-rCrArU rGrUrC rCrGrC rArGrC rGrCrArUrG-Iowa Black RQ quencher 3′) added, giving a final concentration of enzyme of 0.325 nM and of substrate of 100 nM. After 20 minutes incubation at room temperature the reactions were stopped by added 5 μL of 5M urea, incubated at room temperature for 10 minutes and fluorescence measured on a plate reader (EnVision, PerkinElmer). IC₅₀ values calculated by fitting a sigmoidal curve to percent inhibition of control versus compound concentration.

### Cellular In Vitro Assays

### Cellular XBP1 Splicing Assay

ARPE-19 cells stably expressing XBP1 (a.a. 1-376) with nano-luciferase gene sequence linked so it is in frame when XBP1 is spliced, were cultured in F12 media, 10% FBS, 0.044% sodium bicarbonate, 150 μg/ml hygromycin B and seeded for assays at 5,000 cells in 384 well plates in culture media without hygromycin B and incubated at 37° C./5% CO₂. After overnight incubation test compounds were added to the cell plate in a 10-point ½ log dilution series in duplicate (final DMSO concentration 0.117%). After further incubation of 30 minutes thapsigargin was added (final concentration 150 nM) and then another 4 hour incubation. A NanoLuc luciferase assay (Promega) was used according to the manufacturer's instructions to detect the luciferase and luminescence measured on a luminometer (EnVision, PerkinElmer). IC₅₀ values calculated by fitting a sigmoidal curve to percent inhibition of control of compound concentration.

### Cellular Apoptosis Assay

INS-1 cells expressing mIRE1 were grown in RPMI, 10% FCS, 0.0003% β-mercaptoethanol and 150 pg/mL hygromycin B and for assays seeded at 10,000 cells/well in 384 well plates in media without with hygromycin B. After 24 hours incubation test compounds were added to the plate 10-point ½ log dilution series in duplicate and incubated for 30 minutes. Doxycycline (final concentration 100 nM) was added and plates incubated for a further 72 hours. To determine the proportion of apoptotic cells Hoechst 33342 (final concentration 10 ug/mL) was added, then after 30 minutes incubation cells imaged and analyzed on an InCell high content imager.

Biological results are summarized in Table 26.

TABLE 26

| | | | IRE1α | IRE1α |
|---|---|---|---|---|
| | | XBP1 | RNase | Kinase |
| | | IC50 | IC50 | IC50 |
| Cmpd | Structure | (μM) | (μM) | (μM) |
| A1 | N-(4-(4-amino-1-isopropyl-7-((1r,4r)-4-(oxetan-3- | 0.0079 | 0.0057 | 0.0019 |
| | ylamino)cyclohexyl)-1H-pyrazolo[4,3-c]pyridin-3- | | | |
| | yl)-2-fluorophenyl)-2-fluorobenzenesulfonamide | | | |
| A2 | N-(4-(4-amino-1-isopropyl-7-((1r,4r)-4-((2- | 0.003 | 0.0046 | NT |
| | methoxyethyl)amino)cyclohexyl)-1H-pyrazolo[4,3- | | | |
| | c]pyridin-3-yl)-2,5-difluorophenyl)-2-chloro-5- | | | |
| | methoxybenzenesulfonamide | | | |
| A3 | N-(4-(4-amino-1-isopropyl-7-((1r,4r)-4- | 0.015 | 0.016 | NT |
| | morpholinocyclohexyl)-1H-pyrazolo[4,3-c]pyridin-3- | | | |
| | yl)-2,5-difluorobenzenesulphonamide | | | |
| A4 | N-(4-(4-amino-1-isopropyl-7-((1r,4r)-4-((2- | 0.01 | 0.0013 | NT |
| | methoxyethyl)amino)cyclohexyl)-1H-pyrazolo[4,3- | | | |
| | c]pyridin-3-yl)-2,5-difluorophenyl)-5-chloro-2- | | | |
| | fluorobenzenesulfonamide | | | |
| A5 | N-(4-(4-amino-1-isopropyl-7-((1r,4r)-4-((2- | 0.025 | 0.0019 | NT |
| | methoxyethyl)amino)cyclohexyl)-1H-pyrazolo[4,3- | | | |
| | c]pyridin-3-yl)-2,5-difluorophenyl)-5- | | | |
| | (difluoromethoxy)-2-fluorobenzenesulfonamide | | | |
| A6 | N-(4-(4-amino-1-isopropyl-7-((1r,4r)-4-((2- | 0.0027 | 0.0026 | NT |
| | methoxyethyl)amino)cyclohexyl)-1H-pyrazolo[4,3- | | | |
| | c]pyridin-3-yl)-2-fluorophenyl)-2- | | | |
| | fluorobenzenesulfonamide | | | |
| A7 | N-(4-(4-amino-1-isopropyl-7-((1r,4r)-4-((2- | 0.024 | 0.0029 | NT |
| | methoxyethyl)amino)cyclohexyl)-1H-pyrazolo[4,3- | | | |
| | c]pyridin-3-yl)-2,5-difluorophenyl)-2-fluoro-3- | | | |
| | methoxybenzenesulfonamide | | | |
| A8 | N-(4-(4-amino-1-isopropyl-7-((1r,4r)-4-((2- | 0.0039 | 0.0019 | NT |
| | methoxyethyl)amino)cyclohexyl)-1H-pyrazolo[4,3- | | | |
| | c]pyridin-3-yl)-2,5-difluorophenyl)-5-ethoxy-2- | | | |
| | fluorobenzenesulfonamide | | | |
| A9 | N-(4-(4-amino-1-isopropyl-7-((1r,4r)-4-((2- | 0.11 | 0.020 | NT |
| | methoxyethyl)amino)cyclohexyl)-1H-pyrazolo[4,3- | | | |
| | c]pyridin-3-yl)-2,5-difluorophenyl)-4- | | | |
| | methoxybenzenesulfonamide | | | |
| A10 | N-(4-(4-amino-1-isopropyl-7-((1r,4r)-4-((2- | 0.019 | 0.24 | NT |
| | methoxyethyl)amino)cyclohexyl)-1H-pyrazolo[4,3- | | | |
| | c]pyridin-3-yl)-2,5-difluorophenyl)-2- | | | |
| | methoxybenzenesulfonamide | | | |
| A11 | N-(4-(4-amino-1-isopropyl-7-((1r,4r)-4-((2- | 0.015 | 0.0089 | NT |
| | methoxyethyl)amino)cyclohexyl)-1H-pyrazolo[4,3- | | | |
| | c]pyridin-3-yl)-2,5-difluorophenyl)-3- | | | |
| | methoxybenzenesulfonamide | | | |
| A12 | N-(4-(4-amino-1-isopropyl-7-((1s,4s)-4-(oxetan-3- | 1.4 | 0.44 | NT |
| | ylamino)cyclohexyl)-1H-pyrazolo[4,3-c]pyridin-3- | | | |
| | yl)-2-fluorophenyl)-2-fluorobenzenesulfonamide | | | |
| A13 | N-(4-(4-amino-1-isopropyl-7-((1r,4r)-4-((2- | 0.078 | 0.017 | NT |
| | methoxyethyl)amino)cyclohexyl)-1H-pyrazolo[4,3- | | | |
| | c]pyridin-3-yl)-2,5-difluorophenyl)-4-fluoro-3- | | | |
| | methoxybenzenesulfonamide | | | |
| A14 | N-(4-(4-amino-1-isopropyl-7-((1r,4r)-4-((2- | 0.24 | 0.021 | NT |
| | methoxyethyl)amino)cyclohexyl)-1H-pyrazolo[4,3- | | | |
| | c]pyridin-3-yl)-2,5-difluorophenyl)-4- | | | |
| | chlorobenzenesulfonamide | | | |
| A15 | N-(4-(4-amino-1-isopropyl-7-((1r,4r)-4-((2- | 0.013 | 0.0066 | NT |
| | methoxyethyl)amino)cyclohexyl)-1H-pyrazolo[4,3- | | | |
| | c]pyridin-3-yl)-2,5-difluorophenyl)-2-chloro-4- | | | |
| | fluorobenzenesulfonamide | | | |
| A16 | N-(4-(4-amino-1-isopropyl-7-((1r,4r)-4-((2- | 0.077 | 0.0078 | NT |
| | methoxyethyl)amino)cyclohexyl)-1H-pyrazolo[4,3- | | | |
| | c]pyridin-3-yl)-2,5-difluorophenyl)-4-chloro-2- | | | |
| | fluorobenzenesulfonamide | | | |
| A17 | N-(4-(4-amino-1-isopropyl-7-((1r,4r)-4-((2- | 0.012 | 0.0009 | NT |
| | methoxyethyl)amino)cyclohexyl)-1H-pyrazolo[4,3- | | | |
| | c]pyridin-3-yl)-2,5-difluorophenyl)-1-(4- | | | |
| | fluorophenyl)methanesulfonamide | | | |
| A18 | N-(4-(4-amino-1-isopropyl-7-((1r,4r)-4-((2- | 0.037 | 0.012 | NT |
| | methoxyethyl)amino)cyclohexyl)-1H-pyrazolo[4,3- | | | |
| | c]pyridin-3-yl)-3-fluorophenyl)-2- | | | |
| | fluorobenzenesulfonamide | | | |
| A19 | N-(4-(4-amino-1-isopropyl-7-((1r,4r)-4-((2- | 0.004 | 0.0061 | NT |
| | methoxyethyl)amino)cyclohexyl)-1H-pyrazolo[4,3- | | | |
| | c]pyridin-3-yl)-2,5-difluorophenyl)-2- | | | |
| | chlorobenzenesulfonamide | | | |
| A20 | N-(4-(4-amino-1-isopropyl-7-((1r,4r)-4-((2- | 0.013 | 0.0012 | NT |
| | methoxyethyl)amino)cyclohexyl)-1H-pyrazolo[4,3- | | | |
| | c]pyridin-3-yl)-2,5-difluorophenyl)-1-(2- | | | |
| | chlorophenyl)methanesulfonamide | | | |
| A21 | N-(4-(4-amino-1-isopropyl-7-((1r,4r)-4-((2- | 3.8 | 0.0047 | NT |
| | methoxyethyl)amino)cyclohexyl)-1H-pyrazolo[4,3- | | | |
| | c]pyridin-3-yl)-2,5-difluorophenyl)-5-cyano-2- | | | |
| | fluorobenzenesulfonamide | | | |
| A22 | N-(4-(4-amino-7-((1r,4r)-4- | 0.015 | 0.0018 | NT |
| | (dimethylamino)cyclohexyl)-1-isopropyl-1H- | | | |
| | pyrazolo[4,3-c]pyridin-3-yl)-2,5-difluorophenyl)-1- | | | |
| | (2-fluorophenyl)methanesulfonamide | | | |
| A23 | N-(4-(4-amino-1-isopropyl-7-((1r,4r)-4-((2- | 0.16 | 0.0097 | NT |
| | methoxyethyl)amino)cyclohexyl)-1H-pyrazolo[4,3- | | | |
| | c]pyridin-3-yl)-2,5-difluorophenyl)-2,6- | | | |
| | difluorobenzenesulfonamide | | | |
| A24 | N-(4-(4-amino-1-isopropyl-7-((1r,4r)-4-((2- | 0.082 | 0.0054 | NT |
| | methoxyethyl)amino)cyclohexyl)-1H-pyrazolo[4,3- | | | |
| | c]pyridin-3-yl)-2,5-difluorophenyl)-2,4- | | | |
| | difluorobenzenesulfonamide | | | |
| A25 | N-(4-(4-amino-1-isopropyl-7-((1r,4r)-4-((2- | 0.025 | 0.0090 | NT |
| | methoxyethyl)amino)cyclohexyl)-1H-pyrazolo[4,3- | | | |
| | c]pyridin-3-yl)-2,5-difluorophenyl)-2,3- | | | |
| | difluorobenzenesulfonamide | | | |
| A26 | N-(4-(4-amino-1-isopropyl-7-((1r,4r)-4-((2- | 0.013 | 0.0015 | NT |
| | methoxyethyl)amino)cyclohexyl)-1H-pyrazolo[4,3- | | | |
| | c]pyridin-3-yl)-2,5-difluorophenyl)-1-(2- | | | |
| | fluorophenyl)methanesulfonamide | | | |
| A27 | N-(4-(4-amino-7-((1r,4r)-4-(bis(2- | 0.013 | 0.013 | NT |
| | methoxyethyl)amino)cyclohexyl)-1-isopropyl-1H- | | | |
| | pyrazolo[4,3-c]pyridin-3-yl)-2,5-difluorophenyl)-2- | | | |
| | fluoro-5-methoxybenzenesulfonamide | | | |
| A28 | N-(4-(4-amino-1-isopropyl-7-((1r,4r)-4-((2- | 0.0069 | 0.0029 | NT |
| | methoxyethyl)amino)cyclohexyl)-1H-pyrazolo[4,3- | | | |
| | c]pyridin-3-yl)-2,5-difluorophenyl)-2-fluoro-5- | | | |
| | methoxybenzenesulfonamide | | | |
| A29 | N-(4-(4-amino-7-((1r,4r)-4-(bis(2- | 0.50 | 0.032 | NT |
| | methoxyethyl)amino)cyclohexyl)-1-isopropyl-1H- | | | |
| | pyrazolo[4,3-c]pyridin-3-yl)-2,5-difluorophenyl)-3- | | | |
| | (trifluoromethyl)benzenesulfonamide | | | |
| A30 | N-(4-(4-amino-1-isopropyl-7-((1r,4r)-4-((2- | 0.093 | 0.0038 | NT |
| | methoxyethyl)amino)cyclohexyl)-1H-pyrazolo[4,3- | | | |
| | c]pyridin-3-yl)-2,5-difluorophenyl)-3- | | | |
| | (trifluoromethyl)benzenesulfonamide | | | |
| A31 | N-(4-(4-amino-1-isopropyl-7-((1r,4r)-4-((2- | 0.039 | 0.0086 | NT |
| | methoxyethyl)amino)cyclohexyl)-1H-pyrazolo[4,3- | | | |
| | c]pyridin-3-yl)-2,5-difluorophenyl)-2,5- | | | |
| | difluorobenzenesulfonamide | | | |
| A32 | N-(4-(4-amino-1-isopropyl-7-((1r,4r)-4-((2- | 0.11 | 0.017 | NT |
| | methoxyethyl)amino)cyclohexyl)-1H-pyrazolo[4,3- | | | |
| | c]pyridin-3-yl)-2,5-difluorophenyl)-4- | | | |
| | fluorobenzenesulfonamide | | | |
| A33 | N-(4-(4-amino-1-isopropyl-7-((1r,4r)-4-((2- | 0.049 | NT | NT |
| | methoxyethyl)amino)cyclohexyl)-1H-pyrazolo[4,3- | | | |
| | c]pyridin-3-yl)-2,5-difluorophenyl)-3- | | | |
| | fluorobenzenesulfonamide | | | |
| A34 | N-(4-(4-amino-1-isopropyl-7-((1r,4r)-4-(oxetan-3- | 0.0043 | 0.0035 | 0.003 |
| | ylamino)cyclohexyl)-1H-pyrazolo[4,3-c]pyridin-3- | | | |
| | yl)-2-fluorophenyl)-2-chlorobenzenesulfonamide | | | |
| A35 | N-(4-(4-amino-7-((1r,4r)-4-(bis(2- | 0.055 | 0.060 | NT |
| | methoxyethyl)amino)cyclohexyl)-1-isopropyl-1H- | | | |
| | pyrazolo[4,3-c]pyridin-3-yl)-2,5-difluorophenyl)-2- | | | |
| | fluorobenzenesulfonamide | | | |
| A36 | N-(4-(4-amino-1-isopropyl-7-((1r,4r)-4-((2- | 0.018 | 0.013 | NT |
| | methoxyethyl)(methyl)amino)cyclohexyl)-1H- | | | |
| | pyrazolo[4,3-c]pyridin-3-yl)-2,5-difluorophenyl)-2- | | | |
| | fluorobenzenesulfonamide | | | |
| A37 | N-(4-(4-amino-1-isopropyl-7-((1r,4r)-4-((2- | 0.020 | 0.012 | NT |
| | methoxyethyl)amino)cyclohexyl)-1H-pyrazolo[4,3- | | | |
| | c]pyridin-3-yl)-2,5-difluorophenyl)-2- | | | |
| | fluorobenzenesulfonamide | | | |
| A38 | N-(4-(4-amino-1-isopropyl-7-((1r,4r)-4- | 0.013 | 0.012 | NT |
| | (methyl(oxetan-3-y0amino)cyclohexyl)-1H- | | | |
| | pyrazolo[4,3-c]pyridin-3-yl)-2,5-difluorophenyl)-2- | | | |
| | fluorobenzenesulfonamide | | | |
| A39 | N-(4-(4-amino-1-isopropyl-7-((1r,4r)-4-(oxetan-3- | 0.0017 | 0.0003 | NT |
| | ylamino)cyclohexyl)-1H-pyrazolo[4,3-c]pyridin-3- | | | |
| | yl)-2-fluorophenyl)-1-(2- | | | |
| | chlorophenyl)methanesulfonamide | | | |
| A40 | N-(4-(4-amino-7-((1r,4r)-4- | 0.027 | 0.0056 | NT |
| | (dimethylamino)cyclohexyl)-1-isopropyl-1H- | | | |
| | pyrazolo[4,3-c]pyridin-3-yl)-2,5-difluorophenyl)-2- | | | |
| | fluorobenzenesulfonamide | | | |
| A41 | N-(4-(4-amino-1-cyclopropyl-7-((1r,4r)-4-(oxetan-3- | 0.016 | 0.0080 | 0.0043 |
| | ylamino)cyclohexyl)-1H-pyrazolo[4,3-c]pyridin-3- | | | |
| | yl)-2-fluorophenyl)-2-fluorobenzenesulfonamide | | | |
| A42 | N-(4-(4-amino-1-ethyl-7-((1r,4r)-4-(oxetan-3- | 0.019 | 0.0032 | 0.003 |
| | ylamino)cyclohexyl)-1H-pyrazolo[4,3-c]pyridin-3- | | | |
| | yl)-2,5-difluorophenyl)-1-(2- | | | |
| | chlorophenyl)methanesulfonamide | | | |
| A43 | N-(4-(4-amino-1-ethyl-7-((1r,4r)-4-(oxetan-3- | 0.052 | 0.0054 | 0.0032 |
| | ylamino)cyclohexyl)-1H-pyrazolo[4,3-c]pyridin-3- | | | |
| | yl)-2,5-difluorophenyl)-1-(2- | | | |
| | fluorophenyl)methanesulfonamide | | | |
| A44 | N-(4-(4-amino-1-ethyl-7-((1r,4r)-4-(oxetan-3- | 0.031 | 0.0082 | 0.0028 |
| | ylamino)cyclohexyl)-1H-pyrazolo[4,3-c]pyridin-3- | | | |
| | yl)-2,5-difluorophenyl)-2-chlorobenzenesulfonamide | | | |
| A45 | N-(4-(4-amino-1-ethyl-7-((1r,4r)-4-(oxetan-3- | 0.094 | 0.016 | 0.0065 |
| | ylamino)cyclohexyl)-1H-pyrazolo[4,3-c]pyridin-3- | | | |
| | yl)-2,5-difluorophenyl)-2-fluorobenzenesulfonamide | | | |
| A46 | N-(4-(4-amino-1-isopropyl-7-((1r,4r)-4- | 0.0084 | 0.0077 | 0.0036 |
| | (methyl(oxetan-3-y0amino)cyclohexyl)-1H- | | | |
| | pyrazolo[4,3-c]pyridin-3-yl)-2-fluorophenyl)-2- | | | |
| | fluorobenzenesulfonamide | | | |
| A47 | N-(4-(4-amino-1-isopropyl-7-((1r,4r)-4-(oxetan-3- | 0.0056 | 0.0013 | 0.002 |
| | ylamino)cyclohexyl)-1H-pyrazolo[4,3-c]pyridin-3- | | | |
| | yl)-2,5-difluorophenyl)-1-(2- | | | |
| | chlorophenyl)methanesulfonamide | | | |
| A48 | N-(4-(4-amino-1-isopropyl-7-((1r,4r)-4-(oxetan-3- | 0.0064 | 0.0019 | 0.0016 |
| | ylamino)cyclohexyl)-1H-pyrazolo[4,3-c]pyridin-3- | | | |
| | yl)-2,5-difluorophenyl)-1-(2- | | | |
| | fluorophenyl)methanesulfonamide | | | |
| A49 | N-(4-(4-amino-1-isopropyl-7-((1r,4r)-4-(oxetan-3- | 0.0043 | 0.0060 | 0.0021 |
| | ylamino)cyclohexyl)-1H-pyrazolo[4,3-c]pyridin-3- | | | |
| | yl)-2,5-difluorophenyl)-2-chlorobenzenesulfonamide | | | |
| A50 | N-(4-(4-amino-1-isopropyl-7-((1r,4r)-4-(oxetan-3- | 0.017 | 0.0069 | 0.0033 |
| | ylamino)cyclohexyl)-1H-pyrazolo[4,3-c]pyridin-3- | | | |
| | yl)-2,5-difluorophenyl)-2-fluorobenzenesulfonamide | | | |
| A51 | N-(4-(4-amino-7-((1r,4r)-4- | 0.002 | 0.0005 | 0.0027 |
| | (dimethylamino)cyclohexyl)-1-isopropyl-1H- | | | |
| | pyrazolo[4,3-c]pyridin-3-yl)-2-fluorophenyl)-1-(2- | | | |
| | chlorophenyl)methanesulfonamide | | | |
| A52 | N-(4-(4-amino-1-ethyl-7-((1r,4r)-4-(oxetan-3- | 0.015 | 0.0061 | 0.0046 |
| | ylamino)cyclohexyl)-1H-pyrazolo[4,3-c]pyridin-3- | | | |
| | yl)-2-fluorophenyl)-2-chlorobenzenesulfonamide | | | |
| A53 | N-(4-(4-amino-1-ethyl-7-((1r,4r)-4-(oxetan-3- | 0.0092 | 0.0015 | 0.0019 |
| | ylamino)cyclohexyl)-1H-pyrazolo[4,3-c]pyridin-3- | | | |
| | yl)-2-fluorophenyl)-1-(2- | | | |
| | fluorophenyl)methanesulfonamide | | | |
| A54 | N-(4-(4-amino-1-cyclopropyl-7-((1r,4r)-4-(oxetan-3- | 0.009 | 0.0009 | NT |
| | ylamino)cyclohexyl)-1H-pyrazolo[4,3-c]pyridin-3- | | | |
| | yl)-2-fluorophenyl)-1-(2- | | | |
| | fluorophenyl)methanesulfonamide | | | |
| A55 | N-(4-(4-amino-1-cyclopropyl-7-((1r,4r)-4-(oxetan-3- | 0.0072 | 0.0007 | NT |
| | ylamino)cyclohexyl)-1H-pyrazolo[4,3-c]pyridin-3- | | | |
| | yl)-2-fluorophenyl)-1-(2- | | | |
| | chlorophenyl)methanesulfonamide | | | |
| A56 | N-(4-(4-amino-1-cyclopropyl-7-((1r,4r)-4-(oxetan-3- | 0.015 | 0.0035 | NT |
| | ylamino)cyclohexyl)-1H-pyrazolo[4,3-c]pyridin-3- | | | |
| | yl)-2-fluorophenyl)-2-chlorobenzenesulfonamide | | | |
| A57 | N-(4-(4-amino-1-isopropyl-7-((1r,4r)-4-(oxetan-3- | 0.0025 | 0.0004 | 0.0009 |
| | ylamino)cyclohexyl)-1H-pyrazolo[4,3-c]pyridin-3- | | | |
| | yl)-2-fluorophenyl)-1-(2- | | | |
| | fluorophenyl)methanesulfonamide | | | |
| A58 | N-(4-(4-amino-1-isopropyl-7-((1r,4r)-4-((1- | 0.0049 | 0.0006 | NT |
| | methoxypropan-2-y0amino)cyclohexyl)-1H- | | | |
| | pyrazolo[4,3-c]pyridin-3-yl)-2-fluorophenyl)-1-(2- | | | |
| | chlorophenyl)methanesulfonamide | | | |
| A59 | N-(4-(4-amino-1-isopropyl-7-((1r,4r)-4-((2- | 0.044 | NT | NT |
| | methoxyethyl)amino)cyclohexyl)-1H-pyrazolo[4,3- | | | |
| | c]pyridin-3-yl)-2,5-difluorophenyl)-2-fluoro-5- | | | |
| | (trifluoromethoxy)benzenesulfonamide | | | |
| A60 | N-(4-(4-amino-1-isopropyl-7-((1r,4r)-4-((2- | 0.016 | NT | NT |
| | methoxyethyl)amino)cyclohexyl)-1H-pyrazolo[4,3- | | | |
| | c]pyridin-3-yl)-2,5-difluorophenyl)-2-fluoro-5- | | | |
| | propoxybenzenesulfonamide | | | |
| A61 | N-(4-(4-amino-1-isopropyl-7-((1r,4r)-4-((2- | 0.005 | NT | NT |
| | methoxyethyl)amino)cyclohexyl)-1H-pyrazolo[4,3- | | | |
| | c]pyridin-3-yl)-2,5-difluorophenyl)-2,5- | | | |
| | dichlorobenzenesulfonamide | | | |
| A62 | N-(4-(4-amino-1-isopropyl-7-((1r,4r)-4-((2- | 0.006 | NT | NT |
| | methoxyethyl)amino)cyclohexyl)-1H-pyrazolo[4,3- | | | |
| | c]pyridin-3-yl)-2,5-difluorophenyl)-2-chloro-5- | | | |
| | ethylbenzenesulfonamide | | | |
| A63 | N-(4-(4-amino-7-((1r,4r)-4-((2- | 0.013 | NT | NT |
| | fluoropropyl)amino)cyclohexyl)-1-isopropyl-1H- | | | |
| | pyrazolo[4,3-c]pyridin-3-yl)-2,5-difluorophenyl)-2- | | | |
| | fluorobenzenesulfonamide | | | |
| B1 | N-(4-(4-amino-1-isopropyl-7-(4-(oxetan-3- | 0.030 | 0.0024 | NT |
| (first | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B2 | N-(4-(4-amino-1-isopropyl-7-(4-(oxetan-3- | 0.0065 | 0.0008 | 0.0019 |
| (second | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B3 | N-(4-(4-amino-1-isopropyl-7-(4-((1-methoxypropan- | 0.011 | 0.0070 | NT |
| | 2-y0amino)cyclohex-1-en-1-yl)-1H-pyrazolo[4,3- | | | |
| | c]pyridin-3-yl)-2-fluorophenyl)-2- | | | |
| | chlorobenzenesulfonamide | | | |
| B4 | N-(4-(4-amino-1-isopropyl-7-(4-((1-methoxypropan- | 0.0068 | 0.0012 | NT |
| (Second | 2-yl)amino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B5 | N-(4-(4-amino-1-isopropyl-7-(4-((1-methoxypropan- | 0.0065 | 0.0012 | NT |
| (First | 2-yl)amino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B6 | N-(4-(4-amino-1-isopropyl-7-(4-(oxetan-3- | 0.047 | 0.0051 | 0.0052 |
| (Second | ylamino)cyclohex-1-en-1-yl)-1H-pyrazolo[4,3- | | | |
| eluting | c]pyridin-3-yl)-2,5-difluorophenyl)-1-(2- | | | |
| isomer) | chlorophenyl)methanesulfonamide | | | |
| B7 | N-(4-(4-amino-1-isopropyl-7-(4-(oxetan-3- | 0.41 | 0.021 | NT |
| (First | ylamino)cyclohex-1-en-1-yl)-1H-pyrazolo[4,3- | | | |
| eluting | c]pyridin-3-yl)-2,5-difluorophenyl)-1-(2- | | | |
| isomer) | chlorophenyl)methanesulfonamide | | | |
| racemic | N-(4-(4-amino-1-isopropyl-7-(4-(oxetan-3- | 0.24 | 0.11 | NT |
| mixture | ylamino)cyclohex-1-en-1-yl)-1H-pyrazolo[4,3- | | | |
| of B8 & | c]pyridin-3-yl)-2-fluorophenyl)-2-chloro-5- | | | |
| B9 | methoxybenzenesulfonamide | | | |
| B8 | N-(4-(4-amino-1-isopropyl-7-(4-(oxetan-3- | 0.019 | 0.0067 | NT |
| (Second | ylamino)cyclohex-1-en-1-yl)-1H-pyrazolo[4,3- | | | |
| eluting | c]pyridin-3-yl)-2-fluorophenyl)-2-chloro-5- | | | |
| isomer) | methoxybenzenesulfonamide | | | |
| B9 | N-(4-(4-amino-1-isopropyl-7-(4-(oxetan-3- | 0.15 | 0.070 | NT |
| (First | ylamino)cyclohex-1-en-1-yl)-1H-pyrazolo[4,3- | | | |
| eluting | c]pyridin-3-yl)-2-fluorophenyl)-2-chloro-5- | | | |
| isomer) | methoxybenzenesulfonamide | | | |
| racemic | N-(4-(4-amino-1-isopropyl-7-(4-(oxetan-3- | 0.16 | 0.06 | NT |
| mixture | ylamino)cyclohex-1-en-1-yl)-1H-pyrazolo[4,3- | | | |
| of B10 | c]pyridin-3-yl)-2,5-difluorophenyl)-2- | | | |
| & B11 | chlorobenzenesulfonamide | | | |
| B10 | N-(4-(4-amino-1-isopropyl-7-(4-(oxetan-3- | 0.057 | 0.013 | 0.007 |
| (Second | ylamino)cyc1ohex-1-en-1-yl)-1H-pyrazolo[4,3- | | | |
| eluting | c]pyridin-3-yl)-2,5-difluorophenyl)-2- | | | |
| isomer) | chlorobenzenesulfonamide | | | |
| B11 | N-(4-(4-amino-1-isopropyl-7-(4-(oxetan-3- | 0.50 | 0.034 | NT |
| (First | ylamino)cyclohex-1-en-1-yl)-1H-pyrazolo[4,3- | | | |
| eluting | c]pyridin-3-yl)-2,5-difluorophenyl)-2- | | | |
| isomer) | chlorobenzenesulfonamide | | | |
| B12 | N-(4-(4-amino-1-isopropyl-7-(4-(oxetan-3- | 0.014 | 0.0016 | 0.0018 |
| (Second | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B13 | N-(4-(4-amino-1-isopropyl-7-(4-(oxetan-3- | 0.090 | 0.0052 | NT |
| (First | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| racemic | N-(4-(4-amino-1-isopropyl-7-(4-(oxetan-3- | 0.0033 | 0.06 | NT |
| mixture | ylamino)cyclohex-1-en-1-yl)-1H-pyrazolo[4,3- | | | |
| of B14 | c]pyridin-3-yl)-2-fluorophenyl)-5-chloro-2- | | | |
| & B15 | fluorobenzenesulfonamide | | | |
| B14 | N-(4-(4-amino-1-isopropyl-7-(4-(oxetan-3- | 0.16 | 0.0068 | NT |
| (First | ylamino)cyclohex-1-en-1-yl)-1H-pyrazolo[4,3- | | | |
| eluting | c]pyridin-3-yl)-2-fluorophenyl)-5-chloro-2- | | | |
| isomer) | fluorobenzenesulfonamide | | | |
| B15 | N-(4-(4-amino-1-isopropyl-7-(4-(oxetan-3- | 0.024 | 0.0014 | NT |
| (Second | ylamino)cyclohex-1-en-1-yl)-1H-pyrazolo[4,3- | | | |
| eluting | c]pyridin-3-yl)-2-fluorophenyl)-5-chloro-2- | | | |
| isomer) | fluorobenzenesulfonamide | | | |
| racemic | N-(4-(4-amino-1-isopropyl-7-(4-(oxetan-3- | 0.094 | 0.013 | NT |
| mixture | ylamino)cyclohex-1-en-1-yl)-1H-pyrazolo[4,3- | | | |
| of B16 | c]pyridin-3-yl)-2-fluorophenyl)-2-fluoro-5- | | | |
| & B17 | methylbenzenesulfonamide | | | |
| B16 | N-(4-(4-amino-1-isopropyl-7-(4-(oxetan-3- | 0.11 | 0.013 | NT |
| (First | ylamino)cyclohex-1-en-1-yl)-1H-pyrazolo[4,3- | | | |
| eluting | c]pyridin-3-yl)-2-fluorophenyl)-2-fluoro-5- | | | |
| isomer) | methylbenzenesulfonamide | | | |
| B17 | N-(4-(4-amino-1-isopropyl-7-(4-(oxetan-3- | 0.021 | 0.0022 | NT |
| (Second | ylamino)cyclohex-1-en-1-yl)-1H-pyrazolo[4,3- | | | |
| eluting | c]pyridin-3-yl)-2-fluorophenyl)-2-fluoro-5- | | | |
| isomer) | methylbenzenesulfonamide | | | |
| B18 | N-(4-(4-amino-1-cyclopropyl-7-(4-(oxetan-3- | 0.082 | 0.0099 | NT |
| (First | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| B19 | N-(4-(4-amino-1-cyclopropyl-7-(4-(oxetan-3- | 0.020 | 0.0035 | NT |
| (Second | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| B20 | N-(4-(4-amino-1-cyclopropyl-7-(4-(oxetan-3- | 0.038 | NT | NT |
| (First | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B21 | N-(4-(4-amino-1-cyclopropyl-7-(4-(oxetan-3- | 0.011 | NT | NT |
| (Second | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B22 | N-(4-(4-amino-1-cyclopropyl-7-(4-(oxetan-3- | 0.01 | 0.0009 | NT |
| (Second | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B23 | N-(4-(4-amino-1-cyclopropyl-7-(4-(oxetan-3- | 0.046 | 0.0031 | NT |
| (First | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B24 | N-(4-(4-amino-1-ethyl-7-(4-(oxetan-3- | 0.58 | 0.053 | NT |
| (First | ylamino)cyclohex-1-en-1-yl)-1H-pyrazolo[4,3- | | | |
| eluting | c]pyridin-3-yl)phenyl)-1-(2- | | | |
| isomer) | chlorophenyl)methanesulfonamide | | | |
| B25 | N-(4-(4-amino-1-ethyl-7-(4-(oxetan-3- | 0.17 | NT | NT |
| (Second | ylamino)cyclohex-1-en-1-yl)-1H-pyrazolo[4,3- | | | |
| eluting | c]pyridin-3-yl)phenyl)-1-(2- | | | |
| isomer) | chlorophenyl)methanesulfonamide | | | |
| B26 | N-(4-(4-amino-1-ethyl-7-(4-(oxetan-3- | 0.022 | 0.0027 | 0.0041 |
| (Second | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B27 | N-(4-(4-amino-1-ethyl-7-(4-(oxetan-3- | 0.078 | 0.0059 | NT |
| (First | ylamino)cyclohex-1-en-1-yl)-1H-pyrazolo[4,3- | | | |
| eluting | c]pyridin-3-yl)-2-fluorophenyl)-1-(2- | | | |
| isomer) | fluorophenyl)methanesulfonamide | | | |
| B28 | N-(4-(4-amino-1-ethyl-7-(4-(oxetan-3- | 0.027 | 0.0024 | 0.0039 |
| (Second | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B29 | N-(4-(4-amino-1-ethyl-7-(4-(oxetan-3- | 0.068 | 0.0074 | NT |
| (First | ylamino)cyclohex-1-en-1-yl)-1H-pyrazolo[4,3- | | | |
| eluting | c]pyridin-3-yl)-2-fluorophenyl)-1-(2- | | | |
| isomer) | chlorophenyl)methanesulfonamide | | | |
| B30 | N-(4-(4-amino-1-ethyl-7-(4-(oxetan-3- | 0.74 | 0.082 | NT |
| | ylamino)cyclohex-1-en-1-yl)-1H-pyrazolo[4,3- | | | |
| | c]pyridin-3-yl)-2,5-difluorophenyl)-2- | | | |
| | fluorobenzenesulfonamide | | | |
| B31 | N-(4-(4-amino-1-ethyl-7-(4-(oxetan-3- | 0.13 | 0.047 | NT |
| | ylamino)cyclohex-1-en-1-yl)-1H-pyrazolo[4,3- | | | |
| | c]pyridin-3-yl)-2,5-difluorophenyl)-2- | | | |
| | chlorobenzenesulfonamide | | | |
| B32 | N-(4-(4-amino-1-ethyl-7-(4-(oxetan-3- | 0.34 | 0.021 | NT |
| | ylamino)cyclohex-1-en-1-yl)-1H-pyrazolo[4,3- | | | |
| | c]pyridin-3-yl)-2,5-difluorophenyl)-1-(2- | | | |
| | chlorophenyl)methanesulfonamide | | | |
| B33 | N-(4-(4-amino-1-ethyl-7-(4-(oxetan-3- | 0.10 | 0.060 | NT |
| (Second | ylamino)cyclohex-1-en-1-yl)-1H-pyrazolo[4,3- | | | |
| eluting | c]pyridin-3-yl)-2-fluorophenyl)-2- | | | |
| isomer) | fluorobenzenesulfonamide | | | |
| B34 | N-(4-(4-amino-1-ethyl-7-(4-(oxetan-3- | 0.40 | 0.10 | NT |
| (First | ylamino)cyclohex-1-en-1-yl)-1H-pyrazolo[4,3- | | | |
| eluting | c]pyridin-3-yl)-2-fluorophenyl)-2- | | | |
| isomer) | fluorobenzenesulfonamide | | | |
| B35 | N-(4-(4-amino-1-ethyl-7-(4-(oxetan-3- | 0.052 | 0.014 | NT |
| (Second | ylamino)cyclohex-1-en-1-yl)-1H-pyrazolo[4,3- | | | |
| eluting | c]pyridin-3-yl)-2-fluorophenyl)-2-fluoro-5- | | | |
| isomer) | methylbenzenesulfonamide | | | |
| B36 | N-(4-(4-amino-1-ethyl-7-(4-(oxetan-3- | 0.21 | 0.051 | NT |
| (First | ylamino)cyclohex-1-en-1-yl)-1H-pyrazolo[4,3- | | | |
| eluting | c]pyridin-3-yl)-2-fluorophenyl)-2-fluoro-5- | | | |
| isomer) | methylbenzenesulfonamide | | | |
| B37 | N-(4-(4-amino-1-ethyl-7-(4-(oxetan-3- | 0.074 | 0.020 | NT |
| | ylamino)cyclohex-1-en-1-yl)-1H-pyrazolo[4,3- | | | |
| | c]pyridin-3-yl)-2-fluorophenyl)-2-chloro-5- | | | |
| | methoxybenzenesulfonamide | | | |
| B38 | N-(4-(4-amino-1-ethyl-7-(4-(oxetan-3- | 0.097 | 0.0056 | NT |
| (Second | ylamino)cyclohex-1-en-1-yl)-1H-pyrazolo[4,3- | | | |
| eluting | c]pyridin-3-yl)-2-fluorophenyl)-5-chloro-2- | | | |
| isomer) | fluorobenzenesulfonamide | | | |
| B39 | N-(4-(4-amino-1-ethyl-7-(4-(oxetan-3- | 0.22 | NT | NT |
| (First | ylamino)cyclohex-1-en-1-yl)-1H-pyrazolo[4,3- | | | |
| eluting | c]pyridin-3-yl)-2-fluorophenyl)-5-chloro-2- | | | |
| isomer) | fluorobenzenesulfonamide | | | |
| B40 | N-(4-(4-amino-7-(4-((1-fluoropropan-2- | 0.067 | 0.014 | NT |
| (first | yl)amino)cyclohex-1-en-1-yl)-1-isopropyl-1H- | | | |
| eluting | pyrazolo[4,3-c]pyridin-3-yl)-2-fluorophenyl)-2- | | | |
| isomer) | chlorobenzenesulfonamide | | | |
| B41 | N-(4-(4-amino-7-(4-((1-fluoropropan-2- | 0.010 | 0.0061 | NT |
| (second | yl)amino)cyclohex-1-en-1 -yl)-1-isopropyl-1H- | | | |
| eluting | pyrazolo[4,3-c]pyridin-3-yl)-2-fluorophenyl)-2- | | | |
| isomer) | chlorobenzenesulfonamide | | | |
| B42 | N-(4-(4-amino-7-(4-((2- | 0.018 | 0.0017 | NT |
| (Second | fluoropropyl)amino)cyclohex-1-en-1-yl)-1-isopropyl- | | | |
| eluting | 1H-pyrazolo[4,3-c]pyridin-3-yl)-2-fluorophenyl)-1- | | | |
| isomer) | (2-chlorophenyl)methanesulfonamide | | | |
| B43 | N-(4-(4-amino-7-(4-((2- | 0.0051 | 0.0005 | NT |
| (First | fluoropropyl)amino)cyclohex-1-en-1-yl)-1-isopropyl- | | | |
| eluting | 1H-pyrazolo[4,3-c]pyridin-3-yl)-2-fluorophenyl)-1- | | | |
| isomer) | (2-chlorophenyl)methanesulfonamide | | | |
| B44 | N-(4-(4-amino-7-(4-((1-fluoropropan-2- | 0.010 | 0.0016 | NT |
| (Second | yl)amino)cyclohex-1-en-1-yl)-1-isopropyl-1H- | | | |
| eluting | pyrazolo[4,3-c]pyridin-3-yl)-2-fluorophenyl)-1-(2- | | | |
| isomer) | chlorophenyl)methanesulfonamide | | | |
| B45 | N-(4-(4-amino-7-(4-((1-fluoropropan-2- | 0.047 | 0.0034 | NT |
| (First | yl)amino)cyclohex-1-en-1-yl)-14sopropyl-1H- | | | |
| eluting | pyrazolo[4,3-c]py ridin-3-yl)-2-fluorophenyl)-1-(2- | | | |
| isomer) | chlorophenyl)methanesulfonamide | | | |
| B46 | N-(4-(4-amino-7-(4-((2- | 0.022 | 0.0047 | NT |
| (Second | fluoropropyl)amino)cyclohex-1-en-1-yl)-1-isopropyl- | | | |
| eluting | 1H-pyrazolo[4,3-c]pyridin-3-yl)-2-fluorophenyl)-2- | | | |
| isomer) | chlorobenzenesulfonamide | | | |
| B47 | N-(4-(4-amino-7-(4-((2- | 0.017 | 0.0045 | NT |
| (First | fluoropropyl)amino)cyclohex-1-en-1-yl)-1-isopropyl- | | | |
| eluting | 1H-pyrazolo[4,3-c]pyridin-3-yl)-2-fluorophenyl)-2- | | | |
| isomer) | chlorobenzenesulfonamide | | | |
| B48 | N-(4-(4-amino-7-(4-((3,3- | 0.013 | 0.0013 | NT |
| (Second | difluorocyclobutyl)amino)cyclohex-1-en-1-yl)-1- | | | |
| eluting | isopropyl-1H-pyrazolo[4,3-c]pyridin-3-yl)-2- | | | |
| isomer) | fluorophenyl)-1-(2- | | | |
| | chlorophenyl)methanesulfonamide | | | |
| B49 | N-(4-(4-amino-7-(4-((3,3- | 0.064 | 0.0041 | NT |
| (First | difluorocyclobutyl)amino)cyclohex-1-en-1-yl)-1- | | | |
| eluting | isopropyl-1H-pyrazolo[4,3-c]pyridin-3-yl)-2- | | | |
| isomer) | fluorophenyl)-1-(2- | | | |
| | chlorophenyl)methanesulfonamide | | | |
| B50 | N-(4-(4-amino-7-(4-((3,3- | 0.025 | 0.0050 | NT |
| (Second | difluorocyclobutypamino)cyclohex-1-en-1-yl)-1- | | | |
| eluting | isopropyl-1H-pyrazolo[4,3-c]pyridin-3-yl)-2- | | | |
| isomer) | fluorophenyl)-2-chlorobenzenesulfonamide | | | |
| B51 | N-(4-(4-amino-7-(4-((3,3- | 0.19 | 0.031 | NT |
| (First | difluorocyclobutypamino)cyclohex-1-en-1-yl)-1- | | | |
| eluting | isopropyl-1H-pyrazolo[4,3-c]pyridin-3-yl)-2- | | | |
| isomer) | fluorophenyl)-2-chlorobenzenesulfonamide | | | |
| B52 | N-(4-(4-amino-1-isopropyl-7-(4-((2- | 0.0098 | 0.0078 | NT |
| (Second | methoxyethyl)amino)cyclohex-1-en-1-yl)-1H- | | | |
| eluting | pyrazolo[4,3-c]pyridin-3-yl)-2-fluorophenyl)-2- | | | |
| isomer) | chlorobenzenesulfonamide | | | |
| B53 | N-(4-(4-amino-1-isopropyl-7-(4-((2- | 0.070 | 0.017 | NT |
| (First | methoxyethyl)amino)cyclohex-1-en-1-yl)-1H- | | | |
| eluting | pyrazolo[4,3-c]pyridin-3-yl)-2-fluorophenyl)-2- | | | |
| isomer) | chlorobenzenesulfonamide | | | |
| B54 | N-(4-(4-amino-1-isopropyl-7-(4-((2- | 0.028 | 0.0020 | NT |
| (First | methoxyethyl)amino)cyclohex-1-en-1-yl)-1H- | | | |
| eluting | pyrazolo[4,3-c]pyridin-3-yl)-2-fluorophenyl)-1-(2- | | | |
| isomer) | chlorophenyl)methanesulfonamide | | | |
| B55 | N-(4-(4-amino-1-isopropyl-7-(4-((2- | 0.009 | 0.0009 | NT |
| (Second | methoxyethyl)amino)cyclohex-1-en-1-yl)-1H- | | | |
| eluting | pyrazolo[4,3-c]pyridin-3-yl)-2-fluorophenyl)-1-(2- | | | |
| isomer) | chlorophenyl)methanesulfonamide | | | |
| racemic | N-(4-(4-amino-1-isopropyl-7-(4-(oxetan-3- | 0.052 | 0.026 | NT |
| mixture | ylamino)cyclohex-1-en-1-yl)-1H-pyrazolo[4,3- | | | |
| of B56 | c]pyridin-3-yl)-2-fluorophenyl)-2- | | | |
| & B57 | chlorobenzenesulfonamide | | | |
| B56 | N-(4-(4-amino-1-isopropyl-7-(4-(oxetan-3- | 0.20 | NT | NT |
| (first | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| B57 | N-(4-(4-amino-1-isopropyl-7-(4-(oxetan-3- | 0.020 | NT | NT |
| (second | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| racemic | N-(4-(4-amino-1-isopropyl-7-(4-(oxetan-3- | 0.092 | 0.061 | NT |
| mixture | ylamino)cyclohex-1-en-1-yl)-1H-pyrazolo[4,3- | | | |
| of B58 | c]pyridin-3-yl)-2-fluorophenyl)-2- | | | |
| & B59 | fluorobenzenesulfonamide | | | |
| B58 | N-(4-(4-amino-1-isopropyl-7-(4-(oxetan-3- | 0.25 | 0.12 | NT |
| (First | ylamino)cyclohex-1-en-1-yl)-1H-pyrazolo[4,3- | | | |
| eluting | c]pyridin-3-yl)-2-fluorophenyl)-2- | | | |
| isomer) | fluorobenzenesulfonamide | | | |
| B59 | N-(4-(4-amino-1-isopropyl-7-(4-(oxetan-3- | 0.036 | NT | 0.0059 |
| (Second | ylamino)cyclohex-1-en-1-yl)-1H-pyrazolo[4,3- | | | |
| eluting | c]pyridin-3-yl)-2-fluorophenyl)-2- | | | |
| isomer) | fluorobenzenesulfonamide | | | |
| B60 | N-(4-(4-amino-1-isopropyl-7-(4-(oxetan-3- | 0.24 | 0.079 | NT |
| | ylamino)cyclohex-1-en-1-yl)-1H-pyrazolo[4,3- | | | |
| | c]pyridin-3-yl)-2,5-difluorophenyl)-2- | | | |
| | fluorobenzenesulfonamide | | | |
| racemic | N-(4-(4-amino-1-ethyl-7-(4-(oxetan-3- | 0.10 | 0.040 | NT |
| mixture | ylamino)cyclohex-1-en-1-yl)-1H-pyrazolo[4,3- | | | |
| of B61 | c]pyridin-3-yl)-2-fluorophenyl)-2- | | | |
| & B62 | chlorobenzenesulfonamide | | | |
| B61 | N-(4-(4-amino-1-ethyl-7-(4-(oxetan-3- | 0.11 | 0.034 | NT |
| (Second | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| B62 | N-(4-(4-amino-1-ethyl-7-(4-(oxetan-3- | 0.40 | 0.075 | NT |
| (First | ylamino)cyclohex-1-en-1-yl)-1H-pyrazolo[4,3- | | | |
| eluting | c]pyridin-3-yl)-2-fluorophenyl)-2- | | | |
| isomer) | chlorobenzenesulfonamide | | | |
| B63 | N-(4-(4-amino-7-(4-((2-fluoroethyl)amino)cyclohex- | 0.021 | 0.016 | NT |
| | 1-en-1-yl)-1-isopropyl-1H-pyrazolo[4,3-c]pyridin-3- | | | |
| | yl)-2-fluorophenyl)-2-chlorobenzenesulfonamide | | | |

### Enumerated Embodiments

The following exemplary embodiments are provided, the numbering of which is not to be construed as designating levels of importance:

Embodiment 1 provides a compound of formula (Ia), or a salt, solvate, enantiomer, diastereoisomer, isotopologue, or tautomer thereof:

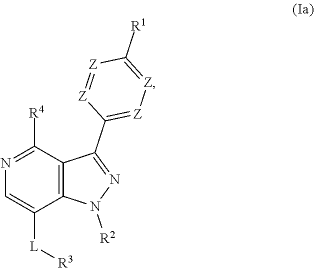

wherein:

R¹ is

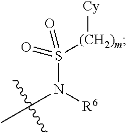

R² is selected from the group consisting of H, methyl, ethyl, propyl, isopropyl, butyl, isobutyl, sec-butyl, tert-butyl, CF₃, CHF₂, cyclopropyl, cyclobutyl, cyclopentyl, cyclohexyl, cycloheptyl, cyclooctyl, and 1-methylcyclopropyl;

L is a bond;

R³ is selected from the group consisting of optionally substituted C₃-C₈ cycloalkyl, optionally substituted C₃-C₈ cycloalkenyl, optionally substituted C₃-C₈ heterocycloalkyl, and optionally substituted C₂-C₈ cycloheteroalkenyl;

R⁴ is —NH₂;

0-3 instances of Z are N and the remaining instances of Z are independently CR⁵; each instance of R⁵ is independently selected from the group consisting of halogen, —OH, optionally substituted C₁-C₆ alkyl, and optionally substituted C₁-C₆ alkoxy;

R⁶ is H;

Cy is phenyl, thiophenyl, pyridinyl, pyridazinyl, pyrimidinyl, or pyrazinyl;
wherein Cy is substituted with 0 to ‘n’ instances of X, each instance of X being independently selected from the group consisting of H, halogen, nitrile, optionally substituted C₁-C₄ alkyl, C₁-C₄ haloalkyl, optionally substituted C₁-C₄ alkoxy, optionally substituted phenyl, optionally substituted naphthyl, optionally substituted heteroaryl, and

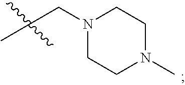 [SMILES: *CN1CCN(C)CC1]

m is an integer selected from the group consisting of 0, 1, and 2;

n is an integer selected from the group consisting of 0, 1, 2, 3, 4, and 5.

Embodiment 2 provides the compound of Embodiment 1, wherein each occurrence of optionally substituted alkyl, optionally substituted alkoxy, optionally substituted heterocycloalkyl, optionally substituted cycloalkyl, optionally substituted cycloalkenyl, or optionally substituted cycloheteroalkenyl is independently optionally substituted with at least one substituent selected from the group consisting of C₁-C₆ alkyl, halogen, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 3 provides the compound of any of Embodiments 1-2, wherein each occurrence of optionally substituted phenyl, optionally substituted naphthyl, or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

Embodiment 4 provides the compound of any of Embodiments 1-3, wherein each occurrence of optionally substituted phenyl, optionally substituted naphthyl, or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —N(R^{c})(R^{c}), and C₁-C₆ alkoxycarbonyl, wherein each occurrence of R^{c} is independently H, C₁-C₆ alkyl, or C₃-C₈ cycloalkyl.

Embodiment 5 provides the compound of any of Embodiments 1-4, wherein R¹ is selected from the group consisting of:

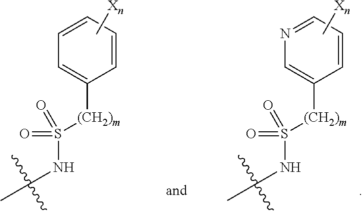

Embodiment 6 provides the compound of any of Embodiments 1-5, wherein R¹ is selected from the group consisting of:

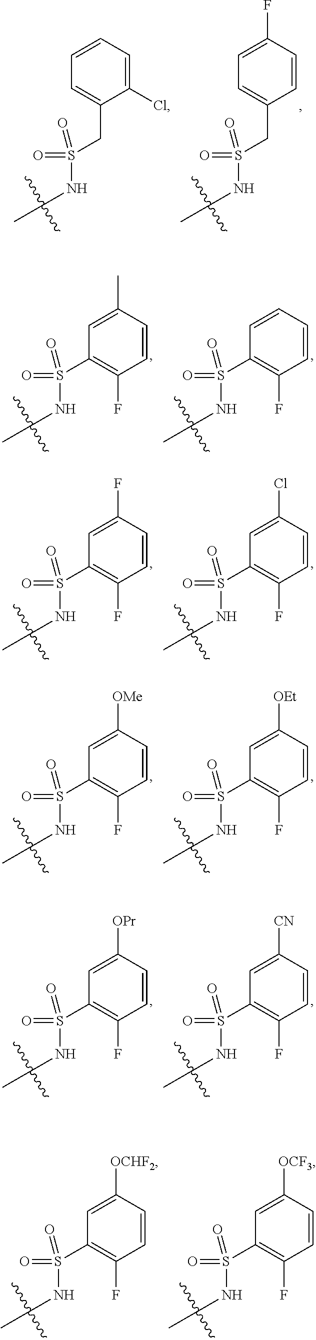

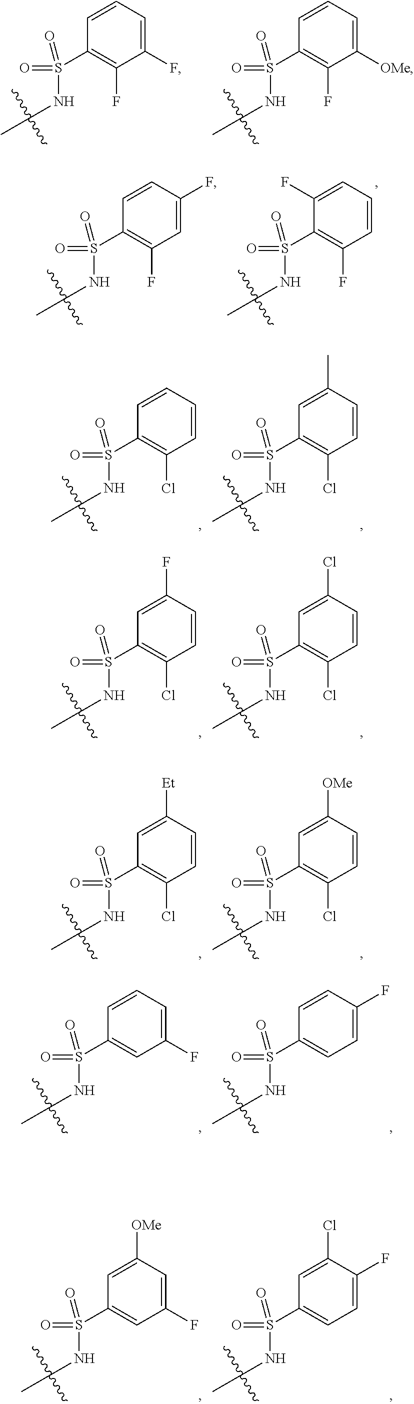

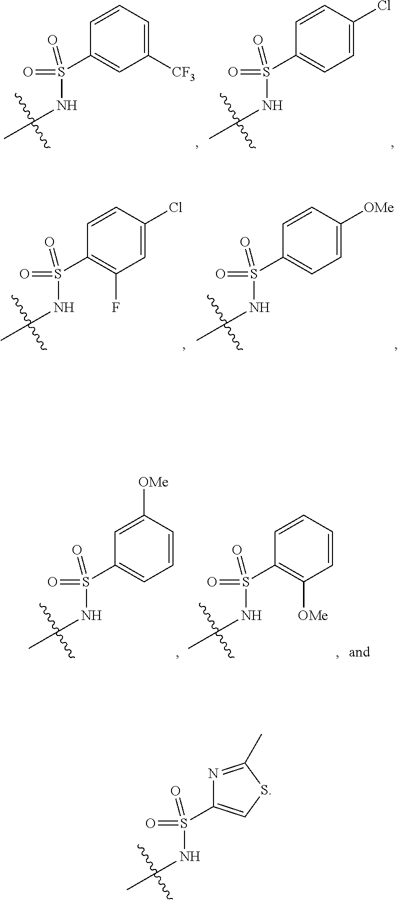

Embodiment 7 provides the compound of any of Embodiments 1-6, wherein R² is selected from the group consisting of methyl, ethyl, isopropyl, and cyclopropyl.

Embodiment 8 provides the compound of any of Embodiments 1-7, wherein R³ is:

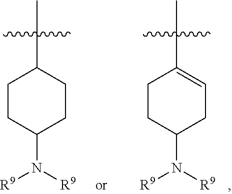

wherein each occurrence of R⁹ is independently selected from the group consisting of H, oxetanyl, C₁-C₆ alkyl, C₃-C₈ cycloalkyl, C₁-C₆ hydroxyalkyl, C₁-C₆ (C₁-C₆ alkoxy)alkyl, C₁-C₆ haloalkyl, C₁-C₆ carboxamido alkyl, C₁-C₆ carboxy alkyl, C₁-C₆ [carboxy(C₁-C₆)alkyl] alkyl, C₁-C₆ cyano alkyl, and C₁-C₆ sulfonyl alkyl, or the two R⁹ combine with the N to which they are bound to form a 3-8 heterocyclyl ring; wherein each R⁹ is independently optionally substituted with at least one of OH, C₁-C₆ alkoxy, halogen, NH₂, NH(C₁-C₆ alkyl), N(C₁-C₆ alkyl)(C₁-C₆ alkyl), cyano, carboxamide, carboxy, and sulfonyl.

Embodiment 9 provides the compound of any of Embodiments 1-8, wherein each occurrence of R⁹ is independently selected from the group consisting of H, methyl, ethyl, propyl, isopropyl, butyl, sec-butyl, isobutyl, tert-butyl, cyclobutyl, fluorocyclobutyl, difluorocyclobutyl, oxetanyl,

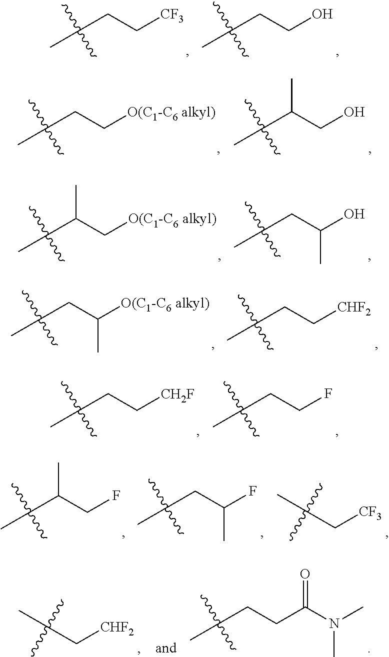

Embodiment 10 provides the compound of any of Embodiments 1-9, wherein R³ is selected from the group consisting of:

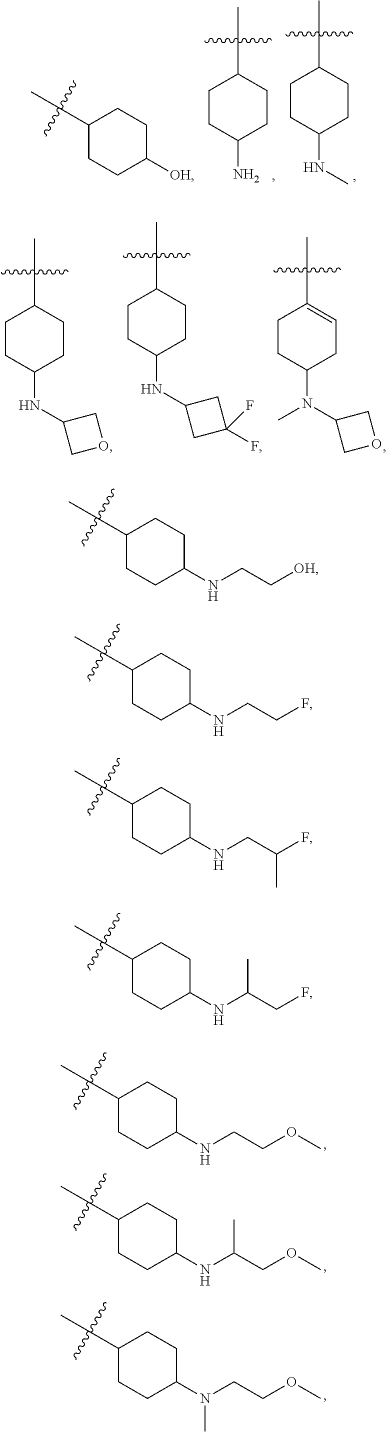

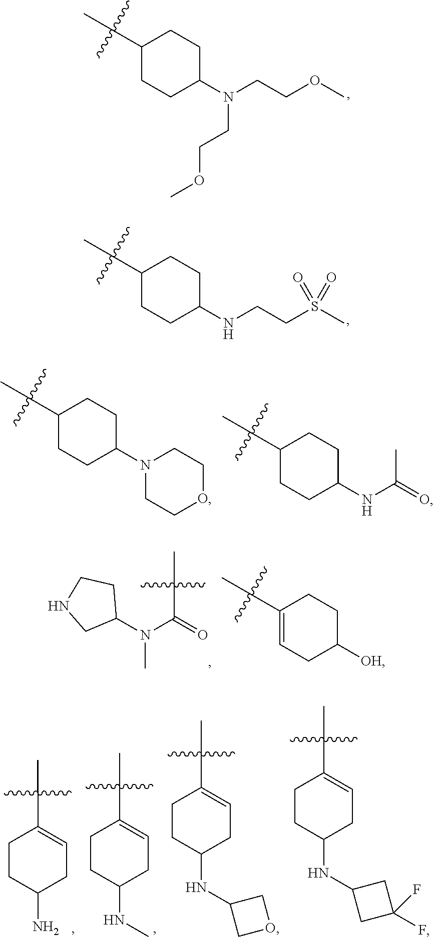

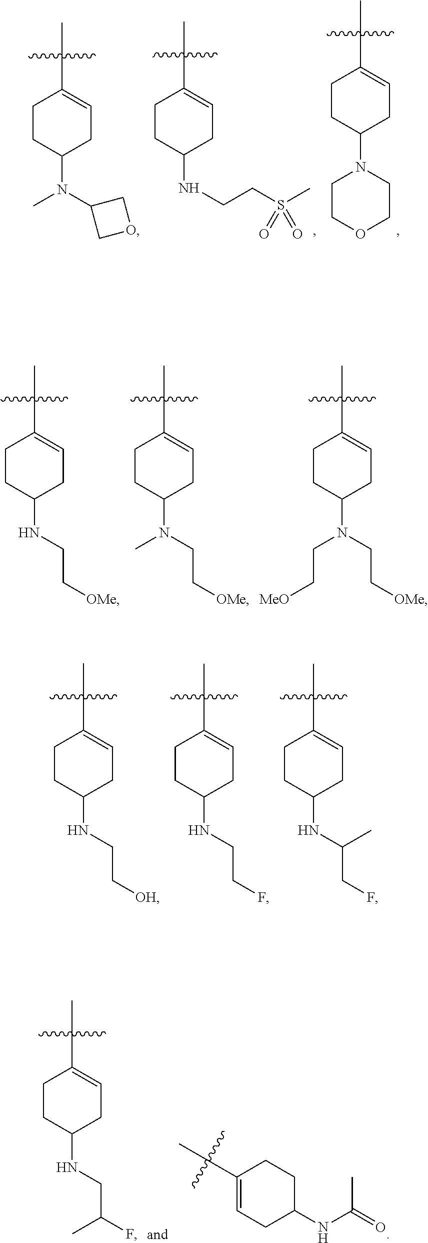

Embodiment 11 provides the compound of any of Embodiments 1-10, wherein R³ is selected from the group consisting of:

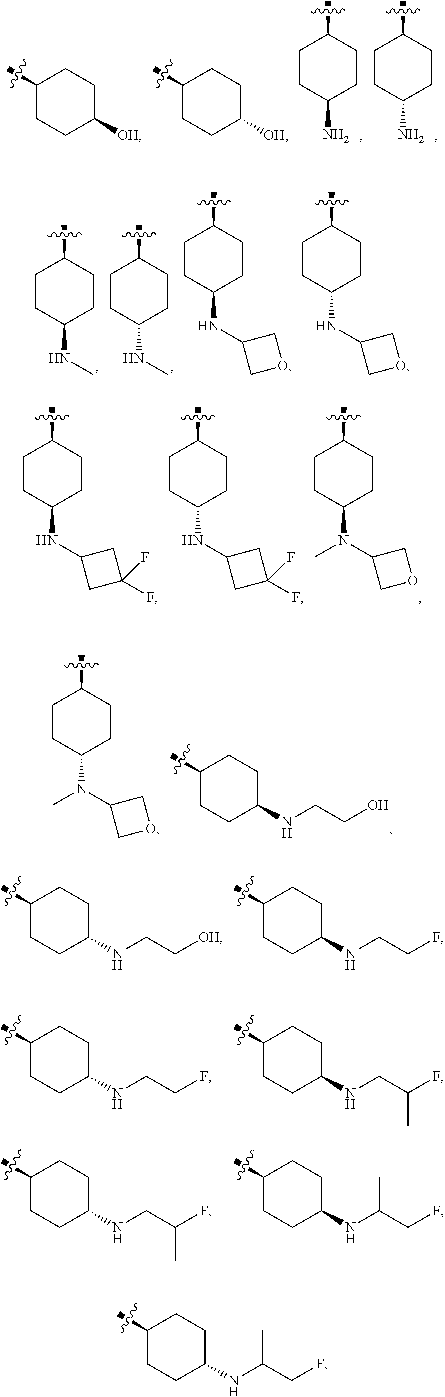

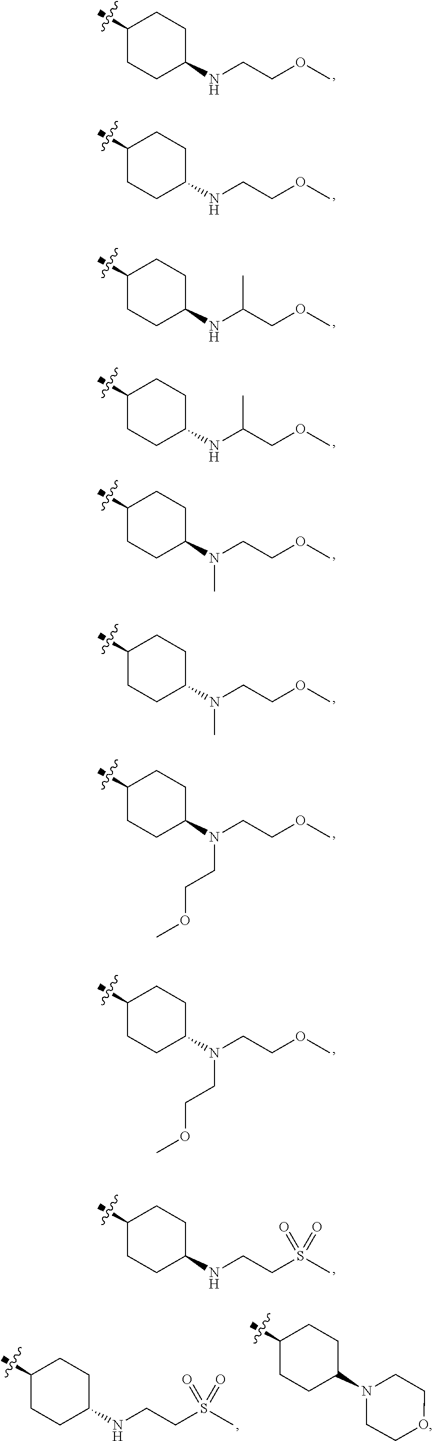

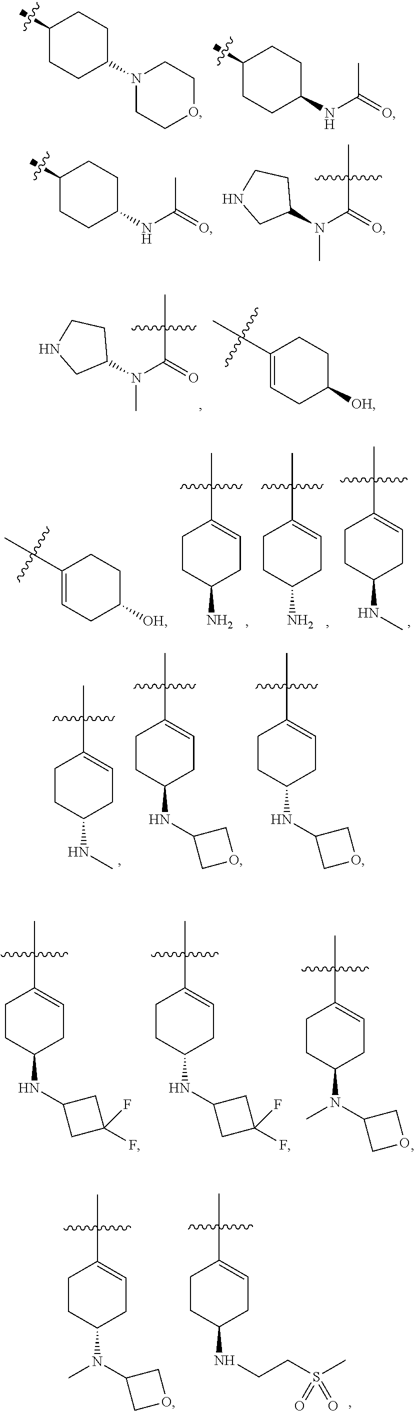

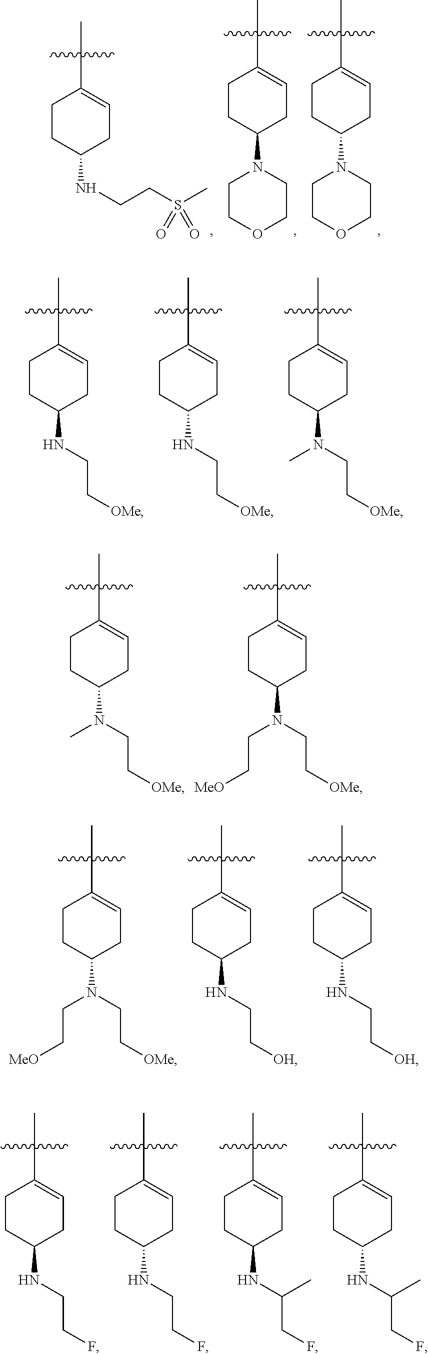

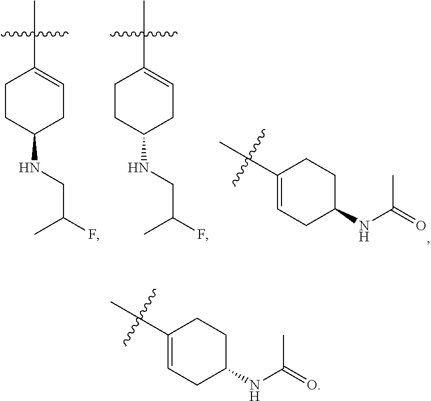 [SMILES: *C1=CC[C@@H](NCC(C)F)CC1 ; *C1=CC[C@H](NCC(C)F)CC1 ; *C1=CC[C@H](NC(C)=O)CC1 ; *C1=CC[C@@H](NC(C)=O)CC1]

Embodiment 12 provides the compound of any of Embodiments 1-11, wherein R⁵, if present, is F.

Embodiment 13 provides the compound of any of Embodiments 1-12, which is:

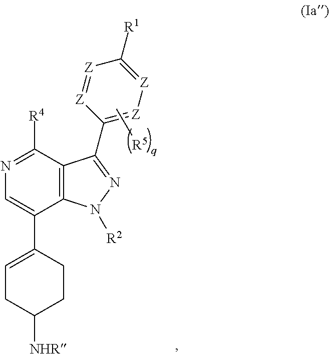

wherein R′″ is H, optionally substituted C₁-C₆ alkyl, optionally substituted C₃-C₈ cycloalkyl, or optionally substituted heterocyclyl.

Embodiment 14 provides the compound of any of Embodiments 1-13, which is:

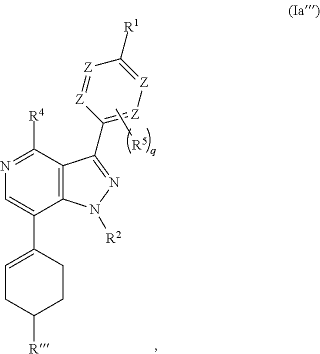

wherein each occurrence of R′″ is independently selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl), wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, and —C₁-C₆ alkoxy.

Embodiment 15 provides the compound of any of Embodiments 1-14, wherein R″ is selected from the group consisting of —OH, —NH₂, —NHCH₃, —N(CH₃)₂, —NHCH₂CH₂F, —N(Me)CH₂CH₂F, —NHCH₂CHF₂, —N(Me)CH₂CHF₂, —NHCH₂CF₃, —N(Me)CH₂CF₃, —NHCH₂CH₂CF₃, —N(Me)CH₂CH₂CF₃, —NHCH₂CH₂C(═O)NMe₂, —N(Me)CH₂CH₂C(═O)NMe₂, —NHCH₂CH₂C(═O)NH₂, —N(Me)CH₂CH₂C(═O)NH₂, —NHCH₂CH₂C(═O)NHMe, —N(Me)CH₂CH₂C(═O)NHMe₂, and

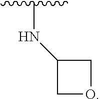 [SMILES: *NC1COC1]

Embodiment 16 provides the compound of any of Embodiments 1-15, which is selected from the group consisting of:
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-chloro-5-methoxybenzenesulfonamide;
N-(4-(4-amino-1-isopropyl-7-((1r,4r)-4-morpholinocyclohexyl)-1H-pyrazolo[4,3-c]pyridin-3-yl)-2,5-difluorobenzenesulphonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-chloro-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-(difluoromethoxy)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-fluoro-3-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-5-ethoxy-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-3-methoxybenzenesulfonamide;
N-(4-(4-amino-isopropyl-7-((1s,4 s)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-fluoro-3-methoxybenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-chlorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-chloro-4-fluorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-4-chloro-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(4-fluorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-3-fluorophenyl)-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-5-cyano-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,6-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,4-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,3-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-1-(2-fluorophenyl)methanesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluoro-5-methoxybenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-fluoro-5-methoxybenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,5-difluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-4-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-3-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-((1r,4r)-4-((2-methoxyethyl)(methyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-2-fluorobenzenesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2-fluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-isopropyl-7-((1r,4r)-4-((1-methoxypropan-2-yl)amino)cyclohexyl)-1H-pyrazolo[4,3-c]pyridin-3-yl)-2-fluorophenyl)-1-(2-chlorophenyl)methanesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-fluoro-5-(trifluoromethoxy)benzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2-fluoro-5-propoxybenzenesulfonamide;
N-(4-(4-amino-1-isopropyl-7-((1r,4r)-4-((2-methoxyethyl)amino)cyclohexyl)-1H-pyrazolo[4,3-c]pyridin-3-yl)-2,5-difluorophenyl)-2,5-dichlorobenzenesulfonamide;
N-(4-(4-amino-1-isopropyl-7-((1r,4r)-4-((2-methoxy ethyl)amino)cyclohexyl)-1H-pyrazolo[4,3-c]pyridin-3-yl)-2,5-difluorophenyl)-2-chloro-5-ethylbenzenesulfonamide;
N-(4-(4-amino-7-((1r,4r)-4-((2-fluoropropyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-isopropyl-7-(4-((l-methoxy propan-2-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((l-methoxy propan-2-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)phenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chloro-5-methoxy benzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-7-(4(R)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((1-fluoropropan-2-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4-((3,3-difluorocyclobutyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((3,3-difluorocyclobutyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-1-isopropyl-7-(4-((2-methoxyethyl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((2-methoxy ethyl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-7-(4-((2-fluoro ethyl)amino)cyclohex-1-en-1-yl)-1-isopropyl-1H-pyrazolo[4,3-c]pyridin-3-yl)-2-fluorophenyl)-2-chlorobenzenesulfonamide;
   or a salt, solvate, enantiomer, diastereoisomer, isotopologue or tautomer thereof.

Embodiment 17 provides a pharmaceutical composition comprising at least one compound of any of Embodiments 1-16 and at least one pharmaceutically acceptable carrier.

Embodiment 18 provides a method of treating a IRE1α-related disease in a subject, the method comprising administering to the subject a therapeutically effective amount of a compound, or a pharmaceutically acceptable salt, solvate, enantiomer, diastereoisomer, or tautomer thereof, of any of Embodiments 1-16 and/or the composition of Embodiment 17.

Embodiment 19 provides the method of Embodiment 18, wherein the disease is selected from the group consisting of a neurodegenerative disease, a demyelinating disease, cancer, an eye disease, a fibrotic disease, and diabetes.

Embodiment 20 provides the method of any of Embodiments 18-19, wherein the neurodegenerative disease is selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru.

Embodiment 21 provides the method of any of Embodiments 18-19, wherein the demyelinating disease is selected from the group consisting of Wolfram Syndrome, Pelizaeus-Merzbacher Disease, Transverse Myelitis, Charcot-Marie-Tooth Disease, and Multiple Sclerosis.

Embodiment 22 provides the method of any of Embodiments 18-19, wherein the cancer is multiple myeloma.

Embodiment 23 provides the method of any of Embodiments 18-19, wherein the diabetes is selected from the group consisting of type I diabetes and type II diabetes.

Embodiment 24 provides the method of any of Embodiments 18-19, wherein the eye disease is selected from the group consisting of retinitis pigmentosa, retinal degeneration, macular degeneration, and Wolfram Syndrome.

Embodiment 25 provides the method of any of Embodiments 18-19, wherein the fibrotic disease is selected from the group consisting of idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

Embodiment 26 provides a method of inhibiting the activity of an IRE1 protein, the method comprising contacting the IRE1 protein with an effective amount of a compound, or a pharmaceutically acceptable salt thereof, of any of Embodiments 1-16 and/or the composition of Embodiment 17.

Embodiment 27 provides the method of Embodiment 26, wherein the activity is selected from the group consisting of kinase activity, oligomerization activity, and RNase activity.

Embodiment 28 provides the method of any of Embodiments 26-27, wherein the IRE1 protein is within a cell.

Embodiment 29 provides the method of Embodiment 28, wherein apoptosis of the cell is prevented or minimized.

Embodiment 30 provides the method of any of Embodiments 28-29, wherein the cell is in an organism that has an IRE1α-related disease or disorder.

Embodiment 31 provides the method of Embodiment 30, wherein the disease or disorder is a neurodegenerative disease, demyelinating disease, cancer, eye disease, fibrotic disease, or diabetes.

Embodiment 32 provides the method of any of Embodiments 18-31, wherein the subject is need of the treatment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (Ia), or a salt, solvate, enantiomer, diastereoisomer, isotopologue, or tautomer thereof: 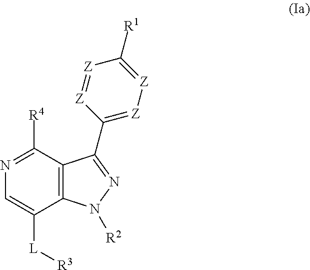
wherein
R¹ is 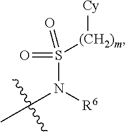
R² is selected from the group consisting of H, methyl, ethyl, propyl, isopropyl, butyl, isobutyl, sec-butyl, tert-butyl, CF₃, CHF₂, cyclopropyl, cyclobutyl, cyclopentyl, cyclohexyl, cycloheptyl, cyclooctyl, and 1-methylcyclopropyl;
L is a bond;
R³ is selected from the group consisting of optionally substituted C₃-C₈ cycloalkyl, optionally substituted C₃-C₈ cycloalkenyl, optionally substituted C₃-C₈ heterocycloalkyl, and optionally substituted C₂-C₈ cycloheteroalkenyl;
R⁴ is —NH₂;
0-3 instances of Z are N and the remaining instances of Z are independently CR⁵;
each instance of R⁵ is independently selected from the group consisting of H, halogen, —OH, optionally substituted C₁-C₆ alkyl, and optionally substituted C₁-C₆ alkoxy;
R⁶ is H;
Cy is phenyl, thiophenyl, pyridinyl, pyridazinyl, pyrimidinyl, or pyrazinyl;
wherein Cy is substituted with 0 to ‘n’ instances of X, each instance of X being independently selected from the group consisting of H, halogen, nitrile, optionally substituted C₁-C₄ alkyl, C₁-C₄ haloalkyl, optionally substituted C₁-C₄ alkoxy, optionally substituted phenyl, optionally substituted naphthyl, optionally substituted heteroaryl, and 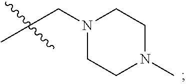 [SMILES: *CN1CCN(C)CC1]
m is an integer selected from the group consisting of 0, 1, and 2;
n is an integer selected from the group consisting of 0, 1, 2, 3, 4, and 5.

2. The compound of claim 1, wherein each occurrence of optionally substituted alkyl, optionally substituted alkoxy, optionally substituted heterocycloalkyl, optionally substituted cycloalkyl, optionally substituted cycloalkenyl, or optionally substituted cycloheteroalkenyl is independently optionally substituted with at least one substituent selected from the group consisting of C₁-C₆ alkyl, halogen, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two IV groups combine with the N to which they are bound to form a heterocycle.

3. The compound of claim 1, wherein each occurrence of optionally substituted phenyl, optionally substituted naphthyl, or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

4. The compound of claim 1, wherein each occurrence of optionally substituted phenyl, optionally substituted naphthyl, or optionally substituted heteroaryl is independently optionally substituted with at least one substituent selected from the group consisting of C₁-C₆ alkyl, C₁-C₆ haloalkyl, C₁-C₆ haloalkoxy, halogen, —CN, —OR^{c}, —N(R^{c})(R^{c}), and C₁-C₆ alkoxycarbonyl, wherein each occurrence of R^{c} is independently H, C₁-C₆ alkyl, or C₃-C₈ cycloalkyl.

5. The compound of claim 1, wherein R¹ is selected from the group consisting of:

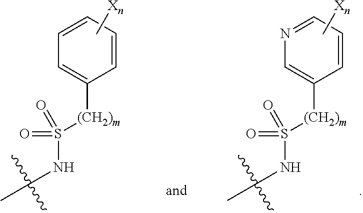

6. The compound of claim 1, wherein R¹ is selected from the group consisting of:

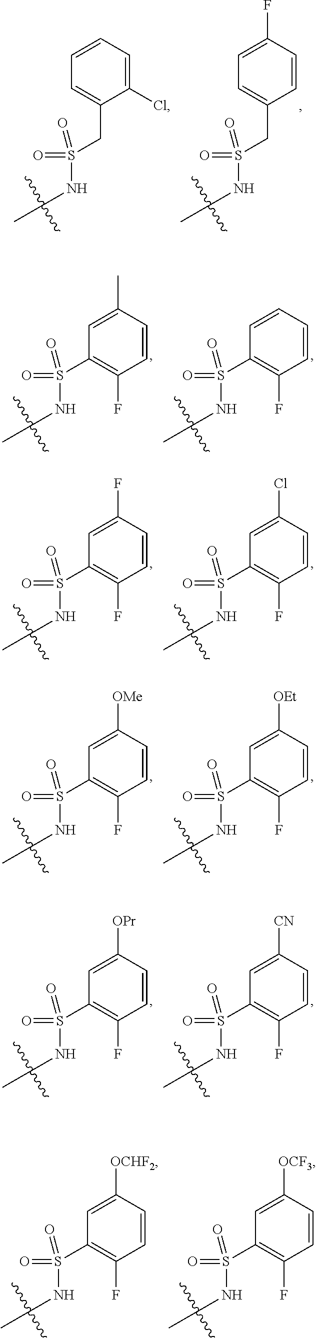

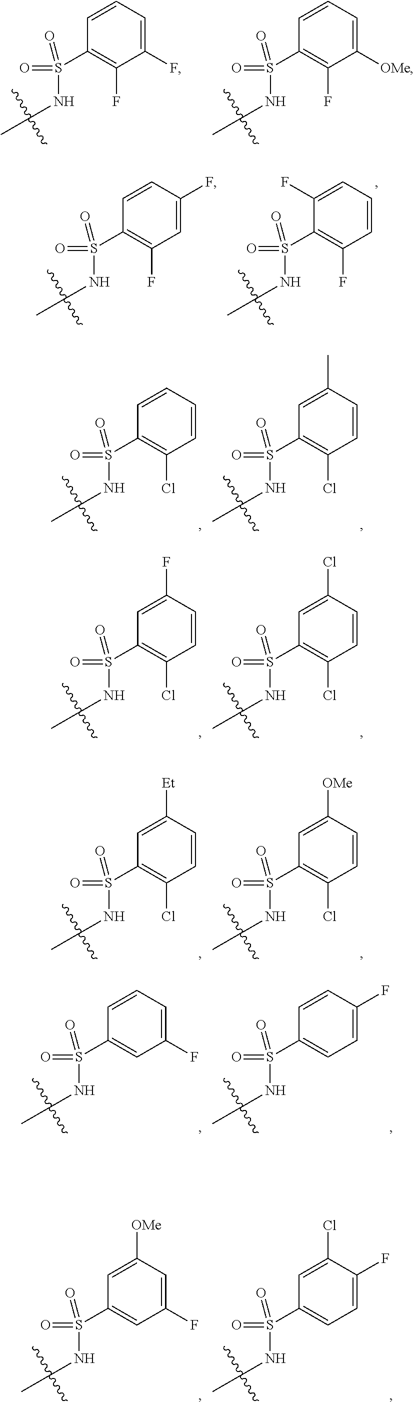

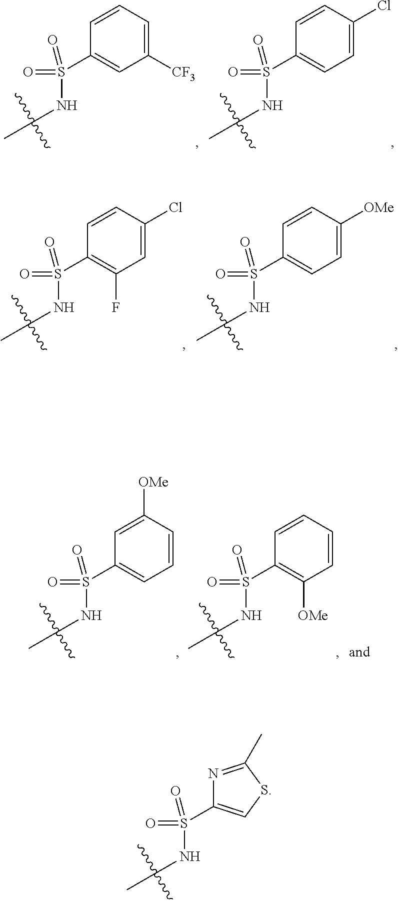

7. The compound of claim 1, wherein R² is selected from the group consisting of methyl, ethyl, isopropyl, and cyclopropyl.

8. The compound of claim 1, wherein R³ is: 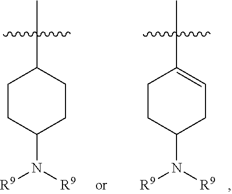
wherein each occurrence of R⁹ is independently selected from the group consisting of H, oxetanyl, C₁-C₆ alkyl, C₃-C₈ cycloalkyl, C₁-C₆ hydroxyalkyl, C₁-C₆ (C₁-C₆ alkoxy)alkyl, C₁-C₆ haloalkyl, C₁-C₆ carboxamido alkyl, C₁-C₆ carboxy alkyl, C₁-C₆ [carboxy(C₁-C₆)alkyl] alkyl, C₁-C₆ cyano alkyl, and C₁-C₆ sulfonyl alkyl, or the two R⁹ combine with the N to which they are bound to form a 3-8 heterocyclyl ring,
wherein each R⁹ is independently optionally substituted with at least one of OH, C₁-C₆ alkoxy, halogen, NH₂, NH(C₁-C₆ alkyl), N(C₁-C₆ alkyl)(C₁-C₆ alkyl), cyano, carboxamide, carboxy, and sulfonyl.

9. The compound of claim 8, wherein each occurrence of R⁹ is independently selected from the group consisting of H, methyl, ethyl, propyl, isopropyl, butyl, sec-butyl, isobutyl, tert-butyl, cyclobutyl, fluorocyclobutyl, difluorocyclobutyl, oxetanyl,

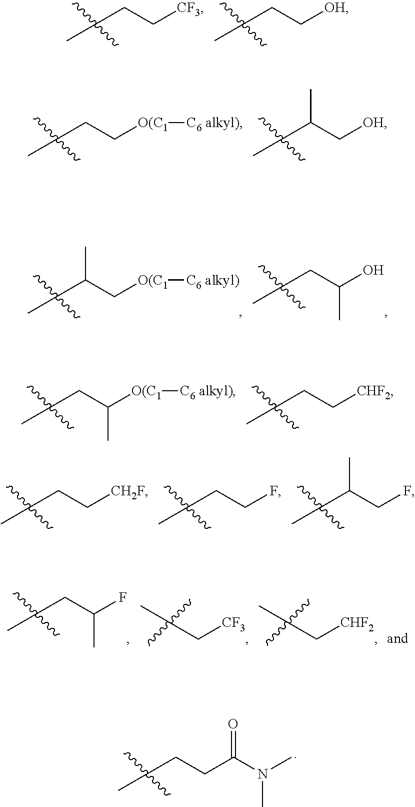

10. The compound of claim 1, wherein R³ is selected from the group consisting of:

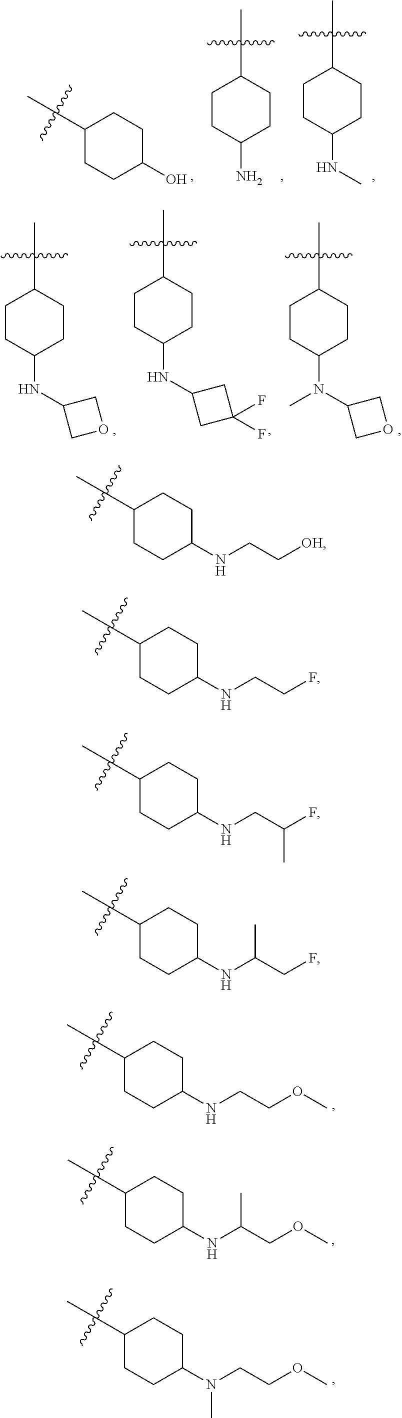

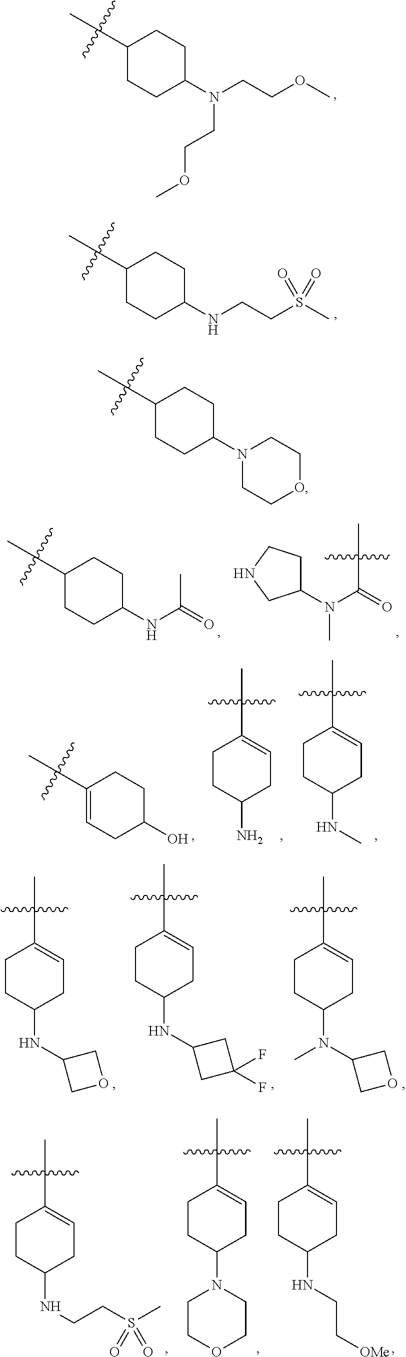

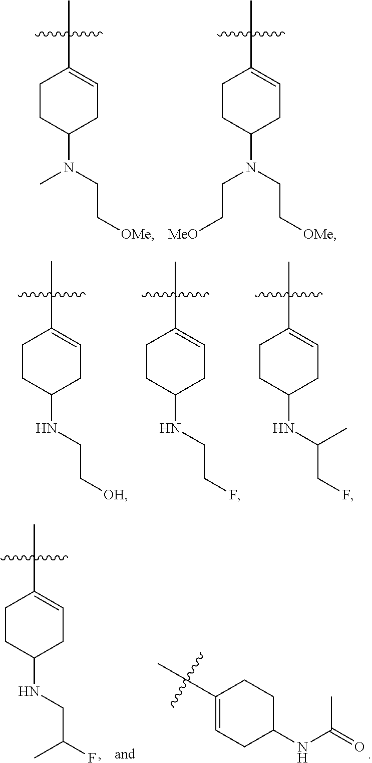

11. The compound of claim 1, wherein R³ is selected from the group consisting of:

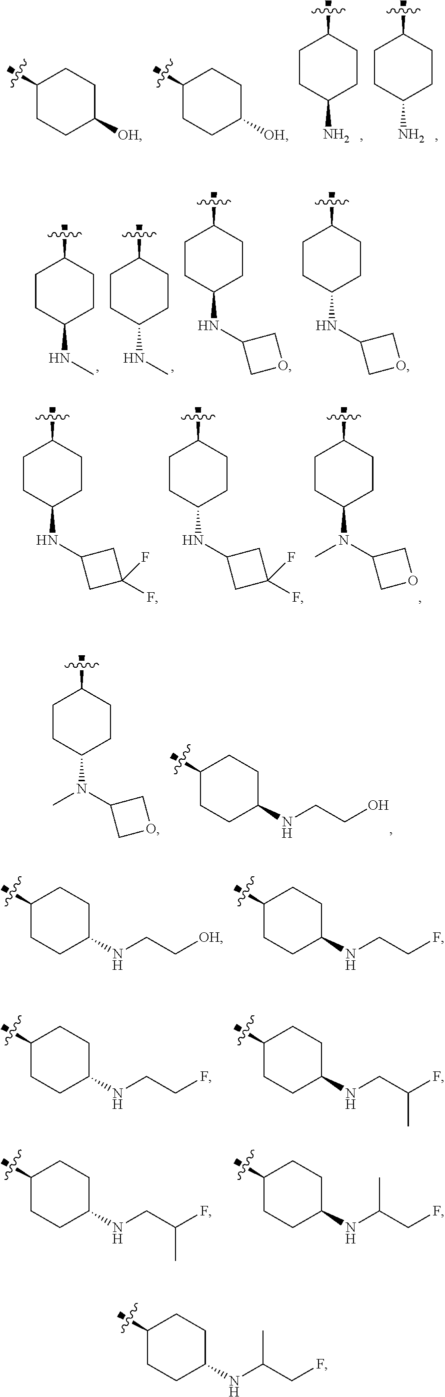

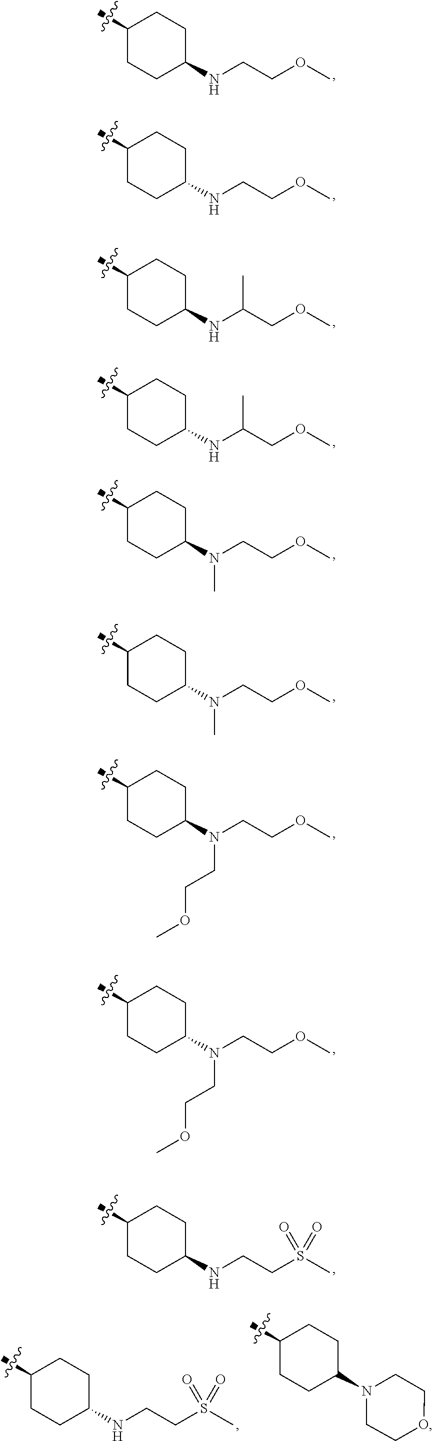

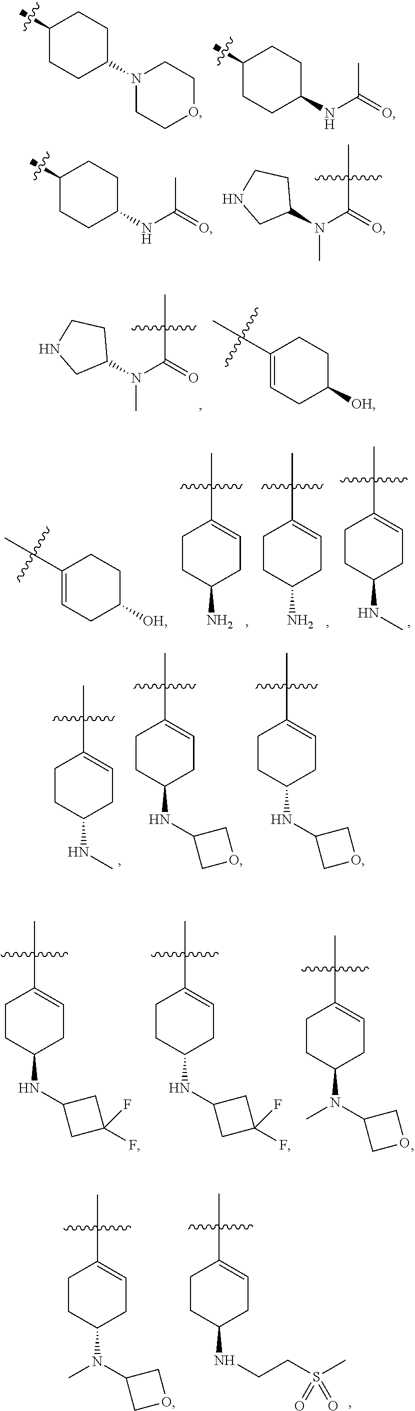

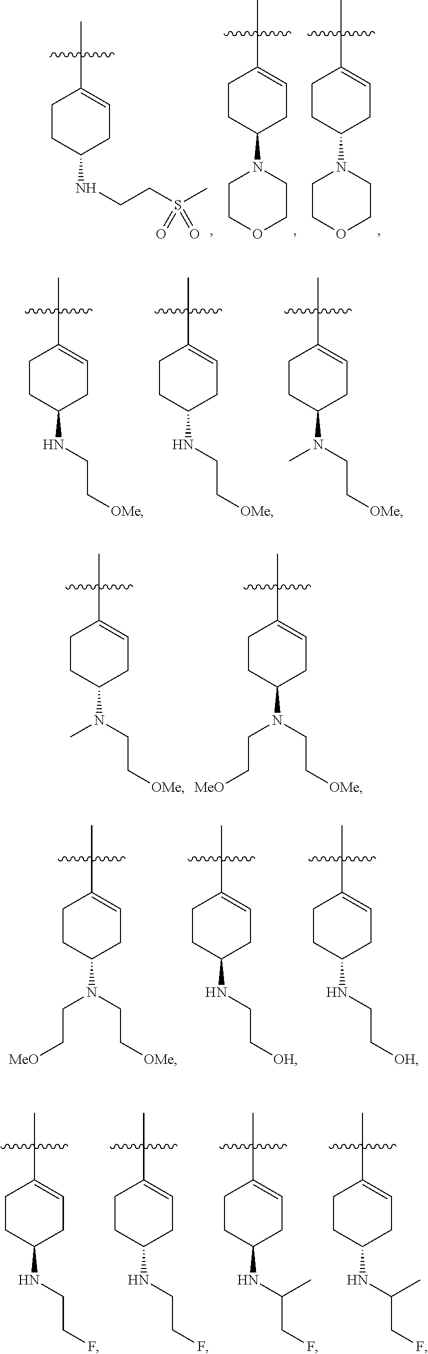

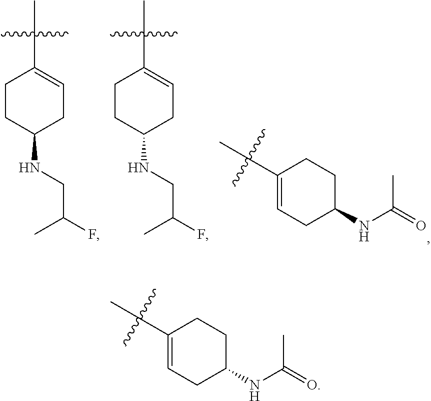 [SMILES: *C1=CC[C@@H](NCC(C)F)CC1 ; *C1=CC[C@H](NCC(C)F)CC1 ; *C1=CC[C@H](NC(C)=O)CC1 ; *C1=CC[C@@H](NC(C)=O)CC1]

12. The compound of claim 1, wherein R⁵, if present, is —F.

13. The compound of claim 1, which is: 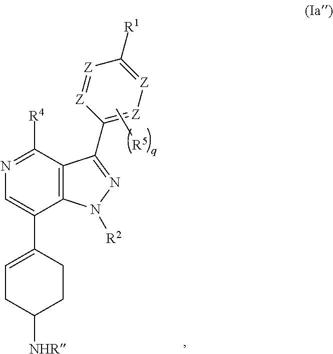
wherein R″ is H, optionally substituted C₁-C₆ alkyl, optionally substituted C₃-C₈ cycloalkyl, or optionally substituted heterocyclyl.

14. The compound of claim 1, which is: 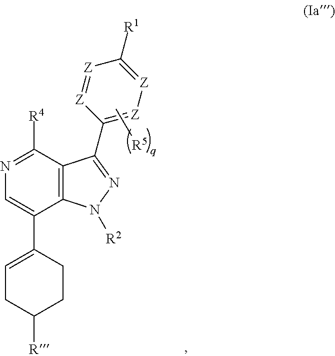
wherein each occurrence of R′″ is independently selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl),
wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, and —C₁-C₆ alkoxy.

15. The compound of claim 14, wherein R′″ is selected from the group consisting of —OH, —NH₂, —NHCH₃, —N(CH₃)₂, —NHCH₂CH₂F, —N(Me)CH₂CH₂F, —NHCH₂CHF₂, —N(Me)CH₂CHF₂, —NHCH₂CF₃, —N(Me)CH₂CF₃, —NHCH₂CH₂CF₃, —N(Me)CH₂CH₂CF₃, —NHCH₂CH₂C(—₀)NMe₂, —N(Me)CH₂CH₂C(═O)NMe₂, —NHCH₂CH₂C(═O)NH₂, —N(Me)CH₂CH₂C(═O)NH₂, —NHCH₂CH₂C(═O)NHMe, —N(Me)CH₂CH₂C(═O)NHMe₂, and

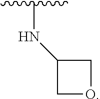 [SMILES: *NC1COC1]

16. The compound of claim 1, which is selected from the group consisting of:
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-chloro-5-methoxybenzenesulfonamide;
N-(4-(4-amino-1-isopropyl-7-((1r,4r)-4-morpholinocyclohexyl)-1H-pyrazolo[4,3-c]pyridin-3-yl)-2,5-difluorobenzenesulphonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-5-chloro-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-5-(difluoromethoxy)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-fluoro-3-methoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-5-ethoxy-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-4-methoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-methoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-3-methoxybenzenesulfonamide;
N-(4-(4-amino-1-isopropyl-7-((1s,4s)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-4-fluoro-3-methoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-4-chlorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-chloro-4-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-4-chloro-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-1-(4-fluorophenyl)methanesulfonamide;
N-(4-(4-amino-1-isopropyl-7-((1r,4r)-4-(2-methoxyethyl)amino)cyclohexyl)-1H-pyrazolo[4,3-c]pyridin-3-yl)-3-fluorophenyl)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-5-cyano-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2,6-difluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2,4-difluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2,3-difluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-1-(2-fluorophenyl)methanesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluoro-5-methoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-fluoro-5-methoxybenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-3-(trifluoromethyl)benzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2,5-difluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-4-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-3-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-7-((1r,4r)-4-(bis(2-methoxyethyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(2-methoxyethyl)(methyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-7-((1r,4r)-4-(dimethylamino)cyclohexyl)-1-isopropyl-1H-pyrazolo[4,3-c]pyridin-3-yl)-2,5-difluorophenyl)-2-fluorobenzenesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-fluorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-ethyl-74(1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-1-isopropyl-7-((1r,4r)-4-(methyl(oxetan-3-yl)amino)cyclohexyl)-1H-pyrazolo[4,3-c]pyridin-3-yl)-2-fluorophenyl)-2-fluorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-chlorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-1-(2-fluorophenyl)methanesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2,5-difluorophenyl)-2-fluorobenzenesulfonamide;
N-(4-(4-amino-7-((1r,4r)-4-(dimethylamino)cyclohexyl)-1-isopropyl-1H-pyrazolo[4,3-c]pyridin-3-yl)-2-fluorophenyl)-1-(2-chlorophenyl)methanesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-ethyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-1-(2-chlorophenyl)methanesulfonamide;
N-(4-(4-amino-1-cyclopropyl-7-((1r,4r)-4-(oxetan-3-ylamino)cyclohexyl)-1H-pyrazolo[4,3-c]pyridin-3-yl)-2-fluorophenyl)-2-chlorobenzenesulfonamide;
N-(4-(4-amino-1-isopropyl-7-((1r,4r)-4-(oxetan-3-ylamino)cyclohexyl)-1H-pyrazolo[4,3-c]pyridin-3-yl)-2-fluorophenyl)-1-(2-fluorophenyl)methanesulfonamide;
N-(4-(4-amino-1-isopropyl-7-((1r,4r)-4-(1-methoxypropan-2-yl)amino)cyclohexyl)-1H-pyrazolo[4,3-c]pyridin-3-yl)-2-fluorophenyl)-1-(2-chlorophenyl)methanesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-fluoro-5-(trifluoromethoxy)benzenesulfonamide;
N-(4-(4-amino-isopropyl-7-((1r,4r)-4-(2-methoxyethyl)amino)cyclohexyl)-1H-pyrazolo[4,3-c]pyridin-3-yl)-2,5-difluorophenyl)-2-fluoro-5-propoxybenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2,5-dichlorobenzenesulfonamide;
N-(4-(4-amino-1-isopropyl-7-((1r,4r)-4-(2-methoxyethyl)amino)cyclohexyl)-1H-pyrazolo[4,3-c]pyridin-3-yl)-2,5-difluorophenyl)-2-chloro-5-ethylbenzenesulfonamide;
N-(4-(4-amino-7-((1r,4r)-4-(2-fluoropropyl)amino)cyclohexyl)-1-isopropyl-1H-pyrazolo[4,3-c]pyridin-3-yl)-2,5-difluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-isopropyl-7-(4-((1-methoxypropan-2-yl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((1-methoxypropan-2-yl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chloro-5-methoxybenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-cyclopropyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)phenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-fluorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-2-chlorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2,5-difluorophenyl)-1-(2-chlorophenyl)methanesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-fluoro-5-methylbenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chloro-5-methoxybenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-5-chloro-2-fluorobenzenesulfonamide;
N-(4-(4-amino-7-(4(R)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(R)-((1-fluoropropan-2(S)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(R)-yl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4(S)-((1-fluoropropan-2(S)-yl)amino)cyclohex-1-en-1-yl)-isopropyl-1H-pyrazolo[4,3-c]pyridin-3-yl)-2-fluorophenyl)-2-chlorobenzenesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((1-fluoropropan-2-yl)amino)cyclohex-1-en-1-yl)-1-isopropyl-1H-pyrazolo[4,3-c]pyridin-3-yl)-2-fluorophenyl)-1-(2-chlorophenyl)methanesulfonamide;
N-(4-(4-amino-7-(4-((2-fluoropropyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-7-(4-((3,3-difluorocyclobutyl)amino)cyclohex-1-en-1-yl)-1-isopropyl-1H-pyrazolo[4,3-c]pyri din-3-yl)-2-fluorophenyl)-1-(2-chlorophenyl)methanesulfonamide;
N-(4-(4-amino-7-(4-((3,3-difluorocyclobutyl)amino)cyclohex-1-en-1-yl)-1-isopropyl-1H-pyrazolo[4,3-c]pyridin-3-yl)-2-fluorophenyl)-2-chlorobenzenesulfonamide;
N-(4-(4-amino-1-isopropyl-7-(4-((2-methoxyethyl)amino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-((2-methoxyethyl)amino)cyclohex-1-en-1-yl)-1H-pyrazolo[4,3-c]pyridin-3-yl)-2-fluorophenyl)-1-(2-chlorophenyl)methanesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2-fluorophenyl)-2-fluorobenzenesulfonamide;
N-(4-(4-amino-1-isopropyl-7-(4-(oxetan-3-ylamino)cyclohex-1-en-1-yl)-1H-pyrazolo[4,3-c]pyridin-3-yl)-2,5-difluorophenyl)-2-fluorobenzenesulfonamide;
N-(4-(4-amino-1-ethyl-7-(4-(oxetan-3-ylamino)cyclohex-1-en-1-yl)-1H-pyrazolo[4,3-c]pyridin-3-yl)-2-fluorophenyl)-2-chlorobenzenesulfonamide;
N-(4-(4-amino-7-(4-((2-fluoroethyl)amino)cyclohex-1-en-1-yl)-1-isopropyl-1H-pyrazolo[4,3-c]pyridin-3-yl)-2-fluorophenyl)-2-chlorobenzenesulfonamide;
or a salt, solvate, enantiomer, diastereoisomer, isotopologue or tautomer thereof.

17. A pharmaceutical composition comprising at least one compound of claim 1 and at least one pharmaceutically acceptable carrier.

18. A method of treating a IRE1α-related disease in a subject, the method comprising administering to the subject a therapeutically effective amount of a compound of claim 1, or a pharmaceutically acceptable salt, solvate, enantiomer, diastereoisomer, or tautomer thereof.

19. The method of claim 18, wherein the disease is selected from the group consisting of a neurodegenerative disease, a demyelinating disease, cancer, an eye disease, a fibrotic disease, and diabetes.

20. The method of claim 19, wherein at least one of the following applies:
(a) the neurodegenerative disease is selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru;
(b) the demyelinating disease is selected from the group consisting of Wolfram Syndrome, Pelizaeus-Merzbacher Disease, Transverse Myelitis, Charcot-Marie-Tooth Disease, and Multiple Sclerosis;
(c) the cancer is multiple myeloma;
(d) the diabetes is selected from the group consisting of type I diabetes and type II diabetes;
(e) the eye disease is selected from the group consisting of retinitis pigmentosa, retinal degeneration, macular degeneration, and Wolfram Syndrome; and
(f) the fibrotic disease is selected from the group consisting of idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

21-25. (canceled)

26. A method of inhibiting the activity of an IRE1 protein, the method comprising contacting the IRE1 protein with an effective amount of a compound of claim 1, or a pharmaceutically acceptable salt thereof.

27. The method of claim 26, wherein the activity is selected from the group consisting of kinase activity, oligomerization activity, and RNase activity.

28. The method of claim 26, wherein at least one of the following applies:
(a) the IRE1 protein is within a cell;
(b) the IRE1 protein is within a cell and apoptosis of the cell is prevented or minimized;
(c) the IRE1 protein is within a cell and the cell is in an organism that has an IRE1α-related disease or disorder.

29-30. (canceled)

31. The method of claim 30, wherein the disease or disorder is a neurodegenerative disease, demyelinating disease, cancer, eye disease, fibrotic disease, or diabetes.

32. The method of claim 18, wherein the subject is need of the treatment.
